(19) United States
(12) Patent Application Publication
(10) Pub. No.: US 2026/0022343 A1
(43) Pub. Date: Jan. 22, 2026

(54) GENETICALLY MODIFIED CELLS COMPRISING A NUCLEIC ACID ENCODING A CD40L BINDING AGENT AND USES THEREOF

(71) Applicant: AffyXell Therapeutics Co., Ltd., Gimhae-si (KR)

(72) Inventors: Kyong-Hoon Anh, Seoul (KR); Sung Hyun Choi, Anyang-Si Gyeonggi-Do (KR); Kinam Kim, Yongin-Si Gyeonggi-Do (KR); Jong Sang Ryu, Yongin-Si Gyeonggi-Do (KR); Sengho Jeon, Seongnam-Si Gyeonggi-Do (KR)

(21) Appl. No.: 19/143,797

(22) PCT Filed: Dec. 29, 2023
(86) PCT No.: PCT/IB23/00795

§ 371 Date: Jun. 26, 2025

(30) Foreign Application Priority Data
Dec. 29, 2022 (KR) 10-2022-0188961
Mar. 31, 2023 (WO) PCT/KR2023/004407

(51) Int. Cl. C12N 5/0775; A61K 35/28; A61P 35/00; C07K 16/40; C12N 15/10; C12N 15/63; C12N 15/86

(52) U.S. Cl. CPC C12N 5/0662; A61K 35/28; A61P 35/00; C07K 16/40; C12N 15/10; C12N 15/635; C12N 15/86; C07K 2317/53; C07K 2319/03; C12N 2510/00; C12N 2740/15043; C12N 2840/203

(57) ABSTRACT

The present disclosure relates to a genetically modified stem cells (e.g., mesenchymal stromal cells (MSCs) or pluripotent stem cells (PSCs)) and populations thereof, that comprise an exogenous nucleic acid that encodes a binding protein that binds to a target. Targets include, for example, proteins expressed on activated immune cells. Binding proteins expressed by genetically modified stem cells as described herein can include one or more binding domains from an antibody or antibody mimetic. Also provided are methods of making genetically modified stem cells, pharmaceutical preparations including genetically modified stem cells, and methods of using the same, for example, in the treatment of immune diseases, including inflammatory, autoimmunity and cancer.

### CROSS-REFERENCE TO RELATED APPLICATIONS

This application claims the benefit of priority to Korean Patent Application No. 10-2022-0188961, filed Dec. 29, 2022; International Patent Application No. PCT/KR2023/004407, filed Mar. 31, 2023; the contents of each of which are incorporated herein by reference.

### FIELD OF THE INVENTION

The present invention relates to genetically modified stem cells (e.g., mesenchymal stromal cells) that express an exogenous binding agent (e.g., binding protein, e.g., antibody, antibody mimetic and/or a fusion protein including the same), and uses thereof.

### BACKGROUND

Cell therapeutic agents based on differentiated immune cells, such as CAR-T or CAR-NK, are expensive due to the use of autologous cells and have limited targets. Mesenchymal stromal cells (MSCs) have beneficial characteristics for cell therapy including low immunogenicity due to the absence of HLA-II. However, MSCs are currently associated with low therapeutic effect relative to the cost of treatment. The present inventors have made great efforts to develop new engineered stem cells (e.g., MSCs) with beneficial characteristics for use as cell therapeutic agents and/or production thereof.

### SUMMARY

The present disclosure provides, at least in part, genetically modified stem cells (e.g., pluripotent stem cells (PSCs) or mesenchymal stromal cells (MSCs)) that express an exogenous binding agent (e.g., a binding protein that specifically binds a target). In some embodiments, provided are genetically modified stem cells (e.g., PSCs or MSCs) and populations thereof, comprising exogenous nucleic acid that includes a coding sequence encoding a binding agent (e.g., a binding protein that specifically binds a target).

In some embodiments, provided are genetically modified MSCs and populations of genetically modified MSCs, where the MSCs comprise an exogenous nucleic acid comprising a coding sequence that encodes a binding agent (e.g., a binding protein that specifically binds a target). In some embodiments, the binding agent comprises one or more binding domains from an antibody or antibody mimetic.

The present disclosure describes that provided genetically modified MSCs may have unexpected benefits, for example the genetically modified cells may exhibit highly enhanced immunomodulatory activity compared to non-modified MSCs, may have a lower immune rejection response due to allogeneic cell transplantation, and/or the genetically modified cells may exhibit excellent therapeutic effects on immune-related diseases such as inflammatory diseases and autoimmune diseases, or cancer.

In some embodiments, provided are methods of producing a population of genetically modified MSCs comprising an exogenous nucleic acid comprising a coding sequence that encodes a binding agent, where the binding agent comprises one or more binding domains from an antibody or antibody mimetic. In some embodiments, provided methods comprise contacting a population of MSCs with a lentiviral vector comprising an exogenous nucleic acid comprising a coding sequence that encodes a binding agent, and culturing the population of MSCs. In some embodiments, the population of MSCs are unattached when the contacting is initiated. In some embodiments, provided methods comprise a step of selecting those MSCs that include the exogenous nucleic acid, e.g., by antibiotic selection gene. In some embodiments, MSCs produced by the provided methods exhibit superior immunomodulatory effects and therapeutic effects on immune-related diseases.

In some embodiments, provided are genetically modified PSCs and populations of genetically modified PSCs, where the PSCs comprise an exogenous nucleic acid comprising a coding sequence that encodes a binding agent (e.g., a binding protein that specifically binds a target). In some embodiments, the binding agent comprises one or more binding domains from an antibody or antibody mimetic. In some embodiments, the PSCs are induced PSCs or embryonic stem cells.

In some embodiments, the binding agent is or comprises a stefin A protein variant, Fab, Fab′, F(ab′)2, Fv, Fd, scFv, sdFv), VL, VH, Camel Ig, V-NAR, VHH, trispecific (Fab3), bispecific (Fab2), diabody ((VL-VH)2 or (VH-VL)2), triabody (trivalent), tetrabody (tetravalent), minibody ((scFv-CH3)2), bispecific single-chain Fv (Bis-scFv), a shark heavy-chain-only antibody (VNAR), a microprotein (cysteine knot protein, knottin), affibody, aptamer, avimer, nanobody, unibody, a single domain antibody, affilin, affitin, adnectin, atrimer, evasin, DARPin, anticalin, avimer, fynomer, versabody, repebody, or a duocalin.

In some embodiments, the binding agent is or comprises a binding protein selected from: a stefin A protein variant, Fab, Fab′, F(ab′)2, Fv, Fd, scFv, sdFv), VL, VH, Camel Ig, V-NAR, VHH, trispecific (Fab3), bispecific (Fab2), diabody ((VL-VH)2 or (VH-VL)2), triabody (trivalent), tetrabody (tetravalent), minibody ((scFv-CH3)2), bispecific single-chain Fv (Bis-scFv), a shark heavy-chain-only antibody (VNAR), a microprotein (cysteine knot protein, knottin), affibody, avimer, nanobody, unibody, a single domain antibody, affilin, affitin, adnectin, atrimer, evasin, DARPin, anticalin, avimer, fynomer, versabody, repebody, and duocalin.

In certain embodiments, the binding agent comprises a ligand binding domain of a receptor that acts as an inhibitor/antagonist of the ligand (i.e., target) to which it binds, e.g., an “inhibitory receptor trap” or “decoy receptor”. Inhibitory receptor traps binds to and/or sequesters its target, which in essence inhibits the target from carrying out its function which may contribute to a disease and/or disorder to be treated. Exemplary receptor traps include interleukin-1 (IL-1) inhibitory receptor traps (such as IL-1β inhibitory traps), interleukin-6 (IL-6) inhibitory receptor traps, interleukin-10 (IL-10) inhibitory receptor traps, interleukin-13 (IL-13) inhibitory receptor traps, interleukin-35 (IL-35) inhibitory receptor traps, TNFα inhibitory receptor traps, TGFβ inhibitory receptor traps, CXCL8 inhibitory receptor traps, CXCL9 inhibitory receptor traps, CXCL12 inhibitory receptor traps, and CCL2 inhibitory receptor traps.

In some embodiments, the binding agent exhibits a Kd value of 1×10⁻⁶ M or less for its target. In some embodiments, the binding agent exhibits a Kd value of 1×10⁻⁷ M or less for its target. In some embodiments, the binding agent exhibits a Kd value of 1×10⁻⁸ M or less for its target.

In some embodiments, a binding agent is based on stefin A protein variant technology. The present disclosure describes, among other things, development of genetically modified stems cells that express a stefin A protein variant that specifically binds to its target. In some embodiments, provided genetically modified stem cells are produced by introducing a gene encoding the binding agent (e.g., stefin A protein variant) into the stem cells (e.g., PSCs or MSCs).

In some embodiments, the binding agent is secreted by the genetically modified stem cells (e.g., PSCs or MSCs).

In some embodiments, where the binding agent is secreted extracellularly, the population of genetically modified stem cells (e.g., PSCs or MSCs) express and secrete the binding agent at an average level of 200 fg/cell/day or greater. In some embodiments, where the binding agent is secreted extracellularly, the population of genetically modified stem cells (e.g., PSCs or MSCs) express and secrete the binding agent at an average level of 300 fg/cell/day or greater. In some embodiments, where the binding agent is secreted extracellularly, the population of genetically modified stem cells (e.g., PSCs or MSCs) express and secrete the binding agent at an average level of 400 fg/cell/day or greater.

In some embodiments, where the binding agent is secreted extracellularly, the population of genetically modified stem cells (e.g., PSCs or MSCs) express and secrete the binding agent at an average level of at least 200 fg/cell/day, and more preferably at least at an average level of at least 300 fg/cell/day, 400 fg/cell/day, 500 fg/cell/day, 750 fg/cell/day or even at least 1000 fg/cell/day. In some embodiments, where the binding agent is secreted extracellularly, the population of genetically modified stem cells (e.g., PSCs or MSCs) express and secrete the binding agent at an average level of 200 to 1000 fg/cell/day. In some embodiments, where the binding agent is secreted extracellularly, the population of genetically modified stem cells (e.g., PSCs or MSCs) express and secrete the binding agent at an average level of 300 to 800 fg/cell/day. In some embodiments, where the binding agent is secreted extracellularly, the population of genetically modified stem cells (e.g., PSCs or MSCs) express and secrete the binding agent at an average level of 400 to 700 fg/cell/day.

In some embodiments, the binding agent is membrane-anchored or cell-surface-expressed.

In some embodiments, the genetically modified stem cells (e.g., PSCs or MSCs) express the binding agent as a membrane-anchored or otherwise cell-surface-retained protein at a level averaging at least 10,000 molecules or more of the cell surface retained binding agent molecules per cell. In some embodiments, the genetically modified stem cells (e.g., PSCs or MSCs) express the cell surface retained binding agent at a level averaging at least 20,000 molecules or more of the binding agent molecules per cell. In some embodiments, the genetically modified stem cells (e.g., PSCs or MSCs) express the cell surface retained binding agent at a level averaging at least 30,000 molecules or more of the binding agent molecules per cell. In some embodiments, the genetically modified stem cells (e.g., PSCs or MSCs) express the cell surface retained binding agent at a level averaging at least 40,000 molecules or more of the binding agent molecules per cell.

In some embodiments, provided are a population of genetically modified stem cells (e.g., PSCs or MSCs) that express a binding agent that is a cell surface retained binding protein, where the cells express the cell surface retained binding protein on their cell surface at a level averaging 10,000 to 150,000 binding protein molecules per cell. In some embodiments, provided are a population of genetically modified stem cells (e.g., PSCs or MSCs) that express a binding agent that is a cell surface retained binding protein, where the cells express the cell surface retained binding protein on their cell surface at a level averaging 20,000 to 100,000 binding protein molecules per cell. In some embodiments, provided are a population of genetically modified stem cells (e.g., PSCs or MSCs) that express a binding agent that is a binding protein, where the cells express the cell surface retained binding protein on their cell surface at a level averaging 30,000 to 90,000 binding protein molecules per cell.

In some embodiments, the genetically modified stem cells (e.g., PSCs or MSCs) in the population may express the binding agent and/or fusion protein, and the binding agent and/or the fusion protein may be secreted extracellularly, may be anchored on a cell membrane, and/or may be presented on the cell surface.

In some embodiments, the genetically modified stem cells (e.g., PSCs or MSCs) in the population may express and secrete extracellularly the binding agent and/or the fusion protein at an average level of 1 fg/cell/day or greater, 10 fg/cell/day or greater, 50 fg/cell/day or greater, 100 fg/cell/day or greater, 200 fg/cell/day or greater, 300 fg/cell/day or greater, 400 fg/cell/day or greater, or 500 fg/cell/day or greater. In some embodiments, the average level of expression is maintained for at least 5 days, at least 7 days, at least 10 days, at least 12 days, at least 14 days, at least 18 days, at least 21 days, at least 24 days, at least 28 days, at least 30 days, or more.

In some embodiments, the genetically modified stem cells (e.g., PSCs or MSCs) in the population may express and secrete extracellularly the binding agent and/or the fusion protein at an average level of preferably 100 fg/cell/day or greater.

In some embodiments, the genetically modified stem cells (e.g., PSCs or MSCs) in the population may express and secrete extracellularly the binding agent and/or the fusion protein at an average level of 1˜5,000 fg/cell/day, 10˜3,000 fg/cell/day, 50˜2,000 fg/cell/day, 100˜1,100 fg/cell/day, or 200˜800 fg/cell/day. In some embodiments, the average level of expression is maintained for at least 5 days, at least 7 days, at least 10 days, at least 12 days, at least 14 days, at least 18 days, at least 21 days, at least 24 days, at least 28 days, at least 30 days, or more.

In some embodiments, the genetically modified stem cells (e.g., PSCs or MSCs) in the population may express and secrete extracellularly the binding agent and/or the fusion protein at an average level of preferably about 100 fg/cell/day to about 1,100 fg/cell/day. In some embodiments, the average level of expression is maintained for at least 5 days, at least 7 days, at least 10 days, at least 12 days, at least 14 days, at least 18 days, at least 21 days, at least 24 days, at least 28 days, at least 30 days, or more.

In some embodiments, the genetically modified stem cells (e.g., PSCs or MSCs) in the population may express and secrete extracellularly the binding agent and/or the fusion protein at an average level of preferably about 1×10⁶ molecules/cell/day to about 1.2×10⁷ molecules/cell/day. In some embodiments, the average level of expression is maintained for at least 5 days, at least 7 days, at least 10 days, at least 12 days, at least 14 days, at least 18 days, at least 21 days, at least 24 days, at least 28 days, at least 30 days, or more.

In some embodiments, the genetically modified stem cells (e.g., PSCs or MSCs) express and present the binding agent and/or the fusion protein on the cell surface (e.g., anchored and/or presented on a cell membrane), at an average level of 0.001 fg/cell or greater, 0.005 fg/cell or greater, 0.01 fg/cell or greater, 0.05 fg/cell or greater, 0.07 fg/cell or greater, 0.1 fg/cell/day or greater or 0.5 fg/cell/day or greater. In some embodiments, the average level of expression is maintained for at least 5 days, at least 7 days, at least 10 days, at least 12 days, at least 14 days, at least 18 days, at least 21 days, at least 24 days, at least 28 days, at least 30 days, or more.

In some embodiments, the genetically modified stem cells (e.g., PSCs or MSCs) express and present the binding agent and/or the fusion protein on the cell surface, at an average level of preferably 0.07 fg/cell or greater.

In some embodiments, the genetically modified stem cells (e.g., PSCs or MSCs) express and present the binding agent and/or the fusion protein on the cell surface, at an average level of 0.001˜10 fg/cell, 0.005˜5 fg/cell, 0.01˜3 fg/cell, 0.05˜1.5 fg/cell, or 0.07˜1 fg/cell.

In some embodiments, the genetically modified stem cells (e.g., PSCs or MSCs) express and present the binding protein and/or the fusion protein on the cell surface, at an average level of preferably 0.07˜1 fg/cell.

In some embodiments, the genetically modified stem cells (e.g., PSCs or MSCs) express and present the binding protein and/or the fusion protein on the cell surface, at an average level of 100 molecules/cell or greater, 500 molecules/cell or greater, 1,000 molecules/cell or greater, 1,500 molecules/cell or greater, 2,000 molecules/cell or greater, 2,500 molecules/cell or greater, 3,000 molecules/cell or greater, 4,000 molecules/cell or greater, or 5,000 molecules/cell or greater.

In some embodiments, the genetically modified stem cells (e.g., PSCs or MSCs) express and present the binding protein and/or the fusion protein on the cell surface, at an average level of preferably 2,500 molecules/cell or greater. In some embodiments, the average level of expression is maintained for at least 5 days, at least 7 days, at least 10 days, at least 12 days, at least 14 days, at least 18 days, at least 21 days, at least 24 days, at least 28 days, at least 30 days, or more.

In some embodiments, the genetically modified stem cells (e.g., PSCs or MSCs) express and present the binding protein and/or the fusion protein on the cell surface, at an average level of 100˜100,000 molecules/cell, 500˜80,000 molecules/cell, 1,000˜60,000 molecules/cell, 1,500˜50,000 molecules/cell, 2,000˜40,000 molecules/cell, or 2,500˜35,000 molecules/cell. In some embodiments, the average level of expression is maintained for at least 5 days, at least 7 days, at least 10 days, at least 12 days, at least 14 days, at least 18 days, at least 21 days, at least 24 days, at least 28 days, at least 30 days, or more.

In some embodiments, the genetically modified stem cells (e.g., PSCs or MSCs) express and present the binding protein and/or the fusion protein on the cell surface, at an average level of preferably 2,500˜35,000 molecules/cell. In some embodiments, the average level of expression is maintained for at least 5 days, at least 7 days, at least 10 days, at least 12 days, at least 14 days, at least 18 days, at least 21 days, at least 24 days, at least 28 days, at least 30 days, or more.

In some embodiments, the binding agent is a target binding fusion protein.

In some embodiments, the target binding fusion protein comprises at least one selected from the group consisting of a transmembrane domain, a hinge domain, a coiled coil domain, a virus-derived domain, an intracellular signaling domain, and a localization domain. In some embodiments, the target binding fusion protein comprises at least one selected from the group consisting of a signal peptide, a Fc domain, a binding domain, a cytokine, a half-life extension domain, a growth factor, an enzyme, and a cell-penetrating domain. In some embodiments, the target binding fusion protein comprises a therapeutic peptide or protein.

In some embodiments, the binding agent is a target binding fusion protein comprising a transmembrane domain. In some embodiments, the transmembrane domain is derived from group consisted of CD3, CD4, CD5, CD8, CD28, CD99, PDGFR, and PTGFRN. In some embodiments, the target binding fusion protein comprises a hinge domain derived from an immunoglobulin (e.g., IgG1, IgG4, IgD, etc.) In some embodiments, the binding agent is a target binding fusion protein comprising a stefin A protein variant that specifically binds to its target and a transmembrane domain. In some embodiments, the target binding fusion protein comprises or consists of a stefin A protein variant and a PDGFR transmembrane domain.

In some embodiments, the binding agent is a target binding fusion protein comprising or consisting of an antibody fragment that specifically binds to its target and a transmembrane domain.

In some embodiments, the target protein is express on the surface of an immune cell.

In some embodiments, the immune cell is selected from a dendritic cell, peripheral blood mononuclear cell (PBMC), T cell (e.g., effector T cell, memory T cell, cytotoxic T lymphocyte, helper T cell, regulatory T cell), B cell, natural killer (NK) cell (e.g., monocyte, macrophage, neutrophil, granulocyte), or a combination thereof.

In some embodiments, binding of the target protein by the binding agent inhibits inflammatory activity of the activated immune cells.

In some embodiments, the target protein is a TNF Receptor (e.g., TNFR2) or an immunostimulatory TNF receptor ligand (e.g., CD27L, CD40L, 41BBL, or GITRL).

In some embodiments, the target protein is a proinflammatory cytokine (e.g., IL-1, IL-6, IL-12, and IL-18, TNF-α, IFNγ, GM-CSF).

In some embodiments, the binding agent comprises a trimer or tetramer of stefin A protein variants.

In some embodiments, the exogenous nucleic acid comprises a transcriptional regulatory sequence that is operably linked to the coding sequence. In some embodiments, the transcriptional regulatory sequence is a promoter selected from a cytomegalovirus (CMV) promoter, a PGK promoter, an EF1β promoter, an EFS promoter, a CBh promoter, an MSCV promoter, an SFFV promoter, and a UbC promoter.

In some embodiments, an exogenous nucleic acid comprises a selection gene. In some embodiments, the exogenous nucleic acid comprises an antibiotic selection gene. In some embodiments, an exogenous nucleic acid comprises an IRES or 2A sequence. In some embodiments, an exogenous nucleic acid comprises, in order, a promoter, a coding sequence that encodes a binding agent, a IRES or 2A sequence, and an antibiotic selection gene.

In some embodiments, provided genetically modified stem cells (e.g., PSCs or MSCs) do not express the target protein.

In some embodiments, provided are MSCs that are derived from pluripotent stem cells. In some embodiments, the MSCs are derived or differentiated from induced pluripotent stem cells. In some embodiments, the MSCs are derived or differentiated from embryonic stem cells.

In some embodiments, the MSCs express at least one cell surface marker selected from CD29, CD44, CD73, CD90, and CD105. In some embodiments, the MSCs express at least two cell surface markers selected from CD29, CD44, CD73, CD90, and CD105. In some embodiments, the MSCs express at least three cell surface markers selected from CD29, CD44, CD73, CD90, and CD105. In some embodiments, the MSCs express all of the following cell surface markers: CD29, CD44, CD73, CD90, and CD105.

In some embodiments, at least 85%, at least 90%, at least 91%, at least 92%, at least 93%, at least 94%, at least 95%, at least 96%, at least 97%, or at least 98% of the MSCs express at least one cell surface marker selected from CD29, CD44, CD73, CD90, and CD105. In some embodiments, at least 85%, at least 90%, at least 91%, at least 92%, at least 93%, at least 94%, at least 95%, at least 96%, at least 97%, or at least 98% of the MSCs express at least two cell surface markers selected from CD29, CD44, CD73, CD90, and CD105. In some embodiments, at least 85%, at least 90%, at least 91%, at least 92%, at least 93%, at least 94%, at least 95%, at least 96%, at least 97%, or at least 98% of the MSCs express at least three cell surface markers selected from CD29, CD44, CD73, CD90, and CD105. In some embodiments, at least 85%, at least 90%, at least 91%, at least 92%, at least 93%, at least 94%, at least 95%, at least 96%, at least 97%, or at least 98% of the MSCs express all of the following cell surface markers: CD29, CD44, CD73, CD90, and CD105.

In some embodiments, provided are genetically modified MSCs that express CD90. In some embodiments, at least 85%, at least 90%, at least 91%, at least 92%, at least 93%, at least 94%, at least 95%, at least 96%, at least 97%, or at least 98% of the cells of a population of MSCs express CD90.

In some embodiments, at least 85%, at least 90%, at least 91%, at least 92%, at least 93%, at least 94%, or at least 95% of expression of the cell surface marker is maintained in the population of MSCs after at least 5 passages. In some embodiments, at least 85%, at least 90%, at least 91%, at least 92%, at least 93%, at least 94%, or at least 95% of expression of the cell surface marker is maintained in the population of MSCs after at least 15 passages.

In some embodiments, less than 1% of the MSCs express or the MSCs do not express at least one cell surface marker selected from CD14, CD19, CD34, CD45, HLA-DR, SSEA-3, TRA-1-60, TRA-1-81, Nanog and Oct3/4. In some embodiments, less than 1% of the MSCs express or the MSCs do not express at least two cell surface markers selected from CD14, CD19, CD34, CD45, HLA-DR, SSEA-3, TRA-1-60, TRA-1-81, Nanog and Oct3/4. In some embodiments, less than 1% of the MSCs express or the MSCs do not express at least three cell surface markers selected from CD14, CD19, CD34, CD45, HLA-DR, SSEA-3, TRA-1-60, TRA-1-81, Nanog and Oct3/4. In some embodiments, less than 1% of the MSCs express or the MSCs do not express at least four cell surface markers selected from CD14, CD19, CD34, CD45, HLA-DR, SSEA-3, TRA-1-60, TRA-1-81, Nanog and Oct3/4.

In some embodiments, less than 1% of the MSCs express or the MSCs do not express at least one cell surface marker selected from CD34, CD45, HLA-DR, TRA-1-60, and TRA-1-81. In some embodiments, less than 1% of the MSCs express or the MSCs do not express at least two cell surface markers selected from CD34, CD45, HLA-DR, TRA-1-60, and TRA-1-81. In some embodiments, less than 1% of the MSCs express or the MSCs do not express at least three cell surface markers selected from CD34, CD45, HLA-DR, TRA-1-60, and TRA-1-81. In some embodiments, less than 1% of the MSCs express or the MSCs do not express at least four cell surface markers selected from CD34, CD45, HLA-DR, TRA-1-60, and TRA-1-81. In some embodiments, less than 1% of the MSCs express or the MSCs do not express any of CD34, CD45, HLA-DR, TRA-1-60, and TRA-1-81.

In some embodiments, at least 95% of the MSCs are CD73+ and CD105+, and less than 1% express CD45, SSEA-3, TRA-1-60, TRA-1-81, and HLA-DR. In some embodiments, at least 98% of the MSCs are CD73+ and CD105+, and less than 1% express CD45, SSEA-3, TRA-1-60, TRA-1-81, and HLA-DR.

In some embodiments, provided are uses of genetically modified stem cells (e.g., PSCs or MSCs) as described herein, for preventing or treating an immune disease.

In some embodiments, provided are pharmaceutical compositions comprising genetically modified stem cells (e.g., PSCs or MSCs) as described herein. In some embodiments, provided pharmaceutical compositions are useful for preventing or treating an immune disease.

In some embodiments, providing are methods of treating and/or preventing an immune disease using genetically modified stem cells (e.g., PSCs or MSCs) as described herein and/or pharmaceutical compositions comprising the same.

In some embodiments, the immune disease is selected from the group consisting of lupus (SLE), lupus nephritis (e.g., drug-induced lupus nephritis), immune thrombocytopenia (ITP), rheumatoid arthritis (RA), multiple sclerosis (MS), inflammatory bowel disease (IBD) (e.g. Crohn's disease and colitis/ulcerative colitis), graft-versus-host disease (GVHD) or allograft rejection, transplantation/solid organ transplantation (SOT), primary biliary cholangitis (PBC), psoriasis, psoriatic arthritis, collagen-induced arthritis, oophoritis, allergic rhinitis, asthma, Sjogren's syndrome, atopic eczema, myasthenia gravis, Graves' disease, and glomerulosclerosis.

The present disclosure also provides a method of preventing or treating a cancer including administering the genetically modified stem cells (e.g., PSCs or MSCs) that express a binding agent and/or the conditioned cell culture medium thereof to a subject.

The present disclosure also provides a composition for immunomodulation comprising the genetically modified stem cells (e.g., PSCs or MSCs) that express a binding agent and/or the conditioned cell culture medium thereof.

The present disclosure also provides use of the genetically modified stem cells (e.g., MSCs or PSCs) that express a binding agent and/or the conditioned cell culture medium thereof for immunomodulation.

The present disclosure also provides a method for immunomodulation comprising administering the genetically modified stem cells (e.g., PSCs or MSCs) that express a binding agent and/or the conditioned cell culture medium thereof to a subject.

The present disclosure also provides a composition or medium for culturing regulatory T cell comprising the genetically modified stem cells (e.g., PSCs or MSCs) and/or the conditioned cell culture medium thereof.

The present disclosure also provides a method for culturing regulatory T cell comprising, culturing regulatory T cell in presence of the genetically modified stem cells (e.g., PSCs or MSCs) that express a binding agent.

The present disclosure also provides use of the genetically modified stem cells (e.g., PSCs or MSCs) that express a binding agent and/or the conditioned cell culture medium thereof for culturing regulatory T cell.

The present disclosure also provides use of the genetically modified stem cells (e.g., PSCs or MSCs) that express a binding agent and/or the conditioned cell culture medium thereof for manufacturing a composition or medium for culturing regulatory T cell.

The present disclosure also provides a composition for drug delivery including the genetically modified stem cells (e.g., PSCs or MSCs) that express a binding agent or the conditioned cell culture medium thereof.

The present disclosure also provides the use of the genetically modified MSCs that express a binding agent or the conditioned cell culture medium thereof for drug delivery.

In some embodiments, provided are uses of genetically modified PSCs, for producing a cell therapeutic agent. In some embodiments, provided are pluripotent stem cells suitable for generating a population of MSCs. In some embodiments, provided are a population of PSCs for use in generating a population of MSCs.

In some embodiments, provided are lentiviral vectors suitable for use in preparing and/or transfecting MSCs of the present disclosure. In some embodiments, provided are lentiviral vectors comprising a nucleic acid comprising a transcriptional regulatory sequence operably linked to a sequence that encodes a binding agent as described herein, an IRES or 2A sequence and a selection gene. In some embodiments, a transcriptional regulatory sequence is a promoter selected from a CMV promoter, a EFS promoter, a CBh promoter, a MSCV promoter, a SFFV promoter, and a E1FA promoter. In some embodiments, a nucleic acid comprises, in order, a promoter, the sequence that encodes a binding agent, a IRES or 2A sequence, and an antibiotic selection gene. In some embodiments, the present disclosure provides methods of genetically modifying stem cells (e.g., MSCs or PSCs) to comprise a nucleic acid encoding a target binding agent (e.g., a binding protein, e.g., an antibody, an antibody fragment, a stefin A protein variant, or fusion protein thereof).

### BRIEF DESCRIPTION OF THE DRAWINGS

The drawings included herein are for illustration purposes only and not for limitation.

FIG. 1 shows the results of comparing the gene introduction efficiency of transduction enhancers upon transduction of mesenchymal stromal cells using a lentiviral vector (SEQ ID NO: 94) including an eGFP gene, in which eGFP gene introduction efficiency was measured based on the fluorescence wavelength of GFP after treatment with polybrene (2, 4, and 8 μg/mL), protamine sulfate (5, 10, and 20 μg/mL), and LentiBOOST (1:500, 1:100, and 1:20).

FIG. 2 shows the results confirming the gene introduction efficiency based on the fluorescence wavelength of GFP when using a process of treating attached mesenchymal stromal cells with the lentiviral vector including the eGFP gene and a process of treating non-attached mesenchymal stromal cells with the lentiviral vector (reverse transduction).

FIG. 3A shows the results of evaluating the concentration conditions of G418 for selecting gene-introduced cells, in which Naïve MSC was treated with 500, 250, 125, 62.5, 31.25, 15.625, and 7.8125 μg/mL of G418, and cell death was observed at intervals of 1, 3, 5, and 7 days using CCK-8 assay.

FIG. 3B shows the results of selecting cells expressing the eGFP gene (Vector sequence=SEQ ID NO: 94) using G418 and of analyzing the cells by FACS before (left) and after (right) selection.

FIG. 4A shows the results of comparing the gene introduction efficiency using a fluorescence microscope after transduction of mesenchymal stromal cells using a Lentivirus Promoter Blast™ kit (Applied Biological Materials Inc.) in order to evaluate the expression efficiency of the GFP gene depending on the type of promoter.

FIG. 4B shows the results of comparing the gene introduction efficiency for each promoter using the GFP gene by FACS analysis (CMV, Vector sequence=SEQ ID NO: 94).

FIG. 5A shows the results of comparing the expression of the anti-CD40L stefin A protein variant by evaluating the combination of the promoter and the gene-linked peptide, in which a lentiviral vector expressing the anti-CD40L stefin A protein variant was synthesized using CMV, EFS, CBh, MSCV, SFFV, and EF1A promoters and was then introduced into mesenchymal stromal cells to construct a cell line, the anti-CD40L stefin A protein variant secreted by the cell line thus constructed was quantified using an ELISA kit, and a difference in expression was represented in multiples, and FIG. 5B shows the results of analysis of the amount of the anti-CD40L stefin A protein variant that is secreted by constructing a cell line in which the expression of the anti-CD40L stefin A protein variant was regulated by an EF1A promoter (Vector sequence=SEQ ID NO: 98) or a CBh promoter (Vector sequence=SEQ ID NO: 102) and then subjecting the cells to continuous subculture.

FIGS. 6A, 6B, and 6C show results confirming the passage stability of a cell line in which the expression of the anti-CD40L stefin A protein variant was regulated by the EF1A promoter (Vector sequence=SEQ ID NO: 98) or the CBh promoter (Vector sequence=SEQ ID NO: 102), FIG. 6A showing results of comparative analysis of the cell size through continuous subculture from PN9 to PN19, FIG. 6B showing results of cell proliferation time (PDT), and FIG. 6C showing results of comparative analysis of cell proliferation rate (PDL).

FIG. 7 shows the results of analyzing the ability of the anti-CD40L stefin A protein variant secreted out of the cells for each promoter to bind to CD40L through binding ELISA, *5C8: anti-CD40L monoclonal antibody, *eMSC (XT75, w/EF1A), eMSC (XT75, w/CBh).

FIG. 8 shows the results of analyzing the ability of the anti-CD40L stefin A protein variant secreted out of the cells for each promoter to inhibit the binding between CD40L and CD40 through cell-based assay (HEK-Blue Assay), *5C8: anti-CD40L monoclonal antibody, *eMSC (XT75, w/EF1A), eMSC (XT75, w/CBh).

FIG. 9 shows the results confirming whether cells secreting the anti-CD40L stefin A protein variant are effective at inhibiting activation of PBMC (PBMC clustering assay), in which each of Naïve MSC, XT73 gene (SEQ ID NO: 104)-introduced cell line, and XT75 gene (SEQ ID NO: 105)-introduced cell line was co-cultured with PBMC at different ratios (1:20 to 1:1), and the effect of inhibiting clustering of activated PBMC was confirmed, *eMSC (XT73, SEQ ID NO: 104), eMSC (XT75, SEQ ID NO: 105).

FIG. 10 shows the results of comparing whether cells secreting the anti-CD40L stefin A protein variant inhibit an increase in CD3+ T-cell activity through FACS analysis, *eMSC (XT73, SEQ ID NO: 104), eMSC (XT75, SEQ ID NO: 105).

FIG. 11 shows the results of analyzing whether a protein secreted by cells into which the anti-CD40L stefin A protein variant gene (SEQ ID NO: 105) was introduced is effective at inhibiting clustering of B cells.

FIG. 12 shows the results of analyzing whether the protein secreted by the cells into which the anti-CD40L stefin A protein variant gene (SEQ ID NO: 105) was introduced inhibits the activation of B cells, using a CD86 surface marker.

FIG. 13 shows the results of comparing the expression of immunomodulatory factors in Naïve MSC and MSC into which the anti-CD40L stefin A protein variant gene (SEQ ID NO: 105) was introduced through Western blot.

FIG. 14 shows the results of analyzing the purity of the cells into which the anti-CD40L stefin A protein variant gene (SEQ ID NO: 105) was introduced by FACS.

FIG. 15 shows a schematic view (left) and an experimental group design (right) of a xenograft GVHD animal model.

FIG. 16 shows clinical scores representing the efficacy of cells into which the anti-CD40L stefin A protein variant gene (SEQ ID NO: 105) was introduced using the xenograft GVHD mouse model, *G1: normal control, *G2: CS10, *G3: 5c8, *G4: Naïve MSC, *G5: XT75 (eMSC).

FIG. 17 and FIG. 18 show characterization of eMSCs (engineered Mesenchymal Stromal Cells). Expression of mesenchymal stromal cell specific markers by eMSCs CD29, CD44, CD73, CD90, and CD105 (FIG. 6), but not cell markers CD45, CD14, CD19, HLA-DR, SSEA-3, TRA-1-60, and TRA-1-81 (FIG. 7) (Data represent shown from a representative experiment).

FIG. 19 shows analysis of stefin A protein variant specifically binding to TNFR2 expressed on the cell surface. Cell lysates from eMSCs transduced with stefin A protein variant were probed for surface expression by Western blotting and compared to signals from each stefin A protein variant clone on the same gel. R-actin Western blot serves as loading control for the protein lysates from various eMSCs.

FIG. 20A and FIG. 20B show stefin A protein variant quantification on cell surface. The number of stefin A protein variant on the cell surface was quantified by QuantiBRITE PE fluorescence quantification kit for flow cytometric analysis. Antibody for stefin A protein variant conjugated to phycoerythrin (PE) were used for the flow cytometric analysis.

FIG. 21 shows SEAP release of HEK-Blue TNFα reporter cell line triggered by 7 best stefin A protein variants on the eMSCs surface. HEK-Blue TNFα cells seed 20,000 cells/well. eMSCs seed at 1/2 serial dilution from 40,960 cells/well. Data represents the mean of triplicate (Mean±SD).

### DETAILED DESCRIPTION

The present disclosure describes, among other things, development of genetically modified stem cells (e.g., MSCs or PSCs) having excellent immunomodulatory activity through genetic modification. The present disclosure provides, inter alia, genetically modified stem cells (e.g., MSCs or PSCs) that stably expresses a target-binding agent (e.g., a target binding protein, e.g., an antibody, an antibody fragment, a stefin A protein variant, or fusion protein thereof), and populations of such genetically modified stem cells (e.g., MSCs or PSCs). In some embodiments, the present disclosure relates to a genetically modified stem cells (e.g., MSCs or PSCs) comprising a nucleic acid encoding a target binding protein (e.g., an antibody, an antibody fragment, a stefin A protein variant, or fusion protein thereof). The present disclosure also provides methods of producing genetically modified stem cells (e.g., MSCs or PSCs) by introducing an exogenous nucleic acid encoding a target-binding protein e.g., an antibody fragment, a stefin A protein variant, and/or a fusion protein).

The examples of the present disclosure, describe production of genetically engineered MSCs expressing example target binding proteins, including a secreted target-binding protein and a cell-surface expressed target binding protein. The examples confirm that the genetically modified MSCs are capable of specifically binding to a target cell through expression of a target binding protein, and that passage stability and immunomodulatory effect of the MSCs are maintained despite the gene introduction.

Example genetically modified MSCs as exemplified herein show beneficial characteristics. For example, exemplified genetically modified MSCs expressing stefin A protein variants that specifically bind to TNFR2 showed an excellent agonistic effect on TNFR2. Also exemplified are MSCs that express a stefin A protein variant that specifically binds to CD40L or a fusion protein including the same on the cell membrane, and demonstrates that these genetically modified MSCs exhibit a vastly superior immunomodulatory effect compared to unmodified MSCs. The present disclosure encompasses a recognition that provided genetically modified MSCs of the present disclosure may be useful for culturing or activating immune cells.

### Certain Definitions

Unless otherwise defined, all technical and scientific terms used herein have the same meanings as those typically understood by those skilled in the relevant art. In general, the nomenclature used herein is well known in the art and is typical.

The term, “protein” or “polypeptides” are polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labelling component. Also included within the definition are, for example, polypeptides containing at least one analog of an amino acid (including, for example, unnatural amino acids), as well as other modifications known in the art.

For the most part, the amino acids and amino acid sequences used in the application are those naturally occurring amino acids found in proteins, or the naturally occurring anabolic or catabolic products of such amino acids which contain amino and carboxyl groups, and isomer thereof (e.g. D- or L- stereoisomers).

Amino acid residues further include analogs, derivatives and congeners of any specific amino acid referred to herein, as for instance, the subject AFFIMER® polypeptide (particularly if generated by chemical synthesis) can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminiopimelic acid, ornithine, or diaminobutyric acid.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. In the present disclosure, two nucleic acid or amino acid sequences can be “substantially identical”, which means that the two sequences are at least 70%, 75%, 80%, 85%, 90%, 95%, 96%, 97%, 98%, or 99% identical. In some embodiments, identity may exist over a length of at least about 10, 20, 40-60, 60-80, 80-100 or more residues. When calculating percent identity, any residues defined as ‘Xaa’ or ‘X’ in a reference sequence herein are included in the percentage identity calculation, i.e. any amino acid in this position in a comparison sequence matches the reference sequence.

The protein or polypeptide described herein, for example, a binding protein, e.g., an antibody fragment, a stefin A protein variant, and/or a fusion protein, may include not only the amino acid sequence described in regard thereto, but also a protein or polypeptide in which a portion of the amino acid sequence is substituted through conservative substitution.

As used herein, “conservative substitution” refers to a modification of a polypeptide comprising substituting one or more amino acids with amino acids having similar biochemical properties that do not cause loss of biological or biochemical functions of the polypeptide.

A conservative amino acid substitution is one in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, proteins of the present disclosure do not cause functional loss, for example, a stefin A protein variant that specifically binds to a target does not abrogate its binding to its target by a conservative substitution. Methods of identifying amino acid conservative substitutions which do not eliminate binding are well-known in the art.

A polypeptide, soluble protein, antibody, polynucleotide, vector, cell, or composition which is “isolated” is a polypeptide, soluble protein, antibody, polynucleotide, vector, cell, or composition which is in a form not found in nature. Isolated polypeptides, soluble proteins, antibodies, polynucleotides, vectors, cells, or compositions include those which have been purified to a degree that they are no longer in a form in which they are found in nature. In some embodiments, a polypeptide, soluble protein, antibody, polynucleotide, vector, cell, or composition which is isolated is substantially pure.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

As used herein, reference to “about” or “approximately” a value or parameter includes (and describes) embodiments that are directed to that value or parameter. For example, description referring to “about X” includes description of “X”.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The phrase “at least one” may be used interchangeably with “one or more.” It should be understood that “a” is not limited to one but rather means “at least one.”

The term “exogenous” as used herein refers to a substance present in the cell other than its native source. In the context of nucleic acids and/or proteins, “exogenous” means that the nucleic acid or protein is introduced by a process involving the human hand into a biological system such as a cell or organism in which the nucleic acid or protein is not normally found or is found in lower amounts. A substance (e.g., a nucleic acid encoding a protein) is considered exogenous if it has been introduced into a cell or an ancestor of a cell that inherits the substance.

### Targets

In some embodiments, a target binding protein (e.g., an antibody, an antibody fragment, or a stefin A protein variant) specifically binds to a target protein as described herein.

In some embodiments, a target protein is expressed on an immune cell. In some embodiments, a target protein is expressed on the surface of an immune cell.

Immune cells include, but are not limited to, dendritic cells (such as immature dendritic cells and mature dendritic cells), peripheral blood mononuclear cells (PBMC), T lymphocytes (naive T cells, effector T cells, memory T cells, cytotoxic T lymphocytes), helper T cells, natural killer T cells, regulatory T cells (Treg cells), tumor infiltrating lymphocytes (TIL), lymphokine activated killer (LAK) cells), B cells, eosinophils, natural killer (NK) cells, monocytes, macrophages, neutrophils, granulocytes, and combinations thereof.

In some embodiments, a target protein is expressed by activated immune cells. In some embodiments, a target protein is expressed by an activated T cell, B cell, NK cell, or combination thereof.

In some embodiments, a target protein expressed on the surface of an immune system can be, but is not limited to, CD3, CD4, CD8, CD19, CD20, CD11c, CD123, CD56, CD34, CD14, or CD33.

In some embodiments, a target protein is an immunomodulating protein. An immunomodulating protein refers to any protein that has an effect (e.g., an inhibitory or stimulatory effect) on the immune system.

In some embodiments, the target protein is expressed by activated immune cells and binding of the target protein by the binding agent inhibits inflammatory activity of the activated immune cells.

In some embodiments, a target protein is immunostimulatory TNF receptor ligand. In some embodiments, a target protein is CD27L, CD40L, 41BBL, or GITRL.

In some embodiments, a target protein is a TNF receptor. In some embodiments, a target protein is a TNFR2. In some embodiments, binding of the target protein by the binding agent inhibits receptor function. In some embodiments, binding of the target protein by the binding agent activates receptor function.

In some embodiments, a target protein is a proinflammatory cytokine or a receptor thereof. In some embodiments, binding of the target protein by the binding agent inhibits the proinflammatory cytokine. In some embodiments, a target protein is a proinflammatory cytokine selected from: IL-1, IL-6, IL-12, and IL-18, TNF-α, IFNγ, and GM-CSF.

### Target Binding Agents

The present disclosure provides, among other things, stem cells (e.g., PSCs and/or MSCs) that comprise an exogenous nucleic acid encoding a target binding agent (e.g., a target binding protein).

In some embodiments, a target-binding protein binds to and has an agonistic effect on a target protein. In some embodiments, a target protein is protein that is present at a site of inflammation and/or functions to promote regeneration. In some embodiments, the target-binding protein binds to the target protein to promote the target protein's function in tissue regeneration.

In some embodiments, a target-binding protein binds to and has an antagonistic effect on a target protein. In some embodiments, a target protein is protein that is present at a site of inflammation and/or functions to promote inflammation. In some embodiments, the target-binding protein binds to the target protein and inhibits the target protein's function in promoting inflammation.

In some embodiments, a target binding protein is a recombinant protein comprising one or more binding domains from an antibody or antibody mimetic which bind to a target. In some embodiments, a binding agent is a stefin A protein variant, Fab, Fab′, F(ab′)2, Fv, Fd, scFv, sdFv), VL, VH, Camel Ig, V-NAR, VHH, trispecific (Fab3), bispecific (Fab2), diabody ((VL-VH)2 or (VH-VL)2), triabody (trivalent), tetrabody (tetravalent), minibody ((scFv-CH3)2), bispecific single-chain Fv (Bis-scFv), a shark heavy-chain-only antibody (VNAR), a microprotein (cysteine knot protein, knottin), affibody, aptamer, avimer, nanobody, unibody, a single domain antibody, affilin, affitin, adnectin, atrimer, evasin, DARPin, anticalin, avimer, fynomer, versabody, repebody, or a duocalin.

In some embodiments, a target binding protein is a recombinant protein comprising one or more binding domains from an antibody or antibody mimetic which bind to a target. In some embodiments, a binding agent is a stefin A protein variant, Fab, Fab′, F(ab′)2, Fv, Fd, scFv, sdFv), VL, VH, Camel Ig, V-NAR, VHH, trispecific (Fab3), bispecific (Fab2), diabody ((VL-VH)2 or (VH-VL)2), triabody (trivalent), tetrabody (tetravalent), minibody ((scFv-CH3)2), bispecific single-chain Fv (Bis-scFv), a shark heavy-chain-only antibody (VNAR), a microprotein (cysteine knot protein, knottin), affibody, avimer, nanobody, unibody, a single domain antibody, affilin, affitin, adnectin, atrimer, evasin, DARPin, anticalin, avimer, fynomer, versabody, repebody, or a duocalin.

In some embodiments, a target binding protein may be selected from the group consisting of, for example, a stefin A protein variant, an antibody or fragment thereof, an antibody-like material, an antigen-binding peptide, a ligand-binding site of a receptor (e.g. a receptor trap polypeptide), a receptor-binding ligand (e.g. a cytokine or a growth factor), an engineered T-cell receptor, and an enzyme or a catalytic fragment thereof, but is not limited thereto.

In some embodiments, a target binding protein is an antibody. As used herein, the term “antibody” includes not only a complete antibody form that specifically binds to a target (antigen), but also an antigen-binding fragment of the antibody molecule. The complete antibody has a structure having two full-length light chains and two full-length heavy chains, and each light chain is connected to the heavy chain by a disulfide bond. As used herein, the term “heavy chain” refers to a full-length heavy chain including a variable region domain VH including an amino acid sequence having a sufficient variable region sequence to confer specificity to an antigen and three constant region domains CH1, CH2, and CH3, and fragments thereof. In addition, as used herein, the term “light chain” refers to a full-length light chain including a variable region domain VL including an amino acid sequence having a sufficient variable region sequence to confer specificity to an antigen and a constant region domain CL, and fragments thereof. The whole antibody includes subtypes of IgA, IgD, IgE, IgM, and IgG, and in particular, IgG includes IgG1, IgG2, IgG3, and IgG4. The heavy-chain constant region has gamma (γ), mu (μ), alpha (α), delta (δ), and epsilon (ε) types, and subclasses such as gamma 1 (γ1), gamma 2 (γ2), gamma 3 (γ3), gamma 4 (γ4), alpha 1 (α1), and alpha 2 (α2). The constant region of the light chain has kappa (κ) and lambda (λ) types.

In some embodiments, a target binding protein is an antigen-binding antibody fragment. The antigen-binding fragment of an antibody or antibody fragment refers to a fragment having an antigen-binding function, and includes Fab, F(ab′), F(ab′)2, and Fv. Among the antibody fragments, Fab has a structure having variable regions of light and heavy chains, a constant region of a light chain, and a first constant region (CH1) of a heavy chain, and has one antigen-binding site. Fab′ differs from Fab in that Fab′ has a hinge region including at least one cysteine residue at the C-terminus of the heavy-chain CH1 domain. F(ab′)2 is formed by a disulfide bond between cysteine residues in the hinge region of Fab′.

Fv is a minimal antibody fragment having only a heavy-chain variable region and a light-chain variable region. A two-chain Fv is a fragment in which a heavy-chain variable region and a light-chain variable region are linked by a non-covalent bond, and a single-chain Fv (scFv) is a fragment in which a heavy-chain variable region and a light-chain variable region are generally linked by a covalent bond via a peptide linker therebetween, or are directly linked at the C-terminus, forming a dimeric structure, like the two-chain Fv. Such antibody fragments may be obtained using proteases (for example, Fab may be obtained by restriction-cleaving a whole antibody with papain, and the F(ab′)2 fragment may be obtained by restriction-cleaving a whole antibody with pepsin), or may be constructed through genetic recombination technology.

An “Fv” fragment is an antibody fragment that contains a complete antibody recognition and binding site. This region is a dimer in which one heavy-chain variable domain and one light-chain variable domain are joined.

A “Fab” fragment includes variable and constant domains of a light chain and variable and first constant domains (CH1) of a heavy chain. An F(ab′)2 antibody fragment generally includes a pair of Fab′ fragments covalently linked by cysteines in the hinge region present at the C-terminus of the Fab′ fragment.

A “single-chain Fv (scFv)” antibody fragment is a construct composed of a single polypeptide chain including the VH and VL domains of an antibody. scFv may further include a polypeptide linker between the VH domain and the VL domain so as form the desired structure for antigen binding.

Examples of antibodies suitable for expression in MSCs of the present disclosure may include, but are not limited to, monoclonal antibodies, multispecific antibodies, human antibodies, humanized antibodies, chimeric antibodies, scFv, Fab fragments, F(ab′)2 fragments, disulfide-linked Fvs (sdFv), and anti-idiotype (anti-Id) antibodies, epitope-binding fragments of such antibodies, and the like.

The heavy-chain constant region may be selected from among isotypes such as gamma (γ), mu (μ), alpha (α), delta (δ), and epsilon (ε). For example, the constant region is gamma 1 (IgG1), gamma 2 (IgG2), gamma 3 (IgG3), or gamma 4 (IgG4). The light-chain constant region may be a kappa or lambda type.

A monoclonal antibody is an antibody obtained from a population of substantially homogeneous antibodies, in which the individual antibodies that make up the population are identical, except for possible naturally-occurring mutations that may be present in small amounts. A monoclonal antibody is highly specific and is induced against a single epitope on the antigen. In contrast to typical (polyclonal) antibodies, which typically include different antibodies directed against different determinants (epitopes), each monoclonal antibody is directed against a single determinant on the antigen.

In some embodiments, a target binding protein (e.g., an antibody, an antibody fragment, a stefin A protein variant, or fusion protein thereof) may bind to its target as a monomer with a dissociation constant (K_{D}) of about 1 uM or less, about 100 nM or less, about 40 nM or less, about 20 nM or less, about 10 nM or less, about 1 nM or less, or about 0.1 nM or less.

In some embodiments, a target binding protein (e.g., an antibody, an antibody fragment, a stefin A protein variant, or fusion protein thereof) may bind to its target as a monomer with an off-rate constant (Koff), such as measured by BIACORE™ assay, of about 10⁻³ s⁻¹ (e.g., unit of 1/second) or slower; of about 10⁻⁴ s⁻¹ or slower or even of about 10⁻⁵ s⁻¹ or slower.

In some embodiments, a target binding protein (e.g., an antibody, an antibody fragment, a stefin A protein variant, or fusion protein thereof) may bind to its target as a monomer with an association constant (Kon), such as measured by BIACORE™ assay, of at least about 10³ M⁻¹s⁻¹ or faster; at least about 10⁴ M⁻¹s⁻¹ or faster; at least about 10⁵ M⁻¹s⁻¹ or faster; or even at least about 10⁶ M⁻¹s⁻¹ or faster.

In some embodiments, a target binding protein (e.g., an antibody, an antibody fragment, a stefin A protein variant, or fusion protein thereof) may bind to its target as a monomer with an IC50 in a competitive binding assay with its target of 1 uM or less, about 100 nM or less, about 40 nM or less, about 20 nM or less, about 10 nM or less, about 1 nM or less, or about 0.1 nM or less.

In some embodiments, a target binding protein (e.g., an antibody, an antibody fragment, a stefin A protein variant, or fusion protein thereof) may be expressed in the genetically modified MSC, and may be secreted and/or anchored to the membrane and presented on the cell surface.

In some embodiments, a target binding protein (e.g., an antibody, an antibody fragment, a stefin A protein variant, or fusion protein thereof) is anchored to the membrane of the genetically modified MSC or presented on the cell surface of a MSC. In some embodiments, when the target binding protein is presented on the membrane or surface of the genetically modified MSC, the genetically modified MSC may have agonistic effect on its target.

In some embodiments, a binding agent (e.g., a binding protein, e.g., an antibody, an antibody fragment, a stefin A protein variant, or fusion protein thereof) may be expressed in the genetically modified MSC, and may be localized to a specific organelle or location within the cell.

In some embodiments, when a binding agent (e.g., a binding protein, e.g., an antibody, an antibody fragment, a stefin A protein variant, or fusion protein thereof) is anchored to a membrane or expressed on a cell surface, the binding agent may be directly/indirectly linked (or fused) to a transmembrane domain, and may be expressed in the form of a fusion protein including a transmembrane domain. Membrane-anchored or cell-surface-expressed target binding fusion proteins are described in detail in the section on fusion proteins below.

In some embodiments, a target binding protein is monomer.

In some embodiments, a target binding protein may form a multimer.

In some embodiments, a target protein may form a dimer, trimer, tetramer, pentamer or more multimer. In some embodiments, the multimer may be formed by covalently or non-covalently linked by an interaction between amino acid residues of the binding agent.

In some embodiments, the multimer may be a fusion protein formed through a fusion domain fused with a target-binding antibody or antigen-binding antibody fragment.

In some embodiments, the multimer may be formed by in-line fusion of a target-binding antibody or antigen-binding antibody fragment.

### Stefin A Protein Variants Specifically Binding to Targets

As used herein, the term “stefin A protein variant (or stefin A protein variants)” a scaffold based on a stefin A polypeptide, meaning that it has a sequence which is derived from a stefin A polypeptide, for example, a mammalian stefin A polypeptide, for example, a human stefin A polypeptide. In the present disclosure, the “stefin A protein Variant” may be used interchangeably in substantially the same sense as “stefin A protein variants” or “AFFIMER® protein”. In the present disclosure, the “stefin A protein Variant” may be used interchangeably in substantially the same sense as “stefin A polypeptide variant” or “AFFIMER®”. Unless otherwise specified, the stefin A protein variant refers to the stefin A protein variant specifically binding to a target of the present disclosure.

AFFIMER®, developed by Avacta Life Sciences Limited, is a small stable protein molecule engineered based on a stefin A protein, which is an in-vivo protein. AFFIMER® includes two short peptide sequences having a random sequence and an N-terminal sequence, and is able to bind to a target material with high affinity and specificity in a manner similar to a monoclonal antibody. AFFIMER® shows remarkably improved binding affinity and specificity compared to the free peptide library, and has a very small size and high stability compared to antibodies, and is therefore receiving great attention as a next-generation alternative pharmaceutical platform to replace antibodies (U.S. Pat. Nos. 9,447,170, 8,853,131, etc.).

The terms “stefin A protein variant” and “AFFIMER” may be used interchangeably in substantially the same meaning.

As used herein, the term “stefin A protein” or (Stefin polypeptides)” encompass a subgroup of proteins in the cystatin superfamily, a family which encompasses proteins that contain multiple cystatin-like sequences. The Stefin subgroup of the cystatin family includes relatively small (around 100 amino acids) single domain proteins. They receive no known post-translational modification, and lack disulfide bonds, suggesting that they will be able to fold identically in a wide range of extracellular and intracellular environments. Stefin A itself is a monomeric, single chain, single domain protein of 98 amino acids. The structure of stefin A has been solved, facilitating the rational mutation of stefin A into the AFFIMER® polypeptide. The only known biological activity of cystatins is the inhibition of cathepsin activity, which allowed for exhaustive testing for residual biological activity of the engineered proteins.

As used herein, the term “stefin A protein variant” refers to a small, highly stable protein that is an engineered variant of a Stefin polypeptide. AFFIMER® proteins display two peptide loops and an N-terminal sequence that can all be randomized to bind to desired target proteins with high affinity and specificity, in a similar manner to monoclonal antibodies. Stabilization of the two peptides by the stefin A protein scaffold constrains the possible conformations that the peptides can take, increasing the binding affinity and specificity compared to libraries of free peptides. These engineered non-antibody binding proteins are designed to mimic the molecular recognition characteristics of monoclonal antibodies in different applications. Variations to other parts of the stefin A polypeptide sequence can be carried out, with such variations improving the properties of these affinity reagents, such as increase stability, make them robust across a range of temperatures and pH and the like. In some embodiments, an AFFIMER® polypeptide includes a sequence derived from stefin A, sharing substantial identify with a stefin A wild type sequence, such as human stefin A. It will be apparent to a person skilled in the art that modifications may be made to the scaffold sequence without departing from the disclosure. In particular, an AFFIMER® polypeptide can have an amino acid sequences that is at least 25%, 35%, 45%, 55% or 60% identity to the corresponding sequences to human stefin A, for example, at least 70%, at least 80%, at least 85%, at least 90%, at least 92%, at least 94%, at least 95% identical, e.g., where the sequence variations do not adversely affect the ability of the scaffold to bind to the desired target, and e.g., which do not restore or generate biological functions such as those which are possessed by wild type stefin A but which are abolished in mutational changes described herein. A target protein-specific binding platform using such a stefin A protein variant is disclosed in detail in U.S. Pat. Nos. 9,447,170 and 8,853,131. In some embodiments, the stefin A protein variant may be the fragment of the stefin A protein variant can specifically bind to a target.

In some embodiments, the stefin A protein variant can specifically bind to a target protein with high affinity and specificity through engineering of the stefin A protein.

In some embodiments, the stefin A protein variant specifically binding to target protein may exhibit an agonistic effect on the target.

“Agonist” and “agonistic” refer to agents that are capable of, directly or indirectly, substantially inducing, activating, promoting, increasing, or enhancing the biological activity of a target or target pathway. “Agonist” is used herein to include any agent that partially or fully induces, activates, promotes, increases, or enhances the activity of a protein or other target of interest.

In some embodiments, the stefin A protein variant specifically binding to a target may bind to and activate the target or target signaling pathway.

In some embodiments, the stefin A protein variant specifically binding to a target may bind to the target with Kd of 10⁻⁶ M or less.

In some embodiments, the stefin A protein variant specifically binding to a target is derived from the wild-type human stefin A polypeptide having a backbone sequence and in which one or both of loop 2 [designated (Xaa)n] and loop 4 [designated (Xaa)m] are replaced with alternative loop sequences (Xaa)n and (Xaa)m. In some embodiments, the stefin A protein variant specifically binds to target may comprise an amino acid sequence represented by Formula (I):

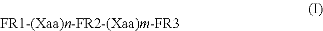

wherein
FR1 comprises or is a sequence represented by MIPGGLSEAK PATPEIQEIV DKVKPQLEEK TGETYGKLEA VQYKTQVX (SEQ ID NO: 1) or MIPGGLSEAK PATPEIQEIV DKVKPQLEEK TNETYGKLEA VQYKTQVLA (SEQ ID NO: 6); or a polypeptide sequence having at least 70% (e.g., at least 75%, at least 80%, at least 85%, at least 90%, at least 95%, or 100%) identity to the amino acid sequence of SEQ ID NO: 1, wherein X is any number of independently selected amino acids, more suitably three or fewer independently selected amino acids, or more suitably X is V or D; and/or
FR2 comprises or is a sequence comprising the amino acid sequence of GTNYYIKVRA GDNKYMHLKV FKSL (SEQ ID NO: 2) or a polypeptide sequence having at least 70% (e.g., at least 75%, at least 80%, at least 85%, at least 90%, at least 95%, or 100%) identity to the amino acid sequence of SEQ ID NO: 2;
FR3 comprises or is a sequence comprising the amino acid sequence of EDLVLTGYQV DKNKDDELTG F (SEQ ID NO: 3) or a polypeptide sequence having at least 70% (e.g., at least 75%, at least 80%, at least 85%, at least 90%, at least 95%, or 100%) identity to the amino acid sequence of SEQ ID NO: 3; and
Xaa, individually for each occurrence, is an amino acid residue; and
n and m are each, independently, an integer from 3 to 20.

In some embodiments, FR1 is a polypeptide sequence having at least 80%, 85%, 90%, 95% or even 98% homology with SEQ ID NO: 1. In some embodiments, FR1 is a polypeptide sequence having at least 80%, 85%, 90%, 95% or even 98% identity with SEQ ID NO: 1; In some embodiments, FR2 is a polypeptide sequence having at least 80%, 85%, 90%, 95% or even 98% homology with SEQ ID NO: 2. In some embodiments, FR2 is a polypeptide sequence having at least 80%, 85%, 90%, 95% or even 98% identity with SEQ ID NO: 2; In some embodiments, FR3 is a polypeptide sequence having at least 80%, 85%, 90%, 95% or even 98% homology with SEQ ID NO: 3. In some embodiments, FR3 is a polypeptide sequence having at least 80%, 85%, 90%, 95% or even 98% identity with SEQ ID NO: 3.

In some embodiments, the stefin A protein variant specifically binding to a target comprises an amino acid sequence represented in the general Formula (II):

(SEQ ID NO: 4)
MIP-Xaa1-GLSEAKPATPEIQEIVDKVKPQLEEKTGETYGKLEAVQYKTQV-
Xaa2-(Xaa)n-Xaa3-TNYYIKVRAGDNKYMHLKVF-Xaa4-Xaa5-Xaa6-(Xaa)m-Xaa7-D-
Xaa8-VLTGYQVDKNKDDELTGF

wherein
Xaa, individually for each occurrence, is any number of an amino acid residue, more suitably three or fewer (preferably, one or two) independently selected amino acids, and
n and m are each, independently, an integer from 3-20.

In some embodiments, the stefin A protein variant specifically binding to a target comprises an amino acid sequence having at least 90% (e.g., at least 95%, at least 96%, at least 97%, at least 98%, at least 99%, or 100%) identity to the amino acid sequence of:

(SEQ ID NO: 7)
MIPGGLSEAKPATPEIQEIVDKVKPQLEEKTGETYGKLEAVQYKTQVV-
(Xaa)n-GTNYYIKVRAGDNKYMHLKVFKSL-(Xaa)m-
EDLVLTGYQVDKNKDDELTGF;
(SEQ ID NO: 5)
MIPGGLSEAKPATPEIQEIVDKVKPQLEEKTGETYGKLEAVQYKTQVD-
(Xaa)n-GTNYYIKVRAGDNKYMHLKVFKSL-(Xaa)m-
EDLVLTGYQVDKNKDDELTGF;
or
(SEQ ID NO: 8)
MIPGGLSEAKPATPEIQEIVDKVKPQLEEKTGETYGKLEAVQYKTQVLA-
(Xaa)n-GTNYYIKVRAGDNKYMHLKVFKSL-(Xaa)m-
EDLVLTGYQVDKNKDDELTGF,

wherein
Xaa, individually for each occurrence, is any number of an amino acid residue, more suitably three or fewer (preferably, one or two) independently selected amino acids, and
n and m are each, independently, an integer from 3-20.

In some embodiments, Xaa1 is Gly, Ala, Val, Arg, Lys, Asp, or Glu, more preferably Gly, Ala, Arg or Lys, and more even more preferably Gly or Arg; Xaa2 is Val, Asp or ‘Leu-Ala’; Xaa3 is Gly, Ala, Val, Ser or Thr, more preferably Gly or Ser; Xaa4 is Arg, Lys, Asn, Gln, Ser, Thr, more preferably Arg, Lys, Asn or Gln, and even more preferably Lys or Asn; Xaa5 is Gly, Ala, Val, Ser or Thr, more preferably Gly or Ser; Xaa6 is Ala, Val, Ile, Leu, Gly or Pro, more preferably Ile, Leu or Pro, and even more preferably Leu or Pro; Xaa7 is Gly, Ala, Val, Asp or Glu, more preferably Ala, Val, Asp or Glu, and even more preferably Ala or Glu; and Xaa8 is Ala, Val, Ile, Leu, Arg or Lys, more preferably Ile, Leu or Arg, and even more preferably Leu or Arg.

In some embodiments, n is 3 to 15, 3 to 12, 3 to 9, 3 to 7, 5 to 7, 5 to 9, 5 to 12, 5 to 15, 7 to 12 or 7 to 9.

In some embodiments, m is 3 to 15, 3 to 12, 3 to 9, 3 to 7, 5 to 7, 5 to 9, 5 to 12, 5 to 15, 7 to 12 or 7 to 9.

In some embodiments, Xaa, independently for each occurrence, is an amino acid that can be added to a polypeptide by recombinant expression in a prokaryotic or eukaryotic cell, and even more preferably one of the 20 naturally occurring amino acids.

In some embodiments, the stefin protein A variant optional includes an amino acid sequence to facilitate purification.

In some embodiments, the stefin A protein variant specifically binding to a target may bind to the target as a monomer with a dissociation constant (KD) of about 1 uM or less, about 100 nM or less, about 40 nM or less, about 20 nM or less, about 10 nM or less, about 1 nM or less, or about 0.1 nM or less.

In some embodiments, the stefin A protein variant specifically binding to a target may bind to the target as a monomer with an off-rate constant (Koff), such as measured by BIACORE™ assay, of about 10³ s⁻¹ (e.g., unit of 1/second) or slower; of about 10⁻⁴ s⁻¹ or slower or even of about 10⁻⁵ s⁻¹ or slower.

In some embodiments, the stefin A protein variant specifically binding to a target may bind to the target as a monomer with an association constant (Ko), such as measured by BIACORE™ assay, of at least about 10³ M⁻¹s⁻¹ or faster; at least about 10⁴ M⁻¹s⁻¹ or faster; at least about 10⁵ M⁻¹s⁻¹ or faster; or even at least about 10⁶ M⁻¹s⁻¹ or faster.

In some embodiments, the stefin A protein variant specifically binding to a target may bind to the target as a monomer with an IC₅₀ in a competitive binding assay with the target of 1 uM or less, about 100 nM or less, about 40 nM or less, about 20 nM or less, about 10 nM or less, about 1 nM or less, or about 0.1 nM or less.

In some embodiments, the stefin A protein variant specifically binding to a target has a melting temperature (Tm, e.g., temperature at which both the folded and unfolded states are equally populated) of 65° C. or higher, and preferably at least 70° C., 75° C., 80° C. or even 85° C. or higher. Melting temperature is a particularly useful indicator of protein stability. The relative proportions of folded and unfolded proteins can be determined by many techniques known to the skilled person, including differential scanning calorimetry, UV difference spectroscopy, fluorescence, circular dichroism (CD), and NMR (Pace et al. (1997) “Measuring the conformational stability of a protein” in Protein structure: A practical approach 2: 299-321).

In some embodiments, the stefin A protein variant specifically binding to a target may be expressed in the genetically modified MSC, and may be secreted and/or anchored to the membrane and presented on the cell surface.

In some embodiments, the stefin A protein variant specifically binds to a target, preferably anchored to the membrane of the genetically modified MSC or presented to the cell surface. In particular, when the stefin A protein variant specifically binds to a target is presented on the membrane or cell surface, the genetically modified MSC may have agonistic effect on the target.

In some embodiments, the stefin A protein variant specifically binding to a target may be expressed in the genetically modified MSC, and may be localized to a specific organelle or location within the cell.

In some embodiments, when the stefin A protein variant is anchored to a membrane or expressed on a cell surface, the stefin A protein variant may be directly/indirectly linked (or fused) to a transmembrane domain, and may be expressed in the form of a fusion protein including a transmembrane domain. In the case of membrane-anchored or cell-surface-expressed stefin A protein variant, the fusion protein comprising the transmembrane will be described in detail in the section on fusion proteins below.

In some embodiments, the stefin A protein variant specifically binding to a target is monomer.

In some embodiments, the stefin A protein variant specifically binding to a target may form a multimer.

In some embodiments, the stefin A protein variant specifically binding to a target may form a dimer, trimer, tetramer, pentamer or more multimer.

In some embodiments, the multimer may be formed by covalently or non-covalently linked by an interaction between amino acid residues of the stefin A protein variant.

In some embodiments, the multimer may be a fusion protein formed through a fusion domain fused with a stefin A protein variant.

In some embodiments, the multimer may be formed by in-line fusion of a stefin A protein variant, and the in-line fusion protein in the form of such a multimer will be described in detail in the section on fusion proteins below.

### Nucleic Acids Encoding the Binding Agents

As used herein, “nucleic acid” is a polynucleotide of any length and may comprise DNA, RNA or a combination of DNA and RNA. The nucleotides can be deoxyribonucleotides, ribonucleotides, modified nucleotides or bases, and/or their analogs, or any substrate that can be incorporated into a polymer by DNA or RNA polymerase. Nucleic acids include, for example but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, a-LNA having an a-L-ribo configuration (a diastereomer of LNA), 2′-amino-LNA having a 2′-amino functionalization, and 2′-amino-a-LNA having a 2′-amino functionalization), ethylene nucleic acids (ENA), cyclohexenyl nucleic acids (CeNA) or hybrids or combinations thereof.

As used herein, “nucleic acid encoding ˜” refers to a nucleic acid sequence encoding a specific protein or polypeptide. As used herein, “nucleic acid encoding ˜” is nucleic acid sequence encoding a specific protein or polypeptide. In the art, when the sequence of a specific protein or polypeptide has been known, methods for designing or deriving a nucleic acid encoding the same are well known.

Therefore, nucleic acids encoding a binding agent (e.g., a binding protein, e.g., a stefin A protein variant that specifically binds to a target) can be easily understood from the description above (for example, under the heading of the “stefin A protein variants specifically binding to targets”).

### Fusions Proteins—General

In some embodiments, the genetically modified cell may be introduced with a nucleic acid encoding a fusion protein comprising the binding agent. In some embodiments, the fusion protein may be one in which an additional peptide sequence (a fusion domain) is fused to one end and/or the other end of the binding agent (e.g., an antibody, an antibody fragment, or a stefin A protein variant).

As used herein, the term “fusion domain” refers to an additional domain or moiety that may be incorporated directly or indirectly by being fused to the binding agent (e.g., an antibody, an antibody fragment, or a stefin A protein variant) of the present disclosure.

In some embodiments, the fusion protein may comprise at least one fusion domain.

In some embodiments, the fusion protein may further comprise an additional insertion, substitution and/or deletion that modulates biological activity of the binding agent (e.g., an antibody, an antibody fragment, or a stefin A protein variant) or that gives additional biological functions. For example, the additions, substitutions and/or deletions may modulate at least one property or activity of modified the binding agent (e.g., an antibody, an antibody fragment, or a stefin A protein variant). For example, the additions, substitutions or deletions may modulate affinity of the binding agent (e.g., an antibody, an antibody fragment, or a stefin A protein variant), e.g., for binding to a target, modulate the circulating half-life, modulate the therapeutic half-life, modulate the stability of the binding agent, modulate cleavage by proteases, modulate dose, modulate release or bioavailability, facilitate purification, decrease deamidation, improve shelf-life, or improve or alter a particular route of administration. For examples, the fusion protein may further comprise protease cleavage sequences, reactive groups, antibody-binding domains (including but not limited to, FLAG or poly-His) or other affinity-based sequences (including but not limited to, FLAG, poly-His, GST, etc.) or linked molecules (including but not limited to, biotin) that improve detection, purification or other traits of the polypeptide, but is not limited thereto.

In some embodiments, the fusion domain, for example, may be fused to confer expression properties such as secretion from the cell, or anchoring to the cell surface (e.g. cell membrane), or intracellular localization; to serve as substrate or other recognition sequences for post-translational modifications; to create multimeric structures aggregating through protein-protein interactions; to alter (often to extend) serum half-life; or to alter tissue localization or tissue exclusion and other ADME properties; to add other functional proteins or peptides.

For example, some fusion domains are particularly useful for isolation and/or purification of the fusion proteins, such as by affinity chromatography. Well known examples of such fusion domains that facilitate expression or purification include, merely to illustrate, affinity tags such as polyhistidine (e.g., a His6 tag), Strep II tag, streptavidin-binding peptide (SBP) tag, calmodulin-binding peptide (CBP), glutathione S-transferase (GST), maltose-binding protein (MBP), S-tag, HA tag, c-Myc tag, thioredoxin, protein A and protein G.

In some embodiments, wherein a binding agent (e.g., a binding protein, e.g., an antibody, an antibody fragment, or a stefin A protein variant) or the fusion protein to be secreted, it will generally contain a signal sequence that directs the transport of the protein to the lumen of the endoplasmic reticulum and ultimately to be secreted (or retained on the cell surface if a transmembrane domain or other cell surface retention signal). Signal sequences (also referred to as signal peptides or leader sequences) are located at the N-terminus of nascent polypeptides. They target the polypeptide to the endoplasmic reticulum and the proteins are sorted to their destinations, for example, to the inner space of an organelle, to an interior membrane, to the cell outer membrane, or to the cell exterior via secretion. Many signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence.

In some embodiments, the signal peptide is about 5 to about 40 amino acids in length (such as about 5 to about 7, about 7 to about 10, about 10 to about 15, about 15 to about 20, about 20 to about 25, or about 25 to about 30, about 30 to about 35, or about 35 to about 40 amino acids in length).

In some embodiments, the signal peptide is a native signal peptide from a human protein. In other embodiments, the signal peptide is a non-native signal peptide. For example, in some embodiments, the non-native signal peptide is a mutant native signal peptide from the corresponding native secreted human protein, and can include at least one (such as 2, 3, 4, 5, 6, 7, 8, 9, or 10 or more) substitution, insertions and/or deletions.

In some embodiments, the signal peptide is a signal peptide or mutant thereof from a non-IgSF protein family, such as a signal peptide from an immunoglobulin (such as IgG heavy chain or IgG-kappa light chain), a cytokine (such as interleukin-2 (IL-2), or CD33), a serum albumin protein (e.g. HSA or albumin), a human azurocidin preprotein signal sequence, a luciferase, a trypsinogen (e.g. chymotrypsinogen or trypsinogen) or other signal peptide able to efficiently secrete a protein from a cell. Exemplary signal peptides include but are not limited to.

Table 1 below lists examples of signal peptides that can be used for secretion of binding proteins (e.g., an antibody, an antibody fragment, or a stefin A protein variant) or fusion proteins thereof of the present disclosure, but is not limited thereto.

TABLE 1
Signal peptide sequences

| Native Protein | Signal Sequence | SEQ ID NO: |
|---|---|---|
| Human Serum Albumin | MKWVTFISLLFLFSSAYS | 9 |
| (HSA) | | |
| Ig kappa light chain | MDMRAPAGIFGFLLVLFPGYRS | 10 |
| Human azurocidin | MTRLTVLALLAGLLASSRA | 11 |
| preprotein | | |
| IgG heavy chain | MELGLSWIFLLAILKGVQC | 12 |
| IgG heavy chain | MELGLRWVFLVAILEGVQC | 13 |
| IgG heavy chain | MKHLWFFLLLVAAPRWVLS | 14 |
| IgG heavy chain | MDWTWRILFLVAAATGAHS | 15 |
| IgG heavy chain | MDWTWRFLFVVAAATGVQS | 16 |
| IgG heavy chain | MEFGLSWLFLVAILKGVQC | 17 |
| IgG heavy chain | MEFGLSWVFLVALFRGVQC | 18 |
| IgG heavy chain | MDLLHKNMKHLWFFLLLVAAPRWVLS | 19 |
| IgG Kappa light | MDMRVPAQLLGLLLLWLSGARC | 20 |
| IgG Kappa light | MKYLLPTAAAGLLLLAAQPAMA | 21 |
| Gaussia luciferase | MGVKVLFALICIAVAEA | 22 |
| Human | MAFLWLLSCWALLGTTFG | 23 |
| chymotrypsinogen | | |
| sHuman interleukin-2 | MQLLSCIALILALV | 24 |
| Human trypsinogen-2 | MNLLLILTFVAAAVA | 25 |
| Human CD33 | MPLLLLLPLLWAGALA | 26 |
| Prolactin | MDSKGSSQKGSRLLLLLVVSNLLLCQGVVS | 27 |
| Human tPA | MDAMKRGLCCVLLLCGAVFVSPS | 28 |
| Synthetic/Consensus | MLLLLLLLLLLALALA | 29 |
| Synthetic/Consensus | MWWRLWWLLLLLLLLWPMVWA | 30 |

In some embodiments, the fusion protein may further comprise at least one linker separating the binding agent (e.g., an antibody, an antibody fragment, or a stefin A protein variant) and/or the fusion domains. In some embodiments, the “linker” may inserted between a first polypeptide (e.g. binding protein (e.g., an antibody, an antibody fragment, or a stefin A protein variant)) and a second polypeptide (e.g. other binding protein or a fusion domain).

In some embodiments, one or more binding proteins (e.g., antibodies, antibody fragments, or stefin A protein variants) and one or more fusion domains can be directly linked without a linker.

Many natural linkers exhibited α-helical structures. The α-helical structure was rigid and stable, with intra-segment hydrogen bonds and a closely packed backbone. Therefore, the stiff α-helical linkers can act as rigid spacers between protein domains. George et al. (2002) “An analysis of protein domain linkers: their classification and role in protein folding” Protein Eng. 15(11):871-9. In general, rigid linkers exhibit relatively stiff structures by adopting α-helical structures or by containing multiple Pro residues. Under many circumstances, they separate the functional domains more efficiently than the flexible linkers. The length of the linkers can be easily adjusted by changing the copy number to achieve an optimal distance between domains. As a result, rigid linkers are chosen when the spatial separation of the domains is critical to preserve the stability or bioactivity of the fusion proteins. In this regard, alpha helix-forming linkers with the sequence of (EAAAK)n (SEQ ID NO: 37) have been applied to the construction of many recombinant fusion proteins. Another type of rigid linkers has a Pro-rich sequence, (XP)n, with X designating any amino acid, preferably Ala, Lys, or Glu.

Merely to illustrate, exemplary linkers include GRA, poly(Gly), poly(Ala) and those provided in Table 2.

TABLE 2
Linker sequences.

| Sequence | SEQ ID NO: |
|---|---|
| (GGGGS)ₙ (e.g., n = 1-6) | 31 |
| (Gly)₈ | 32 |
| (Gly)₆ | 33 |
| KESGSVSSEQLAQFRSLD | 34 |
| EGKSSGSGSESKST | 35 |
| GSAGSAAGSGEF | 36 |
| (EAAAK)ₙ (e.g., n = 1-6) | 37 |
| A(EAAAK)₄ALEA(EAAAK)₄A | 38 |
| PAPAP | 39 |
| AEAAAKEAAAKA | 40 |
| (Ala-Pro)n (10 to 34 aa) | 41 |
| GGSG | 42 |
| GSGS | 43 |
| GGGS | 44 |
| S(GGS)n (n = 1-7) | 45 |
| GGGSGGG | 46 |
| ESGGGGVT | 47 |
| LESGGGGVT | 48 |
| GRAQVT | 49 |
| WRAQVT | 50 |
| ARGRAQVT | 51 |
| SGTTSGTTRLLSGHTCFTLTGLLGTLVTMGLLT | 52 |
| SGTSPGLSAGATVGIMIGVLVGVALI | 53 |
| SAPVLSAVATVGITIGVLARVALI | 54 |
| SSPDLSAGTAVSIMIGVLAGMALI | 55 |
| TLGGNSASYTFVSLLFSAVTLLLLC | 56 |
| SGTSPGLSAGATVGIMIGVLVGVALI | 57 |

In some embodiments, besides the basic role in linking the fusion domains together, the linker may offer many other advantages for the production of fusion proteins, such as improving biological activity, increasing expression yield, and achieving desirable pharmacokinetic profiles.

In some embodiments, the linkers, preferably, should not adversely affect the expression, secretion, or activity of each domain. In some embodiments, the linkers, preferably, should not be antigenic and should not elicit an immune response.

In some embodiments, the fusion protein may include a fusion domain selected from the group consisting of an antigen-binding protein (domain), a cytokine, a half-life extension domain, a growth factor, an enzyme, and a cell-penetrating domain, but is not limited thereto. In some embodiments, the fusion protein may further include a therapeutic peptide or protein.

In some embodiments, the “therapeutic peptide or protein” refers to any peptide or protein having a preventive or therapeutic effect on a specific disease. In some embodiments, the therapeutic peptide or protein may be a stefin A protein variant (which may be the same as or different from a stefin A protein variant specifically binding to target protein). It includes, without limitation, peptides and proteins reported to have preventive or therapeutic effects on a specific disease in the art.

In some embodiments, the fusion protein may include a binding domain.

In some embodiments, when the fusion protein includes a binding domain, it may have multispecificity capable of binding to at least one additional target molecule.

In some embodiments, the binding domain may be selected from the group consisting of, for example, a stefin A protein variant (which may be the same as or different from a stefin A protein variant specifically binding to a target), an antibody or fragment thereof, an antibody-like material, an antigen-binding peptide, a ligand-binding site of a receptor (e.g. a receptor trap polypeptide), a receptor-binding ligand (e.g. a cytokine or a growth factor), an engineered T-cell receptor, and an enzyme or a catalytic fragment thereof, but is not limited thereto.

In some embodiments, examples of the binding domain may include, but are not limited to, adnectins/monobodies, affilins, affibodies, affitins, anticalin, atrimers, avimers, bicyclic peptides, C7 peptide, centyrin, carbohydrate-binding module (CBM), cys-knots, darpin, el-tandem, fynomers, knottin, Kunitz domains, O bodies, pronectin, scFv, Sac7d, Sso7d, Tn3, and the like.

In some embodiments, the fusion domain fused to the binding agent (e.g., an antibody, an antibody fragment, or a stefin A protein variant), may be the same or different type binding protein that specifically binds to another target.

In some embodiments, when the fusion domain is a stefin A protein variant specifically binding to a target, the two or more stefin A protein variants included in the fusion protein of the present disclosure may bind to the same or different sites of target. In some embodiments, the fusion protein may bind to two sites (biparatopic) or two or more sites (multiparatopic) of a target.

In some embodiments, the fusion domain may be an immune checkpoint protein, an immune costimulatory receptor, a receptor (or receptor agonist), a cytokine, a growth factor, or a tumor-associated antigen, but is not limited thereto.

In some embodiments, the cytokine is used as a generic term for secretory proteins that play an important role in signaling between cells. Examples of the cytokines may include, but are not limited to, chemokines, interferons, lymphokines, interleukins, tumor necrosis factors, and the like.

In some embodiments, the growth factor refers to a naturally-occurring material or a variant thereof that may stimulate cell proliferation, wound healing, and/or cell differentiation. Examples of the growth factors may include, but are not limited to, GH, EGF, VEGF, FGF, bFGF, HGF, BMPs, M-CSF, G-CSF, GM-CSF, EPO, GDNF, IGF, KGF, BDNF, NGF, PDGF, TPO, TGF, and the like.

In some embodiments, the enzyme is used as a generic term for proteins that catalyze a biological reaction. Examples of the enzyme may include, but are not limited to, α-chymotrypsin, lysozyme, urate oxidase, acetylcholinesterase, Thermomyces lanuginosus lipase, glucose oxidase, superoxie dismutase, caspase, β-glucosidase, Trametes versicolor laccase, alcohol oxidase, Cas9, Cas12, Cas13, Cas14, zinc-finger nuclease, TALLEN, dimethyl sulfoxide, uricase, agalsidase beta, agalsidase alfa, imiglucerase, taliglucerase alfa, velaglucerase alfa, alglucerase, sebelipase alpha, laronidase, idursulfase, elosulfase alpha, galsulfase, alglucosidase alpha, and the like.

In some embodiments, the cell-penetrating peptide is a short peptide that promotes cellular uptake and absorption of various molecules. In some embodiments, when the fusion protein includes a cell-penetrating peptide, the binding agent (e.g., stefin A protein variant) may be absorbed into the cell. Examples of the cell-penetrating peptide may include, but are not limited thereto, Tat, penetratin, transporant, Pept1, Pept 2, pVEC, DPV3, DPV6, R8, R9, MPG, MAP, Bip4, C105Y, melittin, and the like.

In some embodiments, a nucleic acid encoding the fusion protein is introduced into a genetically modified cell. the genetically modified cell may be may express a fusion protein and secrete extracellularly, and/or preferably may be anchored on a cell membrane and presented on the cell surface.

In some embodiments, the fusion protein is expressed in a genetically modified cell, and may be localized to a specific organ or location within the cell.

In some embodiments, the fusion protein is a secretory fusion protein and/or a membrane-anchored fusion protein.

In some embodiments, when the fusion protein is fixed to a cell membrane or is expressed on a cell surface, the fusion protein may further include a transmembrane domain. As used herein, the term “transmembrane domain” refers to a protein domain that spans the width of a cell membrane. In some embodiments, the transmembrane domain preferably has an alpha-helical structure, but is not limited thereto.

In some embodiments, the transmembrane domain may be a transmembrane domain derived from, for example, CD3, CD4, CD5, CD8, CD28, CD99, PDGFR, PTGFRN, etc. or a variant thereof, but is not limited thereto.

In some embodiments, the fusion protein may further include a hinge domain in addition to the transmembrane domain. As used herein, the term “hinge domain” refers to a series of amino acid sequences that exist between the extracellular domain and the transmembrane domain of a membrane-anchoring protein. In some embodiments, the hinge domain may be located between the binding agent (e.g., an antibody, an antibody fragment, or a stefin A protein variant) and the transmembrane domain.

In some embodiments, the hinge domain may be a hinge domain derived from, for example, CD3, CD4, CD5, CD8, CD28, CD99, immunoglobulin (e.g. IgG1, IgG4, IgD, etc.), PDGFR, PTGFRN, etc., or a variant thereof, but is not limited thereto.

In some embodiments, the fusion protein may further include a coiled coil domain. As used herein, the term “coiled coil domain” refers to a structural motif of a protein in which two to seven alpha helices are wound like a rope strand. Preferably, the coiled coil domain is configured such that two or three alpha helices are wound.

In some embodiments, the coiled coil domain may be a coiled coil domain derived from a leucine zipper, foldon, cardiac phospholamban, a water-soluble analogue of a membrane phospholamban, COMP (cartilage oligomeric matrix protein), thrombospondin 3, thrombospondin 4, or VASP (vasodilator-stimulated phosphoprotein), or a variant thereof, but is not limited thereto.

In some embodiments, the coiled coil domain may be located between the binding agent (e.g., an antibody, an antibody fragment, or a stefin A protein variant) and the transmembrane domain.

In some embodiments, the fusion protein may further include a virus-derived peptide or protein. In some embodiments, examples of the virus-derived peptide or protein may include, but are not limited to, syncytin-1, syncytin-2, VSVG (vesicular stomatitis virus glycoprotein), F and G proteins of Nipah virus, F and H proteins of measles virus, F and H proteins of Tupaia paramyxovirus, F and G proteins, F and H proteins, or F and HN proteins of paramyxovirus, F and G proteins of Hendra virus, F and G proteins of Henipavirus, F and H proteins of Morbillivirus, F and HN proteins of respirovirus, F and HN proteins of Sendai virus, F and HN proteins of rubulavirus, F and HN proteins of avulavirus, variants thereof, and combinations thereof.

In some embodiments, the fusion protein may further include an immunomodulatory domain or an intracellular signaling domain.

In some embodiments, the immunomodulatory domain or intracellular signaling domain is a domain located in the cytoplasmic direction of a membrane-anchoring protein, and indicates a site that activates or inhibits an immune response when a target antigen is bound to the extracellular domain.

In some embodiments, the immunomodulatory domain or intracellular signaling domain may be an immunomodulatory domain derived from CD3, CD28, CD40L, ICOS, OX40, 4-1BB, TNFR2, etc., but is not limited thereto.

In some embodiments, the fusion protein may be a chimeric antigen receptor (CAR). In some embodiments, when the fusion protein is a chimeric antigen receptor, the binding agent (e.g., an antibody, an antibody fragment, or a stefin A protein variant) may function as an extracellular binding domain.

In some embodiments, when the fusion protein is a chimeric antigen receptor, it may further include the above-described transmembrane domain, hinge domain, and intracellular signaling domain, but the present disclosure is not limited thereto. The fusion protein may be easily designed and prepared by changing the extracellular antigen-binding domain of various chimeric antigen receptors known in the art or analogues thereof to the binding agent (e.g., an antibody, an antibody fragment, or a stefin A protein variant).

In some embodiments, the fusion protein may further include a localization domain. In some embodiments, when the fusion protein is expressed intracellularly, it is preferable to further include a localization domain. As used herein, the term “localization domain” refers to a peptide or protein sequence that functions to localize a protein to a specific organ within a cell or a specific location within a cell. In some embodiments, the localization domain may be an organ-specific localization domain or an intracellular protein localization domain.

In some embodiments, the localization domain may be a nucleus-specific localization domain derived from VACM-1/CUL5, CXCR4, VP1, 53BP1, ING4, IER5, ERK5, Hrp1, UL79, EWS, PTHrP, Pho4, and rpL23a, a mitochondria-specific localization domain derived from ATP synthase Fib, cytochrome c oxidase polypeptide VIII, SOD2, citrate synthase, Tu translation elongation factor, etc., or a peroxisome localization domain derived from PTS1, PTS2, etc., but is not limited thereto.

In some embodiments, the fusion protein may further include a half-life extension domain. In some embodiments, the half-life extension domain is a domain or moiety that is fused to extend the half-life of the binding agent (e.g., an antibody, an antibody fragment, or a stefin A protein variant), and examples of the half-life extension domain may include, but are not limited to, an Fc domain, an Fc-binding protein or peptide, albumin (e.g. HSA), an albumin-binding protein or peptide, transferrin, transferrin-binding protein or peptide, etc.

Still other modifications that can be made to the binding agent (e.g., an antibody, an antibody fragment, or a stefin A protein variant) or the fusion protein sequence or to a flanking polypeptide moiety provided as part of a fusion protein is at least one sequence that is a site for post-translational modification by an enzyme. These can include, but are not limited to, glycosylation, acetylation, acylation, lipid-modification, palmitoylation, palmitate addition, phosphorylation, glycolipid-linkage modification, and the like.

### Multispecific Fusion Proteins

In some embodiments, the fusion protein is a multispecific polypeptide including, for example, a first binding protein (e.g., an antibody, an antibody fragment, or a stefin A protein variant that specifically bind to a first target) and at least one additional binding domain. The additional binding domain may be a polypeptide sequence selected from amongst, to illustrate, a second binding domain (e.g. a second stefin A protein variant (which may be the same or different than the first the stefin A protein variant)), an antibody or fragment thereof or other antigen binding polypeptide, a ligand binding portion of a receptor (such as a receptor trap polypeptide), a receptor-binding ligand (such as a cytokine, growth factor or the like), engineered T-cell receptor, an enzyme or catalytic fragment thereof.

In some embodiments, the fusion protein includes at least two binding proteins. The fusion protein can bind the same or overlapping sites on a target or can bind two different sites such that the fusion protein can simultaneously bind two sites on the same target protein (biparatopic) or more than two sites (multiparatopic). In some embodiments, the fusion protein includes at least one additional the stefin A protein variant sequence that is also directed to the same target. An additional the stefin A protein variant(s) specifically binding to the target may be the same or different (or a mixture thereof) as the first stefin A protein variant specifically binding to the target in order to create a multispecific fusion protein.

In some embodiments, the fusion protein includes at least one antigen binding site from an antibody. The resulting fusion protein can be a single chain including both the stefin A protein variant specifically binding to the target and the antigen binding site (such as in the case of an scFv) or can be a multimeric protein complex such as in antibody assembled with heavy and/or light chains to which the sequence of the antibody has also been fused.

In some embodiments, with respect to a multispecific fusion protein comprising a full-length immunoglobulin, the fusion of the AFFIMER® polypeptide sequence to the antibody will preserve the Fc function of the Fc region of the immunoglobulin. For example, the fusion protein may be capable of binding, via its Fc portion, to the Fc receptor of Fc receptor-positive cells. In some further embodiments, the fusion protein may activate the Fc receptor-positive cell by binding to the Fc receptor-positive cell, thereby initiating or increasing the expression of cytokines and/or co-stimulatory antigens. Furthermore, the AFFIMER® agent may transfer at least a second activation signal required for physiological activation of the T cell to the T cell via the co-stimulatory antigens and/or cytokines.

In some embodiments, resulted from the binding of its Fc portion to other cells that express Fc receptors present on the surface of effector cells from the immune system, such as immune cells, hepatocytes, and endothelial cells, the AFFIMER® agent may possess antibody-dependent cellular cytotoxicity (ADCC) function, a mechanism of cell-mediated immune defense whereby an effector cell of the immune system actively lyses a target cell, whose membrane-surface antigen has been bound by an antibody, and therefore, trigger tumor cell death via ADCC. In some further embodiments, the AFFIMER® agent is capable of demonstrating ADCC function.

As described above, apart from the Fc-mediated cytotoxicity, the Fc portion may contribute to maintaining the serum levels of the fusion protein, critical for its stability and persistence in the body. For example, when the Fc portion binds to Fc receptors on endothelial cells and on phagocytes, the fusion protein may become internalized and recycled back to the blood stream, enhancing its half-life within the body.

Exemplary targets of the additional binding domain (e.g., an antibody, an antibody fragment, or a stefin A protein variant) include but are not limited to, another immune checkpoint protein, and immune co-stimulatory receptor (particularly if the additional stefin A protein variant (s) can agonize the co-stimulatory receptor), a receptor, a cytokine, a growth factor, or a tumor-associated antigen, merely to illustrate. In some embodiments, the immunoglobulin portion may be a monoclonal antibody against at least one autoimmune target. In some embodiments, the target binding domain is part of a fusion protein that includes one or more binding domains that bind to a protein upregulated in autoimmune conditions.

See William B A et al. J. Clin. Med. 2019; 8(8): 1261 (incorporated by reference) for a review of stefin A protein variants and the fusion protein formats encompassed by the present disclosure.

In some embodiments, the multispecific fusion protein may further comprise a half-life extension moiety, such as any of those described herein. For example, the fusion protein may comprise at least one target binding domain (e.g., an antibody, an antibody fragment, or a stefin A protein variant) linked through a peptide linker to a binding domain specific for at least one immune cell (e.g., T cell and/or NK cell) binding domain (e.g., CD3ε chain or CD16) further linked to a half-life extension moiety, such as a fragment crystallizable (Fc) domain (e.g., an FcγR null-binding Fc), human serum albumin (HSA), or an stefin A protein variant specifically binding to HSA. In some embodiments, the half-life extension moiety is a fragment crystallizable (Fc) domain. In some embodiments, the half-life extension moiety is a human serum albumin (HSA). In some embodiments, the half-life extension moiety is a stefin A protein variant specifically binding to HSA.

### Engineering PK and ADME Properties

In some embodiment, the fusion protein may not have a half-life and/or PK profile that is optimal for the route of administration, such as parenteral therapeutic dosing. A “half-life” is the amount of time it takes for a substance, such as a fusion protein or binding protein (e.g., stefin A protein variant) of the present disclosure, to lose half of its pharmacologic or physiologic activity or concentration. Biological half-life can be affected by elimination, excretion, degradation (e.g., enzymatic) of the substance, or absorption and concentration in certain organs or tissues of the body. In some embodiments, biological half-life can be assessed by determining the time it takes for the blood plasma concentration of the substance to reach half its steady state level (“plasma half-life”). To address this shortcoming, there are a variety of general strategies for prolongation of half-life that have been used in the case of other protein therapeutics, including the incorporation of half-life extending moieties as part of the fusion protein.

The term “half-life extending moiety” refers to a pharmaceutically acceptable moiety, domain, or molecule covalently linked (chemically conjugated or fused) to a binding agent and/or binding protein (e.g., stefin A protein variant) to form an fusion protein described herein, optionally via a non-naturally encoded amino acid, directly or via a linker, that prevents or mitigates in vivo proteolytic degradation or other activity-diminishing modification of the binding agent (e.g., stefin A protein variant), increases half-life, and/or improves or alters other pharmacokinetic or biophysical properties including but not limited to increasing the rate of absorption, reducing toxicity, improving solubility, reducing protein aggregation, increasing biological activity and/or target selectivity of the modified binding protein (e.g., modified stefin A protein variant), increasing manufacturability, and/or reducing immunogenicity. For example, increasing manufacturability and/or reducing immunogenicity of a modified AFFIMER® polypeptide, compared to a comparator such as an unconjugated form of the modified AFFIMER© polypeptide. The term “half-life extending moiety” includes non-proteinaceous, half-life extending moieties, such as a water soluble polymer such as polyethylene glycol (PEG) or discrete PEG, hydroxyethyl starch (HES), a lipid, a branched or unbranched acyl group, a branched or unbranched C8-C30 acyl group, a branched or unbranched alkyl group, and a branched or unbranched C8-C30 alkyl group; and proteinaceous half-life extending moieties, such as serum albumin, transferrin, adnectins (e.g., albumin-binding or pharmacokinetics extending (PKE) adnectins), Fc domain, and unstructured polypeptide, such as XTEN and PAS polypeptide (e.g. conformationally disordered polypeptide sequences composed of the amino acids Pro, Ala, and/or Ser), and a fragment of any of the foregoing.

Half-life extending moieties that can be used in the production of the fusion proteins of the present disclosure are well known in the art and can be used by one skilled in the art to prepare fusion proteins using them without limitation.

In some embodiments, the half-life extending moiety extends the half-life of the resulting fusion protein circulating in mammalian blood serum compared to the half-life of the protein that is not so conjugated to the moiety (such as relative to the AFFIMER® polypeptide alone).

In some embodiments, half-life is extended by greater than or greater than about 1.2-fold, 1.5-fold, 2.0-fold, 3.0-fold, 4.0-fold., 5.0-fold, or 6.0-fold. In some embodiments, half-life is extended by more than 6 hours, more than 12 hours, more than 24 hours, more than 48 hours, more than 72 hours, more than 96 hours or more than 1 week after in vivo administration compared to the protein without the half-life extending moiety.

As means for further exemplification, half-life extending moieties that can be used in the generation of fusion protein of the disclosure include:
Genetic fusion of the binding agent sequence (e.g., AFFIMER® sequence) to a naturally long-half-life protein or protein domain (e.g., Fc fusion, transferrin [Tf] fusion, or albumin fusion. See, for example, Beck et al. (2011) “Therapeutic Fc-fusion proteins and peptides as successful alternatives to antibodies. MAbs. 3:1-2; Czajkowsky et al. (2012) “Fc-fusion proteins: new developments and future perspectives. EMBO Mol Med. 4:1015-28; Huang et al. (2009) “Receptor-Fc fusion therapeutics, traps, and Mimetibody technology” Curr Opin Biotechnol. 2009; 20:692-9; Keefe et al. (2013) “Transferrin fusion protein therapies: acetylcholine receptor-transferrin fusion protein as a model. In: Schmidt S, editor. Fusion protein technologies for biopharmaceuticals: applications and challenges. Hoboken: Wiley; p. 345-56; Weimer et al. (2013) “Recombinant albumin fusion proteins. In: Schmidt S, editor. Fusion protein technologies for biopharmaceuticals: applications and challenges. Hoboken: Wiley; 2013. p. 297-323; Walker et al. (2013) “Albumin-binding fusion proteins in the development of novel long-acting therapeutics. In: Schmidt S, editor. Fusion protein technologies for biopharmaceuticals: applications and challenges. Hoboken: Wiley; 2013. p. 325-43.
Genetic fusion of the binding agent sequence (e.g., sequence encoding a binding protein that specifically binds a target, e.g., AFFIMER® sequence) to an inert polypeptide, e.g., XTEN (also known as recombinant PEG or “rPEG”), a homoamino acid polymer (HAP; HAPylation), a proline-alanine-serine polymer (PAS; PASylation), or an elastin-like peptide (ELP; ELPylation). See, for example, Schellenberger et al. (2009) “A recombinant polypeptide extends the in vivo half-life of peptides and proteins in a tunable manner. Nat Biotechnol. 2009; 27:1186-90; Schlapschy et al. Fusion of a recombinant antibody fragment with a homo-amino-acid polymer: effects on biophysical properties and prolonged plasma half-life. Protein Eng Des Sel. 2007; 20:273-84; Schlapschy (2013) PASylation: a biological alternative to PEGylation for extending the plasma half-life of pharmaceutically active proteins. Protein Eng Des Sel. 26:489-501. Floss et al. (2012) “Elastin-like polypeptides revolutionize recombinant protein expression and their biomedical application. Trends Biotechnol. 28:37-45. Floss et al. “ELP-fusion technology for biopharmaceuticals. In: Schmidt S, editor. Fusion protein technologies for biopharmaceuticals: application and challenges. Hoboken: Wiley; 2013. p. 372-98.
Increasing the hydrodynamic radius by chemical conjugation of the pharmacologically active peptide or protein to repeat chemical moieties, e.g., to PEG (PEGylation) or hyaluronic acid. See, for example, Caliceti et al. (2003) “Pharmacokinetic and biodistribution properties of poly(ethylene glycol)-protein conjugates” Adv Drug Delivery Rev. 55:1261-77; Jevsevar et al. (2010) PEGylation of therapeutic proteins. Biotechnol J 5:113-28; Kontermann (2009) “Strategies to extend plasma half-lives of recombinant antibodies” BioDrugs. 23:93-109; Kang et al. (2009) “Emerging PEGylated drugs” Expert Opin Emerg Drugs. 14:363-80; and Mero et al. (2013) “Conjugation of hyaluronan to proteins” Carb Polymers. 92:2163-70.
Significantly increasing the negative charge of fusing the pharmacologically active peptide or protein by polysialylation; or, alternatively, (b) fusing a negatively charged, highly sialylated peptide (e.g., carboxy-terminal peptide [CTP; of chorionic gonadotropin (CG) b-chain]), known to extend the half-life of natural proteins such as human CG b-subunit, to the biological drug candidate. See, for example, Gregoriadis et al. (2005) “Improving the therapeutic efficacy of peptides and proteins: a role for polysialic acids” Int J Pharm. 2005; 300:125-30; Duijkers et al. “Single dose pharmacokinetics and effects on follicular growth and serum hormones of a long-acting recombinant FSH preparation (FSHCTP) in healthy pituitary-suppressed females” (2002) Hum Reprod. 17:1987-93; and Fares et al. “Design of a longacting follitropin agonist by fusing the C-terminal sequence of the chorionic gonadotropin beta subunit to the follitropin beta subunit” (1992) Proc Natl Acad Sci USA. 89:4304-8. 35; and Fares “Half-life extension through O-glycosylation.
Binding non-covalently, via attachment of a peptide or protein-binding domain to the bioactive protein, to normally long-half-life proteins such as HSA, human IgG, transferrin or fibronectin. See, for example, Andersen et al. (2011) “Extending half-life by indirect targeting of the neonatal Fc receptor (FcRn) using a minimal albumin binding domain” J Biol Chem. 286:5234-41; O'Connor-Semmes et al. (2014) “GSK2374697, a novel albumin-binding domain antibody (albudAb), extends systemic exposure of extendin-4: first study in humans-PK/PD and safety” Clin Pharmacol Ther. 2014; 96:704-12. Sockolosky et al. (2014) “Fusion of a short peptide that binds immunoglobulin G to a recombinant protein substantially increases its plasma half-life in mice” PLoS One. 2014; 9:e102566.

Classical genetic fusions to long-lived serum proteins offer an alternative method of half-life extension distinct from chemical conjugation to PEG or lipids. Two major proteins have traditionally been used as fusion partners: antibody Fc domains and human serum albumin (HSA). Fc fusions involve the fusion of peptides, proteins or receptor exodomains to the Fc portion of an antibody. Both Fc and albumin fusions achieve extended half-lives not only by increasing the size of the peptide drug, but both also take advantage of the body's natural recycling mechanism: the neonatal Fc receptor, FcRn. The pH-dependent binding of these proteins to FcRn prevents degradation of the fusion protein in the endosome. Fusions based on these proteins can have half-lives in the range of 3-16 days, much longer than typical PEGylated or lipidated peptides. Fusion to antibody Fc domains can improve the solubility and stability of the peptide or protein drug. An example of a peptide Fc fusion is dulaglutide, a GLP-1 receptor agonist currently in late-stage clinical trials. Human serum albumin, the same protein exploited by the fatty acylated peptides is the other popular fusion partner. Albiglutide is a GLP-1 receptor agonist based on this platform. A major difference between Fc and albumin is the dimeric nature of Fc versus the monomeric structure of HSA leading to presentation of a fused peptide as a dimer or a monomer depending on the choice of fusion partner. The dimeric nature of an antibody and/or AFFIMER®-Fc fusion can produce an avidity effect if the antibody and/or AFFIMER® targets are spaced closely enough together or are themselves dimers. This may be desirable or not depending on the target.

### Fc Fusions

The fusion protein may include an immunoglobulin Fc domain (an Fc domain) or a fragment or variant thereof, for example, a functional Fc region. In some embodiments, the Fc region is a FcγR null-binding Fc region. In some embodiments, the fusion protein may comprise at least one target binding domain (e.g., an antibody, an antibody fragment, or a stefin A protein variant) covalently linked through a peptide backbone (directly or indirectly) to an Fc region of an immunoglobulin. In some embodiments, the fusion protein may comprise the Fc region of an antibody (which facilitates effector functions and pharmacokinetics) and the target binding protein (e.g., an antibody, an antibody fragment, or a stefin A protein variant) as part of the same polypeptide. An immunoglobulin Fc region may also be linked indirectly to the target binding domain. Various linkers are known in the art for use in the fusion proteins of the disclosure. In some embodiments, the fusion protein comprising Fc domain may be used as dimer, and may be used as a homodimer or a heterodimer.

In some embodiments, various Fc domain sequences that can be used for Fc domain fusion and functionality thereof are well known in the art, and a person skilled in the art can selects an appropriate Fc domain according to the purpose and fuses it to the binding agent (e.g., stefin A protein variant) of the present disclosure.

In some embodiments, the binding agent (e.g., an antibody, an antibody fragment, or a stefin A protein variant) may be part of a fusion protein with an immunoglobulin Fc domain (“Fc domain”), or a fragment or variant thereof, such as a functional Fc region. In some embodiments, the Fc region is a FcγR null-binding Fc region. In some embodiments, an Fc fusion (“Fc-fusion”), such as a Fc fusion protein, is a polypeptide comprising at least one binding protein sequence covalently linked through a peptide backbone (directly or indirectly) to an Fc region of an immunoglobulin. An Fc-fusion may comprise, for example, the Fc region of an antibody (which facilitates effector functions and pharmacokinetics) and a binding protein sequence as part of the same polypeptide. An immunoglobulin Fc region may also be linked indirectly to at least one binding protein. Various linkers are known in the art and can optionally be used to link an Fe to a polypeptide including a binding protein sequence to generate an Fc-fusion. In some embodiments, Fc-fusions can be dimerized to form Fc-fusion homodimers, or using non-identical Fe domains, to form Fc-fusion heterodimers.

In some embodiments, an Fc-fusion homodimer comprises a dimer of a fusion protein that comprises a stefin A protein variant linked to an Fc domain linked to stefin A protein variant (AFFIMER® polypeptide—Fc domain—AFFIMER® polypeptide).

There are several reasons for choosing the Fc region of human antibodies for use in generating fusion protein. The principle rationale is to produce a stable protein, large enough to demonstrate a similar pharmacokinetic profile compared with those of antibodies, and to take advantage of the properties imparted by the Fc region; this includes the salvage neonatal FcRn receptor pathway involving FcRn-mediated recycling of the fusion protein to the cell surface post endocytosis, avoiding lysosomal degradation and resulting in release back into the bloodstream, thus contributing to an extended serum half-life. Another obvious advantage is the Fc domain's binding to Protein A, which can simplify downstream processing during production of the fusion protein and permit generation of highly pure preparation of the fusion protein.

In general, an Fc domain will include the constant region of an antibody excluding the first constant region immunoglobulin domain. Thus, Fc domain refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cγ2 and Cγ3 and the hinge between Cγ1 and Cγ2. Although the boundaries of the Fc domain may vary, the human IgG heavy chain Fc region is usually defined to comprise residues C226 or P230 to its carboxyl-terminus, wherein the numbering is according to the EU index as set forth in Kabat (Kabat et al., Sequences of Proteins of Immunological Interest, 5th Ed. Public Health Service, NIH, Bethesda, Md. (1991)). Fc may refer to this region in isolation, or this region in the context of a whole antibody, antibody fragment, or Fc fusion protein. Polymorphisms have been observed at a number of different Fc positions and are also included as Fc domains as used herein.

In some embodiments, the Fc As used herein, a “functional Fc region” refers to an Fc domain or fragment thereof which retains the ability to bind FcRn. A functional Fc region binds to FcRn but does not possess effector function. The ability of the Fc region or fragment thereof to bind to FcRn can be determined by standard binding assays known in the art. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions can be assessed using various assays known in the art for evaluating such antibody effector functions.

In an exemplary embodiment, the Fc domain is derived from an IgG1 subclass, however, other subclasses (e.g., IgG2, IgG3, and IgG4) may also be used. An exemplary sequence of a human IgG1 immunoglobulin Fc domain which can be used is:

(SEQ ID NO: 58)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPGK

In some embodiments, the Fc region used in the fusion protein may comprise the hinge region of an Fc molecule. An exemplary hinge region comprises the core hinge residues spanning positions 1-16 (e.g., DKTHTCPPCPAPELLG ((SEQ ID NO: 59)) of the exemplary human IgG1 immunoglobulin Fc domain sequence provided above. In some embodiments, the AFFIMER*-containing fusion protein may adopt a multimeric structure (e.g., dimer) owing, in part, to the cysteine residues at positions 6 and 9 within the hinge region of the exemplary human IgG1 immunoglobulin Fc domain sequence provided above. In other embodiments, the hinge region as used herein, may further include residues derived from the CH1 and CH2 regions that flank the core hinge sequence of the exemplary human IgG1 immunoglobulin Fc domain sequence provided above. In yet other embodiments, the hinge sequence may comprise or consist of GSTHTCPPCPAPELLG (SEQ ID NO: 60) or EPKSCDKTHTCPPCPAPELLG (SEQ ID NO: 61).

In some embodiments, the hinge sequence may include at least one substitution that confer desirable pharmacokinetic, biophysical, and/or biological properties. Some exemplary hinge sequences include:

(SEQ ID NO: 62)
EPKSCDKTHTCPPCPAPELLGGPS;
(SEQ ID NO: 63)
EPKSSDKTHTCPPCPAPELLGGPS;
(SEQ ID NO: 64)
EPKSSDKTHTCPPCPAPELLGGSS;
(SEQ ID NO: 65)
EPKSSGSTHTCPPCPAPELLGGSS;
(SEQ ID NO: 66)
DKTHTCPPCPAPELLGGPS;
and
(SEQ ID NO: 67)
DKTHTCPPCPAPELLGGSS.

In some embodiments, the residue P at position 18 of the exemplary human IgG1 immunoglobulin Fc domain sequence provided above may be replaced with S to ablate Fc effector function; this replacement is exemplified in hinges having the sequences

(SEQ ID NO: 68)
EPKSSDKTHTCPPCPAPELLGGSS,
(SEQ ID NO: 69)
EPKSSGSTHTCPPCPAPELLGGSS,
and
(SEQ ID NO: 70)
DKTHTCPPCPAPELLGGSS.

In another embodiment, the residues DK at positions 1-2 of the exemplary human IgG1 immunoglobulin Fc domain sequence provided above may be replaced with GS to remove a potential clip site; this replacement is exemplified in the sequence EPKSSGSTHTCPPCPAPELLGGSS (SEQ ID NO: 65). In another embodiment, the C at the position 103 of the heavy chain constant region of human IgG1 (e.g., domains CH1-CH3), may be replaced with S to prevent improper cysteine bond formation in the absence of a light chain; this replacement is exemplified by EPKSSDKTHTCPPCPAPELLGGPS (SEQ ID NO: 446), EPKSSDKTHTCPPCPAPELLGGSS (SEQ ID NO: 68), and EPKSSGSTHTCPPCPAPELLGGSS (SEQ ID NO: 69).

In some embodiments, the Fc is a mammalian Fc such as a human Fc, including Fc domains derived from IgG1, IgG2, IgG3 or IgG4. The Fc region may possess at least about 80%, 85%, 90%, 95%, 96%, 97%, 98%, or 99% sequence identity with a native Fc region and/or with an Fc region of a parent polypeptide. In some embodiments, the Fc region may have at least about 90% sequence identity with a native Fc region and/or with an Fc region of a parent polypeptide.

In some embodiments, the Fc domain comprises an amino acid sequence selected from SEQ ID NOs: 71-84 or an Fc sequence from the examples provided by SEQ ID NOs: 71-84. It should be understood that the C-terminal lysine of an Fc domain is an optional component of a fusion protein comprising an Fc domain. In some embodiments, the Fc domain comprises an amino acid sequence selected from SEQ ID NOs: 71-84, except that the C-terminal lysine thereof is omitted. In some embodiments, the Fc domain comprises the amino acid sequence selected from SEQ ID NOs: 71-84. In some embodiments, the Fc domain comprises the amino acid sequence selected from SEQ ID NOs: 71-84 except the C-terminal lysine thereof is omitted.

TABLE 3
Fc domain sequences.

| | | SEQ ID |
|---|---|---|
| Name | Sequence | NO: |
| hIgGla_191 | DKTHTCPPCPAPELLGGPSVFLFPPKPKDTLMISRTPEVTCVV | 71 |
| [A subtype] | VDVSHEDPEVKFNWYVDGVEVHNAKTKPREEQYNSTYRVVSVL | |
| | TVLHQDWLNGKEYKCKVSNKALPAPIEKTISKAKGQPREPQVY | |
| | TLPPSRDELTKNQVSLTCLVKGFYPSDIAVEWESNGQPENNYK | |
| | TTPPVLDSDGSFFLYSKLTVDKSRWQQGNVFSCSVMHEALHNH | |
| | YTQKSLSLSPGK | |
| hIgG1a_189 [hIgG1a_191 | DKTHTCPPCPAPELLGGPSVFLFPPKPKDTLMISRTPEVTCVV | 72 |
| sans ″GK″ on C term; A | VDVSHEDPEVKFNWYVDGVEVHNAKTKPREEQYNSTYRVVSVL | |
| subtype] | TVLHQDWLNGKEYKCKVSNKALPAPIEKTISKAKGQPREPQVY | |
| | TLPPSRDELTKNQVSLTCLVKGFYPSDIAVEWESNGQPENNYK | |
| | TTPPVLDSDGSFFLYSKLTVDKSRWQQGNVFSCSVMHEALHNH | |
| | YTQKSLSLSE | |
| hIgG1a_191b | DKTHTCPPCPAPELLGGPSVFLFPPKPKDTLMISRTPEVTCVV | 73 |
| [A/F subtype] | VDVSHEDPEVKFNWYVDGVEVHNAKTKPREEQYNSTYRVVSVL | |
| | TVLHQDWLNGKEYKCKVSNKALPAPIEKTISKAKGQPREPQVY | |
| | TLPPSRDEMTKNQVSLTCLVKGFYPSDIAVEWESNGQPENNYK | |
| | TTPPVLDSDGSFFLYSKLTVDKSRWQQGNVFSCSVMHEALHNH | |
| | YTQKSLSLSPGK | |
| hIgG1f_1.1_191 | DKTHTCPPCPAPEAEGAPSVFLFPPKPKDTLMISRTPEVTCVV | 74 |
| [Contains five point- | VDVSHEDPEVKFNWYVDGVEVHNAKTKPREEQYNSTYRVVSVL | |
| mutations to alter ADCC | TVLHQDWLNGKEYKCKVSNKALPSSIEKTISKAKGQPREPQVY | |
| function, F subtype] | TLPPSREEMTKNQVSLTCLVKGFYPSDIAVEWESNGQPENNYK | |
| | TTPPVLDSDGSFFLYSKLTVDKSRWQQGNVFSCSVMHEALHNH | |
| | YTQKSLSLSPGK | |
| hIgG1f_1.1_186 | EPKSSDKTHTCPPCPAPEAEGAPSVFLFPPKPKDTLMISRTPE | 75 |
| [Contains five point- | VTCVVVDVSHEDPEVKFNWYVDGVEVHNAKTKPREEQYNSTYR | |
| mutations to alter ADCC | VVSVLTVLHQDWLNGKEYKCKVSNKALPSSIEKTISKAKGQPR | |
| function and C225S | EPQVYTLPPSREEMTKNQVSLTCLVKGFYPSDIAVEWESNGQP | |
| (Edlemen numbering); F | ENNYKTTPPVLDSDGSFFLYSKLTVDKSRWQQGNVFSCSVMHE | |
| subtype] | ALHNHYTQKSLSLSPGK | |
| hIgG1a_(N297G)_191 | DKTHTCPPCPAPELLGGPSVFLFPPKPKDTLMISRTPEVTCVV | 76 |
| [A subtype] | VDVSHEDPEVKFNWYVDGVEVHNAKTKPREEQYGSTYRVVSVL | |
| | TVLHQDWLNGKEYKCKVSNKALPAPIEKTISKAKGQPREPQVY | |
| | TLPPSRDELTKNQVSLTCLVKGFYPSDIAVEWESNGQPENNYK | |
| | TTPPVLDSDGSFFLYSKLTVDKSRWQQGNVFSCSVMHEALHNH | |
| | YTQKSLSLSPGK | |
| hIgG1a_190 | DKTHTCPPCPAPELLGGPSVFLFPPKPKDTLMISRTPEVTCVV | 77 |
| [hIgG1a_190 sans ″K″ on C | VDVSHEDPEVKFNWYVDGVEVHNAKTKPREEQYNSTYRVVSVL | |
| term; A subtype] | TVLHQDWLNGKEYKCKVSNKALPAPIEKTISKAKGQPREPQVY | |
| | TLPPSRDELTKNQVSLTCLVKGFYPSDIAVEWESNGQPENNYK | |
| | TTPPVLDSDGSFFLYSKLTVDKSRWQQGNVFSCSVMHEALHNH | |
| | YTQKSLSLSPG | |
| hIgG1a_(N297Q)_191 | DKTHTCPPCPAPELLGGPSVFLFPPKPKDTLMISRTPEVTCVV | 78 |
| [A subtype] | VDVSHEDPEVKFNWYVDGVEVHNAKTKPREEQYQSTYRVVSVL | |
| | TVLHQDWLNGKEYKCKVSNKALPAPIEKTISKAKGQPREPQVY | |
| | TLPPSRDELTKNQVSLTCLVKGFYPSDIAVEWESNGQPENNYK | |
| | TTPPVLDSDGSFFLYSKLTVDKSRWQQGNVFSCSVMHEALHNH | |
| | YTQKSLSLSPGK | |
| hIgG1a_(N297S)_191 | DKTHTCPPCPAPELLGGPSVFLFPPKPKDTLMISRTPEVTCVV | 79 |
| [A subtype] | VDVSHEDPEVKFNWYVDGVEVHNAKTKPREEQYSSTYRVVSVL | |
| | TVLHQDWLNGKEYKCKVSNKALPAPIEKTISKAKGQPREPQVY | |
| | TLPPSRDELTKNQVSLTCLVKGFYPSDIAVEWESNGQPENNYK | |
| | TTPPVLDSDGSFFLYSKLTVDKSRWQQGNVFSCSVMHEALHNH | |
| | YTQKSLSLSPGK | |
| hIgG1a_(N297A)_191 | DKTHTCPPCPAPELLGGPSVFLFPPKPKDTLMISRTPEVTCVV | 80 |
| [A subtype] | VDVSHEDPEVKFNWYVDGVEVHNAKTKPREEQYASTYRVVSVL | |
| | TVLHQDWLNGKEYKCKVSNKALPAPIEKTISKAKGQPREPQVY | |
| | TLPPSRDELTKNQVSLTCLVKGFYPSDIAVEWESNGQPENNYK | |
| | TTPPVLDSDGSFFLYSKLTVDKSRWQQGNVFSCSVMHEALHNH | |
| | YTQKSLSLSPGK | |
| hIgG1a_(N297H)_191 | DKTHTCPPCPAPELLGGPSVFLFPPKPKDTLMISRTPEVTCVV | 81 |
| [A subtype] | VDVSHEDPEVKFNWYVDGVEVHNAKTKPREEQYHSTYRVVSVL | |
| | TVLHQDWLNGKEYKCKVSNKALPAPIEKTISKAKGQPREPQVY | |
| | TLPPSRDELTKNQVSLTCLVKGFYPSDIAVEWESNGQPENNYK | |
| | TTPPVLDSDGSFFLYSKLTVDKSRWQQGNVFSCSVMHEALHNH | |
| | YTQKSLSLSPGK | |
| hIgG4 | DKRVESKYGPPCPSCPAPEFLGGPSVFLFPPKPKDTLMISRTP | 82 |
| | EVTCVVVDVSQEDPEVQFNWYVDGVEVHNAKTKPREEQENSTY | |
| | RVVSVLTVLHQDWLNGKEYKCKVSNKGLPSSIEKTISKAKGQP | |
| | REPQVYTLPPSQEEMTKNQVSLTCLVKGFYPSDIAVEWESNGQ | |
| | PENNYKTTPPVLDSDGSFFLYSRLTVDKSRWQEGNVFSCSVMH | |
| | EALHNHYTQKSLSLSLGK | |
| hIgG4_(S241P) | DKRVESKYGPPCPPCPAPEFLGGPSVFLFPPKPKDTLMISRTP | 83 |
| | EVTCVVVDVSQEDPEVQFNWYVDGVEVHNAKTKPREEQFNSTY | |
| | RVVSVLTVLHQDWLNGKEYKCKVSNKGLPSSIEKTISKAKGQP | |
| | REPQVYTLPPSQEEMTKNQVSLTCLVKGFYPSDIAVEWESNGQ | |
| | PENNYKTTPPVLDSDGSFFLYSRLTVDKSRWQEGNVFSCSVMH | |
| | EALHNHYTQKSLSLSLGK | |
| hIgG1 (Contain two point- | SEPKSCDKTHTCPPCPAPEAAGGPSVFLFPPKPKDTLMISRTP | 84 |
| mutations to alter ADCC | EVTCVVVDVSHEDPEVKFNWYVDGVEVHNAKTKPREEQYNSTY | |
| function L20A, L21A) | RVVSVLTVLHQDWLNGKEYKCKVSNKALPAPIEKTISKAKGQP | |
| | REPQVYTLPPSREEMTKNQVSLTCLVKGFYPSDIAVEWESNGQ | |
| | PENNYKTTPPVLDSDGSFFLYSKLTVDKSRWQQGNVFSCSVMH | |
| | EALHNHYTQKSLSLSPGK | |

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enables these cytotoxic effector cells to bind specifically to an antigen-bearing target cell and subsequently kill the target cell with cytotoxins.

In some embodiments, the fusion protein includes an Fc domain sequence for which the resulting fusion protein has no (or reduced) ADCC and/or complement activation or effector functionality. For example, the Fc domain may comprise a naturally disabled constant region of IgG2 or IgG4 isotype or a mutated IgG1 constant region. Examples of suitable modifications are described in EP0307434. One example comprises the substitutions of alanine residues at positions 235 and 237 (EU index numbering).

In other embodiments, the fusion protein includes an Fc domain sequence for which the resulting fusion protein will retain some or all Fc functionality for example will be capable of one or both of ADCC and CDC activity, as for example if the fusion protein comprises the Fc domain from human IgG1 or IgG3. Levels of effector function can be varied according to known techniques, for example by mutations in the CH2 domain, for example wherein the IgG1 CH2 domain has at least one mutation at positions selected from 239 and 332 and 330, for example the mutations are selected from S239D and I332E and A330L such that the antibody has enhanced effector function, and/or for example altering the glycosylation profile of the antigen-binding protein of the disclosure such that there is a reduction in fucosylation of the Fc region.

### Albumin Fusions

In some embodiments, the fusion protein is a fusion protein comprising, in addition to at least one binding protein (e.g., an antibody, an antibody fragment, or a stefin A protein variant), an albumin sequence or an albumin fragment. In some embodiments, a binding protein (e.g., an antibody, an antibody fragment, or a stefin A protein variant) is conjugated to the albumin sequence or an albumin fragment through chemical linkage other than incorporation into the polypeptide sequence including the binding agent (e.g., an antibody, an antibody fragment, or a stefin A protein variant). In some embodiments, the albumin, albumin variant, or albumin fragment is human serum albumin (HSA), a human serum albumin variant, or a human serum albumin fragment. Albumin serum proteins comparable to HSA are found in, for example, cynomolgus monkeys, cows, dogs, rabbits and rats. Of the non-human species, bovine serum albumin (BSA) is the most structurally similar to HSA. See, e.g., Kosa et al., (2007) J Pharm Sci. 96(11):3117-24. The present disclosure contemplates the use of albumin from non-human species, including, but not limited to, albumin sequence derived from cyno serum albumin or bovine serum albumin.

Mature HSA, a 585 amino acid polypeptide (approx. 67 kDa) having a serum half-life of about 20 days, is primarily responsible for the maintenance of colloidal osmotic blood pressure, blood pH, and transport and distribution of numerous endogenous and exogenous ligands. The protein has three structurally homologous domains (domains I, II and III), is almost entirely in the alpha-helical conformation, and is highly stabilized by 17 disulfide bridges. In some embodiments, the fusion protein can be an albumin fusion protein including at least one stefin A protein variant sequence and the sequence for mature human serum albumin or a variant or fragment thereof which maintains the PK and/or biodistribution properties of mature albumin to the extent desired in the fusion protein.

The albumin sequence can be set off from the binding agent sequence (e.g., stefin A protein variant sequence) or other flanking sequences in the fusion protein by use of linker sequences as described above.

While unless otherwise indicated, reference herein to “albumin” or to “mature albumin” is meant to refer to HSA. However, it is noted that full-length HSA has a signal peptide of 18 amino acids (MKWVTFISLLFLFSSAYS (SEQ ID NO: 85) followed by a pro-domain of 6 amino acids (RGVFRR) (SEQ ID NO: 86); these 24 amino acid residue peptide may be referred to as the pre-pro domain. The AFFIMER®-HSA fusion proteins can be expressed and secreted using the HSA pre-pro-domain in the recombinant proteins coding sequence. Alternatively, the AFFIMER®-HSA fusion can be expressed and secreted through inclusion of other secretion signal sequences, such as described above.

In alternative embodiments, rather than provided as part of a fusion protein with the stefin A protein variant, the serum albumin polypeptide can be covalently coupled to the stefin A protein variant containing polypeptide through a bond other than a backbone amide bond, such as cross-linked through chemical conjugation between amino acid sidechains on each of the albumin polypeptide and the stefin A protein variant-containing polypeptide.

In some embodiments, a chemical modification method that can be applied in the generation of the subject a binding agent (e.g., a binding protein, e.g., an antibody, antibody fragment, or stefin A protein variant that specifically bind a target or fusion protein thereof) to increase protein half-life is lipidation, which involves the covalent binding of fatty acids to peptide side chains. Originally conceived of and developed as a method for extending the half-life of insulin, lipidation shares the same basic mechanism of half-life extension as PEGylation, namely increasing the hydrodynamic radius to reduce renal filtration. However, the lipid moiety is itself relatively small and the effect is mediated indirectly through the non-covalent binding of the lipid moiety to circulating albumin. One consequence of lipidation is that it reduces the water-solubility of the peptide but engineering of the linker between the peptide and the fatty acid can modulate this, for example by the use of glutamate or mini PEGs within the linker. Linker engineering and variation of the lipid moiety can affect self-aggregation which can contribute to increased half-life by slowing down biodistribution, independent of albumin. See, for example, Jonassen et al. (2012) Pharm Res. 29(8):2104-14.

Other examples of albumin binding moieties for use in the generation of certain fusion protein include albumin-binding (PKE2) adnectins (See WO2011140086 “Serum Albumin Binding Molecules”, WO2015143199 “Serum albumin-binding Fibronectin Type III Domains” and WO2017053617 “Fast-off rate serum albumin binding fibronectin type iii domains”), the albumin binding domain 3 (ABD3) of protein G of Streptococcus strain G148, and the albumin binding domain antibody GSK2374697 (“AlbudAb”) or albumin binding nanobody portion of ATN-103 (Ozoralizumab).

### AFFIMER® XT

In some embodiments, the molecule that binds a serum protein such as HSA comprises a stefin A protein variant specifically binding to HSA. Examples of such stefin A protein variant specifically binding to HSA can be found in WO2022/023540. The stefin A protein variant specifically binding to HSA provided herein, in some embodiments, is linked to another molecule and extend the half-life of that molecule (e.g., a therapeutic polypeptide). These stefin A protein variant specifically binding to HSA have been shown in in vivo pharmacokinetic (PK) studies to extend, in a controlled manner, the serum half-life of any other stefin A protein variant therapeutic to which it is conjugated in a single genetic fusion, for example, that can be made in E. coli. AFFIMER® XT™ polypeptides can also be used to extend the half-life of other peptide or protein therapeutics, such as the stefin A protein variant specifically binding to a target of the present disclosure.

In some embodiments, a stefin A protein variant specifically binding to HSA extends the serum half-life of the target AFFIMER® polypeptide in vivo. For example, a stefin A protein variant specifically binding to HSA may extend the half-life of the target AFFIMER® polypeptide by at least 2-fold, relative to the half-life of the molecule not linked to a stefin A protein variant specifically binding to HSA. In some embodiments, a stefin A protein variant specifically binding to HSA extends the half-life of the stefin A protein variant specifically binding to target by at least 3-fold, at least 4-fold, at least 5-fold, at least 6-fold, at least 7-fold, at least 8-fold, at least 9-fold, at least 10-fold, at least 20-fold, or at least 30-fold, relative to the half-life of the stefin A protein variant specifically binding to target not linked to an stefin A protein variant specifically binding to HSA. In some embodiments, an stefin A protein variant specifically binding to HSA extends the half-life of the stefin A protein variant specifically binding to target by 2-fold to 5-fold, 2-fold to 10-fold, 3-fold to 5-fold, 3-fold to 10-fold, 15-fold to 5-fold, 4-fold to 10-fold, or 5-fold to 10-fold, relative to the half-life of the stefin A protein variant specifically binding to target not linked to an stefin A protein variant specifically binding to HSA. In some embodiments, a stefin A protein variant specifically binding to HSA extends the half-life of the stefin A protein variant specifically binding to target by at least 6 hours, at least 12 hours, at least 24 hours, at least 48 hours, at least 72 hours, at least 96 hours, for example, at least 1 week after in vivo administration, relative to the half-life of the molecule not linked to a stefin A protein variant specifically binding to HSA.

A stefin A protein variant specifically binding to HSA comprises an AFFIMER® polypeptide in which at least one of the solvent accessible loops is from the wild-type stefin A protein having amino acid sequences to enable an AFFIMER® polypeptide to bind HSA, selectively, and in some embodiments, with a Kd of 10⁻⁶M or less.

In some embodiments, the stefin A protein variant specifically binding to HSA is derived from the wild-type human stefin A protein having a backbone sequence and in which one or both of loop 2 (designated (Xaa)n) and loop 4 (designated (Xaa)m) are replaced with alternative loop sequences (Xaa)n and (Xaa)m, to have the general Formula (I):

FR1-(Xaa)n-FR2-(Xaa)m-FR3 (I),
wherein FR1 is an amino acid sequence having at least 70% (e.g., at least 75%, at least 80%, at least 85%, at least 90%, at least 95%, or 100%) identity to MIPGGLSEAK PATPEIQEIV DKVKPQLEEK TNETYGKLEA VQYKTQVLA (SEQ ID NO: 6); FR2 is an amino acid sequence having at least 70% (e.g., at least 75%, at least 80%, at least 85%, at least 90%, at least 95%, or 100%) identity to GTNYYIKVRA GDNKYMHLKV FKSL (SEQ ID NO: 2); FR3 is an amino acid sequence having at least 70% (e.g., at least 75%, at least 80%, at least 85%, at least 90%, at least 95%, or 100%) identity to EDLVLTGYQV DKNKDDELTG F (SEQ ID NO: 3); Xaa, individually for each occurrence, is an amino acid; and n is an integer from 3 to 20, and m is an integer from 3 to 20. Additional variations of this general structure can be found in WO 2022/023540.

In all embodiments, each amino acid of (Xaa)n may be the same or selected from any amino acid. The same applies for (Xaa)m.

In some embodiments, the fusion protein further comprises a stefin A protein variant specifically binding to HSA comprising an amino acid sequence selected from any one of SEQ ID NOs: 87-93 (Table 4). In some embodiments, the stefin A protein variant specifically binding to a target protein has an extended serum half-life and comprises an amino acid sequence that is at least 70%, 75% 80%, 85%, 90%, 95% or even 98% identical with a sequence selected from SEQ ID NOs: 87-93 (Table 4). Additional stefin A protein variant specifically binding to HSA sequences for use with the present disclosure can be found in WO2022/023540.

TABLE 4
Exemplary stefin A protein variant specifically binding to HSA Sequences

| Sequence | SEQ ID NO: |
|---|---|
| MIPRGLSEAKPATPEIQEIVDKVKPQLEEKTNETYGKLEAVQYKTQVLADWWQAKWP | 87 |
| HSTNYYIKVRAGDNKYMHLKVFNGPYKVHQSSGGADRVLTGYQVDKNKDDELTGF | |
| MIPRGLSEAKPATPEIQEIVDKVKPQLEEKTNETYGKLEAVQYKTQVLAGYWAAKWP | 88 |
| GSTNYYIKVRAGDNKYMHLKVFNGPFPNTSYDLQADRVLTGYQVDKNKDDELTGF | |
| MIPRGLSEAKPATPEIQEIVDKVKPQLEEKTNETYGKLEAVQYKTQVLANWYQQRWP | 89 |
| GSTNYYIKVRAGDNKYMHLKVFNGPWHNYGESSGADRVLTGYQVDKNKDDELTGF | |
| MIPRGLSEAKPATPEIQEIVDKVKPQLEEKTNETYGKLEAVQYKTQVLAVWGPEYQHQ | 90 |
| STNYYIKVRAGDNKYMHLKVFNGPNAGWPLVPEADRVLTGYQVDKNKDDELTGF | |
| MIPRGLSEAKPATPEIQEIVDKVKPQLEEKTNETYGKLEAVQYKTQVLAGHYARYWPG | 91 |
| STNYYIKVRAGDNKYMHLKVFNGPWAQKSKVHQADRVLTGYQVDKNKDDELTGF | |
| MIPRGLSEAKPATPEIQEIVDKVKPQLEEKTNETYGKLEAVQYKTQVLAGFWASKWPG | 92 |
| STNYYIKVRAGDNKYMHLKVFNGPFTAVSKKDAADRVLTGYQVDKNKDDELTGF | |
| MIPRGLSEAKPATPEIQEIVDKVKPQLEEKTNETYGKLEAVQYKTQVLANFFQRRWPG | 93 |
| STNYYIKVRAGDNKYMHLKVFNGPWKFRNTDRGADRVLTGYQVDKNKDDELTGF | |

### Host Cell and Genetically Modified Cell

In some embodiments, a nucleic acid encoding a binding agent (e.g., a binding protein, e.g., an antibody, antigen-binding antibody fragment, or stefin A protein variant that specifically bind to a target) described above and/or the fusion protein including the same is introduced into a host cell.

As used herein, the term “host cell” refers to a cell before introduction, for introducing a nucleic acid encoding a binding agent (e.g., a binding protein, e.g., an antibody, antigen-binding antibody fragment, or stefin A protein variant that specifically bind to a target) and/or the fusion protein including the same.

As used herein, the term “genetically modified cell” refers to a cell that has been engineered to include an exogenous nucleic acid. Genetically modified cells include cells that contain an exogenous nucleic acid, whether or not such exogenous nucleic acid is integrated into the genome of the cell. In some embodiments, a genetically modified cell is engineered to express a binding agent (e.g., an antibody, antigen-binding antibody fragment, or stefin A protein variant that specifically bind to a target) and/or fusion protein. In some embodiments, a genetically modified cell is produced by introducing a nucleic acid encoding a binding agent (e.g., an antibody, antigen-binding antibody fragment, or stefin A protein variant that specifically bind to a target) and/or the fusion protein including the same.

In some embodiments, the genetically modified cell may include additional genetic modification in addition to the introduction of a nucleic acid encoding the target binding agent (e.g., binding protein, e.g., an antibody, antigen-binding antibody fragment, or stefin A protein variant that specifically bind to a target) and/or the fusion protein including the same.

In some embodiments, host cells may be genetically modified to include a nucleic acid encoding a binding agent (e.g., binding protein, e.g., an antibody, antigen-binding antibody fragment, or stefin A protein variant that specifically bind to a target) and/or fusion protein including the same, in order to produce genetically modified cells that express the binding protein and/or fusion protein including the same. In some embodiments, a host cells used to produce a genetically modified cell of the present disclosure does not express the target protein.

Genetically modified cells in accordance with the present disclosure includes stem cells, including pluripotent stem cells and mesenchymal stem cells.

In some embodiments, the host cell is a cell derived from nature, for example, an animal, preferably a mammal, more preferably a human, or a cell engineered through cell engineering or genetic engineering.

As used herein, the term “stem cell” refers to a cell capable of differentiating into various types of cells constituting a biological tissue, and collectively refers to undifferentiated cells in the pre-differentiation stage which may be obtained from each tissue of embryo, fetus, and adult body. Stem cells are differentiated into specific cells by differentiation stimuli (environment), and unlike cells in which differentiation is completed and cell division is stopped, stem cells are capable of self-renewal by cell division to thus enable proliferation (expansion), and may be differentiated into other cells by different environments or different differentiation stimuli, meaning they have plasticity in differentiation.

In some embodiments, examples of the stem cells may include, but are not limited to, pluripotent stem cells, multipotent stem cells, and unipotent stem cells, depending on the differentiation potential thereof.

In some embodiments, the pluripotent stem cells are stem cells capable of differentiating into three germ layers constituting a living body, and examples thereof may include, but are not limited to, embryonic stem cells, induced pluripotent stem cells (iPS), and the like.

In some embodiments, the present disclosure provides genetically modified pluripotent stem (PS) cells that express a binding agent and/or fusion thereof. In some embodiments, provided PS cells comprise a nucleic acid encoding a binding agent and/or fusion thereof. In some embodiments, provided genetically modified PS cells are iPS cells. In some embodiments, provided genetically modified PS cells are ES cells.

In some embodiments of the present disclosure, a genetically modified PS cell that expresses a binding agent (e.g., binding protein, e.g., a stefin A protein variant) or the fusion protein thereof is produced by introducing a nucleic acid encoding the binding agent or the fusion protein including the same into a PS cell (e.g., an iPS cell or ES cell). In some embodiments, the genetically modified PS cell does not express the target protein.

Multipotent stem cells are cells having the potential to differentiate progenitor cells into cells belonging to a certain family. Examples of the multipotent stem cells may include, but are not limited to, hematopoietic stem cells, mesenchymal stromal cells, neural stem cells, and the like.

In some embodiments, the stem cells may be mesenchymal stromal cells. As used herein, the term “mesenchymal stromal cell” (or interchangably “mesenchymal stem cell” or MSC) refers to a cell capable of differentiating into osteoblasts, adipocytes, chondrocytes, and the like, which may be differentiated from mesoderm among the three germ layers of embryonic tissue. The mesenchymal stromal cells may be extracted from bone marrow, adipose tissue, umbilical cord blood, synovial membrane, trabecular bone, subpatellar fat pad, etc. The mesenchymal stromal cells are known to 1) inhibit the activity and proliferation of T lymphocytes and B lymphocytes, 2) inhibit the activity of natural killer cells (NK cells), and 3) enable allotransplantation and xenotransplantation by virtue of immunomodulatory activity of regulating the functions of dendritic cells and macrophages.

In some embodiments, provided are genetically modified mesenchymal stem cells (MSCs) that comprise a nucleic acid encoding a binding agent (e.g., binding protein) as described herein. In some embodiments, provided are genetically modified MSCs that express a binding protein, wherein the MSCs are derived from induced pluripotent stem cells. In some embodiments, provided are genetically modified MSCs that express a binding protein, wherein the MSCs possess multipotency capable of differentiating into cells selected from the group consisting of adipocytes, osteocytes, chondrocytes, myocytes, nerve cells and cardiomyocytes. In some embodiments, provided are genetically modified MSCs that express a binding protein, wherein the MSCs that are capable of long term storage and/or repeated passages. In some embodiments, provided are genetically modified MSCs that express a binding protein, wherein expression of MSC cell surface markers is maintained at 90% or more in mesenchymal stem cells of 20 or more passages.

In some embodiments, a genetically modified mesenchymal stromal cell that expresses a binding protein (e.g., an antibody, antigen-binding antibody fragment, or stefin A protein variant that specifically bind to a target) and/or fusion protein comprising the same is produced by introducing a nucleic acid encoding the binding agent of or fusion protein into a mesenchymal stromal cell. In some embodiments, provided are a population of MSCs, wherein at least 90% of the MSCs are genetically modified to express a target binding protein or fusion protein comprising the same. In some embodiments, at least 95% of the MSCs of the population express a target binding protein or fusion protein comprising the same.

In some embodiments, a genetically modified mesenchymal stromal cell does not express the target protein.

In some embodiments, a binding protein (e.g., an antibody, antigen-binding antibody fragment, or stefin A protein variant that specifically bind to a target) exhibits an agonistic effect on the target protein. In some embodiments, by introducing a nucleic acid encoding the binding agent into a mesenchymal stromal cell (MSC) as the host cell, novel immunomodulatory activity may be acquired through such a target agonistic effect by the binding agent while maintaining the immunomodulatory effect of the mesenchymal stromal cell (e.g. T-cell activation inhibitory effect).

In some embodiments, the mesenchymal stromal cell may be derived from a pluripotent stem cell. In some embodiments, the mesenchymal stromal cell enables long-term subculture. The method of producing the mesenchymal stromal cell from the pluripotent stem cell and the long-term subculture method are well known in the art, and for example, Korean Patent Application Publication No. 10-2021-0072734, Korean Patent No. 10-1135636, etc. disclose a method of maintaining undifferentiation potency and marker expression characteristics even after tens of passages.

In some embodiments, the mesenchymal stromal cell may express at least one selected from among CD29, CD44, CD73, CD90, and CD105. In some embodiments, the mesenchymal stromal cell may not express at least one cell surface marker selected from among from CD14, CD19, CD34, CD45, HLA-DR, SSEA-3, TRA-1-60, TRA-1-81, Nanog and Oct3/4.

In some embodiments, the mesenchymal stromal cell may express at least one cell surface marker selected from among CD29, CD44, CD73, CD90, and CD105.

In some embodiments, the MSC or population thereof express CD90. In

In some embodiments, the mesenchymal stromal cell is capable of maintaining at least 70%, at least 75%, at least 80%, at least 85%, at least 90%, or at least 95% of expression of the cell surface marker after at least 10 passages, at least 11 passages, at least 12 passages, at least 13 passages, at least 14 passages, at least 15 passages, at least 16 passages, at least 17 passages, at least 18 passages, at least 19 passages, or at least 20 passages.

In some embodiments, the passage may be based on an increase in the number of cells by a certain fold or more, and for example, proliferation of the number of cells at least 2-fold, 3-fold or more, 4-fold or more, 5-fold or more, 6-fold or more, 7-fold or more, or 8-fold or more as compared to the start of the previous passage may be determined to be 1 passage, but the present disclosure is not limited thereto.

In some embodiments, the mesenchymal stromal cell(s) may not express at least one cell surface marker selected from among CD34, CD45, HLA-DR, TRA-1-60, and TRA-1-81. In some embodiments, the mesenchymal stromal cell(s) may not express at least two cell surface markers selected from among CD34, CD45, HLA-DR, TRA-1-60, and TRA-1-81. In some embodiments, the mesenchymal stromal cell(s) may not express at least three cell surface markers selected from among CD34, CD45, HLA-DR, TRA-1-60, and TRA-1-81. In some embodiments, the mesenchymal stromal cell(s) do not express any of the following markers: CD34, CD45, HLA-DR, TRA-1-60, and TRA-1-81.

In some embodiments, less than 1% of the mesenchymal stromal cell(s) express at least one cell surface marker selected from among CD34, CD45, HLA-DR, TRA-1-60, and TRA-1-81. In some embodiments, less than 1% of the mesenchymal stromal cell(s) express at least two cell surface markers selected from among CD34, CD45, HLA-DR, TRA-1-60, and TRA-1-81. In some embodiments, less than 1% of the mesenchymal stromal cell(s) express at least three cell surface markers selected from among CD34, CD45, HLA-DR, TRA-1-60, and TRA-1-81. In some embodiments, less than 1% of the mesenchymal stromal cell(s) express any of the following markers: CD34, CD45, HLA-DR, TRA-1-60, and TRA-1-81.

In some embodiments, the host cell may be an immune cell.

As used herein, the term “immune cell” refers to all types of cells constituting the immune system. A cell therapeutic agent using the immunomodulatory activity of immune cells is used for the treatment of various diseases such as cancer and autoimmune diseases, but due to the non-specific effects thereof, immune cell therapeutic agents engineered to enable target-specific immune regulation, such as chimeric antigen receptors, are of great interest.

The present disclosure provides the insight that expression of a binding protein (e.g., an antibody, antigen-binding antibody fragment, or stefin A protein variant) that specifically bind to a target on immune cells may improve immunomodulatory effects. In some embodiments, the immune cells may be selected from the group consisting of T cells, B cells, natural killer (NK) cells, nkT cells, and dendritic cells, but are not limited thereto.

In some embodiments, the immune cells are those isolated from the human body, blood, or peripheral blood mononuclear cells (PBMCs), or those differentiated from stem cells (preferably pluripotent stem cells), but are not limited thereto.

In some embodiments, the genetically modified cell enables secretion of the binding agent (e.g., an antibody, antigen-binding antibody fragment, or stefin A protein variant that specifically bind to a target) and/or the fusion protein including the same, expression thereof on the cell membrane, and/or localization thereof to a specific site in the cell.

In some embodiments, the genetically modified cell expresses the binding agent (e.g., an antibody, antigen-binding antibody fragment, or stefin A protein variant that specifically bind to a target) and/or the fusion protein and presents it on the cell surface (or membrane).

When the genetically modified cell of the present disclosure expresses and presents the binding agent (e.g., an antibody, antigen-binding antibody fragment, or stefin A protein variant that specifically bind to a target) on the cell surface, clustering of ligand-target complexes and activation of the associated signaling pathway may be mimicked.

In some embodiments, the genetically modified cells in the population may express the binding agent and/or fusion protein, and the binding agent and/or the fusion protein may be secreted extracellularly, may be anchored on a cell membrane, and/or may be presented on the cell surface.

In some embodiments, the genetically modified cells in the population may express and secrete extracellularly the binding agent and/or the fusion protein at an average level of 1 fg/cell/day or greater, 10 fg/cell/day or greater, 50 fg/cell/day or greater, 100 fg/cell/day or greater, 200 fg/cell/day or greater, 300 fg/cell/day or greater, 400 fg/cell/day or greater, or 500 fg/cell/day or greater. In some embodiments, the average level of expression is maintained for at least 5 days, at least 7 days, at least 10 days, at least 12 days, at least 14 days, at least 18 days, at least 21 days, at least 24 days, at least 28 days, at least 30 days, or more.

In some embodiments, the genetically modified cells in the population may express and secrete extracellularly the binding agent and/or the fusion protein at an average level of preferably 100 fg/cell/day or greater.

In some embodiments, the genetically modified cells in the population may express and secrete extracellularly the binding agent and/or the fusion protein at an average level of 1˜5,000 fg/cell/day, 10˜3,000 fg/cell/day, 50˜2,000 fg/cell/day, 100˜1,100 fg/cell/day, or 200˜800 fg/cell/day. In some embodiments, the average level of expression is maintained for at least 5 days, at least 7 days, at least 10 days, at least 12 days, at least 14 days, at least 18 days, at least 21 days, at least 24 days, at least 28 days, at least 30 days, or more.

In some embodiments, the genetically modified cells in the population may express and secrete extracellularly the binding agent and/or the fusion protein at an average level of preferably about 100 fg/cell/day to about 1,100 fg/cell/day. In some embodiments, the average level of expression is maintained for at least 5 days, at least 7 days, at least 10 days, at least 12 days, at least 14 days, at least 18 days, at least 21 days, at least 24 days, at least 28 days, at least 30 days, or more.

Methods for measuring the levels of the binding agent and/or the fusion protein expressed and secreted extracellularly by the genetically modified cell(s) are well known in the art, and include, for example, the methods described in the examples of the instant disclosure.

In some embodiments, the genetically modified cells express and present the binding agent and/or the fusion protein on the cell surface (e.g., anchored and/or presented on a cell membrane), at an average level of 0.001 fg/cell or greater, 0.005 fg/cell or greater, 0.01 fg/cell or greater, 0.05 fg/cell or greater, 0.07 fg/cell or greater, 0.1 fg/cell/day or greater or 0.5 fg/cell/day or greater. In some embodiments, the average level of expression is maintained for at least 5 days, at least 7 days, at least 10 days, at least 12 days, at least 14 days, at least 18 days, at least 21 days, at least 24 days, at least 28 days, at least 30 days, or more.

In some embodiments, the genetically modified cell(s) express and present the binding agent and/or the fusion protein on the cell surface, at an average level of preferably 0.07 fg/cell or greater.

In some embodiments, the genetically modified cell(s) express and present the binding agent and/or the fusion protein on the cell surface, at an average level of 0.001˜10 fg/cell, 0.005˜5 fg/cell, 0.01˜3 fg/cell, 0.05˜1.5 fg/cell, or 0.07˜1 fg/cell.

In some embodiments, the genetically modified cell(s) express and present the binding protein and/or the fusion protein on the cell surface, at an average level of preferably 0.07˜1 fg/cell.

In some embodiments, the genetically modified cell(s) express and present the binding protein and/or the fusion protein on the cell surface, at an average level of 100 molecules/cell or greater, 500 molecules/cell or greater, 1,000 molecules/cell or greater, 1,500 molecules/cell or greater, 2,000 molecules/cell or greater, 2,500 molecules/cell or greater, 3,000 molecules/cell or greater, 4,000 molecules/cell or greater, or 5,000 molecules/cell or greater.

In some embodiments, the genetically modified cell(s) express and present the binding protein and/or the fusion protein on the cell surface, at an average level of preferably 2,500 molecules/cell or greater. In some embodiments, the average level of expression is maintained for at least 5 days, at least 7 days, at least 10 days, at least 12 days, at least 14 days, at least 18 days, at least 21 days, at least 24 days, at least 28 days, at least 30 days, or more.

In some embodiments, the genetically modified cell(s) express and present the binding protein and/or the fusion protein on the cell surface, at an average level of 100˜100,000 molecules/cell, 500˜80,000 molecules/cell, 1,000˜60,000 molecules/cell, 1,500˜50,000 molecules/cell, 2,000˜40,000 molecules/cell, or 2,500˜35,000 molecules/cell. In some embodiments, the average level of expression is maintained for at least 5 days, at least 7 days, at least 10 days, at least 12 days, at least 14 days, at least 18 days, at least 21 days, at least 24 days, at least 28 days, at least 30 days, or more.

In some embodiments, the genetically modified cell(s) express and present the binding protein and/or the fusion protein on the cell surface, at an average level of preferably 2,500˜35,000 molecules/cell. In some embodiments, the average level of expression is maintained for at least 5 days, at least 7 days, at least 10 days, at least 12 days, at least 14 days, at least 18 days, at least 21 days, at least 24 days, at least 28 days, at least 30 days, or more.

Methods for measuring the levels of the binding protein and/or the fusion protein expressed and presented on the genetically modified cell(s) are well known in the art and include for example, the methods described in the examples of the instant disclosure.

Method of Introducing Nucleic Acid into Host Cell

As used herein, the term “introduction” refers to allowing the host cell to receive a foreign gene (nucleic acid) that the host cell does not have.

In some embodiments, a nucleic acid encoding a binding agent (e.g., binding protein, e.g., an antibody, antigen-binding antibody fragment, or stefin A protein variant that specifically bind to a target) or a fusion protein including the same may be introduced into a host cell using a vector including the same.

As used herein, a “vector” is a means for expressing a target gene in a host cell, and examples of the vector may include, but are not limited to, viral vectors such as adenoviral vector, retroviral vector, adeno-associated viral vector, and vectors derived from viruses such as vaccinia virus (Puhlmann M. et al., Human Gene Therapy, 10:649-657 (1999); Ridgeway, 467-492 (1988); Baichwal and Sugden, In: Kucherlapati R., ed. Gene transfer. New York: Plenum Press, 117-148 (1986) and Coupar et al., Gene, 68:1-10(1988)), lentivirus (Wang G. et al., J. Clin. Invest., 104(11):R55-62(1999)), herpes simplex virus (Chamber R., et al., Proc. Natl. Acad. Sci USA, 92:1411-1415 (1995)), poxvirus (GCE, NJL, Krupa M., Esteban M., Curr. Gene Ther. 8(2):97-120 (2008)), reovirus, measles virus, Semliki Forest virus, and poliovirus, and non-viral vectors such as plasmid vectors (Sambrook et al., 1989) and mini circles (Yew et al. 2000 Mol. Ther. 1(3), 255-62).

The vector may typically include at least one component selected from among a signal sequence, an origin of replication, at least one antibiotic resistance marker gene, an enhancer element, a promoter, and a transcription termination sequence, but the present disclosure is not limited thereto. The nucleic acid encoding the binding agent (e.g., stefin A protein variant) of the present disclosure or the fusion protein including the same may be operably linked with a promoter and a transcription termination sequence.

As used herein, the term “operably linked” means a functional linkage between a nucleic acid expression control sequence (e.g. a promoter, a signal sequence, or an array of transcriptional regulator binding sites) and a different nucleic acid sequence, whereby the control sequence serves to control the transcription and/or translation of the different nucleic acid sequence.

When a prokaryotic cell is used as a host, a strong promoter capable of promoting transcription (e.g. a tac promoter, lac promoter, lacUV5 promoter, lpp promoter, pLk promoter, pR, promoter, rac5 promoter, amp promoter, recA promoter, SP6 promoter, trp promoter, or T7 promoter), a ribosome-binding site for initiation of translation, and a transcription/translation termination sequence are generally included. In addition, for example, when a eukaryotic cell is used as a host, a promoter derived from the genome of a mammalian cell (e.g. a metallothionine promoter, R-actin promoter, human hemoglobin promoter or human muscle creatine promoter) or a promoter derived from a mammalian virus (e.g. an adenovirus late promoter, vaccinia virus 7.5 k promoter, SV40 promoter, cytomegalovirus (CMV) promoter, tk promoter of HSV, mouse mammary tumor virus (MMTV) promoter, LTR promoter of HIV, promoter of Moloney virus, promoter of Epstein-Barr virus (EBV), or promoter of Rous sarcoma virus (RSV)) may be used, and a polyadenylation sequence is generally used as a transcription termination sequence

In some embodiments, the promoter may be a eukaryotic promoter, is preferably selected from among a cytomegalovirus (CMV) promoter, a PGK promoter, an EF1α promoter, an EFS promoter, a CBh promoter, an MSCV promoter, an SFFV promoter, and a UbC promoter, and is most preferably selected from among a CMV promoter, an EF1α promoter, and a CBh promoter, but is not limited thereto.

In some embodiments, the promoter may further include an enhancer sequence, but is not limited thereto.

In some cases, the vector may be fused with another sequence in order to facilitate purification of the antibody expressed therefrom. Examples of the sequence that is fused therewith include glutathione S-transferase (Pharmacia, USA), maltose-binding protein (NEB, USA), FLAG (IBI, USA), and 6×His (hexa-histidine; Qiagen, USA)).

The vector may include, as a selective marker, an antibiotic resistance gene that is commonly used in the art, for example, a gene conferring resistance to ampicillin, gentamicin, carbenicillin, chloramphenicol, streptomycin, kanamycin, puromycin, blasticidin, hygromycin, geneticin, neomycin, and tetracycline, but is not limited thereto.

In some embodiments, a nucleic acid encoding the binding agent ((e.g., an antibody, antigen-binding antibody fragment, or stefin A protein variant that specifically bind to a target) or the fusion protein including the same may be incorporated and introduced into the gene of the host cell.

In some embodiments, the nucleic acid encoding the binding agent (e.g., an antibody, antigen-binding antibody fragment, or stefin A protein variant that specifically bind to a target) or the fusion protein including the same may be constructed through chemical synthesis using an oligonucleotide synthesizer. Oligonucleotides may be designed based on the amino acid sequence of the desired polypeptide and by selecting codons that are favored in the host cell in which the recombinant polypeptide of interest will be produced. A polynucleotide sequence encoding the isolated polypeptide of interest may be synthesized using standard methods. For example, a reverse-translated gene may be constructed using a complete amino acid sequence. In addition, a DNA oligomer containing a nucleotide sequence encoding a particular isolated polypeptide may be synthesized. For example, several small oligonucleotides encoding portions of a desired polypeptide may be synthesized and then ligated. Individual oligonucleotides generally contain 5′ or 3′ overhangs for complementary assembly.

In some embodiments, when the nucleic acid sequence encoding the binding agent (e.g., an antibody, antigen-binding antibody fragment, or stefin A protein variant that specifically bind to a target) or the fusion protein including the same is obtained, a vector including the same may be produced through recombinant DNA technology using a technique well known in the art. An expression vector containing a sequence encoding the binding agent (e.g., an antibody, antigen-binding antibody fragment, or stefin A protein variant that specifically bind to a target) or the fusion protein including the same and appropriate transcriptional and translational control signals may be constructed using methods well known to those skilled in the art. Examples of such methods may include in-vitro recombinant DNA techniques, synthesis techniques, and in-vivo genetic recombination (e.g. Sambrook et al., 1990, MOLECULAR CLONING, A LABORATORY MANUAL, 2d Ed., Cold Spring Harbor Laboratory, Cold Spring Harbor, N.Y. and Ausubel et al. eds., 1998, CURRENT PROTOCOLS IN Molecular Biology, John Wiley & Sons, NY).

In some embodiments, the nucleic acid encoding the binding agent (e.g., an antibody, antigen-binding antibody fragment, or stefin A protein variant that specifically bind to a target) or the fusion protein including the same or the non-viral expression vector including the same may be delivered to the host cell using typical techniques (e.g., electroporation, liposome transfection, and calcium phosphate precipitation).

The vector may be introduced into the host cell through a method such as transduction or transfection. As used herein, the term “transduction” refers to introduction of DNA into a host such that the DNA becomes replicable either as an extrachromosomal factor or through chromosomal integration. As used herein, the term “transfection” means that an expression vector is accommodated by the host cell, regardless of whether or not any coding sequence is actually expressed. In order to introduce the vector, a variety of techniques commonly used to introduce exogenous nucleic acids (DNA or RNA) into prokaryotic or eukaryotic host cells, for example, electrophoresis, calcium phosphate precipitation, DEAE-dextran transfection, or lipofection may be used, but the present disclosure is not limited thereto.

It is to be understood that not all vectors and expression control sequences function equally in expressing the DNA sequence of the present disclosure. Likewise, not all hosts function equally for the same expression system. However, those skilled in the art will be able to make an appropriate selection from among various vectors, expression control sequences, and hosts without undue experimentation and without departing from the scope of the present disclosure. For example, a vector may be selected in consideration of the host. This is because the vector has to be able to replicate in the host. Also, the number of copies of a vector, ability to control the number of copies, and expression of another protein encoded by the vector, for example, an antibiotic marker, have to be taken into consideration. In selecting the expression control sequence, various factors have to be considered. For example, the relative strength of the sequences, controllability thereof, compatibility with the DNA sequences of the present disclosure, etc., should be taken into account, particularly with regard to possible secondary structures. The single-celled host should be selected in consideration of factors such as the selected vector, the toxicity and secretory properties of the product encoded by the DNA sequence of the disclosure, the ability to correctly fold the protein, culture and fermentation requirements, ease of purification of the product encoded by the DNA sequence of the present disclosure from the host, and the like. Within the scope of these parameters, those skilled in the art may select various vector/expression control sequence/host combinations capable of expressing the DNA sequence of the present disclosure in fermentation or large-scale animal culture. Examples of a screening method of cloning cDNA by expression cloning may include a binding method, a panning method, a film emulsion method, etc.

In an embodiment of the present disclosure, a nucleic acid encoding the binding agent (e.g., an antibody, antigen-binding antibody fragment, or stefin A protein variant that specifically bind to a target) or the fusion protein including the same was introduced into a host cell using a lentivirus.

In particular, as in an embodiment of the present disclosure, a transduction enhancer may be used when introducing a gene using a lentivirus.

In some embodiments, the transduction enhancer may be selected from among, for example, polybrene, protamine sulfate, and LentiBOOST from Sirion, and is most preferably polybrene, but is not limited thereto.

In some embodiments, the host cell that is attached or not attached may be infected with both a transduction enhancer and a lentivirus, and infection before cell attachment is preferable, but the present disclosure is not limited thereto.

Another aspect of the present disclosure related to a genetically modified MSC expressing the binding agent (e.g., an antibody, antigen-binding antibody fragment, or stefin A protein variant that specifically bind to a target) and/or the fusion protein comprising the same.

### Method of Producing Genetically Modified Cell and Culture of Genetically Modified Cell

Still another aspect of the present disclosure pertains to a method of producing a genetically modified cell into which a nucleic acid encoding the binding agent (e.g., an antibody, antigen-binding antibody fragment, or stefin A protein variant that specifically bind to a target) and/or the fusion protein including the same is introduced, including:
(a) introducing a nucleic acid encoding a binding protein (e.g., an antibody, antigen-binding antibody fragment, or stefin A protein variant that specifically bind to a target) and/or a fusion protein including the same into a host cell; and
(b) selecting and obtaining the host cell into which the nucleic acid encoding the binding agent and/or the fusion protein including the same is introduced.

In some embodiments, step (a) may be performed through various means known in the art.

In some embodiments, step (a) may be performed using a lentivirus containing the nucleic acid encoding the binding agent (e.g., an antibody, antigen-binding antibody fragment, or stefin A protein variant that specifically bind to a target) and/or the fusion protein including the same.

In some embodiments, the lentivirus may be introduced with a vector including the nucleic acid encoding the binding agent (e.g., an antibody, antigen-binding antibody fragment, or stefin A protein variant that specifically bind to a target) and/or the fusion protein including the same.

In some embodiments, the vector may further include at least one selected from among a signal sequence, an origin of replication, at least one antibiotic resistance marker gene, an enhancer element, a promoter, and a transcription termination sequence.

In some embodiments, the promoter may be a eukaryotic promoter, is preferably selected from among a CMV promoter, a PGK promoter, an EF1α promoter, an EFS promoter, a CBh promoter, an MSCV promoter, an SFFV promoter, and a UbC promoter, and is most preferably selected from among a CMV promoter, an EF1α promoter, and a CBh promoter, but is not limited thereto.

In some embodiments, the promoter may further include an enhancer sequence, but is not limited thereto.

In some embodiments, the enhancer is a short DNA region of about 50 to 1500 bp in length that may bind to a transcriptional regulatory protein. The enhancer may be located in the transcription start site or upstream or downstream of the promoter. Enhancers for various promoters are well known in the art, and may be selected and applied without limitation by those skilled in the art.

In some embodiments, step (a) may include transducing the host cell by infecting the host cell with the lentivirus.

In some embodiments, transducing the host cell by infecting the host cell with the lentivirus may be performed by adding a transduction enhancer.

In some embodiments, the transduction enhancer is preferably a cationic polymer, making it easy to incorporate a negatively charged nucleic acid or gene into the host cell.

In some embodiments, the transduction enhancer may be a cationic polymer. For example, the transduction enhancer may be selected from among polybrene, protamine sulfate, and LentiBOOST from Sirion, and is most preferably polybrene, but is not limited thereto.

In some embodiments, transducing the host cell by infecting the host cell with the lentivirus may be performed by treating the host cell with the lentivirus after attaching the host cell, or by treating and infecting the host cell with the lentivirus before attaching the host cell.

In some embodiments, a method of infecting the host cell with the lentivirus during the process of attaching the host cell by treating the host cell with the lentivirus before attaching the host cell is also known as reverse transduction.

In some embodiments, in the transduction of the host cell by infecting the host cell with the lentivirus, it is preferable to infect the host cell through treatment with the lentivirus before attaching the host cell, but the present disclosure is not limited thereto.

In some embodiments, step (b) may be characterized in that the host cell into which the nucleic acid encoding the binding agent (e.g., an antibody, antigen-binding antibody fragment, or stefin A protein variant that specifically bind to a target) and/or the fusion protein including the same is introduced is selected using an antibiotic and a resistance gene thereto.

The method of selecting the transduced cell into which the gene is introduced using a vector including an antibiotic and a resistance gene thereto is well known in the art.

In some embodiments, step (b) may be characterized in that the genetically modified cell into which the nucleic acid is introduced is selected through treatment with an aminoglycoside-based antibiotic.

In some embodiments, examples of the antibiotic may include, but are not limited to, ampicillin, gentamycin, carbenicillin, chloramphenicol, streptomycin, kanamycin, puromycin, blasticidin, hygromycin, geneticin, neomycin, tetracycline, and the like.

In some embodiments, the genetically modified cell may further include a neomycin resistance gene introduced thereto, in addition to the nucleic acid encoding the binding agent (e.g., an antibody, antigen-binding antibody fragment, or stefin A protein variant that specifically bind to a target) and/or the fusion protein including the same.

In some embodiments, treatment with the antibiotic for 3 to 7 days at a concentration of 250 to 500 μg/mL, for 5 days at a concentration of about 125 μg/mL, or for 7 days at a concentration of about 62.5 μg/mL is possible, but the present disclosure is not limited thereto.

Yet another aspect of the present disclosure pertains to a culture fluid of the genetically modified cell. In some embodiments, the conditioned cell culture medium may be prepared by culturing the genetically modified cell using suitable conditions and media depending on the type of host cell.

In some embodiments, the genetically modified cell is capable of expressing and secreting the binding agent (e.g., an antibody, antigen-binding antibody fragment, or stefin A protein variant that specifically bind to a target) and/or the fusion protein including the same. As such, the conditioned cell culture medium of the genetically modified cell may include not only the genetically modified cell, but also the binding agent (e.g., an antibody, antigen-binding antibody fragment, or stefin A protein variant that specifically bind to a target) and/or the fusion protein including the same, which are secreted thereby.

In some embodiments, when the host cell is a mesenchymal stromal cell, a long-term subculture method thereof is well known in the art, and, for example, Korean Patent Application Publication No. 10-2021-0072734, Korean Patent No. 10-No. 1135636, and the like disclose a method of maintaining undifferentiation potency and marker expression characteristics even after tens of passages.

### Use

Uses of the genetically engineered stem cells (e.g., MSCs or PSCs) of the present disclosure include, without limitation, all uses except for the manufacture of stefin A protein variants and/or fusion proteins containing the same. Preferred examples include, but are not limited to, medical, pharmaceutical, and clinical uses.

### Use—Cell Therapeutic Agent or Pharmaceutical Composition

In some embodiments of the present disclosure, the nucleic acid encoding the binding agent (e.g., an antibody, antigen-binding antibody fragment, or stefin A protein variant that specifically bind to a target, and/or fusion protein thereof) is introduced into PSC-derived mesenchymal stromal cells to prepare genetically modified cells. In some embodiments where the binding agent is presented on the cell surface, the binding agent (e.g., an antibody, antigen-binding antibody fragment, or stefin A protein variant that specifically bind to a target) is fused with a transmembrane domain, such as, for example, the human PDGFR (platelet-derived growth factor receptor) transmembrane domain. Example genetically modified stem cells (e.g., MSCs or PSCs) showed a high level of binding protein surface presentation while maintaining the characteristics of cell (e.g., MSC surface marker expression).

In some embodiments, provided are compositions comprising genetically modified stem cells (e.g., MSCs or PSCs) that include or express a binding agent (e.g., a target binding protein).

In some embodiments, provided are pharmaceutical compositions comprising the genetically modified stem cells (e.g., MSCs or PSCs). In some embodiments, the genetically modified stem cells (e.g., MSCs or PSCs) that include or express a binding protein maintain the characteristics of the stem cell (e.g., surface marker expression).

The present disclosure also describes below co-coculture of example genetically modified cells that express a binding protein with HEK-Blue TNF-alpha cells to evaluate the agonistic function. As a result, it was confirmed that an example binding protein (e.g., an antibody, antigen-binding antibody fragment, or stefin A protein variant that specifically bind to a target) presented on the surface of the genetically modified cell (e.g., MSC) binds to an example target protein of HEK-Blue TNF-alpha cells and exhibits an excellent agonistic effect.

Therefore, the genetically modified cells of the present disclosure will likely be able to exhibit Treg proliferation and activation, further reducing the immune response or suppressing the immune system.

Thus, the genetically modified cells of the present disclosure can be used for preventing and/or treating various immune diseases caused by an abnormal homeostasis of immunity, preferably an excessive or abnormal response of the immune system. More specifically, it can be used for the prevention and/or treatment of various diseases that have been reported to be treatable using Treg proliferation and activation as a mechanism.

In another aspect, the present disclosure relates to a cell therapeutic agent including the genetically modified cell and/or the conditioned cell culture medium thereof.

In another aspect, the present disclosure relates to a use of the genetically modified cell and/or the conditioned cell culture medium thereof for the manufacture of a cell therapeutic agent.

In another aspect, the present disclosure relates to a pharmaceutical composition for preventing or treating an immune disease or cancer including the genetically modified cell and/or the conditioned cell culture medium thereof.

In another aspect, the present disclosure relates to a use of the genetically modified stem cells (e.g., MSCs or PSCs) and/or the conditioned cell culture medium thereof for the prevention or treatment of an immune disease or cancer.

In another aspect, the present disclosure relates to a use of the genetically modified stem cells (e.g., MSCs or PSCs) and/or the conditioned cell culture medium thereof for the manufacture of a pharmaceutical composition for the prevention or treatment of an immune disease or cancer.

In another aspect, the present disclosure relates to a method of preventing or treating an immune diseases or cancer including administering the genetically modified stem cells (e.g., MSCs or PSCs) and/or the conditioned cell culture medium thereof to a subject.

Immune diseases for which preventive and/or therapeutic effects can be expected using the genetically modified stem cells (e.g., MSCs or PSCs) of the present disclosure include, for example, inflammatory disease, autoimmune disease or cancer, but is not limited thereto.

As used herein, the term “immune disease” refers to a disease that may be directly caused by an abnormality in the immune system, and may be selected from the group consisting of dermatitis, allergies, rhinitis, gout, ankylosing spondylitis, rheumatic fever, lupus, fibromyalgia, tendonitis, type 1 diabetes, scleroderma, neurodegenerative disease, type 2 diabetes, silicosis, atherosclerosis, vitiligo, conjunctivitis, and autoimmune disease, but is not limited thereto.

As used herein, the term “autoimmune disease” refers to a disease that occurs when immune cells in an organism recognize the organism's own tissues or cells, rather than an external invading antigen, as an antigen and attack the same. The autoimmune disease may be selected from the group consisting of rheumatoid arthritis, systemic scleroderma, atopic dermatitis, psoriasis, asthma, Guillain-Barre syndrome, myasthenia gravis, dermatomyositis, polymyositis, multiple sclerosis, autoimmune encephalomyelitis, polyarteritis nodosa, temporal arteritis, childhood diabetes, alopecia areata, blisters, aphthous stomatitis, Crohn's disease, and Behcet's disease, but is not limited thereto.

As used herein, the term “inflammatory disease” is a generic term for a disease accompanied by inflammation as a main lesion, particularly one selected from the group consisting of edema, allergies, asthma, conjunctivitis, periodontitis, rhinitis, otitis media, sore throat, tonsillitis, pneumonia, gastric ulcer, gastritis, Crohn's disease, colitis, hemorrhoids, gout, ankylosing spondylitis, rheumatic fever, lupus, fibromyalgia, psoriatic arthritis, osteoarthritis, rheumatoid arthritis, periarthritis of the shoulder, tendinitis, tenosynovitis, myositis, hepatitis, cystitis, nephritis, Sjogren's syndrome, severe myasthenia gravis, and multiple sclerosis, but is not limited thereto.

An another example, the disease include, systemic lupus erythermatosis (SLE), lupus nephritis (e.g., drug-induced lupus nephritis), immune thrombocytopenia (ITP), rheumatoid arthritis (RA), multiple sclerosis (MS), inflammatory bowel disease (IBD) (e.g., Crohn's disease and colitis/ulcerative colitis), Graft versus Host Disease (GvHD) (relating to stem cell transplants) also called allograft rejection, transplant/Solid Organ Transplantation (SOT), primary biliary cholangitis (PBC), psoriasis, psoriatic arthritis, collagen-induced arthritis, experimental allergic encephalomyelitis (EAE), oophoritis, allergic rhinitis, asthma, Sjogren's syndrome, atopic eczema, Myasthenia gravis, Grave's disease, glomerulosclerosis and/or cancer, but is not limited thereto.

As used herein, the term “prevention” refers to any action that inhibits or delays the onset of an immune diseases or cancer by administering the pharmaceutical composition provided in the present disclosure to a subject who is expected to develop an immune diseases or cancer.

As used herein, the term “treatment” refers to any action that clinically intervenes to alter the natural process of a subject or cell to be treated, and may be performed during the course of or to prevent a clinical pathology. The desired therapeutic effect includes prevention of occurrence or recurrence of disease, alleviation of symptoms, inhibition of all direct or indirect pathological consequences of disease, prevention of metastasis, reduction of disease progression rate, alleviation or temporary alleviation of disease state, and prognosis improvement. For the purpose of the present disclosure, the treatment may be interpreted as including all actions of ameliorating the symptoms of an autoimmune disease by administering the pharmaceutical composition of the present disclosure to a patient suffering from an autoimmune disease including psoriasis, but the present disclosure is not particularly limited thereto.

### Systemic Lupus Erythermatosis

Systemic lupus erythematosus (SLE), commonly referred to simply as lupus, is a chronic autoimmune disease that can cause swelling (inflammation) and pain throughout the body.

There are several different types of lupus. Systemic lupus erythematosus is the most common. Other types of lupus include:

Cutaneous lupus erythematosus: This type of lupus affects the skin—cutaneous is a term meaning skin. Individuals with cutaneous lupus erythematosus may experience skin issues like a sensitivity to the sun and rashes. Hair loss can also be a symptom of this condition.

Drug-induced lupus: These cases of lupus are caused by certain medications. People with drug-induced lupus may have many of the same symptoms of systemic lupus erythematosus, but it is usually temporary.

Neonatal lupus: A rare type of lupus, neonatal lupus is a condition found in infants at birth. Children born with neonatal lupus have antibodies that were passed to them from their mother—who either had lupus at the time of the pregnancy or may have the condition later in life. Not every baby born to a mother with lupus will have the disease.

Therapies that may be used in combination with the genetically modified cell provided herein include, for example: Steroids (corticosteroids, including prednisone); Hydroxychloroquine (Plaquenil®); Azathioprine (Imuran®); Methotrexate (Rheumatrex®); Cyclophosphamide (Cytoxan®) and mycophenolate mofetil (CellCept®); Belimumab (Benlysta®); and/or Rituximab (Rituxan®).

### Lupus Nephritis

Lupus nephritis is a frequent complication in people who have systemic lupus erythematosus—more commonly known as lupus. Lupus nephritis occurs when lupus autoantibodies affect structures in your kidneys that filter out waste. This causes kidney inflammation and may lead to blood in the urine, protein in the urine, high blood pressure, impaired kidney function or even kidney failure. As many as half of adults with systemic lupus develop lupus nephritis. Systemic lupus causes immune system proteins to damage the kidneys, harming their ability to filter out waste.

### Rheumatoid Arthritis

Rheumatoid arthritis is a type of chronic (ongoing) arthritis that occurs in joints on both sides of the body, such as hands, wrists and knees. The short-term goals of rheumatoid arthritis medications are to reduce joint pain and swelling and/or to improve joint function. The long-term goal is to slow or stop the disease process, particularly joint damage.

Arthritis is a general term that describes inflammation in joints. Rheumatoid arthritis is a type of chronic (ongoing) arthritis (resulting in pain and swelling) that occurs generally in joints symmetrically (on both sides of the body, such as hands, wrists and knees). This involvement of several joints helps distinguish rheumatoid arthritis from other types of arthritis.

In addition to affecting the joints, rheumatoid arthritis may occasionally affect the skin, eyes, lungs, heart, blood, nerves or kidneys.

Therapies that may be used in combination with the genetically modified cell provided herein to treat rheumatoid arthritis can include, for example:

Drugs that decrease pain and inflammation. These products include non-steroidal anti-inflammatory drugs (NSAIDs), such as ibuprofen (MOTRIN®), naproxen (ALEVE®), and other similar products. Another type of drug—the COX-2 inhibitor—also falls into this drug category, providing relief of the signs and symptoms of rheumatoid arthritis. Celecoxib (CELEBREX©), one COX-2 inhibitor, is available and used in the United States. The COX-2 inhibitors were designed to have fewer bleeding side effects on the stomach.

Disease-modifying antirheumatic drugs (DMARDs). Unlike other NSAIDs, DMARDs can actually slow the disease process by modifying the immune system. Older DMARDs include methotrexate (TREXALL®), gold salts, penicillamine (CUPRIMINE®), hydroxychloroquine (PLAQUENIL®), sulfasalazine (AZULFIDINE®), cyclosporine (SANDIMMUNE®), cyclophosphamide (CYTOXAN®) and leflunomide (ARAVA®). Currently, methotrexate, leflunomide, hydroxychloroquine, and sulfasalazine are the most commonly used. (Cyclosporine, cyclophosphamide, gold salts, and penicillamine are not typically used anymore.)

Biologics. Beyond these more “traditional” DMARDs, newer medications have been approved. Currently, there are seven different classes of medications and, in some cases, there are different kinds in each class. (Some of them, such as the class anti-TNFs, have been used since 2000.) Collectively, these DMARDs are known by another name—biologic agents (or biologic response agents). Compared with the traditional DMARDs, these products target the molecules that cause inflammation in rheumatoid arthritis. Inflammatory cells in the joints are involved in the development of rheumatoid arthritis itself. The biologic agents cut down the inflammatory process that ultimately causes the joint damage seen in rheumatoid arthritis. The older DMARDs work one step further out than the biologics; they work by modifying the body's own immune response to the inflammation. By attacking the cells at a more specific level of the inflammation itself, biologics are considered to be more effective and more specifically targeted. The biologic agents include etanercept (ENBREL®), infliximab (REMICADE®), adalimumab (HUMIRA®), anakinra (KINARET®), abatacept (ORENCIA®), rituximab (RITUXAN®), certolizumab pegol (CIMZIA®), golimumab (SYMPONI®), tocilizumab (ACTEMRA®) and tofacitinib (XELJANJ®). Some of the biologics are used in combination with the traditional DMARDs, especially with methotrexate.

### Multiple Sclerosis

Multiple sclerosis (MS) is an autoimmune disease. With these conditions, the immune system mistakenly attacks healthy cells. In people with MS, the immune system attacks cells in the myelin, the protective sheath that surrounds nerves in the brain and spinal cord. Damage to the myelin sheath interrupts nerve signals from the brain to other parts of the body. The damage can lead to symptoms affecting the brain, spinal cord and eyes.

There are four types of multiple sclerosis:

Clinically isolated syndrome (CIS): When someone has a first episode of MS symptoms, healthcare providers often categorize it as CIS. Not everyone who has CIS goes on to develop multiple sclerosis.

Relapsing-remitting MS (RRMS): This is the most common form of multiple sclerosis. People with RRMS have flare-ups—also called relapse or exacerbation—of new or worsening symptoms. Periods of remission follow (when symptoms stabilize or go away).

Primary progressive MS (PPMS): People diagnosed with PPMS have symptoms that slowly and gradually worsen without any periods of relapse or remission.

Secondary progressive MS (SPMS): In many cases, people originally diagnosed with RRMS eventually progress to SPMS. With secondary-progressive multiple sclerosis, one continues to accumulate nerve damage. Symptoms progressively worsen. While one may still experience some relapses or flares (when symptoms increase), one no longer has periods of remission afterward (when symptoms stabilize or go away).

Therapies that may be used in combination with the genetically modified cell provided herein include, for example:

Disease-modifying therapies (DMTs): Several medications have FDA approval for long-term MS treatment. These drugs help reduce relapses (also called flare-ups or attacks). They slow down the disease's progression. And they can prevent new lesions from forming on the brain and spinal cord.

Relapse management medications: If there is a severe attack, a neurologist may recommend a high dose of corticosteroids. The medication can quickly reduce inflammation. They slow damage to the myelin sheath surrounding nerve cells.

Physical rehabilitation: Multiple sclerosis can affect physical function. Staying physically fit and strong will help maintain mobility.

Mental health counseling: Coping with a chronic condition can be emotionally challenging. MS can sometimes affect mood and memory. Working with a neuropsychologist or getting other emotional support is an essential part of managing the disease.

### Inflammatory Bowel Disease

Inflammatory bowel disease (IBD) is a group of disorders that cause chronic inflammation (pain and swelling) in the intestines.

Crohn's disease and ulcerative colitis are the main types of IBD. Types include:

Crohn's disease causes pain and swelling in the digestive tract. It can affect any part from the mouth to the anus. It most commonly affects the small intestine and upper part of the large intestine.

Ulcerative colitis causes swelling and sores (ulcers) in the large intestine (colon and rectum).

Microscopic colitis causes intestinal inflammation that's only detectable with a microscope.

Therapies that may be used in combination with the genetically modified cell provided herein include, for example: aminosalicylates (an anti-inflammatory medicine like sulfasalazine, mesalamine or balsalazide) minimize irritation to the intestines; antibiotics treat infections and abscesses; biologics interrupt signals from the immune system that cause inflammation; corticosteroids, such as prednisone, keep the immune system in check and manage flares; immunomodulators calm an overactive immune system; antidiarrheal medication; nonsteroidal anti-inflammatory drugs (NSAIDs); vitamins and supplements like probiotics.

### Graft Versus Host Disease

Graft versus host disease (GvHD) is a condition that might occur after an allogeneic transplant. In GvHD, the donated bone marrow or peripheral blood stem cells view the recipient's body as foreign, and the donated cells/bone marrow attack the body.

There are two forms of GvHD: Acute graft versus host disease (aGvHD); and Chronic graft versus host disease (cGvHD).

### Psoriasis

Psoriasis is a chronic skin disorder, which means a skin condition that doesn't go away. People with psoriasis have thick, pink or red patches of skin covered with white or silvery scales. The thick, scaly patches are called plaques. Psoriasis usually starts in early adulthood, though it can begin later in life. In addition to red, scaly patches, symptoms of psoriasis include: itchiness, cracked, dry skin, scaly scalp, skin pain, nails that are pitted, cracked or crumbly, and joint pain.

Therapies that may be used in combination with the genetically modified cell provided herein include, for example: Steroid creams, Moisturizers for dry skin, anthralin (a medication to slow skin cell production), Medicated lotions, shampoos and bath solutions to improve scalp psoriasis, Vitamin D3 ointment, Vitamin A or retinoid creams, light therapy, PUVA (a treatment combines a medication called psoralen with exposure to a special form of UV light), methotrexate, retinoids, cyclosporine, and/or immune therapies.

### Sjogren's Syndrome

Sjögren's syndrome is a lifelong autoimmune disorder that reduces the amount of moisture produced by glands in the eyes and mouth. It is named for Henrik Sjögren, a Swedish eye doctor who first described the condition. While dry mouth and dry eyes are the primary symptoms, most people who have these problems don't have Sjögren's syndrome. Dry mouth is also called xerostomia.

There are two forms of Sjögren's syndrome: primary Sjögren's syndrome, which develops on its own, not because of any other health condition, and secondary Sjögren's syndrome, which develops in addition to other autoimmune diseases like rheumatoid arthritis, lupus and psoriatic arthritis.

Therapies that may be used in combination with the genetically modified cell provided herein include, for example, treatments for dry eyes (e.g., artificial tears, prescription eye drops, punctal plugs, surgery, autologous serum drops), treatments for dry mouth (e.g., saliva producers), treatments for joint or organ problems (e.g., pain relievers, anti-rheumatics, immunosuppressants, steroids, antifungals, and treatments for vaginal dryness.

### Myasthenia Gravis

Myasthenia gravis (MG) is an autoimmune disease, meaning the body's immune system mistakenly attacks its own parts. MG affects the communication between nerves and muscles (the neuromuscular junction).

People with MG lose the ability to control muscles voluntarily. They experience muscle weakness and fatigue of various severity. They may not be able to move muscles in the eyes, face, neck and limbs. MG is a lifelong neuromuscular disease.

MG affects about 20 out of every 100,000 people. Experts estimate that 36,000 to 60,000 Americans have this neuromuscular disease. The actual number of people affected may be higher, as some people with mild cases may not know they have the disease. MG mostly affects women aged 20 to 40 and men aged 50 to 80. About one in 10 cases of MG occur in teenagers (juvenile MG). The illness can affect people of all ages but is rare in children.

Autoimmune MG is the most common form of this neuromuscular disease. Autoimmune MG may be:

Ocular: The muscles that move the eyes and eyelids weaken. The eyelids may droop, or you may not be able to keep your eyes open. Some people have double vision. Eye weakness is often the first sign of MG. Nearly half of people with ocular MG evolve into the generalized form within two years of the first symptom.

Generalized: Muscle weakness affects the eye and other body parts such as the face, neck, arms, legs and throat. It may be difficult to speak or swallow, lift the arms over the head, stand up from a seated position, walk long distances and climb stairs.

Therapies that may be used in combination with the genetically modified cell provided herein include, for example, Medications, Monoclonal antibodies, IV immunoglobulin (IVIG), Plasma exchange (plasmapheresis), and/or Surgery.

### Cancer

The genetically modified cell of the present disclosure may be useful in the treatment of cancer, either as monotherapy or in conjunction with other cancer treatments such as immunotherapies, e.g. CAR-T therapies, checkpoint inhibitors, and conventional cancer treatments.

The cancer includes, for example, benign, premalignant, and malignant tumors, or proliferative disease, a precancerous condition, but is not limited thereto.

In some embodiments, the cancer is a hematologic cancer, such as chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or preleukemia, but is not limited thereto.

In some embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers.

Pharmaceutical preparations Formulations are prepared for storage and use by combining the genetically modified cell of the present disclosure with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

In some embodiments, an AFFIMER® agent described herein is lyophilized and/or stored in a lyophilized form. In some embodiments, a formulation comprising the genetically modified cell described herein is lyophilized.

Suitable pharmaceutically acceptable vehicles include but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22.sup.nd Edition, 2012, Pharmaceutical Press, London.).

The pharmaceutical compositions of the present disclosure can be administered in any number of ways for either local or systemic treatment. Administration can be topical by epidermal ortransdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present disclosure, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The genetically modified cell described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22.sup.nd Edition, 2012, Pharmaceutical Press, London.

In some embodiments, pharmaceutical formulations include the genetically modified cell of the present disclosure complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In some embodiments, sustained-release preparations comprising the genetically modified cell described herein can be produced. Suitable examples of sustained-release preparations include semi-permeable matrices of solid hydrophobic polymers containing an AFFIMER® agent, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

In some embodiments, in addition to administering the genetically modified cell described herein, the method or treatment further comprises administering at least one additional therapeutic agent. An additional therapeutic agent can be administered prior to, concurrently with, and/or subsequently to, administration of the genetically modified cell. Pharmaceutical compositions comprising the genetically modified cell and the additional therapeutic agent(s) are also provided. In some embodiments, the at least one additional therapeutic agent comprises 1, 2, 3, or more additional therapeutic agents.

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the genetically modified cell.

In some embodiments of the methods described herein, the combination of the genetically modified cell described herein and at least one additional therapeutic agent results in additive or synergistic results. In some embodiments, the combination therapy results in an increase in the therapeutic index of the genetically modified cell. In some embodiments, the combination therapy results in an increase in the therapeutic index of the additional therapeutic agent(s). In some embodiments, the combination therapy results in a decrease in the toxicity and/or side effects of the genetically modified cell. In some embodiments, the combination therapy results in a decrease in the toxicity and/or side effects of the additional therapeutic agent(s).

Combined administration can include co-administration, either in a single pharmaceutical formulation or using separate formulations, or consecutive administration in either order but generally within a time period such that all active agents can exert their biological activities simultaneously.

It will be appreciated that the combination of the genetically modified cell described herein and at least one additional therapeutic agent may be administered in any order or concurrently. In some embodiments, the genetically modified cell will be administered to patients that have previously undergone treatment with a second therapeutic agent. In certain other embodiments, the genetically modified cell and a second therapeutic agent will be administered substantially simultaneously or concurrently. For example, a subject may be given the genetically modified cell while undergoing a course of treatment with a second therapeutic agent (e.g., chemotherapy). In some embodiments, the genetically modified cell will be administered within 1 year of the treatment with a second therapeutic agent. In certain alternative embodiments, the genetically modified cell will be administered within 10, 8, 6, 4, or 2 months of any treatment with a second therapeutic agent. In certain other embodiments, the genetically modified cell will be administered within 4, 3, 2, or 1 weeks of any treatment with a second therapeutic agent. In some embodiments, the genetically modified cell will be administered within 5, 4, 3, 2, or 1 days of any treatment with a second therapeutic agent. It will further be appreciated that the two (or more) agents or treatments may be administered to the subject within a matter of hours or minutes (e.g., substantially simultaneously).

### Use—a Composition or Method for Immunomodulation

The binding agent (e.g., an antibody, antigen-binding antibody fragment, or stefin A protein variant that specifically bind to a target) and/or the fusion protein of the genetically modified cell binds to and activate a target which has the effect of proliferation and activation of Treg cells, and consequently, the regulation of immunity, that is, the effect of suppressing immune system or immune response.

Therefore, in another aspect, the present disclosure relates to a composition for immunomodulation comprising the genetically modified cell and/or the conditioned cell culture medium thereof.

In another aspect, the present disclosure relates to a use of the genetically modified cell and/or the conditioned cell culture medium thereof for immunomodulation.

In another aspect, the present disclosure relates to a use of the genetically modified cell and/or the conditioned cell culture medium thereof for immunomodulation.

In another aspect, the present disclosure relates to a method for immunomodulation comprising administering the genetically modified cell and/or the conditioned cell culture medium thereof to a subject.

As used herein, the term “immunomodulation” means relieving immune imbalance in the blood and maintaining immune homeostasis. Maintaining immune homeostasis refers to a condition in which immune tolerance which is a mechanism for inhibiting immunity, and immunity which promotes immune response, are balanced, and the maintenance of such a condition is an essential factor in the treatment of immune diseases, especially autoimmune diseases.

The term “immunosuppression” or “suppression of immune system” as used herein refers to various substances used to reduce or block the ability of a host to produce antibodies (humoral immune response) or the ability to elicit a cellular immune response to the action of an antigen.

In some embodiments, the genetically modified cell and/or the conditioned cell culture medium may exhibit a hyper-immunosuppressive effect. The hyper-immunosuppressive effect is an effect of suppressing hyper-immunity by inhibiting lymphocyte overexpression caused by a nonspecific stimulator in splenocytes.

The compositions according to the present disclosure may be administered in combination with immune-related proteins, in particular autoimmune- or allergy-related proteins. Specific examples of the proteins include autoantigens involved in autoimmune diseases. For example, the proteins may include autoantigens involved in rheumatoid arthritis, such as heat-shock proteins (HSPs), citrullinated filaggrin, glucose-6-phosphate isomerase, p205, collagen, and the like; autoantigens involved in Type I diabetes, such as insulin, Zinc transporter 8 protein (ZnT8), Pancreatic and duodenal homeobox 1 (PDXT), Chromogranin A (CHGA), and Islet amyloid polypeptide (IAPP); and autoantigens involved in myasthenia gravis, such as an acetylcholine receptor. In addition to all types of autoantigens known in autoimmune diseases, the proteins may include various allergens known to cause food allergies, such as peanuts, milk, eggs, tree nuts, beans, crustaceans such as shrimp and the like, fish-derived substances, and the like.

### Use—a Composition or Method for Culturing Regulatory T Cell

In some embodiments, the present disclosure relates to a composition or medium for culturing regulatory T cell comprising the genetically modified cell and/or the conditioned cell culture medium thereof.

In some embodiments, the present disclosure relates to a method for culturing regulatory T cell comprising, culturing regulatory T cell in presence of the genetically modified cell and/or the conditioned cell culture medium thereof.

In some embodiments, the present disclosure relates to a use of the genetically modified cell and/or the conditioned cell culture medium thereof for culturing regulatory T cell.

In some embodiments, the present disclosure relates to a use of the genetically modified cell and/or the conditioned cell culture medium thereof for manufacturing a composition or medium for culturing regulatory T cell.

In some embodiments, the composition or medium for culturing T cell further comprises any one selected from the group consisting of energy source, amino acids, sugars, inorganic salts, and vitamins. fetal bovine serum (FBS), hydroxyethyl piperazine ethane sulfonic acid (HEPES), proteins, carbohydrates, mercaptoethanol, and growth factors.

In some embodiments, the composition or medium may further comprises—components required by cells for cell growth and survival in vitro, or comprises components that help cell growth and survival. Specifically, the components may be vitamins, essential or non-essential amino acids, and trace elements.

As used herein, the term “regulatory T cell (Treg or Treg cell)” comprises natural regulatory T cells (nTreg) or induced regulatory T cells (iTreg). The regulatory T cells maintain immune homeostasis and block an autoimmune response, and the like by inhibiting an immune response. In some embodiments, the regulatory T cells may be genetically engineered regulatory T cells. Examples of the genetically engineered regulatory T cells include, but are not limited to, CAR-Treg and TCR-Treg.

In some embodiments, the regulatory T cells are preferably CD4+Foxp3+ regulatory T cells, but are not limited thereto.

In some embodiments, the regulatory T cells express the target protein, preferably the target protein is presented on surface (or membrane) of the regulatory T cells. In some embodiments, the regulatory T cells are isolated regulatory T cells.

In some embodiments, the regulatory T cells are cultured in presence of the genetically modified cell and/or the conditioned cell culture medium thereof.

In some embodiments, culturing the regulatory T cells in presence of the genetically modified cell and/or the conditioned cell culture medium thereof may be performed in vitro or ex vivo. In some embodiments, culturing the regulatory T cells may be performed co-culturing the regulatory T cells and the genetically modified cells. In the context of the present disclosure, the genetically modified cell and/or the conditioned cell culture medium thereof binds to the target protein presented on the Treg cell surface and activates a target-associated signaling pathway.

In some embodiments, the induced regulatory T cells(iTregs) may be obtained by co-culture with tolerogenic antigen-presenting cells with CD4+ T cells, but are not limited thereto.

In some embodiments, the use, composition, medium, or method for culturing Treg cells of the present disclosure may induce proliferation and/or activation of regulatory T cells.

### Use—Composition for Drug Delivery

Even yet a further aspect of the present disclosure pertains to a composition for drug delivery including the genetically modified cell described above.

In some embodiments, the genetically modified cell may include at least one drug that is supported thereby or attached to the surface thereof.

In some embodiments, the drug may be additionally loaded with at least one selected from the group consisting of a gene, a virus, and a small molecule compound.

In some embodiments, the drug may have immunomodulatory activity, preferably an immunosuppressive effect, but is not limited thereto.

The genetically modified cell of the present disclosure is capable of expressing the binding agent (e.g., an antibody, an antibody fragment, or a stefin A protein variant), thereby activating the target protein mediated attachment and activity of immune cells.

Also, in some embodiments, the genetically modified cell may be a mesenchymal stromal cell. When the host cell of the present disclosure is a mesenchymal stromal cell, the mesenchymal stromal cell has a homing function to biologically search for a damaged or infected site in the body, and thus has very high targeting ability, making it possible to effectively and accurately deliver the drug to a desired site of the body.

In some embodiments, the drug may be supported inside the mesenchymal stromal cell, attached to the surface thereof, or supported inside the cell and attached to the cell surface, but the present disclosure may not be limited thereto. The drug may be directly supported inside the mesenchymal stromal cells and/or attached to the surface thereof, but the present disclosure is not limited thereto. The drug may be loaded to a nanostructure, supported inside the mesenchymal stromal cell in a state of being attached to a specific molecule, and/or attached to the cell surface, but the present disclosure is not limited thereto. In some embodiments, the nanostructure may include inorganic nanoparticles, polymer nanoparticles, proteins, or liposomes. Examples of the inorganic nanoparticles may include, but are not limited to, iron oxide nanoparticles, quantum dot nanoparticles, metal oxide nanoparticles, and the like. The nanostructure may include, but is not limited to, a porous nanostructure. For example, the drug may be supported by the pores in the porous nanostructure or loaded to the mesenchymal stromal cell in a form of being attached to the surface of the nanostructure, but the present disclosure is not limited thereto. The polymer nanoparticles are nanoparticles frequently used for drug delivery, and are mainly made of polymers and fats.

In some embodiments, the genetically modified cell may be characterized in that the drug is released at a target site. In some embodiments, the drug carried in the genetically modified cell may be released. In some embodiments, the drug attached to the surface of the genetically modified cell may be detached and released from the surface depending on the target environment. In some embodiments, when the drug is supported by the nanostructure, the drug may be released by temperature-specific or pH-specific structural change of the nanostructure.

### EXAMPLES

Hereinafter, the present disclosure will be described in more detail with reference to the following examples. However, it will be obvious to those skilled in the art that the following examples are provided only for illustration of the present disclosure and should not be construed as limiting the scope of the present disclosure.

### Example 1. Introduction of a Gene Encoding an Example Binding Protein into MSCs Via Lentivirus

### Example 1-1: Selection of Transduction Enhancer

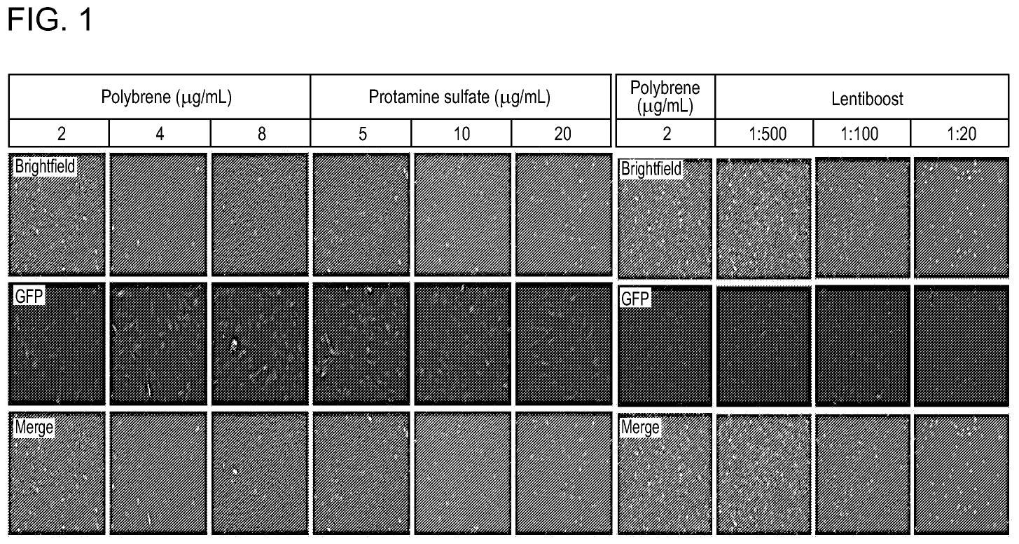

When introducing a gene into PSC-derived MSC (obtained from the Institute of Reproductive Medicine and Population, Seoul National University Medical Research Center) using a lentivirus, a transduction enhancer was selected to increase the introduction efficiency. The transduction enhancer is a cationic polymer and helps the lentivirus to bind to cells through ion neutralization, thereby increasing gene introduction efficiency. Representative transduction enhancers include polybrene and protamine sulfate, which have different efficiencies and sensitivities depending on cell characteristics, so it is necessary to select a suitable transduction enhancer. Hence, in order to select a transduction enhancer suitable for PSC-derived MSC, three types, namely polybrene (Sigma-Aldrich, TR-1003-G), protamine sulfate (Sigma-Aldrich, P3369), and LentiBOOST (SIRION Biotech, SB-P-LV-101-02), were used at different concentrations and introduction efficiency was compared. The cells were treated with 5 MOI of a lentivirus (SIRION Biotech, SEQ ID NO: 94) constructed using a vector including the eGFP gene. Here, polybrene at 2, 4, and 8 μg/mL, protamine sulfate at 5, 10, and 20 μg/mL, and LentiBOOST at concentrations of 1:500, 1:100, and 1:20 according to the manufacturer's recommendations were also used for cell treatment. 16 to 20 hours after treatment with the lentivirus, the culture medium containing the lentivirus was removed and replaced with a fresh culture medium (FUJIFILM Irvine Scientific, 991333), followed by culture for 48 hours. After 48 hours, the cells were harvested and gene introduction efficiency was compared based on the GFP fluorescence-introduced cell population through flow cytometry. Consequently, as shown in FIG. 1, polybrene showed an introduction efficiency of 66.27% at a concentration of 2 g/mL, protamine sulfate showed an introduction efficiency of 63.90% at a concentration of 20 μg/mL, and LentiBOOST showed an introduction efficiency of 21.98% at 1:500, confirming a tendency of increasing cell death with an increase in the treatment concentration. Based on the results thereof, when introducing the gene into PSC-derived MSC using the lentivirus, polybrene, showing the highest introduction efficiency at a low concentration, exhibited vastly superior introduction efficiency.

The vectors used for the construction of the lentivirus used in Examples of the present disclosure are as shown in SEQ ID NOs: 94 to 103 of Table 5 below.

TABLE 5

| | | SEQ |
|---|---|---|
| | | ID |
| Vector | Sequence | NO. |
| 7958_pcLV- | aatattttgaagcatttatcagggttattgtctcatgagcggatacatatttgaa | 94 |
| CMV-eGFP- | tgtatttagaaaaataaacaaataggggttccgcgcacatttccccgaaaagtgc | |
| IRES-Neo- | cacctgacgtctaagaaaccattattatcatgacattaacctataaaaataggcg | |
| WPRE | tatcacgaggccctttcgtctcgcgcgtttcggtgatgacggtgaaaacctctga | |
| (8566 bp) | cacatgcagctcccggagacggtcacagcttgtctgtaagcggatgccgggagca | |
| | gacaagcccgtcagggcgcgtcagcgggtgttggcgggtgtcggggctggcttaa | |
| | ctatgcggcatcagagcagattgtactgagagtgcaccatatgcggtgtgaaata | |
| | ccgcacagatgcgtaaggagaaaataccgcatcaggcgccattcgccattcaggc | |
| | tgcgcaactgttgggaagggcgatcggtgcgggcctcttcgctattacgccagct | |
| | ggcgaaagggggatgtgctgcaaggcgattaagttgggtaacgccagggttttcc | |
| | cagtcacgacgttgtaaaacgacggccagtgccaagctgacgcgtgtagtcttat | |
| | gcaatactcttgtagtcttgcaacatggtaacgatgagttagcaacatgccttac | |
| | aaggagagaaaaagcaccgtgcatgccgattggtggaagtaaggtggtacgatcg | |
| | tgccttattaggaaggcaacagacgggtctgacatggattggacgaaccactgaa | |
| | ttgccgcattgcagagatattgtatttaagtgcctagctcgatacaataaacggg | |
| | tctctctggttagaccagatctgagcctgggagctctctggctaactagggaacc | |
| | cactgcttaagcctcaataaagcttgccttgagtgcttcaagtagtgtgtgcccg | |
| | tctgttgtgtgactctggtaactagagatccctcagacccttttagtcagtgtgg | |
| | aaaatctctagcagtggcgcccgaacagggacctgaaagcgaaagggaaaccaga | |
| | gctctctcgacgcaggactcggcttgctgaagcgcgcacggcaagaggcgagggg | |
| | cggcgactggtgagtacgccaaaaattttgactagcggaggctagaaggagagag | |
| | atgggtgcgagagcgtcagtattaagcgggggagaattagatcgcgatgggaaaa | |
| | aattcggttaaggccagggggaaagaaaaaatataaattaaaacatatagtatgg | |
| | gcaagcagggagctagaacgattcgcagttaatcctggcctgttagaaacatcag | |
| | aaggctgtagacaaatactgggacagctacaaccatcccttcagacaggatcaga | |
| | agaacttagatcattatataatacagtagcaaccctctattgtgtgcatcaaagg | |
| | atagagataaaagacaccaaggaagctttagacaagatagaggaagagcaaaaca | |
| | aaagtaagaccaccgcacagcaagcggccactgatcttcagacctggaggaggag | |
| | atatgagggacaattggagaagtgaattatataaatataaagtagtaaaaattga | |
| | accattaggagtagcacccaccaaggcaaagagaagagtggtgcagagagaaaaa | |
| | agagcagtgggaataggagctttgttccttgggttcttgggagcagcaggaagca | |
| | ctatgggcgcagcctcaatgacgctgacggtacaggccagacaattattgtctgg | |
| | tatagtgcagcagcagaacaatttgctgagggctattgaggcgcaacagcatctg | |
| | ttgcaactcacagtctggggcatcaagcagctccaggcaagaatcctggctgtgg | |
| | aaagatacctaaaggatcaacagctcctggggatttggggttgctctggaaaact | |
| | catttgcaccactgctgtgccttggaatgctagttggagtaataaatctctggaa | |
| | cagattggaatcacacgacctggatggagtgggacagagaaattaacaattacac | |
| | aagcttaatacactccttaattgaagaatcgcaaaaccagcaagaaaagaatgaa | |
| | caagaattattggaattagataaatgggcaagtttgtggaattggtttaacataa | |
| | caaattggctgtggtatataaaattattcataatgatagtaggaggcttggtagg | |
| | tttaagaatagtttttgctgtactttctatagtgaatagagttaggcagggatat | |
| | tcaccattatcgtttcagacccacctcccaaccccgaggggacccgacaggcccg | |
| | aaggaatagaagaagaaggtggagagagagacagagacagatccattcgattagt | |
| | gaacggatctcgacggtatcggttaacttttaaaagaaaaggggggattgggggg | |
| | tacagtgcaggggaaagaatagtagacataatagcaacagacatacaaactaaag | |
| | aattacaaaaacaaattacaaaattcaaaattttatcgatactagtggccctcct | |
| | atagtgagtcgtattatactatgccgatatactatgccgatgattaattgtcaac | |
| | acgtgctgcagactagtattatgcccagtacatgaccttatgggactttcctact | |
| | tggcagtacatctacgtattagtcatcgctattaccatggtgatgcggttttggc | |
| | agtacatcaatgggcgtggatagcggtttgactcacggggatttccaagtctcca | |
| | ccccattgacgtcaatgggagtttgttttggcaccaaaatcaacgggactttcca | |
| | aaatgtcgtaacaactccgccccattgacgcaaatgggcggtaggcgtgtacggt | |
| | gggaggtttatataagcagagctcgtttagtgaaccgtcagatcgcctggagacg | |
| | ccatccacgctgttttgacctccatagaagagctagcctagcggatccgaattct | |
| | cgatcacaagtttgtacaaaaaagcaggctttaaaggaaccaattcagtcgccgc | |
| | caccatggtgagcaagggcgaggagctgttcaccggggtggtgcccatcctggtc | |
| | gagctggacggcgacgtaaacggccacaagttcagcgtgtccggcgagggcgagg | |
| | gcgatgccacctacggcaagctgaccctgaagttcatctgcaccaccggcaagct | |
| | gcccgtgccctggcccaccctcgtgaccaccctgacctacggcgtgcagtgcttc | |
| | agccgctaccccgaccacatgaagcagcacgacttcttcaagtccgccatgcccg | |
| | aaggctacgtccaggagcgcaccatcttcttcaaggacgacggcaactacaagac | |
| | ccgcgccgaggtgaagttcgagggcgacaccctggtgaaccgcatcgagctgaag | |
| | ggcatcgacttcaaggaggacggcaacatcctggggcacaagctggagtacaact | |
| | acaacagccacaacgtctatatcatggccgacaagcagaagaacggcatcaaggt | |
| | gaacttcaagatccgccacaacatcgaggacggcagcgtgcagctcgccgaccac | |
| | taccagcagaacacccccatcggcgacggccccgtgctgctgcccgacaaccact | |
| | acctgagcacccagtccgccctgagcaaagaccccaacgagaagcgcgatcacat | |
| | ggtcctgctggagttcgtgaccgccgccgggatcactctcggcatggacgagctg | |
| | tacaagaattagcgctactagtgatatcaagatgatctagacccagctttcttgt | |
| | acaaagtggtatcgagatatctgcatttaattaatctagagtttaaacgcggccg | |
| | caaattccgcccctctccctcccccccccctaacgttactggccgaagccgcttg | |
| | gaataaggccggtgtgcgtttgtctatatgttattttccaccatattgccgtctt | |
| | ttggcaatgtgagggcccggaaacctggccctgtcttcttgacgagcattcctag | |
| | gggtctttcccctctcgccaaaggaatgcaaggtctgttgaatgtcgtgaaggaa | |
| | gcagttcctctggaagcttcttgaagacaaacaacgtctgtagcgaccctttgca | |
| | ggcagcggaaccccccacctggcgacaggtgcctctgcggccaaaagccacgtgt | |
| | ataagatacacctgcaaaggcggcacaaccccagtgccacgttgtgagttggata | |
| | gttgtggaaagagtcaaatggctctcctcaagcgtattcaacaaggggctgaagg | |
| | atgcccagaaggtaccccattgtatgggatctgatctggggcctcggtgcacatg | |
| | ctttacatgtgtttagtcgaggttaaaaaaacgtctaggccccccgaaccacggg | |
| | gacgtggttttcctttgaaaaacacgatgataatatggccacaaccatggttatt | |
| | gaacaagatggattgcacgcaggttctccggccgcttgggtggagaggctattcg | |
| | gctatgactgggcacaacagacaatcggctgctctgatgccgccgtgttccggct | |
| | gtcagcgcaggggcgcccggttctttttgtcaagaccgacctgtccggtgccctg | |
| | aatgaactgcaggacgaggcagcgcggctatcgtggctggccacgacgggcgttc | |
| | cttgcgcagctgtgctcgacgttgtcactgaagcgggaagggactggctgctatt | |
| | gggcgaagtgccggggcaggatctcctgtcatctcaccttgctcctgccgagaaa | |
| | gtatccatcatggctgatgcaatgcggcggctgcatacgcttgatccggctacct | |
| | gcccattcgaccaccaagcgaaacatcgcatcgagcgagcacgtactcggatgga | |
| | agccggtcttgtcgatcaggatgatctggacgaagagcatcaggggctcgcgcca | |
| | gccgaactgttcgccaggctcaaggcgcgcatgcccgacggcgaggatctcgtcg | |
| | tgacccatggcgatgcctgcttgccgaatatcatggtggaaaatggccgcttttc | |
| | tggattcatcgactgtggccggctgggtgtggcggaccgctatcaggacatagcg | |
| | ttggctacccgtgatattgctgaagagcttggcggcgaatgggctgaccgcttcc | |
| | tcgtgctttacggtatcgccgctcccgattcgcagcgcatcgccttctatcgcct | |
| | tcttgacgagttcttctgagtcgacaatcaacctctggattacaaaatttgtgaa | |
| | agattgactggtattcttaactatgttgctccttttacgctatgtggatacgctg | |
| | ctttaatgcctttgtatcatgctattgcttcccgtatggctttcattttctcctc | |
| | cttgtataaatcctggttgctgtctctttatgaggagttgtggcccgttgtcagg | |
| | caacgtggcgtggtgtgcactgtgtttgctgacgcaacccccactggttggggca | |
| | ttgccaccacctgtcagctcctttccgggactttcgctttccccctccctattgc | |
| | cacggcggaactcatcgccgcctgccttgcccgctgctggacaggggctcggctg | |
| | ttgggcactgacaattccgtggtgttgtcggggaaatcatcgtcctttccttggc | |
| | tgctcgcctgtgttgccacctggattctgcgcgggacgtccttctgctacgtccc | |
| | ttcggccctcaatccagcggaccttccttcccgcggcctgctgccggctctgcgg | |
| | cctcttccgcgtcttcgccttcgccctcagacgagtcggatctccctttgggccg | |
| | cctccccgcctggtacctttaagaccaatgacttacaaggcagctgtagatctta | |
| | gccactttttaaaagaaaaggggggactggaagggctaattcactcccaacgaaa | |
| | ataagatctgctttttgcttgtactgggtctctctggttagaccagatctgagcc | |
| | tgggagctctctggctaactagggaacccactgcttaagcctcaataaagcttgc | |
| | cttgagtgcttcaagtagtgtgtgcccgtctgttgtgtgactctggtaactagag | |
| | atccctcagacccttttagtcagtgtggaaaatctctagcagtagtagttcatgt | |
| | catcttattattcagtatttataacttgcaaagaaatgaatatcagagagtgaga | |
| | ggaacttgtttattgcagcttataatggttacaaataaagcaatagcatcacaaa | |
| | tttcacaaataaagcatttttttcactgcattctagttgtggtttgtccaaactc | |
| | atcaatgtatcttatcatgtctggctctagctatcccgcccctaactccgcccag | |
| | ttccgcccattctccgccccatggctgactaattttttttatttatgcagaggcc | |
| | gaggccgcctcggcctctgagctattccagaagtagtgaggaggcttttttggag | |
| | gcctagacttttgcagagacggcccaaattcgtaatcatggtcatagctgtttcc | |
| | tgtgtgaaattgttatccgctcacaattccacacaacatacgagccggaagcata | |
| | aagtgtaaagcctggggtgcctaatgagtgagctaactcacattaattgcgttgc | |
| | gctcactgcccgctttccagtcgggaaacctgtcgtgccagctgcattaatgaat | |
| | cggccaacgcgcggggagaggcggtttgcgtattgggcgctcttccgcttcctcg | |
| | ctcactgactcgctgcgctcggtcgttcggctgcggcgagcggtatcagctcact | |
| | caaaggcggtaatacggttatccacagaatcaggggataacgcaggaaagaacat | |
| | gtgagcaaaaggccagcaaaaggccaggaaccgtaaaaaggccgcgttgctggcg | |
| | tttttccataggctccgcccccctgacgagcatcacaaaaatcgacgctcaagtc | |
| | agaggtggcgaaacccgacaggactataaagataccaggcgtttccccctggaag | |
| | ctccctcgtgcgctctcctgttccgaccctgccgcttaccggatacctgtccgcc | |
| | tttctcccttcgggaagcgtggcgctttctcatagctcacgctgtaggtatctca | |
| | gttcggtgtaggtcgttcgctccaagctgggctgtgtgcacgaaccccccgttca | |
| | gcccgaccgctgcgccttatccggtaactatcgtcttgagtccaacccggtaaga | |
| | cacgacttatcgccactggcagcagccactggtaacaggattagcagagcgaggt | |
| | atgtaggcggtgctacagagttcttgaagtggtggcctaactacggctacactag | |
| | aaggacagtatttggtatctgcgctctgctgaagccagttaccttcggaaaaaga | |
| | gttggtagctcttgatccggcaaacaaaccaccgctggtagcggtggtttttttg | |
| | tttgcaagcagcagattacgcgcagaaaaaaaggatctcaagaagatcctttgat | |
| | cttttctacggggtctgacgctcagtggaacgaaaactcacgttaagggattttg | |
| | gtcatgagattatcaaaaaggatcttcacctagatccttttaaattaaaaatgaa | |
| | gttttaaatcaatctaaagtatatatgagtaaacttggtctgacagttaccaatg | |
| | cttaatcagtgaggcacctatctcagcgatctgtctatttcgttcatccatagtt | |
| | gcctgactccccgtcgtgtagataactacgatacgggagggcttaccatctggcc | |
| | ccagtgctgcaatgataccgcgagacccacgctcaccggctccagatttatcagc | |
| | aataaaccagccagccggaagggccgagcgcagaagtggtcctgcaactttatcc | |
| | gcctccatccagtctattaattgttgccgggaagctagagtaagtagttcgccag | |
| | ttaatagtttgcgcaacgttgttgccattgctacaggcatcgtggtgtcacgctc | |
| | gtcgtttggtatggcttcattcagctccggttcccaacgatcaaggcgagttaca | |
| | tgatcccccatgttgtgcaaaaaagcggttagctccttcggtcctccgatcgttg | |
| | tcagaagtaagttggccgcagtgttatcactcatggttatggcagcactgcataa | |
| | ttctcttactgtcatgccatccgtaagatgcttttctgtgactggtgagtactca | |
| | accaagtcattctgagaatagtgtatgcggcgaccgagttgctcttgcccggcgt | |
| | caatacgggataataccgcgccacatagcagaactttaaaagtgctcatcattgg | |
| | aaaacgttcttcggggcgaaaactctcaaggatcttaccgctgttgagatccagt | |
| | tcgatgtaacccactcgtgcacccaactgatcttcagcatcttttactttcacca | |
| | gcgtttctgggtgagcaaaaacaggaaggcaaaatgccgcaaaaaagggaataag | |
| | ggcgacacggaaatgttgaatactcatactcttcctttttc | |
| 8461 pcLV- | aatattttgaagcatttatcagggttattgtctcatgagcggatacatatttgaa | 95 |
| CMV-AFX01- | tgtatttagaaaaataaacaaataggggttccgcgcacatttccccgaaaagtgc | |
| 230 | cacctgacgtctaagaaaccattattatcatgacattaacctataaaaataggcg | |
| XT75(C)- | tatcacgaggccctttcgtctcgcgcgtttcggtgatgacggtgaaaacctctga | |
| IRES-Neo | cacatgcagctcccggagacggtcacagcttgtctgtaagcggatgccgggagca | |
| (9590 bp) | gacaagcccgtcagggcgcgtcagcgggtgttggcgggtgtcggggctggcttaa | |
| | ctatgcggcatcagagcagattgtactgagagtgcaccatatgcggtgtgaaata | |
| | ccgcacagatgcgtaaggagaaaataccgcatcaggcgccattcgccattcaggc | |
| | tgcgcaactgttgggaagggcgatcggtgcgggcctcttcgctattacgccagct | |
| | ggcgaaagggggatgtgctgcaaggcgattaagttgggtaacgccagggttttcc | |
| | cagtcacgacgttgtaaaacgacggccagtgccaagctgacgcgtgtagtcttat | |
| | gcaatactcttgtagtcttgcaacatggtaacgatgagttagcaacatgccttac | |
| | aaggagagaaaaagcaccgtgcatgccgattggtggaagtaaggtggtacgatcg | |
| | tgccttattaggaaggcaacagacgggtctgacatggattggacgaaccactgaa | |
| | ttgccgcattgcagagatattgtatttaagtgcctagctcgatacataaacgggt | |
| | ctctctggttagaccagatctgagcctgggagctctctggctaactagggaaccc | |
| | actgcttaagcctcaataaagcttgccttgagtgcttcaagtagtgtgtgcccgt | |
| | ctgttgtgtgactctggtaactagagatccctcagacccttttagtcagtgtgga | |
| | aaatctctagcagtggcgcccgaacagggacttgaaagcgaaagggaaaccagag | |
| | gagctctctcgacgcaggactcggcttgctgaagcgcgcacggcaagaggcgagg | |
| | ggcggcgactggtgagtacgccaaaaattttgactagcggaggctagaaggagag | |
| | agatgggtgcgagagcgtcagtattaagcgggggagaattagatcgcgatgggaa | |
| | aaaattcggttaaggccagggggaaagaaaaaatataaattaaaacatatagtat | |
| | gggcaagcagggagctagaacgattcgcagttaatcctggcctgttagaaacatc | |
| | agaaggctgtagacaaatactgggacagctacaaccatcccttcagacaggatca | |
| | gaagaacttagatcattatataatacagtagcaaccctctattgtgtgcatcaaa | |
| | ggatagagataaaagacaccaaggaagctttagacaagatagaggaagagcaaaa | |
| | caaaagtaagaccaccgcacagcaagcggccactgatcttcagacctggaggagg | |
| | agatatgagggacaattggagaagtgaattatataaatataaagtagtaaaaatt | |
| | gaaccattaggagtagcacccaccaaggcaaagagaagagtggtgcagagagaaa | |
| | aaagagcagtgggaataggagctttgttccttgggttcttgggagcagcaggaag | |
| | cactatgggcgcagcgtcaatgacgctgacggtacaggccagacaattattgtct | |
| | ggtatagtgcagcagcagaacaatttgctgagggctattgaggcgcaacagcatc | |
| | tgttgcaactcacagtctggggcatcaagcagctccaggcaagaatcctggctgt | |
| | ggaaagatacctaaaggatcaacagctcctggggatttggggttgctctggaaaa | |
| | ctcatttgcaccactgctgtgccttggaatgctagttggagtaataaatctctgg | |
| | aacagatttggaatcacacgacctggatggagtgggacagagaaattaacaatta | |
| | cacaagcttaatacactccttaattgaagaatcgcaaaaccagcaagaaaagaat | |
| | gaacaagaattattggaattagataaatgggcaagtttgtggaattggtttaaca | |
| | taacaaattggctgtggtatataaaattattcataatgatagtaggaggcttggt | |
| | aggtttaagaatagtttttgctgtactttctatagtgaatagagttaggcaggga | |
| | tattcaccattatcgtttcagacccacctcccaaccccgaggggacccgacaggc | |
| | ccgaaggaatagaagaagaaggtggagagagagacagagacagatccattcgatt | |
| | agtgaacggatctcgacggtatcggttaacttttaaaagaaaaggggggattggg | |
| | gggtacagtgcaggggaaagaatagtagacataatagcaacagacatacaaacta | |
| | aagaattacaaaaacaaattacaaaattcaaaattttatttccgcgttacataac | |
| | ttacggtaaatggcccgcctggctgaccgcccaacgacccccgcccattgacgtc | |
| | aataatgacgtatgttcccatagtaacgccaatagggactttccattgacgtcaa | |
| | tgggtggagtatttacggtaaactgcccacttggcagtacatcaagtgtatcata | |
| | tgccaagtacgccccctattgacgtcaatgacggtaaatggcccgcctggcatta | |
| | tgcccagtacatgaccttatgggactttcctacttggcagtacatctacgtatta | |
| | gtcatcgctattaccatggtgatgcggttttggcagtacatcaatgggcgtggat | |
| | agcggtttgactcacggggatttccaagtctccaccccattgacgtcaatgggag | |
| | tttgttttggcaccaaaatcaacgggactttccaaaatgtcgtaacaactccgcc | |
| | ccattgacgcaaatgggcggtaggcgtgtacggtgggaggtctatataagcagag | |
| | ctggtttagtgaaccgtcagatccgctagcctagcggatccgaattctcgatcac | |
| | aagtttgtacaaaaaagcaggcttcgctagccgccaccatgcccctgctcctgct | |
| | gctgcctctgctgtgggccggtgcattggccatccctggagggctgtccgaggcc | |
| | aagcctgctacgccggaaattcaagaaatcgtcgataaggttaagccacagctcg | |
| | aagagaaaactggggaaacatacggcaaacttgaggcggttcagtacaaaactca | |
| | ggtcgtgcactactacgtgcactacaacgatcaagggaccaactactatatcaag | |
| | gtccgcgcgggagacaacaaatacatgcatctcaaagtgttcaaatcgctgtggg | |
| | gcgagaaccttttcgcgaagtgggaggatttggtgctgactggatatcaggtcga | |
| | taaaaacaaggatgacgagcttactggctttgccgaggctgcagccaaagaagcc | |
| | gcggcgaaggaagcggcggcaaaggaggcagccgcaaaggaagctgccgctaaag | |
| | aggccgccgcaaagatgattcccggaggcctctcagaagccaaaccagccactcc | |
| | tgagattcaggaaattgtcgacaaggtcaagccgcagctggaagagaagactggc | |
| | gaaacctacgggaagctagaggcggtccagtacaagacccaagtcgtccattact | |
| | atgtacattacaacgaccagggaaccaattactacattaaagtgcgggccggaga | |
| | taataagtatatgcatcttaaggtgtttaagtcgttgtggggcgaaaacctgttt | |
| | gccaagtgggaagatcttgtgctcaccggttaccaagtggacaagaacaaggacg | |
| | acgagctgaccgggttcgctgaggctgccgccaaggaagccgcagcgaaggaggc | |
| | cgctgcgaaagaagcggcagccaaggaggcggctgccaaagaggcggccgcgaag | |
| | atgatacctggcggactgtcggaggcgaaaccggccacccctgaaatccaggaga | |
| | ttgtggacaaggtgaaaccccaactcgaggaaaaaaccggcgaaacgtacggtaa | |
| | actcgaagccgtgcagtataagacccaggtcgtacactactatgtgcactataac | |
| | gatcagggaacgaactactacatcaaggtcagagctggagacaataagtacatgc | |
| | acctcaaggttttcaagtcgttatggggagaaaacctcttcgcgaaatgggagga | |
| | tctcgtgctgaccggctaccaggtggacaaaaataaggatgatgaactcaccggt | |
| | ttcgctgaagcagcggcgaaagaggcagcggccaaggaagccgctgccaaggaag | |
| | ccgccgctaaggaggctgcggctaaggaagcagccgccaagatgatcccacgggg | |
| | tctgtccgaagccaagccagccaccccagagatccaagaaatagtggataaggtg | |
| | aagccccagcttgaggaaaagactggagagacttacggaaagctcgaggctgtcc | |
| | aatacaaaacacaggtgctggcgaacttcttccaaaggagatggccaggcagtac | |
| | taactattacatcaaagtgcgcgcaggagataacaaatatatgcacttgaaggtg | |
| | ttcaatggaccgtggaagttccggaacaccgacagaggcgccgacagggtcttga | |
| | ccgggtaccaagtcgataagaacaaagacgacgaactgaccggattctgaatcta | |
| | gagacccagctttcttgtacaaagtggtatcgagatatctgcatttaattaatct | |
| | agagtttaaacgcggccgcaaattccgcccctctccctcccccccccctaacgtt | |
| | actggccgaagccgcttggaataaggccggtgtgcgtttgtctatatgttatttt | |
| | ccaccatattgccgtcttttggcaatgtgagggcccggaaacctggccctgtctt | |
| | cttgacgagcattcctaggggtctttcccctctcgccaaaggaatgcaaggtctg | |
| | ttgaatgtcgtgaaggaagcagttcctctggaagcttcttgaagacaaacaacgt | |
| | ctgtagcgaccctttgcaggcagcggaaccccccacctggcgacaggtgcctctg | |
| | cggccaaaagccacgtgtataagatacacctgcaaaggcggcacaaccccagtgc | |
| | cacgttgtgagttggatagttgtggaaagagtcaaatggctctcctcaagcgtat | |
| | tcaacaaggggctgaaggatgcccagaaggtaccccattgtatgggatctgatct | |
| | ggggcctcggtgcacatgctttacatgtgtttagtcgaggttaaaaaaacgtcta | |
| | ggccccccgaaccacggggacgtggttttcctttgaaaaacacgatgataatatg | |
| | gccacaaccatggttattgaacaagatggattgcacgcaggttctccggccgctt | |
| | gggtggagaggctattcggctatgactgggcacaacagacaatcggctgctctga | |
| | tgccgccgtgttccggctgtcagcgcaggggcgcccggttctttttgtcaagacc | |
| | gacctgtccggtgccctgaatgaactgcaggacgaggcagcgcggctatcgtggc | |
| | tggccacgacgggcgttccttgcgcagctgtgctcgacgttgtcactgaagcggg | |
| | aagggactggctgctattgggcgaagtgccggggcaggatctcctgtcatctcac | |
| | cttgctcctgccgagaaagtatccatcatggctgatgcaatgcggcggctgcata | |
| | cgcttgatccggctacctgcccattcgaccaccaagcgaaacatcgcatcgagcg | |
| | agcacgtactcggatggaagccggtcttgtcgatcaggatgatctggacgaagag | |
| | catcaggggctcgcgccagccgaactgttcgccaggctcaaggcgcgcatgcccg | |
| | acggcgaggatctcgtcgtgacccatggcgatgcctgcttgccgaatatcatggt | |
| | ggaaaatggccgcttttctggattcatcgactgtggccggctgggtgtggcggac | |
| | cgctatcaggacatagcgttggctacccgtgatattgctgaagagcttggcggcg | |
| | aatgggctgaccgcttcctcgtgctttacggtatcgccgctcccgattcgcagcg | |
| | catcgccttctatcgccttcttgacgagttcttctgagtcgacaatcaacctctg | |
| | gattacaaaatttgtgaaagattgactggtattcttaactatgttgctcctttta | |
| | cgctatgtggatacgctgctttaatgcctttgtatcatgctattgcttcccgtat | |
| | ggctttcattttctcctccttgtataaatcctggttgctgtctctttatgaggag | |
| | ttgtggcccgttgtcaggcaacgtggcgtggtgtgcactgtgtttgctgacgcaa | |
| | cccccactggttggggcattgccaccacctgtcagctcctttccgggactttcgc | |
| | tttccccctccctattgccacggcggaactcatcgccgcctgccttgcccgctgc | |
| | tggacaggggctcggctgttgggcactgacaattccgtggtgttgtcggggaaat | |
| | catcgtcctttccttggctgctcgcctgtgttgccacctggattctgcgcgggac | |
| | gtccttctgctacgtcccttcggccctcaatccagcggaccttccttcccgcggc | |
| | ctgctgccggctctgcggcctcttccgcgtcttcgccttcgccctcagacgagtc | |
| | ggatctccctttgggccgcctccccgcctggtacctttaagaccaatgacttaca | |
| | aggcagctgtagatcttagccactttttaaaagaaaaggggggactggaagggct | |
| | aattcactcccaacgaagataagatctgctttttgcttgtactgggtctctctgg | |
| | ttagaccagatctgagcctgggagctctctggctaactagggaacccactgctta | |
| | agcctcaataaagcttgccttgagtgcttcaagtagtgtgtgcccgtctgttgtg | |
| | tgactctggtaactagagatccctcagacccttttagtcagtgtggaaaatctct | |
| | agcagtagtagttcatgtcatcttattattcagtatttataacttgcaaagaaat | |
| | gaatatcagagagtgagaggaacttgtttattgcagcttataatggttacaaata | |
| | aagcaatagcatcacaaatttcacaaataaagcatttttttcactgcattctagt | |
| | tgtggtttgtccaaactcatcaatgtatcttatcatgtctggctctagctatccc | |
| | gcccctaactccgcccatcccgcccctaactccgcccagttccgcccattctccg | |
| | ccccatggctgactaattttttttatttatgcagaggccgaggccgcctcggcct | |
| | ctgagctattccagaagtagtgaggaggcttttttggaggcctagacttttgcag | |
| | agaccaaattcgtaatcatgtcatagctgtttcctgtgtgaaattgttatccgct | |
| | cacaattccacacaacatacgagccggaagcataaagtgtaaagcctggggtgcc | |
| | taatgagtgagctaactcacattaattgcgttgcgctcactgcccgctttccagt | |
| | cgggaaacctgtcgtgccagctgcattaatgaatcggccaacgcgcggggagagg | |
| | cggtttgcgtattgggcgctcttccgcttcctcgctcactgactcgctgcgctcg | |
| | gtcgttcggctgcggcgagcggtatcagctcactcaaaggcggtaatacggttat | |
| | ccacagaatcaggggataacgcaggaaagaacatgtgagcaaaaggccagcaaaa | |
| | ggccaggaaccgtaaaaaggccgcgttgctggcgtttttccataggctccgcccc | |
| | cctgacgagcatcacaaaaatcgacgctcaagtcagaggtggcgaaacccgacag | |
| | gactataaagataccaggcgtttccccctggaagctccctcgtgcgctctcctgt | |
| | tccgaccctgccgcttaccggatacctgtccgcctttctcccttcgggaagcgtg | |
| | gcgctttctcatagctcacgctgtaggtatctcagttcggtgtaggtcgttcgct | |
| | ccaagctgggctgtgtgcacgaaccccccgttcagcccgaccgctgcgccttatc | |
| | cggtaactatcgtcttgagtccaacccggtaagacacgacttatcgccactggca | |
| | gcagccactggtaacaggattagcagagcgaggtatgtaggcggtgctacagagt | |
| | tcttgaagtggtggcctaactacggctacactagaaggacagtatttggtatctg | |
| | cgctctgctgaagccagttaccttcggaaaaagagttggtagctcttgatccggc | |
| | aaacaaaccaccgctggtagcggtggtttttttgtttgcaagcagcagattacgc | |
| | gcagaaaaaaaggatctcaagaagatcctttgatcttttctacggggtctgacgc | |
| | tcagtggaacgaaaactcacgttaagggattttggtcatgagattatcaaaaagg | |
| | atcttcacctagatccttttaaattaaaaatgaagttttaaatcaatctaaagta | |
| | tatatgagtaaacttggtctgacagttaccaatgcttaatcagtgaggcacctat | |
| | ctcagcgatctgtctatttcgttcatccatagttgcctgactccccgtcgtgtag | |
| | ataactacgatacgggagggcttaccatctggccccagtgctgcaatgataccgc | |
| | gagacccacgctcaccggctccagatttatcagcaataaaccagccagccggaag | |
| | ggccgagcgcagaagtggtcctgcaactttatccgcctccatccagtctattaat | |
| | tgttgccgggaagctagagtaagtagttcgccagttaatagtttgcgcaacgttg | |
| | ttgccattgctacaggcatcgtggtgtcacgctcgtcgtttggtatggcttcatt | |
| | cagctccggttcccaacgatcaaggcgagttacatgatcccccatgttgtgcaaa | |
| | aaagcggttagctccttcggtcctccgatcgttgtcagaagtaagttggccgcag | |
| | tgttatcactcatggttatggcagcactgcataattctcttactgtcatgccatc | |
| | cgtaagatgcttttctgtgactggtgagtactcaaccaagtcattctgagaatag | |
| | tgtatgcggcgaccgagttgctcttgcccggcgtcaatacgggataataccgcgc | |
| | cacatagcagaactttaaaagtgctcatcattggaaaacgttcttcggggcgaaa | |
| | actctcaaggatcttaccgctgttgagatccagttcgatgtaacccactcgtgca | |
| | cccaactgatcttcagcatcttttactttcaccagcgtttctgggtgagcaaaaa | |
| | caggaaggcaaaatgccgcaaaaaagggaataagggcgacacggaaatgttgaat | |
| | actcatactcttcctttttc | |
| VB211001- | aatgtagtcttatgcaatactcttgtagtcttgcaacatggtaacgatgagttag | 96 |
| 1274vjf | caacatgccttacaaggagagaaaaagcaccgtgcatgccgattggtggaagtaa | |
| (9895 bp) | ggtggtacgatcgtgccttattaggaaggcaacagacgggtctgacatggattgg | |
| | acgaaccactgaattgccgcattgcagagatattgtatttaagtgcctagctcga | |
| | tacataaacgggtctctctggttagaccagatctgagcctgggagctctctggct | |
| | aactagggaacccactgcttaagcctcaataaagcttgccttgagtgcttcaagt | |
| | agtgtgtgcccgtctgttgtgtgactctggtaactagagatccctcagacccttt | |
| | tagtcagtgtggaaaatctctagcagtggcgcccgaacagggacttgaaagcgaa | |
| | agggaaaccagaggagctctctcgacgcaggactcggcttgctgaagcgcgcacg | |
| | gcaagaggcgaggggcggcgactggtgagtacgccaaaaattttgactagcggag | |
| | gctagaaggagagagatgggtgcgagagcgtcagtattaagcgggggagaattag | |
| | atcgcgatgggaaaaaattcggttaaggccagggggaaagaaaaaatataaatta | |
| | aaacatatagtatgggcaagcagggagctagaacgattcgcagttaatcctggcc | |
| | tgttagaaacatcagaaggctgtagacaaatactgggacagctacaaccatccct | |
| | tcagacaggatcagaagaacttagatcattatataatacagtagcaaccctctat | |
| | tgtgtgcatcaaaggatagagataaaagacaccaaggaagctttagacaagatag | |
| | aggaagagcaaaacaaaagtaagaccaccgcacagcaagcggccgctgatcttca | |
| | gacctggaggaggagatatgagggacaattggagaagtgaattatataaatataa | |
| | agtagtaaaaattgaaccattaggagtagcacccaccaaggcaaagagaagagtg | |
| | gtgcagagagaaaaaagagcagtgggaataggagctttgttccttgggttcttgg | |
| | gagcagcaggaagcactatgggcgcagcgtcaatgacgctgacggtacaggccag | |
| | acaattattgtctggtatagtgcagcagcagaacaatttgctgagggctattgag | |
| | gcgcaacagcatctgttgcaactcacagtctggggcatcaagcagctccaggcaa | |
| | gaatcctggctgtggaaagatacctaaaggatcaacagctcctggggatttgggg | |
| | ttgctctggaaaactcatttgcaccactgctgtgccttggaatgctagttggagt | |
| | aataaatctctggaacagatttggaatcacacgacctggatggagtgggacagag | |
| | aaattaacaattacacaagcttaatacactccttaattgaagaatcgcaaaacca | |
| | gcaagaaaagaatgaacaagaattattggaattagataaatgggcaagtttgtgg | |
| | aattggtttaacataacaaattggctgtggtatataaaattattcataatgatag | |
| | taggaggcttggtaggtttaagaatagtttttgctgtactttctatagtgaatag | |
| | agttaggcagggatattcaccattatcgtttcagacccacctcccaaccccgagg | |
| | ggacccgacaggcccgaaggaatagaagaagaaggtggagagagagacagagaca | |
| | gatccattcgattagtgaacggatctcgacggtatcgctagcttttaaaagaaaa | |
| | ggggggattggggggtacagtgcaggggaaagaatagtagacataatagcaacag | |
| | acatacaaactaaagaattacaaaaacaaattacaaaaattcaaaattttactag | |
| | tatcaactttgtatagaaaagttgtagttattaatagtaatcaattacggggtca | |
| | ttagttcatagcccatatatggagttccgcgttacataacttacggtaaatggcc | |
| | cgcctggctgaccgcccaacgacccccgcccattgacgtcaataatgacgtatgt | |
| | tcccatagtaacgccaatagggactttccattgacgtcaatgggtggagtattta | |
| | cggtaaactgcccacttggcagtacatcaagtgtatcatatgccaagtacgcccc | |
| | ctattgacgtcaatgacggtaaatggcccgcctggcattatgcccagtacatgac | |
| | cttatgggactttcctacttggcagtacatctacgtattagtcatcgctattacc | |
| | atggtgatgcggttttggcagtacatcaatgggcgtggatagcggtttgactcac | |
| | ggggatttccaagtctccaccccattgacgtcaatgggagtttgttttggcacca | |
| | aaatcaacgggactttccaaaatgtcgtaacaactccgccccattgacgcaaatg | |
| | ggcggtaggcgtgtacggtgggaggtctatataagcagagctggtttagtgaacc | |
| | gtcagatccaagtttgtacaaaaaagcaggctgccaccatgccattgcttcttct | |
| | gctgccactcctttgggctggtgcgctggctattccggggggtttgtcagaagca | |
| | aagcccgcgaccccagaaattcaggagatcgtggataaagtaaaacctcagcttg | |
| | aagaaaaaaccggggagacatacggcaagctggaagcagtacaatataaaacgca | |
| | ggtagtacattactacgtccattataacgatcaaggtacaaattattatataaaa | |
| | gtacgggcaggggacaacaaatacatgcatctgaaagtatttaagagtctgtggg | |
| | gtgagaacttgtttgcaaagtgggaagatttggtgcttaccggctaccaggtcga | |
| | taagaataaggatgacgagcttacaggttttgcagaagctgcagcgaaagaggct | |
| | gccgcaaaagaagcagcggctaaggaagcggccgctaaagaagcggccgcgaagg | |
| | aagcggccgctaagatgatcccgggtggtctgtccgaagccaaaccggcgacgcc | |
| | ggaaatccaggagatagtcgataaggtcaagccacaactcgaagagaagactgga | |
| | gaaacatatggaaaacttgaggccgttcaatacaaaacgcaggtggtccactact | |
| | acgttcactataacgatcaaggaacaaactattacattaaagtcagggcgggaga | |
| | caacaagtacatgcatcttaaagtgtttaaatctctttggggggagaatcttttc | |
| | gccaagtgggaagacttggtcctcaccggataccaggtcgacaaaaacaaagacg | |
| | atgaactcaccggctttgccgaagcggctgcgaaagaagcggctgccaaagaggc | |
| | tgcagccaaagaagctgcggcaaaggaggccgccgccaaagaagcggcggccaaa | |
| | atgatccccggaggacttagcgaggctaaacccgcgacacctgaaatacaggaga | |
| | tcgtggataaggttaaaccacaattggaggagaaaactggtgagacctatggcaa | |
| | gctggaagcggttcagtataaaacgcaagttgtacactactatgtacattacaac | |
| | gatcaggggacgaactactatattaaggtcagggcaggagataataaatatatgc | |
| | acttgaaagttttcaaaagcctgtggggggaaaacttgttcgccaagtgggaaga | |
| | tctcgtcttgactgggtaccaggttgataagaacaaagacgatgaactgacaggc | |
| | ttcgctgaagcggctgcaaaggaggcggcagcgaaggaggctgccgcgaaagaag | |
| | cggcggctaaagaagcggccgcaaaggaagcagcagcgaagatgataccgagggg | |
| | tttgagcgaagcaaagcccgcaacgcctgagatacaagaaatcgttgacaaagta | |
| | aaaccacaattggaagagaagacgggagaaacgtatggaaagcttgaggccgtac | |
| | aatacaagacacaggtgttggcaaattttttccaaaggcgatggcccggatcaac | |
| | gaattactatatcaaggtacgcgcgggggataacaagtacatgcatctcaaggtt | |
| | ttcaacggtccgtggaaattccgaaatacagaccgcggagccgacagggtcctca | |
| | ccggctatcaggtcgacaagaataaggatgatgagttgacaggtttcgccgccgc | |
| | cggtgagcagaaattgatctccgaagaggatctcggtgccgcgcaccaccaccac | |
| | catcattaaacccagctttcttgtacaaagtgggcccctctccctcccccccccc | |
| | taacgttactggccgaagccgcttggaataaggccggtgtgcgtttgtctatatg | |
| | ttattttccaccatattgccgtcttttggcaatgtgagggcccggaaacctggcc | |
| | ctgtcttcttgacgagcattcctaggggtctttcccctctcgccaaaggaatgca | |
| | aggtctgttgaatgtcgtgaaggaagcagttcctctggaagcttcttgaagacaa | |
| | acaacgtctgtagcgaccctttgcaggcagcggaaccccccacctggcgacaggt | |
| | gcctctgcggccaaaagccacgtgtataagatacacctgcaaaggcggcacaacc | |
| | ccagtgccacgttgtgagttggatagttgtggaaagagtcaaatggctctcctca | |
| | agcgtattcaacaaggggctgaaggatgcccagaaggtaccccattgtatgggat | |
| | ctgatctggggcctcggtgcacatgctttacatgtgtttagtcgaggttaaaaaa | |
| | acgtctaggccccccgaaccacggggacgtggttttcctttgaaaaacacgatga | |
| | taatatggccacaaccatgattgaacaagatggattgcacgcaggttctccggcc | |
| | gcttgggtggagaggctattcggctatgactgggcacaacagacaatcggctgct | |
| | ctgatgccgccgtgttccggctgtcagcgcaggggcgcccggttctttttgtcaa | |
| | gaccgacctgtccggtgccctgaatgaactgcaagacgaggcagcgcggctatcg | |
| | tggctggccacgacgggcgttccttgcgcagctgtgctcgacgttgtcactgaag | |
| | cgggaagggactggctgctattgggcgaagtgccggggcaggatctcctgtcatc | |
| | tcaccttgctcctgccgagaaagtatccatcatggctgatgcaatgcggcggctg | |
| | catacgcttgatccggctacctgcccattcgaccaccaagcgaaacatcgcatcg | |
| | agcgagcacgtactcggatggaagccggtcttgtcgatcaggatgatctggacga | |
| | agagcatcaggggctcgcgccagccgaactgttcgccaggctcaaggcgagcatg | |
| | cccgacggcgaggatctcgtcgtgacccatggcgatgcctgcttgccgaatatca | |
| | tggtggaaaatggccgcttttctggattcatcgactgtggccggctgggtgtggc | |
| | ggaccgctatcaggacatagcgttggctacccgtgatattgctgaagagcttggc | |
| | ggcgaatgggctgaccgcttcctcgtgctttacggtatcgccgctcccgattcgc | |
| | agcgcatcgccttctatcgccttcttgacgagttcttctgacaactttattatac | |
| | atagttgatcaattccgataatcaacctctggattacaaaatttgtgaaagattg | |
| | actggtattcttaactatgttgctccttttacgctatgtggatacgctgctttaa | |
| | tgcctttgtatcatgctattgcttcccgtatggctttcattttctcctccttgta | |
| | taaatcctggttgctgtctctttatgaggagttgtggcccgttgtcaggcaacgt | |
| | ggcgtggtgtgcactgtgtttgctgacgcaacccccactggttggggcattgcca | |
| | ccacctgtcagctcctttccgggactttcgctttccccctccctattgccacggc | |
| | ggaactcatcgccgcctgccttgcccgctgctggacaggggctcggctgttgggc | |
| | actgacaattccgtggtgttgtcggggaagctgacgtcctttccatggctgctcg | |
| | cctgtgttgccacctggattctgcgcgggacgtccttctgctacgtcccttcggc | |
| | cctcaatccagcggaccttccttcccgcggcctgctgccggctctgcggcctctt | |
| | ccgcgtcttcgccttcgccctcagacgagtcggatctccctttgggccgcctccc | |
| | cgcatcgggaattcccgcggttcgctttaagaccaatgacttacaaggcagctgt | |
| | agatcttagccactttttaaaagaaaaggggggactggaagggctaattcactcc | |
| | caacgaagacaagatctgctttttgcttgtactgggtctctctggttagaccaga | |
| | tctgagcctgggagctctctggctaactagggaacccactgcttaagcctcaata | |
| | aagcttgccttgagtgcttcaagtagtgtgtgcccgtctgttgtgtgactctggt | |
| | aactagagatccctcagacccttttagtcagtgtggaaaatctctagcagtagta | |
| | gttcatgtcatcttattattcagtatttataacttgcaaagaaatgaatatcaga | |
| | gagtgagaggaacttgtttattgcagcttataatggttacaaataaagcaatagc | |
| | atcacaaatttcacaaataaagcatttttttcactgcattctagttgtggtttgt | |
| | ccaaactcatcaatgtatcttatcatgtctggctctagctatcccgcccctaact | |
| | ccgcccatcccgcccctaactccgcccagttccgcccattctccgccccatggct | |
| | gactaattttttttatttatgcagaggccgaggccgcctcggcctctgagctatt | |
| | ccagaagtagtgaggaggcttttttggaggcctagggacgtacccaattcgccct | |
| | atagtgagtcgtattacgcgcgctcactggccgtcgttttacaacgtcgtgactg | |
| | ggaaaaccctggcgttacccaacttaatcgccttgcagcacatccccctttcgcc | |
| | agctggcgtaatagcgaagaggcccgcaccgatcgcccttcccaacagttgcgca | |
| | gcctgaatggcgaatgggacgcgccctgtagcggcgcattaagcgcggcgggtgt | |
| | ggtggttacgcgcagcgtgaccgctacacttgccagcgccctagcgcccgctcct | |
| | ttcgctttcttcccttcctttctcgccacgttcgccggctttccccgtcaagctc | |
| | taaatcgggggctccctttagggttccgatttagtgctttacggcacctcgaccc | |
| | caaaaaacttgattagggtgatggttcacgtagtgggccatcgccctgatagacg | |
| | gtttttcgccctttgacgttggagtccacgttctttaatagtggactcttgttcc | |
| | aaactggaacaacactcaaccctatctcggtctattcttttgatttataagggat | |
| | tttgccgatttcggcctattggttaaaaaatgagctgatttaacaaaaatttaac | |
| | gcgaattttaacaaaatattaacgcttacaatttaggtggcacttttcggggaaa | |
| | tgtgcgcggaacccctatttgtttatttttctaaatacattcaaatatgtatccg | |
| | ctcatgagacaataaccctgataaatgcttcaataatattgaaaaaggaagagta | |
| | tgagtattcaacatttccgtgtcgcccttattcccttttttgcggcattttgcct | |
| | tcctgtttttgctcacccagaaacgctggtgaaagtaaaagatgctgaagatcag | |
| | ttgggtgcacgagtgggttacatcgaactggatctcaacagcggtaagatccttg | |
| | agagttttcgccccgaagaacgttttccaatgatgagcacttttaaagttctgct | |
| | atgtggcgcggtattatcccgtattgacgccgggcaagagcaactcggtcgccgc | |
| | atacactattctcagaatgacttggttgagtactcaccagtcacagaaaagcatc | |
| | ttacggatggcatgacagtaagagaattatgcagtgctgccataaccatgagtga | |
| | taacactgcggccaacttacttctgacaacgatcggaggaccgaaggagctaacc | |
| | gcttttttgcacaacatgggggatcatgtaactcgccttgatcgttgggaaccgg | |
| | agctgaatgaagccataccaaacgacgagcgtgacaccacgatgcctgtagcaat | |
| | ggcaacaacgttgcgcaaactattaactggcgaactacttactctagcttcccgg | |
| | caacaattaatagactggatggaggcggataaagttgcaggaccacttctgcgct | |
| | cggcccttccggctggctggtttattgctgataaatctggagccggtgagcgtgg | |
| | gtctcgcggtatcattgcagcactggggccagatggtaagccctcccgtatcgta | |
| | gttatctacacgacggggagtcaggcaactatggatgaacgaaatagacagatcg | |
| | ctgagataggtgcctcactgattaagcattggtaactgtcagaccaagtttactc | |
| | atatatactttagattgatttaaaacttcatttttaatttaaaaggatctaggtg | |
| | aagatcctttttgataatctcatgaccaaaatcccttaacgtgagttttcgttcc | |
| | actgagcgtcagaccccgtagaaaagatcaaaggatcttcttgagatcctttttt | |
| | tctgcgcgtaatctgctgcttgcaaacaaaaaaaccaccgctaccagcggtggtt | |
| | tgtttgccggatcaagagctaccaactctttttccgaaggtaactggcttcagca | |
| | gagcgcagataccaaatactgttcttctagtgtagccgtagttaggccaccactt | |
| | caagaactctgtagcaccgcctacatacctcgctctgctaatcctgttaccagtg | |
| | gctgctgccagtggcgataagtcgtgtcttaccgggttggactcaagacgatagt | |
| | taccggataaggcgcagcggtcgggctgaacggggggttcgtgcacacagcccag | |
| | cttggagcgaacgacctacaccgaactgagatacctacagcgtgagctatgagaa | |
| | agcgccacgcttcccgaagagagaaaggcggacaggtatccggtaagcggcaggg | |
| | tcggaacaggagagcgcacgagggagcttccagggggaaacgcctggtatcttta | |
| | tagtcctgtcgggtttcgccacctctgacttgagcgtcgatttttgtgatgctcg | |
| | tcaggggggcggagcctatggaaaaacgccagcaacgcggcctttttacggttcc | |
| | tggccttttgctggccttttgctcacatgttctttcctgcgttatcccctgattc | |
| | tgtggataaccgtattaccgcctttgagtgagctgataccgctcgccgcagccga | |
| | acgaccgagcgcagcgagtcagtgagcgaggaagcggaagagcgcccaatacgca | |
| | aaccgcctctccccgcgcgttggccgattcattaatgcagctggcacgacaggtt | |
| | tcccgactggaaagcgggcagtgagcgcaacgcaattaatgtgagttagctcact | |
| | cattaggcaccccaggctttacactttatgcttccggctcgtatgttgtgtggaa | |
| | ttgtgagcggataacaatttcacacaggaaacagctatgaccatgattacgccaa | |
| | gcgcgcaattaaccctcactaaagggaacaaaagctggagctgcaagctt | |
| VB211001- | aatgtagtcttatgcaatactcttgtagtcttgcaacatggtaacgatgagttag | 97 |
| 1279pfe | caacatgccttacaaggagagaaaaagcaccgtgcatgccgattggtggaagtaa | |
| (9361 bp) | ggtggtacgatcgtgccttattaggaaggcaacagacgggtctgacatggattgg | |
| | acgaaccactgaattgccgcattgcagagatattgtatttaagtgcctagctcga | |
| | tacataaacgggtctctctggttagaccagatctgagcctgggagctctctggct | |
| | aactagggaacccactgcttaagcctcaataaagcttgccttgagtgcttcaagt | |
| | agtgtgtgcccgtctgttgtgtgactctggtaactagagatccctcagacccttt | |
| | tagtcagtgtggaaaatctctagcagtggcgcccgaacagggacttgaaagcgaa | |
| | agggaaaccagaggagctctctcgacgcaggactcggcttgctgaagcgcgcacg | |
| | gcaagaggcgaggggcggcgactggtgagtacgccaaaaattttgactagcggag | |
| | gctagaaggagagagatgggtgcgagagcgtcagtattaagcgggggagaattag | |
| | atcgcgatgggaaaaaattcggttaaggccagggggaaagaaaaaatataaatta | |
| | aaacatatagtatgggcaagcagggagctagaacgattcgcagttaatcctggcc | |
| | tgttagaaacatcagaaggctgtagacaaatactgggacagctacaaccatccct | |
| | tcagacaggatcagaagaacttagatcattatataatacagtagcaaccctctat | |
| | tgtgtgcatcaaaggatagagataaaagacaccaaggaagctttagacaagatag | |
| | aggaagagcaaaacaaaagtaagaccaccgcacagcaagcggccgctgatcttca | |
| | gacctggaggaggagatatgagggacaattggagaagtgaattatataaatataa | |
| | agtagtaaaaattgaaccattaggagtagcacccaccaaggcaaagagaagagtg | |
| | gtgcagagagaaaaaagagcagtgggaataggagctttgttccttgggttcttgg | |
| | gagcagcaggaagcactatgggcgcagcgtcaatgacgctgacggtacaggccag | |
| | acaattattgtctggtatagtgcagcagcagaacaatttgctgagggctattgag | |
| | gcgcaacagcatctgttgcaactcacagtctggggcatcaagcagctccaggcaa | |
| | gaatcctggctgtggaaagatacctaaaggatcaacagctcctggggatttgggg | |
| | ttgctctggaaaactcatttgcaccactgctgtgccttggaatgctagttggagt | |
| | aataaatctctggaacagatttggaatcacacgacctggatggagtgggacagag | |
| | aaattaacaattacacaagcttaatacactccttaattgaagaatcgcaaaacca | |
| | gcaagaaaagaatgaacaagaattattggaattagataaatgggcaagtttgtgg | |
| | aattggtttaacataacaaattggctgtggtatataaaattattcataatgatag | |
| | taggaggcttggtaggtttaagaatagtttttgctgtactttctatagtgaatag | |
| | agttaggcagggatattcaccattatcgtttcagacccacctcccaaccccgagg | |
| | ggacccgacaggcccgaaggaatagaagaagaaggtggagagagagacagagaca | |
| | gatccattcgattagtgaacggatctcgacggtatcgctagcttttaaaagaaaa | |
| | ggggggattggggggtacagtgcaggggaaagaatagtagacataatagcaacag | |
| | acatacaaactaaagaattacaaaaacaaattacaaaaattcaaaattttactag | |
| | tgattatcggatcaactttgtatagaaaagttgtagttattaatagtaatcaatt | |
| | acggggtcattagttcatagcccatatatggagttccgcgttacataacttacgg | |
| | taaatggcccgcctggctgaccgcccaacgacccccgcccattgacgtcaataat | |
| | gacgtatgttcccatagtaacgccaatagggactttccattgacgtcaatgggtg | |
| | gagtatttacggtaaactgcccacttggcagtacatcaagtgtatcatatgccaa | |
| | gtacgccccctattgacgtcaatgacggtaaatggcccgcctggcattatgccca | |
| | gtacatgaccttatgggactttcctacttggcagtacatctacgtattagtcatc | |
| | gctattaccatggtgatgcggttttggcagtacatcaatgggcgtggatagcggt | |
| | ttgactcacggggatttccaagtctccaccccattgacgtcaatgggagtttgtt | |
| | ttggcaccaaaatcaacgggactttccaaaatgtcgtaacaactccgccccattg | |
| | acgcaaatgggcggtaggcgtgtacggtgggaggtctatataagcagagctggtt | |
| | tagtgaaccgtcagatccaagtttgtacaaaaaagcaggctgccaccatgccatt | |
| | gcttcttctgctgccactcctttgggctggtgcgctggctattccggggggtttg | |
| | tcagaagcaaagcccgcgaccccagaaattcaggagatcgtggataaagtaaaac | |
| | ctcagcttgaagaaaaaaccggggagacatacggcaagctggaagcagtacaata | |
| | taaaacgcaggtagtacattactacgtccattataacgatcaaggtacaaattat | |
| | tatataaaagtacgggcaggggacaacaaatacatgcatctgaaagtatttaaga | |
| | gtctgtggggtgagaacttgtttgcaaagtgggaagatttggtgcttaccggcta | |
| | ccaggtcgataagaataaggatgacgagcttacaggttttgcagaagctgcagcg | |
| | aaagaggctgccgcaaaagaagcagcggctaaggaagcggccgctaaagaagcgg | |
| | ccgcgaaggaagcggccgctaagatgatcccgggtggtctgtccgaagccaaacc | |
| | ggcgacgccggaaatccaggagatagtcgataaggtcaagccacaactcgaagag | |
| | aagactggagaaacatatggaaaacttgaggccgttcaatacaaaacgcaggtgg | |
| | tccactactacgttcactataacgatcaaggaacaaactattacattaaagtcag | |
| | ggcgggagacaacaagtacatgcatcttaaagtgtttaaatctctttggggggag | |
| | aatcttttcgccaagtgggaagacttggtcctcaccggataccaggtcgacaaaa | |
| | acaaagacgatgaactcaccggctttgccgaagcggctgcgaaagaagcggctgc | |
| | caaagaggctgcagccaaagaagctgcggcaaaggaggccgccgccaaagaagcg | |
| | gcggccaaaatgatccccggaggacttagcgaggctaaacccgcgacacctgaaa | |
| | tacaggagatcgtggataaggttaaaccacaattggaggagaaaactggtgagac | |
| | ctatggcaagctggaagcggttcagtataaaacgcaagttgtacactactatgta | |
| | cattacaacgatcaggggacgaactactatattaaggtcagggcaggagataata | |
| | aatatatgcacttgaaagttttcaaaagcctgtggggggaaaacttgttcgccaa | |
| | gtgggaagatctcgtcttgactgggtaccaggttgataagaacaaagacgatgaa | |
| | ctgacaggcttcgctgaagcggctgcaaaggaggcggcagcgaaggaggctgccg | |
| | cgaaagaagcggcggctaaagaagcggccgcaaaggaagcagcagcgaagatgat | |
| | accgaggggtttgagcgaagcaaagcccgcaacgcctgagatacaagaaatcgtt | |
| | gacaaagtaaaaccacaattggaagagaagacgggagaaacgtatggaaagcttg | |
| | aggccgtacaatacaagacacaggtgttggcaaattttttccaaaggcgatggcc | |
| | cggatcaacgaattactatatcaaggtacgcgcgggggataacaagtacatgcat | |
| | ctcaaggttttcaacggtccgtggaaattccgaaatacagaccgcggagccgaca | |
| | gggtcctcaccggctatcaggtcgacaagaataaggatgatgagttgacaggttt | |
| | cgccgccgccggtgagcagaaattgatctccgaagaggatctcggtgccgcgcac | |
| | caccaccaccatcatggaagcggagagggcaggggaagtcttctaacatgcgggg | |
| | acgtggaggaaaatcccggccccatgattgaacaagatggattgcacgcaggttc | |
| | tccggccgcttgggtggagaggctattcggctatgactgggcacaacagacaatc | |
| | ggctgctctgatgccgccgtgttccggctgtcagcgcaggggcgcccggttcttt | |
| | ttgtcaagaccgacctgtccggtgccctgaatgaactgcaagacgaggcagcgcg | |
| | gctatcgtggctggccacgacgggcgttccttgcgcagctgtgctcgacgttgtc | |
| | actgaagcgggaagggactggctgctattgggcgaagtgccggggcaggatctcc | |
| | tgtcatctcaccttgctcctgccgagaaagtatccatcatggctgatgcaatgcg | |
| | gcggctgcatacgcttgatccggctacctgcccattcgaccaccaagcgaaacat | |
| | cgcatcgagcgagcacgtactcggatggaagccggtcttgtcgatcaggatgatc | |
| | tggacgaagagcatcaggggctcgcgccagccgaactgttcgccaggctcaaggc | |
| | gagcatgcccgacggcgaggatctcgtcgtgacccatggcgatgcctgcttgccg | |
| | aatatcatggtggaaaatggccgcttttctggattcatcgactgtggccggctgg | |
| | gtgtggcggaccgctatcaggacatagcgttggctacccgtgatattgctgaaga | |
| | gcttggcggcgaatgggctgaccgcttcctcgtgctttacggtatcgccgctccc | |
| | gattcgcagcgcatcgccttctatcgccttcttgacgagttcttctgaacccagc | |
| | tttcttgtacaaagtggtgataatcgaattccgataatcaacctctggattacaa | |
| | aatttgtgaaagattgactggtattcttaactatgttgctccttttacgctatgt | |
| | ggatacgctgctttaatgcctttgtatcatgctattgcttcccgtatggctttca | |
| | ttttctcctccttgtataaatcctggttgctgtctctttatgaggagttgtggcc | |
| | cgttgtcaggcaacgtggcgtggtgtgcactgtgtttgctgacgcaacccccact | |
| | ggttggggcattgccaccacctgtcagctcctttccgggactttcgctttccccc | |
| | tccctattgccacggcggaactcatcgccgcctgccttgcccgctgctggacagg | |
| | ggctcggctgttgggcactgacaattccgtggtgttgtcggggaagctgacgtcc | |
| | tttccatggctgctcgcctgtgttgccacctggattctgcgcgggacgtccttct | |
| | gctacgtcccttcggccctcaatccagcggaccttccttcccgcggcctgctgcc | |
| | ggctctgcggcctcttccgcgtcttcgccttcgccctcagacgagtcggatctcc | |
| | ctttgggccgcctccccgcatcgggaattcccgcggttcgctttaagaccaatga | |
| | cttacaaggcagctgtagatcttagccactttttaaaagaaaaggggggactgga | |
| | agggctaattcactcccaacgaagacaagatctgctttttgcttgtactgggtct | |
| | ctctggttagaccagatctgagcctgggagctctctggctaactagggaacccac | |
| | tgcttaagcctcaataaagcttgccttgagtgcttcaagtagtgtgtgcccgtct | |
| | gttgtgtgactctggtaactagagatccctcagacccttttagtcagtgtggaaa | |
| | atctctagcagtagtagttcatgtcatcttattattcagtatttataacttgcaa | |
| | agaaatgaatatcagagagtgagaggaacttgtttattgcagcttataatggtta | |
| | caaataaagcaatagcatcacaaatttcacaaataaagcatttttttcactgcat | |
| | tctagttgtggtttgtccaaactcatcaatgtatcttatcatgtctggctctagc | |
| | tatcccgcccctaactccgcccatcccgcccctaactccgcccagttccgcccat | |
| | tctccgccccatggctgactaattttttttatttatgcagaggccgaggccgcct | |
| | cggcctctgagctattccagaagtagtgaggaggcttttttggaggcctagggac | |
| | gtacccaattcgccctatagtgagtcgtattacgcgcgctcactggccgtcgttt | |
| | tacaacgtcgtgactgggaaaaccctggcgttacccaacttaatcgccttgcagc | |
| | acatccccctttcgccagctggcgtaatagcgaagaggcccgcaccgatcgccct | |
| | tcccaacagttgcgcagcctgaatggcgaatgggacgcgccctgtagcggcgcat | |
| | taagcgcggcgggtgtggtggttacgcgcagcgtgaccgctacacttgccagcgc | |
| | cctagcgcccgctcctttcgctttcttcccttcctttctcgccacgttcgccggc | |
| | tttccccgtcaagctctaaatcgggggctccctttagggttccgatttagtgctt | |
| | tacggcacctcgaccccaaaaaacttgattagggtgatggttcacgtagtgggcc | |
| | atcgccctgatagacggtttttcgccctttgacgttggagtccacgttctttaat | |
| | agtggactcttgttccaaactggaacaacactcaaccctatctcggtctattctt | |
| | ttgatttataagggattttgccgatttcggcctattggttaaaaaatgagctgat | |
| | ttaacaaaaatttaacgcgaattttaacaaaatattaacgcttacaatttaggtg | |
| | gcacttttcggggaaatgtgcgcggaacccctatttgtttatttttctaaataca | |
| | ttcaaatatgtatccgctcatgagacaataaccctgataaatgcttcaataatat | |
| | tgaaaaaggaagagtatgagtattcaacatttccgtgtcgcccttattccctttt | |
| | ttgcggcattttgccttcctgtttttgctcacccagaaacgctggtgaaagtaaa | |
| | agatgctgaagatcagttgggtgcacgagtgggttacatcgaactggatctcaac | |
| | agcggtaagatccttgagagttttcgccccgaagaacgttttccaatgatgagca | |
| | cttttaaagttctgctatgtggcgcggtattatcccgtattgacgccgggcaaga | |
| | gcaactcggtcgccgcatacactattctcagaatgacttggttgagtactcacca | |
| | gtcacagaaaagcatcttacggatggcatgacagtaagagaattatgcagtgctg | |
| | ccataaccatgagtgataacactgcggccaacttacttctgacaacgatcggagg | |
| | accgaaggagctaaccgcttttttgcacaacatgggggatcatgtaactcgcctt | |
| | gatcgttgggaaccggagctgaatgaagccataccaaacgacgagcgtgacacca | |
| | cgatgcctgtagcaatggcaacaacgttgcgcaaactattaactggcgaactact | |
| | tactctagcttcccggcaacaattaatagactggatggaggcggataaagttgca | |
| | ggaccacttctgcgctcggcccttccggctggctggtttattgctgataaatctg | |
| | gagccggtgagcgtgggtctcgcggtatcattgcagcactggggccagatggtaa | |
| | gccctcccgtatcgtagttatctacacgacggggagtcaggcaactatggatgaa | |
| | cgaaatagacagatcgctgagataggtgcctcactgattaagcattggtaactgt | |
| | cagaccaagtttactcatatatactttagattgatttaaaacttcatttttaatt | |
| | taaaaggatctaggtgaagatcctttttgataatctcatgaccaaaatcccttaa | |
| | cgtgagttttcgttccactgagcgtcagaccccgtagaaaagatcaaaggatctt | |
| | cttgagatcctttttttctgcgcgtaatctgctgcttgcaaacaaaaaaaccacc | |
| | gctaccagcggtggtttgtttgccggatcaagagctaccaactctttttccgaag | |
| | gtaactggcttcagcagagcgcagataccaaatactgttcttctagtgtagccgt | |
| | agttaggccaccacttcaagaactctgtagcaccgcctacatacctcgctctgct | |
| | aatcctgttaccagtggctgctgccagtggcgataagtcgtgtcttaccgggttg | |
| | gactcaagacgatagttaccggataaggcgcagcggtcgggctgaacggggggtt | |
| | cgtgcacacagcccagcttggagcgaacgacctacaccgaactgagatacctaca | |
| | gcgtgagctatgagaaagcgccacgcttcccgaagagagaaaggcggacaggtat | |
| | ccggtaagcggcagggtcggaacaggagagcgcacgagggagcttccagggggaa | |
| | acgcctggtatctttatagtcctgtcgggtttcgccacctctgacttgagcgtcg | |
| | atttttgtgatgctcgtcaggggggcggagcctatggaaaaacgccagcaacgcg | |
| | gcctttttacggttcctggccttttgctggccttttgctcacatgttctttcctg | |
| | cgttatcccctgattctgtggataaccgtattaccgcctttgagtgagctgatac | |
| | cgctcgccgcagccgaacgaccgagcgcagcgagtcagtgagcgaggaagcggaa | |
| | gagcgcccaatacgcaaaccgcctctccccgcgcgttggccgattcattaatgca | |
| | gctggcacgacaggtttcccgactggaaagcgggcagtgagcgcaacgcaattaa | |
| | tgtgagttagctcactcattaggcaccccaggctttacactttatgcttccggct | |
| | cgtatgttgtgtggaattgtgagcggataacaatttcacacaggaaacagctatg | |
| | accatgattacgccaagcgcgcaattaaccctcactaaagggaacaaaagctgga | |
| | gctgcaagctt | |
| VB211001- | aatgtagtcttatgcaatactcttgtagtcttgcaacatggtaacgatgagttag | 98 |
| 1280ykc | caacatgccttacaaggagagaaaaagcaccgtgcatgccgattggtggaagtaa | |
| (9951 bp) | ggtggtacgatcgtgccttattaggaaggcaacagacgggtctgacatggattgg | |
| | acgaaccactgaattgccgcattgcagagatattgtatttaagtgcctagctcga | |
| | tacataaacgggtctctctggttagaccagatctgagcctgggagctctctggct | |
| | aactagggaacccactgcttaagcctcaataaagcttgccttgagtgcttcaagt | |
| | agtgtgtgcccgtctgttgtgtgactctggtaactagagatccctcagacccttt | |
| | tagtcagtgtggaaaatctctagcagtggcgcccgaacagggacttgaaagcgaa | |
| | agggaaaccagaggagctctctcgacgcaggactcggcttgctgaagcgcgcacg | |
| | gcaagaggcgaggggcggcgactggtgagtacgccaaaaattttgactagcggag | |
| | gctagaaggagagagatgggtgcgagagcgtcagtattaagcgggggagaattag | |
| | atcgcgatgggaaaaaattcggttaaggccagggggaaagaaaaaatataaatta | |
| | aaacatatagtatgggcaagcagggagctagaacgattcgcagttaatcctggcc | |
| | tgttagaaacatcagaaggctgtagacaaatactgggacagctacaaccatccct | |
| | tcagacaggatcagaagaacttagatcattatataatacagtagcaaccctctat | |
| | tgtgtgcatcaaaggatagagataaaagacaccaaggaagctttagacaagatag | |
| | aggaagagcaaaacaaaagtaagaccaccgcacagcaagcggccgctgatcttca | |
| | gacctggaggaggagatatgagggacaattggagaagtgaattatataaatataa | |
| | agtagtaaaaattgaaccattaggagtagcacccaccaaggcaaagagaagagtg | |
| | gtgcagagagaaaaaagagcagtgggaataggagctttgttccttgggttcttgg | |
| | gagcagcaggaagcactatgggcgcagcgtcaatgacgctgacggtacaggccag | |
| | acaattattgtctggtatagtgcagcagcagaacaatttgctgagggctattgag | |
| | gcgcaacagcatctgttgcaactcacagtctggggcatcaagcagctccaggcaa | |
| | gaatcctggctgtggaaagatacctaaaggatcaacagctcctggggatttgggg | |
| | ttgctctggaaaactcatttgcaccactgctgtgccttggaatgctagttggagt | |
| | aataaatctctggaacagatttggaatcacacgacctggatggagtgggacagag | |
| | aaattaacaattacacaagcttaatacactccttaattgaagaatcgcaaaacca | |
| | gcaagaaaagaatgaacaagaattattggaattagataaatgggcaagtttgtgg | |
| | aattggtttaacataacaaattggctgtggtatataaaattattcataatgatag | |
| | taggaggcttggtaggtttaagaatagtttttgctgtactttctatagtgaatag | |
| | agttaggcagggatattcaccattatcgtttcagacccacctcccaaccccgagg | |
| | ggacccgacaggcccgaaggaatagaagaagaaggtggagagagagacagagaca | |
| | gatccattcgattagtgaacggatctcgacggtatcgctagcttttaaaagaaaa | |
| | ggggggattggggggtacagtgcaggggaaagaatagtagacataatagcaacag | |
| | acatacaaactaaagaattacaaaaacaaattacaaaaattcaaaattttactag | |
| | tgattatcggatcaactttgtatagaaaagttgggctccggtgcccgtcagtggg | |
| | cagagcgcacatcgcccacagtccccgagaagttggggggaggggtcggcaattg | |
| | aaccggtgcctagagaaggtggcgcggggtaaactgggaaagtgatgtcgtgtac | |
| | tggctccgcctttttcccgagggtgggggagaaccgtatataagtgcagtagtcg | |
| | ccgtgaacgttctttttcgcaacgggtttgccgccagaacacaggtaagtgccgt | |
| | gtgtggttcccgcgggcctggcctctttacgggttatggcccttgcgtgccttga | |
| | attacttccacctggctgcagtacgtgattcttgatcccgagcttcgggttggaa | |
| | gtgggtgggagagttcgaggccttgcgcttaaggagccccttcgcctcgtgcttg | |
| | agttgaggcctggcctgggcgctggggccgccgcgtgcgaatctggtggcacctt | |
| | cgcgcctgtctcgctgctttcgataagtctctagccatttaaaatttttgatgac | |
| | ctgctgcgacgctttttttctggcaagatagtcttgtaaatgcgggccaagatct | |
| | gcacactggtatttcggtttttggggccgcgggcggcgacggggcccgtgcgtcc | |
| | cagcgcacatgttcggcgaggcggggcctgcgagcgcggccaccgagaatcggac | |
| | gggggtagtctcaagctggccggcctgctctggtgcctggtctcgcgccgccgtg | |
| | tatcgccccgccctgggcggcaaggctggcccggtcggcaccagttgcgtgagcg | |
| | gaaagatggccgcttcccggccctgctgcagggagctcaaaatggaggacgcggc | |
| | gctcgggagagcgggcgggtgagtcacccacacaaaggaaaagggcctttccgtc | |
| | ctcagccgtcgcttcatgtgactccacggagtaccgggcgccgtccaggcacctc | |
| | gattagttctcgagcttttggagtacgtcgtctttaggttggggggaggggtttt | |
| | atgcgatggagtttccccacactgagtgggtggagactgaagttaggccagcttg | |
| | gcacttgatgtaattctccttggaatttgccctttttgagtttggatcttggttc | |
| | attctcaagcctcagacagtggttcaaagtttttttcttccatttcaggtgtcgt | |
| | gacaagtttgtacaaaaaagcaggctgccaccatgccattgcttcttctgctgcc | |
| | actcctttgggctggtgcgctggctattccggggggtttgtcagaagcaaagccc | |
| | gcgaccccagaaattcaggagatcgtggataaagtaaaacctcagcttgaagaaa | |
| | aaaccggggagacatacggcaagctggaagcagtacaatataaaacgcaggtagt | |
| | acattactacgtccattataacgatcaaggtacaaattattatataaaagtacgg | |
| | gcaggggacaacaaatacatgcatctgaaagtatttaagagtctgtggggtgaga | |
| | acttgtttgcaaagtgggaagatttggtgcttaccggctaccaggtcgataagaa | |
| | taaggatgacgagcttacaggttttgcagaagctgcagcgaaagaggctgccgca | |
| | aaagaagcagcggctaaggaagcggccgctaaagaagcggccgcgaaggaagcgg | |
| | ccgctaagatgatcccgggtggtctgtccgaagccaaaccggcgacgccggaaat | |
| | ccaggagatagtcgataaggtcaagccacaactcgaagagaagactggagaaaca | |
| | tatggaaaacttgaggccgttcaatacaaaacgcaggtggtccactactacgttc | |
| | actataacgatcaaggaacaaactattacattaaagtcagggcgggagacaacaa | |
| | gtacatgcatcttaaagtgtttaaatctctttggggggagaatcttttcgccaag | |
| | tgggaagacttggtcctcaccggataccaggtcgacaaaaacaaagacgatgaac | |
| | tcaccggctttgccgaagcggctgcgaaagaagcggctgccaaagaggctgcagc | |
| | caaagaagctgcggcaaaggaggccgccgccaaagaagcggcggccaaaatgatc | |
| | cccggaggacttagcgaggctaaacccgcgacacctgaaatacaggagatcgtgg | |
| | ataaggttaaaccacaattggaggagaaaactggtgagacctatggcaagctgga | |
| | agcggttcagtataaaacgcaagttgtacactactatgtacattacaacgatcag | |
| | gggacgaactactatattaaggtcagggcaggagataataaatatatgcacttga | |
| | aagttttcaaaagcctgtggggggaaaacttgttcgccaagtgggaagatctcgt | |
| | cttgactgggtaccaggttgataagaacaaagacgatgaactgacaggcttcgct | |
| | gaagcggctgcaaaggaggcggcagcgaaggaggctgccgcgaaagaagcggcgg | |
| | ctaaagaagcggccgcaaaggaagcagcagcgaagatgataccgaggggtttgag | |
| | cgaagcaaagcccgcaacgcctgagatacaagaaatcgttgacaaagtaaaacca | |
| | caattggaagagaagacgggagaaacgtatggaaagcttgaggccgtacaataca | |
| | agacacaggtgttggcaaattttttccaaaggcgatggcccggatcaacgaatta | |
| | ctatatcaaggtacgcgcgggggataacaagtacatgcatctcaaggttttcaac | |
| | ggtccgtggaaattccgaaatacagaccgcggagccgacagggtcctcaccggct | |
| | atcaggtcgacaagaataaggatgatgagttgacaggtttcgccgccgccggtga | |
| | gcagaaattgatctccgaagaggatctcggtgccgcgcaccaccaccaccatcat | |
| | ggaagcggagagggcaggggaagtcttctaacatgcggggacgtggaggaaaatc | |
| | ccggccccatgattgaacaagatggattgcacgcaggttctccggccgcttgggt | |
| | ggagaggctattcggctatgactgggcacaacagacaatcggctgctctgatgcc | |
| | gccgtgttccggctgtcagcgcaggggcgcccggttctttttgtcaagaccgacc | |
| | tgtccggtgccctgaatgaactgcaagacgaggcagcgcggctatcgtggctggc | |
| | cacgacgggcgttccttgcgcagctgtgctcgacgttgtcactgaagcgggaagg | |
| | gactggctgctattgggcgaagtgccggggcaggatctcctgtcatctcaccttg | |
| | ctcctgccgagaaagtatccatcatggctgatgcaatgcggcggctgcatacgct | |
| | tgatccggctacctgcccattcgaccaccaagcgaaacatcgcatcgagcgagca | |
| | cgtactcggatggaagccggtcttgtcgatcaggatgatctggacgaagagcatc | |
| | aggggctcgcgccagccgaactgttcgccaggctcaaggcgagcatgcccgacgg | |
| | cgaggatctcgtcgtgacccatggcgatgcctgcttgccgaatatcatggtggaa | |
| | aatggccgcttttctggattcatcgactgtggccggctgggtgtggcggaccgct | |
| | atcaggacatagcgttggctacccgtgatattgctgaagagcttggcggcgaatg | |
| | ggctgaccgcttcctcgtgctttacggtatcgccgctcccgattcgcagcgcatc | |
| | gccttctatcgccttcttgacgagttcttctgaacccagctttcttgtacaaagt | |
| | ggtgataatcgaattccgataatcaacctctggattacaaaatttgtgaaagatt | |
| | gactggtattcttaactatgttgctccttttacgctatgtggatacgctgcttta | |
| | atgcctttgtatcatgctattgcttcccgtatggctttcattttctcctccttgt | |
| | ataaatcctggttgctgtctctttatgaggagttgtggcccgttgtcaggcaacg | |
| | tggcgtggtgtgcactgtgtttgctgacgcaacccccactggttggggcattgcc | |
| | accacctgtcagctcctttccgggactttcgctttccccctccctattgccacgg | |
| | cggaactcatcgccgcctgccttgcccgctgctggacaggggctcggctgttggg | |
| | cactgacaattccgtggtgttgtcggggaagctgacgtcctttccatggctgctc | |
| | gcctgtgttgccacctggattctgcgcgggacgtccttctgctacgtcccttcgg | |
| | ccctcaatccagcggaccttccttcccgcggcctgctgccggctctgcggcctct | |
| | tccgcgtcttcgccttcgccctcagacgagtcggatctccctttgggccgcctcc | |
| | ccgcatcgggaattcccgcggttcgctttaagaccaatgacttacaaggcagctg | |
| | tagatcttagccactttttaaaagaaaaggggggactggaagggctaattcactc | |
| | ccaacgaagacaagatctgctttttgcttgtactgggtctctctggttagaccag | |
| | atctgagcctgggagctctctggctaactagggaacccactgcttaagcctcaat | |
| | aaagcttgccttgagtgcttcaagtagtgtgtgcccgtctgttgtgtgactctgg | |
| | taactagagatccctcagacccttttagtcagtgtggaaaatctctagcagtagt | |
| | agttcatgtcatcttattattcagtatttataacttgcaaagaaatgaatatcag | |
| | agagtgagaggaacttgtttattgcagcttataatggttacaaataaagcaatag | |
| | catcacaaatttcacaaataaagcatttttttcactgcattctagttgtggtttg | |
| | tccaaactcatcaatgtatcttatcatgtctggctctagctatcccgcccctaac | |
| | tccgcccatcccgcccctaactccgcccagttccgcccattctccgccccatggc | |
| | tgactaattttttttatttatgcagaggccgaggccgcctcggcctctgagctat | |
| | tccagaagtagtgaggaggcttttttggaggcctagggacgtacccaattcgccc | |
| | tatagtgagtcgtattacgcgcgctcactggccgtcgttttacaacgtcgtgact | |
| | gggaaaaccctggcgttacccaacttaatcgccttgcagcacatccccctttcgc | |
| | cagctggcgtaatagcgaagaggcccgcaccgatcgcccttcccaacagttgcgc | |
| | agcctgaatggcgaatgggacgcgccctgtagcggcgcattaagcgcggcgggtg | |
| | tggtggttacgcgcagcgtgaccgctacacttgccagcgccctagcgcccgctcc | |
| | tttcgctttcttcccttcctttctcgccacgttcgccggctttccccgtcaagct | |
| | ctaaatcgggggctccctttagggttccgatttagtgctttacggcacctcgacc | |
| | ccaaaaaacttgattagggtgatggttcacgtagtgggccatcgccctgatagac | |
| | ggtttttcgccctttgacgttggagtccacgttctttaatagtggactcttgttc | |
| | caaactggaacaacactcaaccctatctcggtctattcttttgatttataaggga | |
| | ttttgccgatttcggcctattggttaaaaaatgagctgatttaacaaaaatttaa | |
| | cgcgaattttaacaaaatattaacgcttacaatttaggtggcacttttcggggaa | |
| | atgtgcgcggaacccctatttgtttatttttctaaatacattcaaatatgtatcc | |
| | gctcatgagacaataaccctgataaatgcttcaataatattgaaaaaggaagagt | |
| | atgagtattcaacatttccgtgtcgcccttattcccttttttgcggcattttgcc | |
| | ttcctgtttttgctcacccagaaacgctggtgaaagtaaaagatgctgaagatca | |
| | gttgggtgcacgagtgggttacatcgaactggatctcaacagcggtaagatcctt | |
| | gagagttttcgccccgaagaacgttttccaatgatgagcacttttaaagttctgc | |
| | tatgtggcgcggtattatcccgtattgacgccgggcaagagcaactcggtcgccg | |
| | catacactattctcagaatgacttggttgagtactcaccagtcacagaaaagcat | |
| | cttacggatggcatgacagtaagagaattatgcagtgctgccataaccatgagtg | |
| | ataacactgcggccaacttacttctgacaacgatcggaggaccgaaggagctaac | |
| | cgcttttttgcacaacatgggggatcatgtaactcgccttgatcgttgggaaccg | |
| | gagctgaatgaagccataccaaacgacgagcgtgacaccacgatgcctgtagcaa | |
| | tggcaacaacgttgcgcaaactattaactggcgaactacttactctagcttcccg | |
| | gcaacaattaatagactggatggaggcggataaagttgcaggaccacttctgcgc | |
| | tcggcccttccggctggctggtttattgctgataaatctggagccggtgagcgtg | |
| | ggtctcgcggtatcattgcagcactggggccagatggtaagccctcccgtatcgt | |
| | agttatctacacgacggggagtcaggcaactatggatgaacgaaatagacagatc | |
| | gctgagataggtgcctcactgattaagcattggtaactgtcagaccaagtttact | |
| | catatatactttagattgatttaaaacttcatttttaatttaaaaggatctaggt | |
| | gaagatcctttttgataatctcatgaccaaaatcccttaacgtgagttttcgttc | |
| | cactgagcgtcagaccccgtagaaaagatcaaaggatcttcttgagatccttttt | |
| | ttctgcgcgtaatctgctgcttgcaaacaaaaaaaccaccgctaccagcggtggt | |
| | ttgtttgccggatcaagagctaccaactctttttccgaaggtaactggcttcagc | |
| | agagcgcagataccaaatactgttcttctagtgtagccgtagttaggccaccact | |
| | tcaagaactctgtagcaccgcctacatacctcgctctgctaatcctgttaccagt | |
| | ggctgctgccagtggcgataagtcgtgtcttaccgggttggactcaagacgatag | |
| | ttaccggataaggcgcagcggtcgggctgaacggggggttcgtgcacacagccca | |
| | gcttggagcgaacgacctacaccgaactgagatacctacagcgtgagctatgaga | |
| | aagcgccacgcttcccgaagagagaaaggcggacaggtatccggtaagcggcagg | |
| | gtcggaacaggagagcgcacgagggagcttccagggggaaacgcctggtatcttt | |
| | atagtcctgtcgggtttcgccacctctgacttgagcgtcgatttttgtgatgctc | |
| | gtcaggggggcggagcctatggaaaaacgccagcaacgcggcctttttacggttc | |
| | ctggccttttgctggccttttgctcacatgttctttcctgcgttatcccctgatt | |
| | ctgtggataaccgtattaccgcctttgagtgagctgataccgctcgccgcagccg | |
| | aacgaccgagcgcagcgagtcagtgagcgaggaagcggaagagcgcccaatacgc | |
| | aaaccgcctctccccgcgcgttggccgattcattaatgcagctggcacgacaggt | |
| | ttcccgactggaaagcgggcagtgagcgcaacgcaattaatgtgagttagctcac | |
| | tcattaggcaccccaggctttacactttatgcttccggctcgtatgttgtgtgga | |
| | attgtgagcggataacaatttcacacaggaaacagctatgaccatgattacgcca | |
| | agcgcgcaattaaccctcactaaagggaacaaaagctggagctgcaagctt | |
| VB211001- | aatgtagtcttatgcaatactcttgtagtcttgcaacatggtaacgatgagttag | 99 |
| 1281ptb | caacatgccttacaaggagagaaaaagcaccgtgcatgccgattggtggaagtaa | |
| (9004 bp) | ggtggtacgatcgtgccttattaggaaggcaacagacgggtctgacatggattgg | |
| | acgaaccactgaattgccgcattgcagagatattgtatttaagtgcctagctcga | |
| | tacataaacgggtctctctggttagaccagatctgagcctgggagctctctggct | |
| | aactagggaacccactgcttaagcctcaataaagcttgccttgagtgcttcaagt | |
| | agtgtgtgcccgtctgttgtgtgactctggtaactagagatccctcagacccttt | |
| | tagtcagtgtggaaaatctctagcagtggcgcccgaacagggacttgaaagcgaa | |
| | agggaaaccagaggagctctctcgacgcaggactcggcttgctgaagcgcgcacg | |
| | gcaagaggcgaggggcggcgactggtgagtacgccaaaaattttgactagcggag | |
| | gctagaaggagagagatgggtgcgagagcgtcagtattaagcgggggagaattag | |
| | atcgcgatgggaaaaaattcggttaaggccagggggaaagaaaaaatataaatta | |
| | aaacatatagtatgggcaagcagggagctagaacgattcgcagttaatcctggcc | |
| | tgttagaaacatcagaaggctgtagacaaatactgggacagctacaaccatccct | |
| | tcagacaggatcagaagaacttagatcattatataatacagtagcaaccctctat | |
| | tgtgtgcatcaaaggatagagataaaagacaccaaggaagctttagacaagatag | |
| | aggaagagcaaaacaaaagtaagaccaccgcacagcaagcggccgctgatcttca | |
| | gacctggaggaggagatatgagggacaattggagaagtgaattatataaatataa | |
| | agtagtaaaaattgaaccattaggagtagcacccaccaaggcaaagagaagagtg | |
| | gtgcagagagaaaaaagagcagtgggaataggagctttgttccttgggttcttgg | |
| | gagcagcaggaagcactatgggcgcagcgtcaatgacgctgacggtacaggccag | |
| | acaattattgtctggtatagtgcagcagcagaacaatttgctgagggctattgag | |
| | gcgcaacagcatctgttgcaactcacagtctggggcatcaagcagctccaggcaa | |
| | gaatcctggctgtggaaagatacctaaaggatcaacagctcctggggatttgggg | |
| | ttgctctggaaaactcatttgcaccactgctgtgccttggaatgctagttggagt | |
| | aataaatctctggaacagatttggaatcacacgacctggatggagtgggacagag | |
| | aaattaacaattacacaagcttaatacactccttaattgaagaatcgcaaaacca | |
| | gcaagaaaagaatgaacaagaattattggaattagataaatgggcaagtttgtgg | |
| | aattggtttaacataacaaattggctgtggtatataaaattattcataatgatag | |
| | taggaggcttggtaggtttaagaatagtttttgctgtactttctatagtgaatag | |
| | agttaggcagggatattcaccattatcgtttcagacccacctcccaaccccgagg | |
| | ggacccgacaggcccgaaggaatagaagaagaaggtggagagagagacagagaca | |
| | gatccattcgattagtgaacggatctcgacggtatcgctagcttttaaaagaaaa | |
| | ggggggattggggggtacagtgcaggggaaagaatagtagacataatagcaacag | |
| | acatacaaactaaagaattacaaaaacaaattacaaaaattcaaaattttactag | |
| | tgattatcggatcaactttgtatagaaaagttgggctccggtgcccgtcagtggg | |
| | cagagcgcacatcgcccacagtccccgagaagttggggggaggggtcggcaattg | |
| | atccggtgcctagagaaggtggcgcggggtaaactgggaaagtgatgtcgtgtac | |
| | tggctccgcctttttcccgagggtgggggagaaccgtatataagtgcagtagtcg | |
| | ccgtgaacgttctttttcgcaacgggtttgccgccagaacacaggcaagtttgta | |
| | caaaaaagcaggctgccaccatgccattgcttcttctgctgccactcctttgggc | |
| | tggtgcgctggctattccggggggtttgtcagaagcaaagcccgcgaccccagaa | |
| | attcaggagatcgtggataaagtaaaacctcagcttgaagaaaaaaccggggaga | |
| | catacggcaagctggaagcagtacaatataaaacgcaggtagtacattactacgt | |
| | ccattataacgatcaaggtacaaattattatataaaagtacgggcaggggacaac | |
| | aaatacatgcatctgaaagtatttaagagtctgtggggtgagaacttgtttgcaa | |
| | agtgggaagatttggtgcttaccggctaccaggtcgataagaataaggatgacga | |
| | gcttacaggttttgcagaagctgcagcgaaagaggctgccgcaaaagaagcagcg | |
| | gctaaggaagcggccgctaaagaagcggccgcgaaggaagcggccgctaagatga | |
| | tcccgggtggtctgtccgaagccaaaccggcgacgccggaaatccaggagatagt | |
| | cgataaggtcaagccacaactcgaagagaagactggagaaacatatggaaaactt | |
| | gaggccgttcaatacaaaacgcaggtggtccactactacgttcactataacgatc | |
| | aaggaacaaactattacattaaagtcagggcgggagacaacaagtacatgcatct | |
| | taaagtgtttaaatctctttggggggagaatcttttcgccaagtgggaagacttg | |
| | gtcctcaccggataccaggtcgacaaaaacaaagacgatgaactcaccggctttg | |
| | ccgaagcggctgcgaaagaagcggctgccaaagaggctgcagccaaagaagctgc | |
| | ggcaaaggaggccgccgccaaagaagcggcggccaaaatgatccccggaggactt | |
| | agcgaggctaaacccgcgacacctgaaatacaggagatcgtggataaggttaaac | |
| | cacaattggaggagaaaactggtgagacctatggcaagctggaagcggttcagta | |
| | taaaacgcaagttgtacactactatgtacattacaacgatcaggggacgaactac | |
| | tatattaaggtcagggcaggagataataaatatatgcacttgaaagttttcaaaa | |
| | gcctgtggggggaaaacttgttcgccaagtgggaagatctcgtcttgactgggta | |
| | ccaggttgataagaacaaagacgatgaactgacaggcttcgctgaagcggctgca | |
| | aaggaggcggcagcgaaggaggctgccgcgaaagaagcggcggctaaagaagcgg | |
| | ccgcaaaggaagcagcagcgaagatgataccgaggggtttgagcgaagcaaagcc | |
| | cgcaacgcctgagatacaagaaatcgttgacaaagtaaaaccacaattggaagag | |
| | aagacgggagaaacgtatggaaagcttgaggccgtacaatacaagacacaggtgt | |
| | tggcaaattttttccaaaggcgatggcccggatcaacgaattactatatcaaggt | |
| | acgcgcgggggataacaagtacatgcatctcaaggttttcaacggtccgtggaaa | |
| | ttccgaaatacagaccgcggagccgacagggtcctcaccggctatcaggtcgaca | |
| | agaataaggatgatgagttgacaggtttcgccgccgccggtgagcagaaattgat | |
| | ctccgaagaggatctcggtgccgcgcaccaccaccaccatcatggaagcggagag | |
| | ggcaggggaagtcttctaacatgcggggacgtggaggaaaatcccggccccatga | |
| | ttgaacaagatggattgcacgcaggttctccggccgcttgggtggagaggctatt | |
| | cggctatgactgggcacaacagacaatcggctgctctgatgccgccgtgttccgg | |
| | ctgtcagcgcaggggcgcccggttctttttgtcaagaccgacctgtccggtgccc | |
| | tgaatgaactgcaagacgaggcagcgcggctatcgtggctggccacgacgggcgt | |
| | tccttgcgcagctgtgctcgacgttgtcactgaagcgggaagggactggctgcta | |
| | ttgggcgaagtgccggggcaggatctcctgtcatctcaccttgctcctgccgaga | |
| | aagtatccatcatggctgatgcaatgcggcggctgcatacgcttgatccggctac | |
| | ctgcccattcgaccaccaagcgaaacatcgcatcgagcgagcacgtactcggatg | |
| | gaagccggtcttgtcgatcaggatgatctggacgaagagcatcaggggctcgcgc | |
| | cagccgaactgttcgccaggctcaaggcgagcatgcccgacggcgaggatctcgt | |
| | cgtgacccatggcgatgcctgcttgccgaatatcatggtggaaaatggccgcttt | |
| | tctggattcatcgactgtggccggctgggtgtggcggaccgctatcaggacatag | |
| | cgttggctacccgtgatattgctgaagagcttggcggcgaatgggctgaccgctt | |
| | cctcgtgctttacggtatcgccgctcccgattcgcagcgcatcgccttctatcgc | |
| | cttcttgacgagttcttctgaacccagctttcttgtacaaagtggtgataatcga | |
| | attccgataatcaacctctggattacaaaatttgtgaaagattgactggtattct | |
| | taactatgttgctccttttacgctatgtggatacgctgctttaatgcctttgtat | |
| | catgctattgcttcccgtatggctttcattttctcctccttgtataaatcctggt | |
| | tgctgtctctttatgaggagttgtggcccgttgtcaggcaacgtggcgtggtgtg | |
| | cactgtgtttgctgacgcaacccccactggttggggcattgccaccacctgtcag | |
| | ctcctttccgggactttcgctttccccctccctattgccacggcggaactcatcg | |
| | ccgcctgccttgcccgctgctggacaggggctcggctgttgggcactgacaattc | |
| | cgtggtgttgtcggggaagctgacgtcctttccatggctgctcgcctgtgttgcc | |
| | acctggattctgcgcgggacgtccttctgctacgtcccttcggccctcaatccag | |
| | cggaccttccttcccgcggcctgctgccggctctgcggcctcttccgcgtcttcg | |
| | ccttcgccctcagacgagtcggatctccctttgggccgcctccccgcatcgggaa | |
| | ttcccgcggttcgctttaagaccaatgacttacaaggcagctgtagatcttagcc | |
| | actttttaaaagaaaaggggggactggaagggctaattcactcccaacgaagaca | |
| | agatctgctttttgcttgtactgggtctctctggttagaccagatctgagcctgg | |
| | gagctctctggctaactagggaacccactgcttaagcctcaataaagcttgcctt | |
| | gagtgcttcaagtagtgtgtgcccgtctgttgtgtgactctggtaactagagatc | |
| | cctcagacccttttagtcagtgtggaaaatctctagcagtagtagttcatgtcat | |
| | cttattattcagtatttataacttgcaaagaaatgaatatcagagagtgagagga | |
| | acttgtttattgcagcttataatggttacaaataaagcaatagcatcacaaattt | |
| | cacaaataaagcatttttttcactgcattctagttgtggtttgtccaaactcatc | |
| | aatgtatcttatcatgtctggctctagctatcccgcccctaactccgcccatccc | |
| | gcccctaactccgcccagttccgcccattctccgccccatggctgactaattttt | |
| | tttatttatgcagaggccgaggccgcctcggcctctgagctattccagaagtagt | |
| | gaggaggcttttttggaggcctagggacgtacccaattcgccctatagtgagtcg | |
| | tattacgcgcgctcactggccgtcgttttacaacgtcgtgactgggaaaaccctg | |
| | gcgttacccaacttaatcgccttgcagcacatccccctttcgccagctggcgtaa | |
| | tagcgaagaggcccgcaccgatcgcccttcccaacagttgcgcagcctgaatggc | |
| | gaatgggacgcgccctgtagcggcgcattaagcgcggcgggtgtggtggttacgc | |
| | gcagcgtgaccgctacacttgccagcgccctagcgcccgctcctttcgctttctt | |
| | cccttcctttctcgccacgttcgccggctttccccgtcaagctctaaatcggggg | |
| | ctccctttagggttccgatttagtgctttacggcacctcgaccccaaaaaacttg | |
| | attagggtgatggttcacgtagtgggccatcgccctgatagacggtttttcgccc | |
| | tttgacgttggagtccacgttctttaatagtggactcttgttccaaactggaaca | |
| | acactcaaccctatctcggtctattcttttgatttataagggattttgccgattt | |
| | cggcctattggttaaaaaatgagctgatttaacaaaaatttaacgcgaattttaa | |
| | caaaatattaacgcttacaatttaggtggcacttttcggggaaatgtgcgcggaa | |
| | cccctatttgtttatttttctaaatacattcaaatatgtatccgctcatgagaca | |
| | ataaccctgataaatgcttcaataatattgaaaaaggaagagtatgagtattcaa | |
| | catttccgtgtcgcccttattcccttttttgcggcattttgccttcctgtttttg | |
| | ctcacccagaaacgctggtgaaagtaaaagatgctgaagatcagttgggtgcacg | |
| | agtgggttacatcgaactggatctcaacagcggtaagatccttgagagttttcgc | |
| | cccgaagaacgttttccaatgatgagcacttttaaagttctgctatgtggcgcgg | |
| | tattatcccgtattgacgccgggcaagagcaactcggtcgccgcatacactattc | |
| | tcagaatgacttggttgagtactcaccagtcacagaaaagcatcttacggatggc | |
| | atgacagtaagagaattatgcagtgctgccataaccatgagtgataacactgcgg | |
| | ccaacttacttctgacaacgatcggaggaccgaaggagctaaccgcttttttgca | |
| | caacatgggggatcatgtaactcgccttgatcgttgggaaccggagctgaatgaa | |
| | gccataccaaacgacgagcgtgacaccacgatgcctgtagcaatggcaacaacgt | |
| | tgcgcaaactattaactggcgaactacttactctagcttcccggcaacaattaat | |
| | agactggatggaggcggataaagttgcaggaccacttctgcgctcggcccttccg | |
| | gctggctggtttattgctgataaatctggagccggtgagcgtgggtctcgcggta | |
| | tcattgcagcactggggccagatggtaagccctcccgtatcgtagttatctacac | |
| | gacggggagtcaggcaactatggatgaacgaaatagacagatcgctgagataggt | |
| | gcctcactgattaagcattggtaactgtcagaccaagtttactcatatatacttt | |
| | agattgatttaaaacttcatttttaatttaaaaggatctaggtgaagatcctttt | |
| | tgataatctcatgaccaaaatcccttaacgtgagttttcgttccactgagcgtca | |
| | gaccccgtagaaaagatcaaaggatcttcttgagatcctttttttctgcgcgtaa | |
| | tctgctgcttgcaaacaaaaaaaccaccgctaccagcggtggtttgtttgccgga | |
| | tcaagagctaccaactctttttccgaaggtaactggcttcagcagagcgcagata | |
| | ccaaatactgttcttctagtgtagccgtagttaggccaccacttcaagaactctg | |
| | tagcaccgcctacatacctcgctctgctaatcctgttaccagtggctgctgccag | |
| | tggcgataagtcgtgtcttaccgggttggactcaagacgatagttaccggataag | |
| | gcgcagcggtcgggctgaacggggggttcgtgcacacagcccagcttggagcgaa | |
| | cgacctacaccgaactgagatacctacagcgtgagctatgagaaagcgccacgct | |
| | tcccgaagagagaaaggcggacaggtatccggtaagcggcagggtcggaacagga | |
| | gagcgcacgagggagcttccagggggaaacgcctggtatctttatagtcctgtcg | |
| | ggtttcgccacctctgacttgagcgtcgatttttgtgatgctcgtcaggggggcg | |
| | gagcctatggaaaaacgccagcaacgcggcctttttacggttcctggccttttgc | |
| | tggccttttgctcacatgttctttcctgcgttatcccctgattctgtggataacc | |
| | gtattaccgcctttgagtgagctgataccgctcgccgcagccgaacgaccgagcg | |
| | cagcgagtcagtgagcgaggaagcggaagagcgcccaatacgcaaaccgcctctc | |
| | cccgcgcgttggccgattcattaatgcagctggcacgacaggtttcccgactgga | |
| | aagcgggcagtgagcgcaacgcaattaatgtgagttagctcactcattaggcacc | |
| | ccaggctttacactttatgcttccggctcgtatgttgtgtggaattgtgagcgga | |
| | taacaatttcacacaggaaacagctatgaccatgattacgccaagcgcgcaatta | |
| | accctcactaaagggaacaaaagctggagctgcaagctt | |
| VB211001- | aatgtagtcttatgcaatactcttgtagtcttgcaacatggtaacgatgagttag | 100 |
| 1282mht | caacatgccttacaaggagagaaaaagcaccgtgcatgccgattggtggaagtaa | |
| (9272 bp) | ggtggtacgatcgtgccttattaggaaggcaacagacgggtctgacatggattgg | |
| | acgaaccactgaattgccgcattgcagagatattgtatttaagtgcctagctcga | |
| | tacataaacgggtctctctggttagaccagatctgagcctgggagctctctggct | |
| | aactagggaacccactgcttaagcctcaataaagcttgccttgagtgcttcaagt | |
| | agtgtgtgcccgtctgttgtgtgactctggtaactagagatccctcagacccttt | |
| | tagtcagtgtggaaaatctctagcagtggcgcccgaacagggacttgaaagcgaa | |
| | agggaaaccagaggagctctctcgacgcaggactcggcttgctgaagcgcgcacg | |
| | gcaagaggcgaggggcggcgactggtgagtacgccaaaaattttgactagcggag | |
| | gctagaaggagagagatgggtgcgagagcgtcagtattaagcgggggagaattag | |
| | atcgcgatgggaaaaaattcggttaaggccagggggaaagaaaaaatataaatta | |
| | aaacatatagtatgggcaagcagggagctagaacgattcgcagttaatcctggcc | |
| | tgttagaaacatcagaaggctgtagacaaatactgggacagctacaaccatccct | |
| | tcagacaggatcagaagaacttagatcattatataatacagtagcaaccctctat | |
| | tgtgtgcatcaaaggatagagataaaagacaccaaggaagctttagacaagatag | |
| | aggaagagcaaaacaaaagtaagaccaccgcacagcaagcggccgctgatcttca | |
| | gacctggaggaggagatatgagggacaattggagaagtgaattatataaatataa | |
| | agtagtaaaaattgaaccattaggagtagcacccaccaaggcaaagagaagagtg | |
| | gtgcagagagaaaaaagagcagtgggaataggagctttgttccttgggttcttgg | |
| | gagcagcaggaagcactatgggcgcagcgtcaatgacgctgacggtacaggccag | |
| | acaattattgtctggtatagtgcagcagcagaacaatttgctgagggctattgag | |
| | gcgcaacagcatctgttgcaactcacagtctggggcatcaagcagctccaggcaa | |
| | gaatcctggctgtggaaagatacctaaaggatcaacagctcctggggatttgggg | |
| | ttgctctggaaaactcatttgcaccactgctgtgccttggaatgctagttggagt | |
| | aataaatctctggaacagatttggaatcacacgacctggatggagtgggacagag | |
| | aaattaacaattacacaagcttaatacactccttaattgaagaatcgcaaaacca | |
| | gcaagaaaagaatgaacaagaattattggaattagataaatgggcaagtttgtgg | |
| | aattggtttaacataacaaattggctgtggtatataaaattattcataatgatag | |
| | taggaggcttggtaggtttaagaatagtttttgctgtactttctatagtgaatag | |
| | agttaggcagggatattcaccattatcgtttcagacccacctcccaaccccgagg | |
| | ggacccgacaggcccgaaggaatagaagaagaaggtggagagagagacagagaca | |
| | gatccattcgattagtgaacggatctcgacggtatcgctagcttttaaaagaaaa | |
| | ggggggattggggggtacagtgcaggggaaagaatagtagacataatagcaacag | |
| | acatacaaactaaagaattacaaaaacaaattacaaaaattcaaaattttactag | |
| | tgattatcggatcaactttgtatagaaaagttgctgcagccccgataaataaaaa | |
| | gattttatttagtctccagaaaaaggggggaatgaaagaccccacctgtaggttt | |
| | ggcaagctagctgcagtaacgccattttgcaaggcatggaaaaataccaaaccaa | |
| | gaatagagaagttcagatcaagggcgggtacatgaaaatagctaacgttgggcca | |
| | aacaggatatctgcggtgagcagtttcggccccggcccggggccaagaacagatg | |
| | gtcaccgcagtttcggccccggcccgaggccaagaacagatggtccccagatatg | |
| | gcccaaccctcagcagtttcttaagacccatcagatgtttccaggctcccccaag | |
| | gacctgaaatgaccctgcgccttatttgaattaaccaatcagcctgcttctcgct | |
| | tctgttcgcgcgcttctgcttcccgagctctataaaagagctcacaacccctcac | |
| | tcggcgcgccagtcctccgacagactgagtcgcccgggcaagtttgtacaaaaaa | |
| | gcaggctgccaccatgccattgcttcttctgctgccactcctttgggctggtgcg | |
| | ctggctattccggggggtttgtcagaagcaaagcccgcgaccccagaaattcagg | |
| | agatcgtggataaagtaaaacctcagcttgaagaaaaaaccggggagacatacgg | |
| | caagctggaagcagtacaatataaaacgcaggtagtacattactacgtccattat | |
| | aacgatcaaggtacaaattattatataaaagtacgggcaggggacaacaaataca | |
| | tgcatctgaaagtatttaagagtctgtggggtgagaacttgtttgcaaagtggga | |
| | agatttggtgcttaccggctaccaggtcgataagaataaggatgacgagcttaca | |
| | ggttttgcagaagctgcagcgaaagaggctgccgcaaaagaagcagcggctaagg | |
| | aagcggccgctaaagaagcggccgcgaaggaagcggccgctaagatgatcccggg | |
| | tggtctgtccgaagccaaaccggcgacgccggaaatccaggagatagtcgataag | |
| | gtcaagccacaactcgaagagaagactggagaaacatatggaaaacttgaggccg | |
| | ttcaatacaaaacgcaggtggtccactactacgttcactataacgatcaaggaac | |
| | aaactattacattaaagtcagggcgggagacaacaagtacatgcatcttaaagtg | |
| | tttaaatctctttggggggagaatcttttcgccaagtgggaagacttggtcctca | |
| | ccggataccaggtcgacaaaaacaaagacgatgaactcaccggctttgccgaagc | |
| | ggctgcgaaagaagcggctgccaaagaggctgcagccaaagaagctgcggcaaag | |
| | gaggccgccgccaaagaagcggcggccaaaatgatccccggaggacttagcgagg | |
| | ctaaacccgcgacacctgaaatacaggagatcgtggataaggttaaaccacaatt | |
| | ggaggagaaaactggtgagacctatggcaagctggaagcggttcagtataaaacg | |
| | caagttgtacactactatgtacattacaacgatcaggggacgaactactatatta | |
| | aggtcagggcaggagataataaatatatgcacttgaaagttttcaaaagcctgtg | |
| | gggggaaaacttgttcgccaagtgggaagatctcgtcttgactgggtaccaggtt | |
| | gataagaacaaagacgatgaactgacaggcttcgctgaagcggctgcaaaggagg | |
| | cggcagcgaaggaggctgccgcgaaagaagcggcggctaaagaagcggccgcaaa | |
| | ggaagcagcagcgaagatgataccgaggggtttgagcgaagcaaagcccgcaacg | |
| | cctgagatacaagaaatcgttgacaaagtaaaaccacaattggaagagaagacgg | |
| | gagaaacgtatggaaagcttgaggccgtacaatacaagacacaggtgttggcaaa | |
| | ttttttccaaaggcgatggcccggatcaacgaattactatatcaaggtacgcgcg | |
| | ggggataacaagtacatgcatctcaaggttttcaacggtccgtggaaattccgaa | |
| | atacagaccgcggagccgacagggtcctcaccggctatcaggtcgacaagaataa | |
| | ggatgatgagttgacaggtttcgccgccgccggtgagcagaaattgatctccgaa | |
| | gaggatctcggtgccgcgcaccaccaccaccatcatggaagcggagagggcaggg | |
| | gaagtcttctaacatgcggggacgtggaggaaaatcccggccccatgattgaaca | |
| | agatggattgcacgcaggttctccggccgcttgggtggagaggctattcggctat | |
| | gactgggcacaacagacaatcggctgctctgatgccgccgtgttccggctgtcag | |
| | cgcaggggcgcccggttctttttgtcaagaccgacctgtccggtgccctgaatga | |
| | actgcaagacgaggcagcgcggctatcgtggctggccacgacgggcgttccttgc | |
| | gcagctgtgctcgacgttgtcactgaagcgggaagggactggctgctattgggcg | |
| | aagtgccggggcaggatctcctgtcatctcaccttgctcctgccgagaaagtatc | |
| | catcatggctgatgcaatgcggcggctgcatacgcttgatccggctacctgccca | |
| | ttcgaccaccaagcgaaacatcgcatcgagcgagcacgtactcggatggaagccg | |
| | gtcttgtcgatcaggatgatctggacgaagagcatcaggggctcgcgccagccga | |
| | actgttcgccaggctcaaggcgagcatgcccgacggcgaggatctcgtcgtgacc | |
| | catggcgatgcctgcttgccgaatatcatggtggaaaatggccgcttttctggat | |
| | tcatcgactgtggccggctgggtgtggcggaccgctatcaggacatagcgttggc | |
| | tacccgtgatattgctgaagagcttggcggcgaatgggctgaccgcttcctcgtg | |
| | ctttacggtatcgccgctcccgattcgcagcgcatcgccttctatcgccttcttg | |
| | acgagttcttctgaacccagctttcttgtacaaagtggtgataatcgaattccga | |
| | taatcaacctctggattacaaaatttgtgaaagattgactggtattcttaactat | |
| | gttgctccttttacgctatgtggatacgctgctttaatgcctttgtatcatgcta | |
| | ttgcttcccgtatggctttcattttctcctccttgtataaatcctggttgctgtc | |
| | tctttatgaggagttgtggcccgttgtcaggcaacgtggcgtggtgtgcactgtg | |
| | tttgctgacgcaacccccactggttggggcattgccaccacctgtcagctccttt | |
| | ccgggactttcgctttccccctccctattgccacggcggaactcatcgccgcctg | |
| | ccttgcccgctgctggacaggggctcggctgttgggcactgacaattccgtggtg | |
| | ttgtcggggaagctgacgtcctttccatggctgctcgcctgtgttgccacctgga | |
| | ttctgcgcgggacgtccttctgctacgtcccttcggccctcaatccagcggacct | |
| | tccttcccgcggcctgctgccggctctgcggcctcttccgcgtcttcgccttcgc | |
| | cctcagacgagtcggatctccctttgggccgcctccccgcatcgggaattcccgc | |
| | ggttcgctttaagaccaatgacttacaaggcagctgtagatcttagccacttttt | |
| | aaaagaaaaggggggactggaagggctaattcactcccaacgaagacaagatctg | |
| | ctttttgcttgtactgggtctctctggttagaccagatctgagcctgggagctct | |
| | ctggctaactagggaacccactgcttaagcctcaataaagcttgccttgagtgct | |
| | tcaagtagtgtgtgcccgtctgttgtgtgactctggtaactagagatccctcaga | |
| | cccttttagtcagtgtggaaaatctctagcagtagtagttcatgtcatcttatta | |
| | ttcagtatttataacttgcaaagaaatgaatatcagagagtgagaggaacttgtt | |
| | tattgcagcttataatggttacaaataaagcaatagcatcacaaatttcacaaat | |
| | aaagcatttttttcactgcattctagttgtggtttgtccaaactcatcaatgtat | |
| | cttatcatgtctggctctagctatcccgcccctaactccgcccatcccgccccta | |
| | actccgcccagttccgcccattctccgccccatggctgactaattttttttattt | |
| | atgcagaggccgaggccgcctcggcctctgagctattccagaagtagtgaggagg | |
| | cttttttggaggcctagggacgtacccaattcgccctatagtgagtcgtattacg | |
| | cgcgctcactggccgtcgttttacaacgtcgtgactgggaaaaccctggcgttac | |
| | ccaacttaatcgccttgcagcacatccccctttcgccagctggcgtaatagcgaa | |
| | gaggcccgcaccgatcgcccttcccaacagttgcgcagcctgaatggcgaatggg | |
| | acgcgccctgtagcggcgcattaagcgcggcgggtgtggtggttacgcgcagcgt | |
| | gaccgctacacttgccagcgccctagcgcccgctcctttcgctttcttcccttcc | |
| | tttctcgccacgttcgccggctttccccgtcaagctctaaatcgggggctccctt | |
| | tagggttccgatttagtgctttacggcacctcgaccccaaaaaacttgattaggg | |
| | tgatggttcacgtagtgggccatcgccctgatagacggtttttcgccctttgacg | |
| | ttggagtccacgttctttaatagtggactcttgttccaaactggaacaacactca | |
| | accctatctcggtctattcttttgatttataagggattttgccgatttcggccta | |
| | ttggttaaaaaatgagctgatttaacaaaaatttaacgcgaattttaacaaaata | |
| | ttaacgcttacaatttaggtggcacttttcggggaaatgtgcgcggaacccctat | |
| | ttgtttatttttctaaatacattcaaatatgtatccgctcatgagacaataaccc | |
| | tgataaatgcttcaataatattgaaaaaggaagagtatgagtattcaacatttcc | |
| | gtgtcgcccttattcccttttttgcggcattttgccttcctgtttttgctcaccc | |
| | agaaacgctggtgaaagtaaaagatgctgaagatcagttgggtgcacgagtgggt | |
| | tacatcgaactggatctcaacagcggtaagatccttgagagttttcgccccgaag | |
| | aacgttttccaatgatgagcacttttaaagttctgctatgtggcgcggtattatc | |
| | ccgtattgacgccgggcaagagcaactcggtcgccgcatacactattctcagaat | |
| | gacttggttgagtactcaccagtcacagaaaagcatcttacggatggcatgacag | |
| | taagagaattatgcagtgctgccataaccatgagtgataacactgcggccaactt | |
| | acttctgacaacgatcggaggaccgaaggagctaaccgcttttttgcacaacatg | |
| | ggggatcatgtaactcgccttgatcgttgggaaccggagctgaatgaagccatac | |
| | caaacgacgagcgtgacaccacgatgcctgtagcaatggcaacaacgttgcgcaa | |
| | actattaactggcgaactacttactctagcttcccggcaacaattaatagactgg | |
| | atggaggcggataaagttgcaggaccacttctgcgctcggcccttccggctggct | |
| | ggtttattgctgataaatctggagccggtgagcgtgggtctcgcggtatcattgc | |
| | agcactggggccagatggtaagccctcccgtatcgtagttatctacacgacgggg | |
| | agtcaggcaactatggatgaacgaaatagacagatcgctgagataggtgcctcac | |
| | tgattaagcattggtaactgtcagaccaagtttactcatatatactttagattga | |
| | tttaaaacttcatttttaatttaaaaggatctaggtgaagatcctttttgataat | |
| | ctcatgaccaaaatcccttaacgtgagttttcgttccactgagcgtcagaccccg | |
| | tagaaaagatcaaaggatcttcttgagatcctttttttctgcgcgtaatctgctg | |
| | cttgcaaacaaaaaaaccaccgctaccagcggtggtttgtttgccggatcaagag | |
| | ctaccaactctttttccgaaggtaactggcttcagcagagcgcagataccaaata | |
| | ctgttcttctagtgtagccgtagttaggccaccacttcaagaactctgtagcacc | |
| | gcctacatacctcgctctgctaatcctgttaccagtggctgctgccagtggcgat | |
| | aagtcgtgtcttaccgggttggactcaagacgatagttaccggataaggcgcagc | |
| | ggtcgggctgaacggggggttcgtgcacacagcccagcttggagcgaacgaccta | |
| | caccgaactgagatacctacagcgtgagctatgagaaagcgccacgcttcccgaa | |
| | gagagaaaggcggacaggtatccggtaagcggcagggtcggaacaggagagcgca | |
| | cgagggagcttccagggggaaacgcctggtatctttatagtcctgtcgggtttcg | |
| | ccacctctgacttgagcgtcgatttttgtgatgctcgtcaggggggcggagccta | |
| | tggaaaaacgccagcaacgcggcctttttacggttcctggccttttgctggcctt | |
| | ttgctcacatgttctttcctgcgttatcccctgattctgtggataaccgtattac | |
| | cgcctttgagtgagctgataccgctcgccgcagccgaacgaccgagcgcagcgag | |
| | tcagtgagcgaggaagcggaagagcgcccaatacgcaaaccgcctctccccgcgc | |
| | gttggccgattcattaatgcagctggcacgacaggtttcccgactggaaagcggg | |
| | cagtgagcgcaacgcaattaatgtgagttagctcactcattaggcaccccaggct | |
| | ttacactttatgcttccggctcgtatgttgtgtggaattgtgagcggataacaat | |
| | ttcacacaggaaacagctatgaccatgattacgccaagcgcgcaattaaccctca | |
| | ctaaagggaacaaaagctggagctgcaagctt | |
| VB211001- | aatgtagtcttatgcaatactcttgtagtcttgcaacatggtaacgatgagttag | 101 |
| 1283mjc | caacatgccttacaaggagagaaaaagcaccgtgcatgccgattggtggaagtaa | |
| (9287 bp) | ggtggtacgatcgtgccttattaggaaggcaacagacgggtctgacatggattgg | |
| | acgaaccactgaattgccgcattgcagagatattgtatttaagtgcctagctcga | |
| | tacataaacgggtctctctggttagaccagatctgagcctgggagctctctggct | |
| | aactagggaacccactgcttaagcctcaataaagcttgccttgagtgcttcaagt | |
| | agtgtgtgcccgtctgttgtgtgactctggtaactagagatccctcagacccttt | |
| | tagtcagtgtggaaaatctctagcagtggcgcccgaacagggacttgaaagcgaa | |
| | agggaaaccagaggagctctctcgacgcaggactcggcttgctgaagcgcgcacg | |
| | gcaagaggcgaggggcggcgactggtgagtacgccaaaaattttgactagcggag | |
| | gctagaaggagagagatgggtgcgagagcgtcagtattaagcgggggagaattag | |
| | atcgcgatgggaaaaaattcggttaaggccagggggaaagaaaaaatataaatta | |
| | aaacatatagtatgggcaagcagggagctagaacgattcgcagttaatcctggcc | |
| | tgttagaaacatcagaaggctgtagacaaatactgggacagctacaaccatccct | |
| | tcagacaggatcagaagaacttagatcattatataatacagtagcaaccctctat | |
| | tgtgtgcatcaaaggatagagataaaagacaccaaggaagctttagacaagatag | |
| | aggaagagcaaaacaaaagtaagaccaccgcacagcaagcggccgctgatcttca | |
| | gacctggaggaggagatatgagggacaattggagaagtgaattatataaatataa | |
| | agtagtaaaaattgaaccattaggagtagcacccaccaaggcaaagagaagagtg | |
| | gtgcagagagaaaaaagagcagtgggaataggagctttgttccttgggttcttgg | |
| | gagcagcaggaagcactatgggcgcagcgtcaatgacgctgacggtacaggccag | |
| | acaattattgtctggtatagtgcagcagcagaacaatttgctgagggctattgag | |
| | gcgcaacagcatctgttgcaactcacagtctggggcatcaagcagctccaggcaa | |
| | gaatcctggctgtggaaagatacctaaaggatcaacagctcctggggatttgggg | |
| | ttgctctggaaaactcatttgcaccactgctgtgccttggaatgctagttggagt | |
| | aataaatctctggaacagatttggaatcacacgacctggatggagtgggacagag | |
| | aaattaacaattacacaagcttaatacactccttaattgaagaatcgcaaaacca | |
| | gcaagaaaagaatgaacaagaattattggaattagataaatgggcaagtttgtgg | |
| | aattggtttaacataacaaattggctgtggtatataaaattattcataatgatag | |
| | taggaggcttggtaggtttaagaatagtttttgctgtactttctatagtgaatag | |
| | agttaggcagggatattcaccattatcgtttcagacccacctcccaaccccgagg | |
| | ggacccgacaggcccgaaggaatagaagaagaaggtggagagagagacagagaca | |
| | gatccattcgattagtgaacggatctcgacggtatcgctagcttttaaaagaaaa | |
| | ggggggattggggggtacagtgcaggggaaagaatagtagacataatagcaacag | |
| | acatacaaactaaagaattacaaaaacaaattacaaaaattcaaaattttactag | |
| | tgattatcggatcaactttgtatagaaaagttgtgaaagaccccacctgtaggtt | |
| | tggcaagctagcttaagtaacgccattttgcaaggcatggaaaatacataactga | |
| | gaatagagaagttcagatcaaggttaggaacagagagacagcagaatatgggcca | |
| | aacaggatatctgtggtaagcagttcctgccccggctcagggccaagaacagatg | |
| | gtccccagatgcggtcccgccctcagcagtttctagagaaccatcagatgtttcc | |
| | agggtgccccaaggacctgaaatgaccctgtgccttatttgaactaaccaatcag | |
| | ttcgcttctcgcttctgttcgcgcgcttctgctccccgagctcaataaaagagcc | |
| | cacaacccctcactcggcgcgccagtcctccgatagactgcgtcgcccgggtacc | |
| | cgtattcccaataaagcctcttgctgtttgcatccgaatcgtggactcgctgatc | |
| | cttgggagggtctcctcagattgattgactgcccacctcgggggtctttcattca | |
| | agtttgtacaaaaaagcaggctgccaccatgccattgcttcttctgctgccactc | |
| | ctttgggctggtgcgctggctattccggggggtttgtcagaagcaaagcccgcga | |
| | ccccagaaattcaggagatcgtggataaagtaaaacctcagcttgaagaaaaaac | |
| | cggggagacatacggcaagctggaagcagtacaatataaaacgcaggtagtacat | |
| | tactacgtccattataacgatcaaggtacaaattattatataaaagtacgggcag | |
| | gggacaacaaatacatgcatctgaaagtatttaagagtctgtggggtgagaactt | |
| | gtttgcaaagtgggaagatttggtgcttaccggctaccaggtcgataagaataag | |
| | gatgacgagcttacaggttttgcagaagctgcagcgaaagaggctgccgcaaaag | |
| | aagcagcggctaaggaagcggccgctaaagaagcggccgcgaaggaagcggccgc | |
| | taagatgatcccgggtggtctgtccgaagccaaaccggcgacgccggaaatccag | |
| | gagatagtcgataaggtcaagccacaactcgaagagaagactggagaaacatatg | |
| | gtgagtcgtattacgcgcgctcactggccgtcgttttacaacgtcgtgactggga | |
| | aaaccctggcgttacccaacttaatcgccttgcagcacatccccctttcgccagc | |
| | tggcgtaatagcgaagaggcccgcaccgatcgcccttcccaacagttgcgcagcc | |
| | tgaatggcgaatgggacgcgccctgtagcggcgcattaagcgcggcgggtgtggt | |
| | ggttacgcgcagcgtgaccgctacacttgccagcgccctagcgcccgctcctttc | |
| | gctttcttcccttcctttctcgccacgttcgccggctttccccgtcaagctctaa | |
| | atcgggggctccctttagggttccgatttagtgctttacggcacctcgaccccaa | |
| | aaaacttgattagggtgatggttcacgtagtgggccatcgccctgatagacggtt | |
| | tttcgccctttgacgttggagtccacgttctttaatagtggactcttgttccaaa | |
| | ctggaacaacactcaaccctatctcggtctattcttttgatttataagggatttt | |
| | gccgatttcggcctattggttaaaaaatgagctgatttaacaaaaatttaacgcg | |
| | aattttaacaaaatattaacgcttacaatttaggtggcacttttcggggaaatgt | |
| | gcgcggaacccctatttgtttatttttctaaatacattcaaatatgtatccgctc | |
| | atgagacaataaccctgataaatgcttcaataatattgaaaaaggaagagtatga | |
| | gtattcaacatttccgtgtcgcccttattcccttttttgcggcattttgccttcc | |
| | tgtttttgctcacccagaaacgctggtgaaagtaaaagatgctgaagatcagttg | |
| | ggtgcacgagtgggttacatcgaactggatctcaacagcggtaagatccttgaga | |
| | gttttcgccccgaagaacgttttccaatgatgagcacttttaaagttctgctatg | |
| | tggcgcggtattatcccgtattgacgccgggcaagagcaactcggtcgccgcata | |
| | cactattctcagaatgacttggttgagtactcaccagtcacagaaaagcatctta | |
| | cggatggcatgacagtaagagaattatgcagtgctgccataaccatgagtgataa | |
| | cactgcggccaacttacttctgacaacgatcggaggaccgaaggagctaaccgct | |
| | tttttgcacaacatgggggatcatgtaactcgccttgatcgttgggaaccggagc | |
| | tgaatgaagccataccaaacgacgagcgtgacaccacgatgcctgtagcaatggc | |
| | aacaacgttgcgcaaactattaactggcgaactacttactctagcttcccggcaa | |
| | caattaatagactggatggaggcggataaagttgcaggaccacttctgcgctcgg | |
| | cccttccggctggctggtttattgctgataaatctggagccggtgagcgtgggtc | |
| | tcgcggtatcattgcagcactggggccagatggtaagccctcccgtatcgtagtt | |
| | atctacacgacggggagtcaggcaactatggatgaacgaaatagacagatcgctg | |
| | agataggtgcctcactgattaagcattggtaactgtcagaccaagtttactcata | |
| | tatactttagattgatttaaaacttcatttttaatttaaaaggatctaggtgaag | |
| | atcctttttgataatctcatgaccaaaatcccttaacgtgagttttcgttccact | |
| | gagcgtcagaccccgtagaaaagatcaaaggatcttcttgagatcctttttttct | |
| | gcgcgtaatctgctgcttgcaaacaaaaaaaccaccgctaccagcggtggtttgt | |
| | ttgccggatcaagagctaccaactctttttccgaaggtaactggcttcagcagag | |
| | cgcagataccaaatactgttcttctagtgtagccgtagttaggccaccacttcaa | |
| | gaactctgtagcaccgcctacatacctcgctctgctaatcctgttaccagtggct | |
| | gctgccagtggcgataagtcgtgtcttaccgggttggactcaagacgatagttac | |
| | cggataaggcgcagcggtcgggctgaacggggggttcgtgcacacagcccagctt | |
| | ggagcgaacgacctacaccgaactgagatacctacagcgtgagctatgagaaagc | |
| | gccacgcttcccgaagagagaaaggcggacaggtatccggtaagcggcagggtcg | |
| | gaacaggagagcgcacgagggagcttccagggggaaacgcctggtatctttatag | |
| | tcctgtcgggtttcgccacctctgacttgagcgtcgatttttgtgatgctcgtca | |
| | ggggggcggagcctatggaaaaacgccagcaacgcggcctttttacggttcctgg | |
| | ccttttgctggccttttgctcacatgttctttcctgcgttatcccctgattctgt | |
| | ggataaccgtattaccgcctttgagtgagctgataccgctcgccgcagccgaacg | |
| | accgagcgcagcgagtcagtgagcgaggaagcggaagagcgcccaatacgcaaac | |
| | cgcctctccccgcgcgttggccgattcattaatgcagctggcacgacaggtttcc | |
| | cgactggaaagcgggcagtgagcgcaacgcaattaatgtgagttagctcactcat | |
| | taggcaccccaggctttacactttatgcttccggctcgtatgttgtgtggaattg | |
| | tgagcggataacaatttcacacaggaaacagctatgaccatgattacgccaagcg | |
| | cgcaattaaccctcactaaagggaacaaaagctggagctgcaagctt | |
| VB211012- | aatgtagtcttatgcaatactcttgtagtcttgcaacatggtaacgatgagttag | 102 |
| 1093gfx | caacatgccttacaaggagagaaaaagcaccgtgcatgccgattggtggaagtaa | |
| (9570 bp) | ggtggtacgatcgtgccttattaggaaggcaacagacgggtctgacatggattgg | |
| | acgaaccactgaattgccgcattgcagagatattgtatttaagtgcctagctcga | |
| | tacataaacgggtctctctggttagaccagatctgagcctgggagctctctggct | |
| | aactagggaacccactgcttaagcctcaataaagcttgccttgagtgcttcaagt | |
| | agtgtgtgcccgtctgttgtgtgactctggtaactagagatccctcagacccttt | |
| | tagtcagtgtggaaaatctctagcagtggcgcccgaacagggacttgaaagcgaa | |
| | agggaaaccagaggagctctctcgacgcaggactcggcttgctgaagcgcgcacg | |
| | gcaagaggcgaggggcggcgactggtgagtacgccaaaaattttgactagcggag | |
| | gctagaaggagagagatgggtgcgagagcgtcagtattaagcgggggagaattag | |
| | atcgcgatgggaaaaaattcggttaaggccagggggaaagaaaaaatataaatta | |
| | aaacatatagtatgggcaagcagggagctagaacgattcgcagttaatcctggcc | |
| | tgttagaaacatcagaaggctgtagacaaatactgggacagctacaaccatccct | |
| | tcagacaggatcagaagaacttagatcattatataatacagtagcaaccctctat | |
| | tgtgtgcatcaaaggatagagataaaagacaccaaggaagctttagacaagatag | |
| | aggaagagcaaaacaaaagtaagaccaccgcacagcaagcggccgctgatcttca | |
| | gacctggaggaggagatatgagggacaattggagaagtgaattatataaatataa | |
| | agtagtaaaaattgaaccattaggagtagcacccaccaaggcaaagagaagagtg | |
| | gtgcagagagaaaaaagagcagtgggaataggagctttgttccttgggttcttgg | |
| | gagcagcaggaagcactatgggcgcagcgtcaatgacgctgacggtacaggccag | |
| | acaattattgtctggtatagtgcagcagcagaacaatttgctgagggctattgag | |
| | gcgcaacagcatctgttgcaactcacagtctggggcatcaagcagctccaggcaa | |
| | gaatcctggctgtggaaagatacctaaaggatcaacagctcctggggatttgggg | |
| | ttgctctggaaaactcatttgcaccactgctgtgccttggaatgctagttggagt | |
| | aataaatctctggaacagatttggaatcacacgacctggatggagtgggacagag | |
| | aaattaacaattacacaagcttaatacactccttaattgaagaatcgcaaaacca | |
| | gcaagaaaagaatgaacaagaattattggaattagataaatgggcaagtttgtgg | |
| | aattggtttaacataacaaattggctgtggtatataaaattattcataatgatag | |
| | taggaggcttggtaggtttaagaatagtttttgctgtactttctatagtgaatag | |
| | agttaggcagggatattcaccattatcgtttcagacccacctcccaaccccgagg | |
| | ggacccgacaggcccgaaggaatagaagaagaaggtggagagagagacagagaca | |
| | gatccattcgattagtgaacggatctcgacggtatcgctagcttttaaaagaaaa | |
| | ggggggattggggggtacagtgcaggggaaagaatagtagacataatagcaacag | |
| | acatacaaactaaagaattacaaaaacaaattacaaaaattcaaaattttactag | |
| | tgattatcggatcaactttgtatagaaaagttgcgttacataacttacggtaaat | |
| | ggcccgcctggctgaccgcccaacgacccccgcccattgacgtcaatagtaacgc | |
| | caatagggactttccattgacgtcaatgggtggagtatttacggtaaactgccca | |
| | cttggcagtacatcaagtgtatcatatgccaagtacgccccctattgacgtcaat | |
| | gacggtaaatggcccgcctggcattgtgcccagtacatgaccttatgggactttc | |
| | ctacttggcagtacatctacgtattagtcatcgctattaccatggtcgaggtgag | |
| | ccccacgttctgcttcactctccccatctcccccccctccccacccccaattttg | |
| | tatttatttattttttaattattttgtgcagcgatgggggcgggggggggggggg | |
| | ggcgcgcgccaggcggggcggggcggggcgaggggcggggcggggcgaggcggag | |
| | aggtgcggcggcagccaatcagagcggcgcgctccgaaagtttccttttatggcg | |
| | aggcggcggcggcggcggccctataaaaagcgaagcgcgcggcgggcgggagtcg | |
| | ctgcgcgctgccttcgccccgtgccccgctccgccgccgcctcgcgccgcccgcc | |
| | ccggctctgactgaccgcgttactcccacaggtgagcgggcgggacggcccttct | |
| | cctccgggctgtaattagctgagcaagaggtaagggtttaagggatggttggttg | |
| | gtggggtattaatgtttaattacctggagcacctgcctgaaatcactttttttca | |
| | ggttggcaagtttgtacaaaaaagcaggctgccaccatgccattgcttcttctgc | |
| | tgccactcctttgggctggtgcgctggctattccggggggtttgtcagaagcaaa | |
| | gcccgcgaccccagaaattcaggagatcgtggataaagtaaaacctcagcttgaa | |
| | gaaaaaaccggggagacatacggcaagctggaagcagtacaatataaaacgcagg | |
| | tagtacattactacgtccattataacgatcaaggtacaaattattatataaaagt | |
| | acgggcaggggacaacaaatacatgcatctgaaagtatttaagagtctgtggggt | |
| | gagaacttgtttgcaaagtgggaagatttggtgcttaccggctaccaggtcgata | |
| | agaataaggatgacgagcttacaggttttgcagaagctgcagcgaaagaggctgc | |
| | cgcaaaagaagcagcggctaaggaagcggccgctaaagaagcggccgcgaaggaa | |
| | gcggccgctaagatgatcccgggtggtctgtccgaagccaaaccggcgacgccgg | |
| | aaatccaggagatagtcgataaggtcaagccacaactcgaagagaagactggaga | |
| | aacatatggaaaacttgaggccgttcaatacaaaacgcaggtggtccactactac | |
| | gttcactataacgatcaaggaacaaactattacattaaagtcagggcgggagaca | |
| | acaagtacatgcatcttaaagtgtttaaatctctttggggggagaatcttttcgc | |
| | caagtgggaagacttggtcctcaccggataccaggtcgacaaaaacaaagacgat | |
| | gaactcaccggctttgccgaagcggctgcgaaagaagcggctgccaaagaggctg | |
| | cagccaaagaagctgcggcaaaggaggccgccgccaaagaagcggcggccaaaat | |
| | gatccccggaggacttagcgaggctaaacccgcgacacctgaaatacaggagatc | |
| | gtggataaggttaaaccacaattggaggagaaaactggtgagacctatggcaagc | |
| | tggaagcggttcagtataaaacgcaagttgtacactactatgtacattacaacga | |
| | tcaggggacgaactactatattaaggtcagggcaggagataataaatatatgcac | |
| | ttgaaagttttcaaaagcctgtggggggaaaacttgttcgccaagtgggaagatc | |
| | tcgtcttgactgggtaccaggttgataagaacaaagacgatgaactgacaggctt | |
| | cgctgaagcggctgcaaaggaggcggcagcgaaggaggctgccgcgaaagaagcg | |
| | gcggctaaagaagcggccgcaaaggaagcagcagcgaagatgataccgaggggtt | |
| | tgagcgaagcaaagcccgcaacgcctgagatacaagaaatcgttgacaaagtaaa | |
| | accacaattggaagagaagacgggagaaacgtatggaaagcttgaggccgtacaa | |
| | tacaagacacaggtgttggcaaattttttccaaaggcgatggcccggatcaacga | |
| | attactatatcaaggtacgcgcgggggataacaagtacatgcatctcaaggtttt | |
| | caacggtccgtggaaattccgaaatacagaccgcggagccgacagggtcctcacc | |
| | ggctatcaggtcgacaagaataaggatgatgagttgacaggtttcgccgccgccg | |
| | gtgagcagaaattgatctccgaagaggatctcggtgccgcgcaccaccaccacca | |
| | tcatggaagcggagagggcaggggaagtcttctaacatgcggggacgtggaggaa | |
| | aatcccggccccatgattgaacaagatggattgcacgcaggttctccggccgctt | |
| | gggtggagaggctattcggctatgactgggcacaacagacaatcggctgctctga | |
| | tgccgccgtgttccggctgtcagcgcaggggcgcccggttctttttgtcaagacc | |
| | gacctgtccggtgccctgaatgaactgcaagacgaggcagcgcggctatcgtggc | |
| | tggccacgacgggcgttccttgcgcagctgtgctcgacgttgtcactgaagcggg | |
| | aagggactggctgctattgggcgaagtgccggggcaggatctcctgtcatctcac | |
| | cttgctcctgccgagaaagtatccatcatggctgatgcaatgcggcggctgcata | |
| | cgcttgatccggctacctgcccattcgaccaccaagcgaaacatcgcatcgagcg | |
| | agcacgtactcggatggaagccggtcttgtcgatcaggatgatctggacgaagag | |
| | catcaggggctcgcgccagccgaactgttcgccaggctcaaggcgagcatgcccg | |
| | acggcgaggatctcgtcgtgacccatggcgatgcctgcttgccgaatatcatggt | |
| | ggaaaatggccgcttttctggattcatcgactgtggccggctgggtgtggcggac | |
| | cgctatcaggacatagcgttggctacccgtgatattgctgaagagcttggcggcg | |
| | aatgggctgaccgcttcctcgtgctttacggtatcgccgctcccgattcgcagcg | |
| | catcgccttctatcgccttcttgacgagttcttctgaacccagctttcttgtaca | |
| | aagtggtgataatcgaattccgataatcaacctctggattacaaaatttgtgaaa | |
| | gattgactggtattcttaactatgttgctccttttacgctatgtggatacgctgc | |
| | tttaatgcctttgtatcatgctattgcttcccgtatggctttcattttctcctcc | |
| | ttgtataaatcctggttgctgtctctttatgaggagttgtggcccgttgtcaggc | |
| | aacgtggcgtggtgtgcactgtgtttgctgacgcaacccccactggttggggcat | |
| | tgccaccacctgtcagctcctttccgggactttcgctttccccctccctattgcc | |
| | acggcggaactcatcgccgcctgccttgcccgctgctggacaggggctcggctgt | |
| | tgggcactgacaattccgtggtgttgtcggggaagctgacgtcctttccatggct | |
| | gctcgcctgtgttgccacctggattctgcgcgggacgtccttctgctacgtccct | |
| | tcggccctcaatccagcggaccttccttcccgcggcctgctgccggctctgcggc | |
| | ctcttccgcgtcttcgccttcgccctcagacgagtcggatctccctttgggccgc | |
| | ctccccgcatcgggaattcccgcggttcgctttaagaccaatgacttacaaggca | |
| | gctgtagatcttagccactttttaaaagaaaaggggggactggaagggctaattc | |
| | actcccaacgaagacaagatctgctttttgcttgtactgggtctctctggttaga | |
| | ccagatctgagcctgggagctctctggctaactagggaacccactgcttaagcct | |
| | caataaagcttgccttgagtgcttcaagtagtgtgtgcccgtctgttgtgtgact | |
| | ctggtaactagagatccctcagacccttttagtcagtgtggaaaatctctagcag | |
| | tagtagttcatgtcatcttattattcagtatttataacttgcaaagaaatgaata | |
| | tcagagagtgagaggaacttgtttattgcagcttataatggttacaaataaagca | |
| | atagcatcacaaatttcacaaataaagcatttttttcactgcattctagttgtgg | |
| | tttgtccaaactcatcaatgtatcttatcatgtctggctctagctatcccgcccc | |
| | taactccgcccatcccgcccctaactccgcccagttccgcccattctccgcccca | |
| | tggctgactaattttttttatttatgcagaggccgaggccgcctcggcctctgag | |
| | ctattccagaagtagtgaggaggcttttttggaggcctagggacgtacccaattc | |
| | gccctatagtgagtcgtattacgcgcgctcactggccgtcgttttacaacgtcgt | |
| | gactgggaaaaccctggcgttacccaacttaatcgccttgcagcacatccccctt | |
| | tcgccagctggcgtaatagcgaagaggcccgcaccgatcgcccttcccaacagtt | |
| | gcgcagcctgaatggcgaatgggacgcgccctgtagcggcgcattaagcgcggcg | |
| | ggtgtggtggttacgcgcagcgtgaccgctacacttgccagcgccctagcgcccg | |
| | ctcctttcgctttcttcccttcctttctcgccacgttcgccggctttccccgtca | |
| | agctctaaatcgggggctccctttagggttccgatttagtgctttacggcacctc | |
| | gaccccaaaaaacttgattagggtgatggttcacgtagtgggccatcgccctgat | |
| | agacggtttttcgccctttgacgttggagtccacgttctttaatagtggactctt | |
| | gttccaaactggaacaacactcaaccctatctcggtctattcttttgatttataa | |
| | gggattttgccgatttcggcctattggttaaaaaatgagctgatttaacaaaaat | |
| | ttaacgcgaattttaacaaaatattaacgcttacaatttaggtggcacttttcgg | |
| | ggaaatgtgcgcggaacccctatttgtttatttttctaaatacattcaaatatgt | |
| | atccgctcatgagacaataaccctgataaatgcttcaataatattgaaaaaggaa | |
| | gagtatgagtattcaacatttccgtgtcgcccttattcccttttttgcggcattt | |
| | tgccttcctgtttttgctcacccagaaacgctggtgaaagtaaaagatgctgaag | |
| | atcagttgggtgcacgagtgggttacatcgaactggatctcaacagcggtaagat | |
| | ccttgagagttttcgccccgaagaacgttttccaatgatgagcacttttaaagtt | |
| | ctgctatgtggcgcggtattatcccgtattgacgccgggcaagagcaactcggtc | |
| | gccgcatacactattctcagaatgacttggttgagtactcaccagtcacagaaaa | |
| | gcatcttacggatggcatgacagtaagagaattatgcagtgctgccataaccatg | |
| | agtgataacactgcggccaacttacttctgacaacgatcggaggaccgaaggagc | |
| | taaccgcttttttgcacaacatgggggatcatgtaactcgccttgatcgttggga | |
| | accggagctgaatgaagccataccaaacgacgagcgtgacaccacgatgcctgta | |
| | gcaatggcaacaacgttgcgcaaactattaactggcgaactacttactctagctt | |
| | cccggcaacaattaatagactggatggaggcggataaagttgcaggaccacttct | |
| | gcgctcggcccttccggctggctggtttattgctgataaatctggagccggtgag | |
| | cgtgggtctcgcggtatcattgcagcactggggccagatggtaagccctcccgta | |
| | tcgtagttatctacacgacggggagtcaggcaactatggatgaacgaaatagaca | |
| | gatcgctgagataggtgcctcactgattaagcattggtaactgtcagaccaagtt | |
| | tactcatatatactttagattgatttaaaacttcatttttaatttaaaaggatct | |
| | aggtgaagatcctttttgataatctcatgaccaaaatcccttaacgtgagttttc | |
| | gttccactgagcgtcagaccccgtagaaaagatcaaaggatcttcttgagatcct | |
| | ttttttctgcgcgtaatctgctgcttgcaaacaaaaaaaccaccgctaccagcgg | |
| | tggtttgtttgccggatcaagagctaccaactctttttccgaaggtaactggctt | |
| | cagcagagcgcagataccaaatactgttcttctagtgtagccgtagttaggccac | |
| | cacttcaagaactctgtagcaccgcctacatacctcgctctgctaatcctgttac | |
| | cagtggctgctgccagtggcgataagtcgtgtcttaccgggttggactcaagacg | |
| | atagttaccggataaggcgcagcggtcgggctgaacggggggttcgtgcacacag | |
| | cccagcttggagcgaacgacctacaccgaactgagatacctacagcgtgagctat | |
| | gagaaagcgccacgcttcccgaagagagaaaggcggacaggtatccggtaagcgg | |
| | cagggtcggaacaggagagcgcacgagggagcttccagggggaaacgcctggtat | |
| | ctttatagtcctgtcgggtttcgccacctctgacttgagcgtcgatttttgtgat | |
| | gctcgtcaggggggcggagcctatggaaaaacgccagcaacgcggcctttttacg | |
| | gttcctggccttttgctggccttttgctcacatgttctttcctgcgttatcccct | |
| | gattctgtggataaccgtattaccgcctttgagtgagctgataccgctcgccgca | |
| | gccgaacgaccgagcgcagcgagtcagtgagcgaggaagcggaagagcgcccaat | |
| | acgcaaaccgcctctccccgcgcgttggccgattcattaatgcagctggcacgac | |
| | aggtttcccgactggaaagcgggcagtgagcgcaacgcaattaatgtgagttagc | |
| | tcactcattaggcaccccaggctttacactttatgcttccggctcgtatgttgtg | |
| | tggaattgtgagcggataacaatttcacacaggaaacagctatgaccatgattac | |
| | gccaagcgcgcaattaaccctcactaaagggaacaaaagctggagctgcaagctt | |
| 8459 pcLV- | aatattttgaagcatttatcagggttattgtctcatgagcggatacatatttgaa | 103 |
| CMV-AFX01- | tgtatttagaaaaataaacaaataggggttccgcgcacatttccccgaaaagtgc | |
| 230 | cacctgacgtctaagaaaccattattatcatgacattaacctataaaaataggcg | |
| XT73(C)- | tatcacgaggccctttcgtctcgcgcgtttcggtgatgacggtgaaaacctctga | |
| IRES-Neo | cacatgcagctcccggagacggtcacagcttgtctgtaagcggatgccgggagca | |
| (9581 bp) | gacaagcccgtcagggcgcgtcagcgggtgttggcgggtgtcggggctggcttaa | |
| | ctatgcggcatcagagcagattgtactgagagtgcaccatatgcggtgtgaaata | |
| | ccgcacagatgcgtaaggagaaaataccgcatcaggcgccattcgccattcaggc | |
| | tgcgcaactgttgggaagggcgatcggtgcgggcctcttcgctattacgccagct | |
| | ggcgaaagggggatgtgctgcaaggcgattaagttgggtaacgccagggttttcc | |
| | cagtcacgacgttgtaaaacgacggccagtgccaagctgacgcgtgtagtcttat | |
| | gcaatactcttgtagtcttgcaacatggtaacgatgagttagcaacatgccttac | |
| | aaggagagaaaaagcaccgtgcatgccgattggtggaagtaaggtggtacgatcg | |
| | tgccttattaggaaggcaacagacgggtctgacatggattggacgaaccactgaa | |
| | ttgccgcattgcagagatattgtatttaagtgcctagctcgatacataaacgggt | |
| | ctctctggttagaccagatctgagcctgggagctctctggctaactagggaaccc | |
| | actgcttaagcctcaataaagcttgccttgagtgcttcaagtagtgtgtgcccgt | |
| | ctgttgtgtgactctggtaactagagatccctcagacccttttagtcagtgtgga | |
| | aaatctctagcagtggcgcccgaacagggacttgaaagcgaaagggaaaccagag | |
| | gagctctctcgacgcaggactcggcttgctgaagcgcgcacggcaagaggcgagg | |
| | ggcggcgactggtgagtacgccaaaaattttgactagcggaggctagaaggagag | |
| | agatgggtgcgagagcgtcagtattaagcgggggagaattagatcgcgatgggaa | |
| | aaaattcggttaaggccagggggaaagaaaaaatataaattaaaacatatagtat | |
| | gggcaagcagggagctagaacgattcgcagttaatcctggcctgttagaaacatc | |
| | agaaggctgtagacaaatactgggacagctacaaccatcccttcagacaggatca | |
| | gaagaacttagatcattatataatacagtagcaaccctctattgtgtgcatcaaa | |
| | ggatagagataaaagacaccaaggaagctttagacaagatagaggaagagcaaaa | |
| | caaaagtaagaccaccgcacagcaagcggccactgatcttcagacctggaggagg | |
| | agatatgagggacaattggagaagtgaattatataaatataaagtagtaaaaatt | |
| | gaaccattaggagtagcacccaccaaggcaaagagaagagtggtgcagagagaaa | |
| | aaagagcagtgggaataggagctttgttccttgggttcttgggagcagcaggaag | |
| | cactatgggcgcagcgtcaatgacgctgacggtacaggccagacaattattgtct | |
| | ggtatagtgcagcagcagaacaatttgctgagggctattgaggcgcaacagcatc | |
| | tgttgcaactcacagtctggggcatcaagcagctccaggcaagaatcctggctgt | |
| | ggaaagatacctaaaggatcaacagctcctggggatttggggttgctctggaaaa | |
| | ctcatttgcaccactgctgtgccttggaatgctagttggagtaataaatctctgg | |
| | aacagatttggaatcacacgacctggatggagtgggacagagaaattaacaatta | |
| | cacaagcttaatacactccttaattgaagaatcgcaaaaccagcaagaaaagaat | |
| | gaacaagaattattggaattagataaatgggcaagtttgtggaattggtttaaca | |
| | taacaaattggctgtggtatataaaattattcataatgatagtaggaggcttggt | |
| | aggtttaagaatagtttttgctgtactttctatagtgaatagagttaggcaggga | |
| | tattcaccattatcgtttcagacccacctcccaaccccgaggggacccgacaggc | |
| | ccgaaggaatagaagaagaaggtggagagagagacagagacagatccattcgatt | |
| | agtgaacggatctcgacggtatcggttaacttttaaaagaaaaggggggattggg | |
| | gggtacagtgcaggggaaagaatagtagacataatagcaacagacatacaaacta | |
| | aagaattacaaaaacaaattacaaaattcaaaattttatttccgcgttacataac | |
| | ttacggtaaatggcccgcctggctgaccgcccaacgacccccgcccattgacgtc | |
| | aataatgacgtatgttcccatagtaacgccaatagggactttccattgacgtcaa | |
| | tgggtggagtatttacggtaaactgcccacttggcagtacatcaagtgtatcata | |
| | tgccaagtacgccccctattgacgtcaatgacggtaaatggcccgcctggcatta | |
| | tgcccagtacatgaccttatgggactttcctacttggcagtacatctacgtatta | |
| | gtcatcgctattaccatggtgatgcggttttggcagtacatcaatgggcgtggat | |
| | agcggtttgactcacggggatttccaagtctccaccccattgacgtcaatgggag | |
| | tttgttttggcaccaaaatcaacgggactttccaaaatgtcgtaacaactccgcc | |
| | ccattgacgcaaatgggcggtaggcgtgtacggtgggaggtctatataagcagag | |
| | ctggtttagtgaaccgtcagatccgctagcctagcggatccgaattctcgatcac | |
| | aagtttgtacaaaaaagcaggcttcgctagccgccaccatgccgctcctgctgct | |
| | gctgcccctgctgtgggcaggagctctggccatccctggagggctgtccgaggcc | |
| | aagcctgctacgccggaaattcaagaaatcgtcgataaggttaagccacagctcg | |
| | aagagaaaactggggaaacatacggcaaacttgaggcggttcagtacaaaactca | |
| | ggtcgtgcactactacgtgcactacaacgatcaagggaccaactactatatcaag | |
| | gtccgcgcgggagacaacaaatacatgcatctcaaagtgttcaaatcgctgtggg | |
| | gcgagaaccttttcgcgaagtgggaggatttggtgctgactggatatcaggtcga | |
| | taaaaacaaggatgacgagcttactggctttggaggaggagggtccggtggagga | |
| | ggctctggtggaggcggaagcggcggaggaggatcgggtggcggaggaagcggtg | |
| | gtggaggctccatgattcccggaggcctctcagaagccaaaccagccactcctga | |
| | gattcaggaaattgtcgacaaggtcaagccgcagctggaagagaagactggcgaa | |
| | acctacgggaagctagaggcggtccagtacaagacccaagtcgtccattactatg | |
| | tacattacaacgaccagggaaccaattactacattaaagtgcgggccggagataa | |
| | taagtatatgcatcttaaggtgtttaagtcgttgtggggcgaaaacctgtttgcc | |
| | aagtgggaagatcttgtgctcaccggttaccaagtggacaagaacaaggacgacg | |
| | agctgaccgggttcggaggaggcggatcgggaggtggaggatccggtggcggtgg | |
| | aagcggaggtggcggatctgggggagggggatcaggaggcggcggatccatgata | |
| | cctggcggactgtcggaggcgaaaccggccacccctgaaatccaggagattgtgg | |
| | acaaggtgaaaccccaactcgaggaaaaaaccggcgaaacgtacggtaaactcga | |
| | agccgtgcagtataagacccaggtcgtacactactatgtgcactataacgatcag | |
| | ggaacgaactactacatcaaggtcagagctggagacaataagtacatgcacctca | |
| | aggttttcaagtcgttatggggagaaaacctcttcgcgaaatgggaggatctcgt | |
| | gctgaccggctaccaggtggacaaaaataaggatgatgaactcaccggtttcgga | |
| | ggcggaggatcaggcggagggggttcaggagggggtggctcaggaggagggggta | |
| | gtggaggcggtggatccggaggaggtggcagcatgatcccacggggtctgtccga | |
| | agccaagccagccaccccagagatccaagaaatagtggataaggtgaagccccag | |
| | cttgaggaaaagactggagagacttacggaaagctcgaggctgtccaatacaaaa | |
| | cacaggtgctggcgaacttcttccaaaggagatggccaggcagtactaactatta | |
| | catcaaagtgcgcgcaggagataacaaatatatgcacttgaaggtgttcaatgga | |
| | ccgtggaagttccggaacaccgacagaggcgccgacagggtcttgaccgggtacc | |
| | aagtcgataagaacaaagacgacgaactgaccggtttctgaatctagagacccag | |
| | ctttcttgtacaaagtggtatcgagatatctgcatttaattaatctagagtttaa | |
| | acgcggccgcaaattccgcccctctccctcccccccccctaacgttactggccga | |
| | agccgcttggaataaggccggtgtgcgtttgtctatatgttattttccaccatat | |
| | tgccgtcttttggcaatgtgagggcccggaaacctggccctgtcttcttgacgag | |
| | cattcctaggggtctttcccctctcgccaaaggaatgcaaggtctgttgaatgtc | |
| | gtgaaggaagcagttcctctggaagcttcttgaagacaaacaacgtctgtagcga | |
| | ccctttgcaggcagcggaaccccccacctggcgacaggtgcctctgcggccaaaa | |
| | gccacgtgtataagatacacctgcaaaggcggcacaaccccagtgccacgttgtg | |
| | agttggatagttgtggaaagagtcaaatggctctcctcaagcgtattcaacaagg | |
| | ggctgaaggatgcccagaaggtaccccattgtatgggatctgatctggggcctcg | |
| | gtgcacatgctttacatgtgtttagtcgaggttaaaaaaacgtctaggccccccg | |
| | aaccacggggacgtggttttcctttgaaaaacacgatgataatatggccacaacc | |
| | atggttattgaacaagatggattgcacgcaggttctccggccgcttgggtggaga | |
| | ggctattcggctatgactgggcacaacagacaatcggctgctctgatgccgccgt | |
| | gttccggctgtcagcgcaggggcgcccggttctttttgtcaagaccgacctgtcc | |
| | ggtgccctgaatgaactgcaggacgaggcagcgcggctatcgtggctggccacga | |
| | cgggcgttccttgcgcagctgtgctcgacgttgtcactgaagcgggaagggactg | |
| | gctgctattgggcgaagtgccggggcaggatctcctgtcatctcaccttgctcct | |
| | gccgagaaagtatccatcatggctgatgcaatgcggcggctgcatacgcttgatc | |
| | cggctacctgcccattcgaccaccaagcgaaacatcgcatcgagcgagcacgtac | |
| | tcggatggaagccggtcttgtcgatcaggatgatctggacgaagagcatcagggg | |
| | ctcgcgccagccgaactgttcgccaggctcaaggcgcgcatgcccgacggcgagg | |
| | atctcgtcgtgacccatggcgatgcctgcttgccgaatatcatggtggaaaatgg | |
| | ccgcttttctggattcatcgactgtggccggctgggtgtggcggaccgctatcag | |
| | gacatagcgttggctacccgtgatattgctgaagagcttggcggcgaatgggctg | |
| | accgcttcctcgtgctttacggtatcgccgctcccgattcgcagcgcatcgcctt | |
| | ctatcgccttcttgacgagttcttctgagtcgacaatcaacctctggattacaaa | |
| | atttgtgaaagattgactggtattcttaactatgttgctccttttacgctatgtg | |
| | gatacgctgctttaatgcctttgtatcatgctattgcttcccgtatggctttcat | |
| | tttctcctccttgtataaatcctggttgctgtctctttatgaggagttgtggccc | |
| | gttgtcaggcaacgtggcgtggtgtgcactgtgtttgctgacgcaacccccactg | |
| | gttggggcattgccaccacctgtcagctcctttccgggactttcgctttccccct | |
| | ccctattgccacggcggaactcatcgccgcctgccttgcccgctgctggacaggg | |
| | gctcggctgttgggcactgacaattccgtggtgttgtcggggaaatcatcgtcct | |
| | ttccttggctgctcgcctgtgttgccacctggattctgcgcgggacgtccttctg | |
| | ctacgtcccttcggccctcaatccagcggaccttccttcccgcggcctgctgccg | |
| | gctctgcggcctcttccgcgtcttcgccttcgccctcagacgagtcggatctccc | |
| | tttgggccgcctccccgcctggtacctttaagaccaatgacttacaaggcagctg | |
| | tagatcttagccactttttaaaagaaaaggggggactggaagggctaattcactc | |
| | ccaacgaagataagatctgctttttgcttgtactgggtctctctggttagaccag | |
| | atctgagcctgggagctctctggctaactagggaacccactgcttaagcctcaat | |
| | aaagcttgccttgagtgcttcaagtagtgtgtgcccgtctgttgtgtgactctgg | |
| | taactagagatccctcagacccttttagtcagtgtggaaaatctctagcagtagt | |
| | agttcatgtcatcttattattcagtatttataacttgcaaagaaatgaatatcag | |
| | agagtgagaggaacttgtttattgcagcttataatggttacaaataaagcaatag | |
| | catcacaaatttcacaaataaagcatttttttcactgcattctagttgtggtttg | |
| | tccaaactcatcaatgtatcttatcatgtctggctctagctatcccgcccctaac | |
| | tccgcccatcccgcccctaactccgcccagttccgcccattctccgccccatggc | |
| | tgactaattttttttatttatgcagaggccgaggccgcctcggcctctgagctat | |
| | tccagaagtagtgaggaggcttttttggaggcctagacttttgcagagaccaaat | |
| | tcgtaatcatgtcatagctgtttcctgtgtgaaattgttatccgctcacaattcc | |
| | acacaacatacgagccggaagcataaagtgtaaagcctggggtgcctaatgagtg | |
| | agctaactcacattaattgcgttgcgctcactgcccgctttccagtcgggaaacc | |
| | tgtcgtgccagctgcattaatgaatcggccaacgcgcggggagaggcggtttgcg | |
| | tattgggcgctcttccgcttcctcgctcactgactcgctgcgctcggtcgttcgg | |
| | ctgcggcgagcggtatcagctcactcaaaggcggtaatacggttatccacagaat | |
| | caggggataacgcaggaaagaacatgtgagcaaaaggccagcaaaaggccaggaa | |
| | ccgtaaaaaggccgcgttgctggcgtttttccataggctccgcccccctgacgag | |
| | catcacaaaaatcgacgctcaagtcagaggtggcgaaacccgacaggactataaa | |
| | gataccaggcgtttccccctggaagctccctcgtgcgctctcctgttccgaccct | |
| | gccgcttaccggatacctgtccgcctttctcccttcgggaagcgtggcgctttct | |
| | catagctcacgctgtaggtatctcagttcggtgtaggtcgttcgctccaagctgg | |
| | gctgtgtgcacgaaccccccgttcagcccgaccgctgcgccttatccggtaacta | |
| | tcgtcttgagtccaacccggtaagacacgacttatcgccactggcagcagccact | |
| | ggtaacaggattagcagagcgaggtatgtaggcggtgctacagagttcttgaagt | |
| | ggtggcctaactacggctacactagaaggacagtatttggtatctgcgctctgct | |
| | gaagccagttaccttcggaaaaagagttggtagctcttgatccggcaaacaaacc | |
| | accgctggtagcggtggtttttttgtttgcaagcagcagattacgcgcagaaaaa | |
| | aaggatctcaagaagatcctttgatcttttctacggggtctgacgctcagtggaa | |
| | cgaaaactcacgttaagggattttggtcatgagattatcaaaaaggatcttcacc | |
| | tagatccttttaaattaaaaatgaagttttaaatcaatctaaagtatatatgagt | |
| | aaacttggtctgacagttaccaatgcttaatcagtgaggcacctatctcagcgat | |
| | ctgtctatttcgttcatccatagttgcctgactccccgtcgtgtagataactacg | |
| | atacgggagggcttaccatctggccccagtgctgcaatgataccgcgagacccac | |
| | gctcaccggctccagatttatcagcaataaaccagccagccggaagggccgagcg | |
| | cagaagtggtcctgcaactttatccgcctccatccagtctattaattgttgccgg | |
| | gaagctagagtaagtagttcgccagttaatagtttgcgcaacgttgttgccattg | |
| | ctacaggcatcgtggtgtcacgctcgtcgtttggtatggcttcattcagctccgg | |
| | ttcccaacgatcaaggcgagttacatgatcccccatgttgtgcaaaaaagcggtt | |
| | agctccttcggtcctccgatcgttgtcagaagtaagttggccgcagtgttatcac | |
| | tcatggttatggcagcactgcataattctcttactgtcatgccatccgtaagatg | |
| | cttttctgtgactggtgagtactcaaccaagtcattctgagaatagtgtatgcgg | |
| | cgaccgagttgctcttgcccggcgtcaatacgggataataccgcgccacatagca | |
| | gaactttaaaagtgctcatcattggaaaacgttcttcggggcgaaaactctcaag | |
| | gatcttaccgctgttgagatccagttcgatgtaacccactcgtgcacccaactga | |
| | tcttcagcatcttttactttcaccagcgtttctgggtgagcaaaaacaggaaggc | |
| | aaaatgccgcaaaaaagggaataagggcgacacggaaatgttgaatactcatact | |
| | cttcctttttc | |

### Example 1-2: Establishment of Cell Introduction Conditions

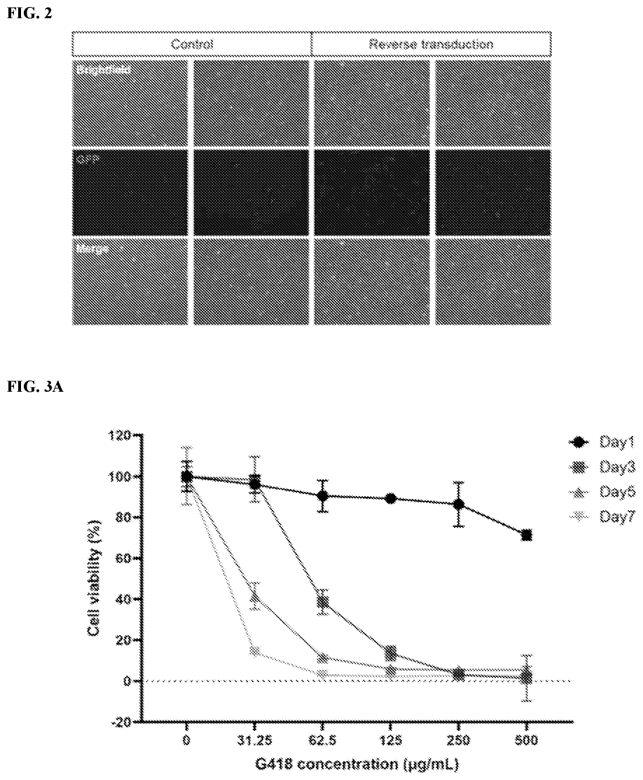

When introducing a gene into PSC-derived MSC using a lentivirus, it is necessary to select an appropriate introduction method in order to increase the introduction efficiency. A typical method of treating cells with a lentivirus is to infect the attached cells with both lentiviral particles and a transduction enhancer. Another method is a reverse transduction method of infecting the cells with the lentivirus during cell attachment by treating the cells with both lentiviral particles and a transduction enhancer before cell attachment. Among these two methods, in order to select a method capable of further increasing the efficiency when introducing the lentivirus into PSC-derived MSC, introduction efficiency was compared using the lentivirus constructed with a vector (SIRION Biotech, SEQ ID NO: 94) including eGFP fluorescence. The frozen PSC-derived MSC was thawed, counted equally, and inoculated. Cell attachment was confirmed the next day, and the cells were then treated with 1-5 MOI of lentivirus and 2-8 μg/mL of polybrene for 16 to 20 hours. Under different conditions, 1-5 MOI of lentivirus and 2-8 μg/mL of polybrene were mixed and placed in a plate, and the same number of cells was inoculated into the plate and treated therewith for 16 to 20 hours. In both conditions, 16 to 20 hours after treatment with the lentivirus, the culture medium containing the lentivirus was removed and replaced with a fresh culture medium, followed by culture for 48 hours. After 48 hours, the cells were harvested and gene introduction efficiency was compared based on the GFP fluorescence-introduced cell population through flow cytometry. Consequently, as shown in FIG. 2, when the attached cells were treated with the lentivirus, the introduction efficiency was 6.61%, whereas when the cells were also treated with the lentivirus during cell inoculation, the introduction efficiency was 21.57%, which was increased about 3.26-fold. Based on these results, it was confirmed that, when introducing a gene into PSC-derived MSC using a lentivirus, treatment with the lentivirus during cell attachment was a method capable of increasing the efficiency.

### Example 1-3: Selection of Genetically Modified Cell into which Gene was Introduced

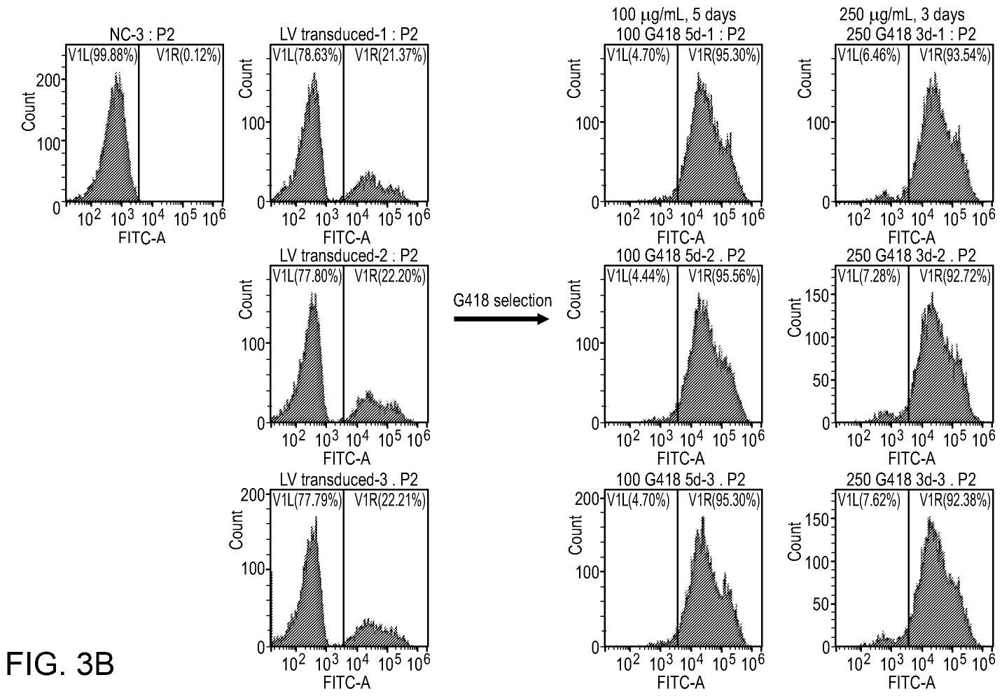

In order to select gene-introduced cells alone after gene introduction through the lentivirus in PSC-derived MSC, the lentivirus was constructed such that a neomycin resistance gene was expressed downstream of the stefin A protein variant (AFFIMER®) gene to be introduced. When the stefin A protein variant (AFFIMER®) gene is introduced into cells, the neomycin resistance gene is also expressed, resulting in antibiotic resistance. However, since treatment with high concentrations of antibiotics for a long time may cause genetic mutation in cells, it is necessary to determine the minimum concentration capable of selecting the gene-introduced cells alone. Since such a concentration varies depending on cell characteristics, in order to determine the minimum G418 concentration that kills PSC-derived MSC, the extent of cell death after cell culture through treatment with the antibiotic G418 at different concentrations was evaluated. The next day after uniform inoculation of PSC-derived MSC into a 96-well plate, the cells were treated with a culture medium containing G418 at 500, 250, 125, 62.5, 31.25, 15.625, and 7.8125 μg/mL. The culture medium containing G418 was thoroughly replaced every 2 days, and cell death was observed after 1, 3, 5, and 7 days from the first treatment day. In order to evaluate cell viability, Cell Counting kit-8 (CCK-8) was diluted 1:10 in the culture medium, and the culture medium containing G418 was thoroughly removed, followed by treatment with 100 μL thereof and then reaction at 37° C. for 1 hour. After reaction, absorbance was measured at 460 nm using a multimode microplate reader. Consequently, as shown in FIG. 3A, when the cells were treated with G418 at a concentration of 500 μg/mL, cell viability was 71.40% on the 1^{st} day but was 1.34% on the 3^{rd} day, indicating that most cells were killed. When the cells were treated with G418 at a concentration of 250 μg/mL, cell viability was 86.29% on the 1^{st} day but was 3.07% on the 3^{rd} day, indicating that most cells were killed, as with 500 μg/mL. In addition, when the cells were treated with G418 at a concentration of 125 μg/mL, cell viability was 89.14% on the 1^{st} day, 13.43% on the 3^{rd} day, and 5.82% on the 5′ day. When the cells were treated with G418 at a concentration of 62.5 μg/mL, cell viability was 90.38% on the 1^{st} day, 38.51% on the 3^{rd} day, 11.52% on the 5^{th} day, and 2.84% on the 7^{th} day.

After setting the G418 concentration and the treatment time, the proportion of the selected gene-introduced cells before and after treatment with G418 was measured. In order to compare suitability for G418 treatment conditions, a lentivirus was constructed (SEQ ID NO: 94) such that a neomycin resistance gene was expressed downstream of the eGFP fluorescent gene. PSC-derived MSC was treated with a mixture of 5 MOI of lentivirus and 2 μg/mL of polybrene, followed by culture for 16 to 20 hours. The culture medium containing the lentivirus was thoroughly removed and then replaced with a fresh culture medium, followed by culture for 48 hours. When cell confluency reached 90% or more, the cells were harvested and then inoculated again at a cell density of 0.4-1.0×10⁴ cells/cm². After culture for 18 to 24 hours, the culture medium was replaced with a culture medium containing G418 at 100 μg/mL and 250 μg/mL, and then replaced with a culture medium containing G418 at each concentration every 2 days. The cells were treated for 5 days in the presence of G418 at 100 μg/mL and for 3 days in the presence of G418 at 250 μg/mL, after which the culture medium was replaced with a culture medium not containing G418. Thereafter, when cell confluency reached 90% or more, the cells were harvested and then the proportion of the eGFP fluorescent gene-introduced cell line was measured through flow cytometry. Consequently, as shown in FIG. 3B, the proportion of the eGFP gene-introduced cell line was 22.00% before treatment with G418, but was 96.01% after treatment for 5 days in the presence of G418 at 100 μg/mL and was 93.47% after treatment for 3 days in the presence of G418 at 250 μg/mL.

### Example 1-4: Promoter Screening

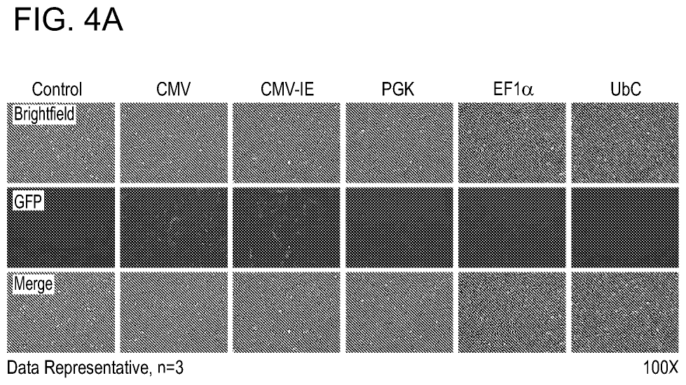

When introducing the stefin A protein variant into PSC-derived MSC, four types of promoters that induce constitutive expression were evaluated in order to select a promoter capable of maintaining stable and high expression. A cell line was constructed with lentiviral particles including a vector (Applied Biological Materials Inc. LV950)) designed to express the eGFP fluorescent protein downstream of four promoters: CMV, PGK, EF1A, and UbC. The cells were treated with a mixture of 1-5 MOI of lentivirus and 2-8 μg/mL of polybrene, followed by culture at 37° C. and 5% CO₂ for 16 to 20 hours. As a positive control, a cell line was constructed and compared under the same conditions using a lentivirus (SIRION Biotech, Table 5) in which an eGFP fluorescent protein was fused downstream of the CMV promoter. The culture medium containing the lentivirus was thoroughly removed and then replaced with a fresh culture medium, followed by culture at 37° C. and 5% CO₂ for 48 hours. After 48 hours, the cells were harvested and gene introduction efficiency was compared based on the GFP fluorescence-introduced cell population through flow cytometry. As shown in FIGS. 4A and 4B, the positive control showed an eGFP gene-introduced cell proportion of 26.9%, whereas the CMV-IE promoter showed 36.5%, which is a 1.4-fold increase. In addition, when using the other PGK, EF1A, and UbC promoters, fluorescence introduction was very low to the levels of 1.6%, 1.5%, and 0.2%, respectively. Therefore, the use of the CMV promoter among the four promoters and the enhancer sequence was confirmed to induce protein expression with the highest efficiency.

### Examples 1-5: Confirmation of Passage Stability of MSC into which Anti-CD40L Stefin A Protein Variant Gene was Introduced

A cell line in which anti-CD40L stefin A protein variant expression was regulated by a vector (VB211001-1274, SEQ ID NO: 96) containing a CMV promoter (CMV-IE) including an enhancer was constructed and compared for long-term subculture stability with a non-introduced cell line (Naïve MSC). A lentivirus was constructed using a vector including an anti-CD40L stefin A protein variant gene and a neomycin resistance gene. The frozen Naïve MSC was thawed, mixed with 1-5 MOI of lentivirus and 2-8 μg/mL of polybrene, and then inoculated into a cell culture dish. After culture at 37° C. and 5% CO₂ for 16 to 20 hours, the culture medium containing the lentivirus was thoroughly removed and then replaced with a fresh culture medium, followed by culture at 37° C. and 5% CO₂ for 48 hours. Thereafter, the cells were harvested and inoculated again at a cell density of 0.4-1.0×10⁴ cells/cm², followed by culture at 37° C. and 5% CO₂ for 18 to 24 hours. The culture medium was replaced with a culture medium containing 100 μg/mL of G418, followed by culture for 5 days, and the culture medium was replaced with a culture medium containing G418 every 2 days. When cell confluency reached 90% or more, the cells were harvested and then frozen. Naïve MSC of the same passage number as the frozen gene-introduced cell line was thawed and inoculated into a T175 flask. After culture for 18 to 24 hours, the culture medium was thoroughly replaced with a fresh culture medium every 2 days, followed by culture. When cell confluency reached 90% or more, the cells were harvested and inoculated again into a T175 flask, followed by culture at 37° C. and 5% CO₂. Continuous culture was carried out until the PDL of the cells dropped to 3.0 or less. The cells remaining after inoculation in each passage were frozen, and after termination of continuous culture, the frozen cells at each passage were thawed and analyzed for purity and immune markers through flow cytometry. Expression of mesenchymal stem cell surface markers CD29, CD44, CD73, and CD105, and expression of cell surface markers for hematopoietic stem cell-specific marker CD45, embryonic stem cell-specific markers SSEA-3, TRA-1-60, and TRA-1-81, and immune marker HLA-DR were comparatively analyzed from passages 10 to 18. As shown in Tables 6 and 7 below, it was confirmed that the expression of the mesenchymal stem cell surface markers CD29, CD44, CD73, and CD105 was maintained at 95% or more until PN18, regardless of gene introduction. Also, expression of CD45, SSEA-3, TRA-1-60, TRA-1-81, and HLA-DR was maintained at less than 1% until PN18, indicating that the characteristics of mesenchymal stem cells were maintained well. Based on these results, it was confirmed that the important characteristics and long-term passage stability of mesenchymal stem cells were maintained even when the anti-CD40L stefin A protein variant gene was introduced.

TABLE 6

| | | Naïve MSC | | | | | | | | |
|---|---|---|---|---|---|---|---|---|---|---|
| Marker | | PN10 | PN11 | PN12 | PN13 | PN14 | PN15 | PN16 | PN17 | PN18 |
| Identification | CD29 | 99.9 | 99.9 | 99.9 | 98.7 | 98.3 | 99.7 | 94.3 | 99.8 | 99.5 |
| Markers | CD44 | 99.9 | 99.9 | 99.9 | 99.8 | 99.9 | 99.9 | 99.9 | 99.9 | 99.9 |
| | CD73 | 99.9 | 99.9 | 99.9 | 99.6 | 98.7 | 99.9 | 98.7 | 99.8 | 99.5 |
| | CD105 | 99.9 | 99.9 | 99.9 | 99.8 | 99.8 | 99.9 | 99.8 | 99.9 | 99.7 |
| Purity | CD45 | 0.8 | 0.9 | 1.3 | 0.6 | 0.6 | 0.5 | 0.6 | 0.5 | 0.5 |
| Markers | SSEA-3 | 0.2 | 0.1 | 0.1 | 0.3 | 0.3 | 0.3 | 0.4 | 0.4 | 0.4 |
| | TRA-1-60 | 0.4 | 0.5 | 0.4 | 0.5 | 0.5 | 0.5 | 0.4 | 0.5 | 0.4 |
| | TRA-1-81 | 0.5 | 0.4 | 0.4 | 0.4 | 0.4 | 0.5 | 0.4 | 0.5 | 0.4 |
| Immune | HLA-DR | 0.3 | 0.3 | 0.4 | 0.4 | 0.5 | 0.5 | 0.5 | 0.4 | 0.4 |
| Makers | | | | | | | | | | |

TABLE 7

| | | eMSC | | | | | | | | |
|---|---|---|---|---|---|---|---|---|---|---|
| Marker | | PN10 | PN11 | PN12 | PN13 | PN14 | PN15 | PN16 | PN17 | PN18 |
| Identification | CD29 | 99.8 | 99.7 | 98.6 | 98.8 | 96.4 | 99.0 | 99.2 | 97.7 | 94.7 |
| Markers | CD44 | 100.0 | 100.0 | 99.9 | 99.7 | 99.9 | 99.9 | 99.9 | 100.0 | 99.9 |
| | CD73 | 100.0 | 100.0 | 99.9 | 100.0 | 99.9 | 99.9 | 99.9 | 99.8 | 99.0 |
| | CD105 | 100.0 | 99.9 | 99.7 | 99.7 | 99.6 | 99.7 | 99.9 | 99.7 | 99.9 |
| Purity | CD45 | 0.1 | 0.6 | 0.3 | 0.4 | 0.4 | 0.3 | 0.2 | 0.1 | 0.5 |
| Markers | SSEA-3 | 0.3 | 0.3 | 0.3 | 0.3 | 0.3 | 0.3 | 0.2 | 0.2 | 0.3 |
| | TRA-1-60 | 0.2 | 0.4 | 0.1 | 0.3 | 0.2 | 0.3 | 0.4 | 0.1 | 0.4 |
| | TRA-1-81 | 0.2 | 0.3 | 0.1 | 0.2 | 0.2 | 0.1 | 0.3 | 0.1 | 0.3 |
| Immune | HLA-DR | 0.7 | 0.4 | 0.3 | 0.3 | 0.2 | 0.1 | 0.3 | 0.1 | 0.4 |
| Makers | | | | | | | | | | |

### Examples 1-6: Additional Screening of Promoter for Expression of Anti-CD40L Stefin A Protein Variant

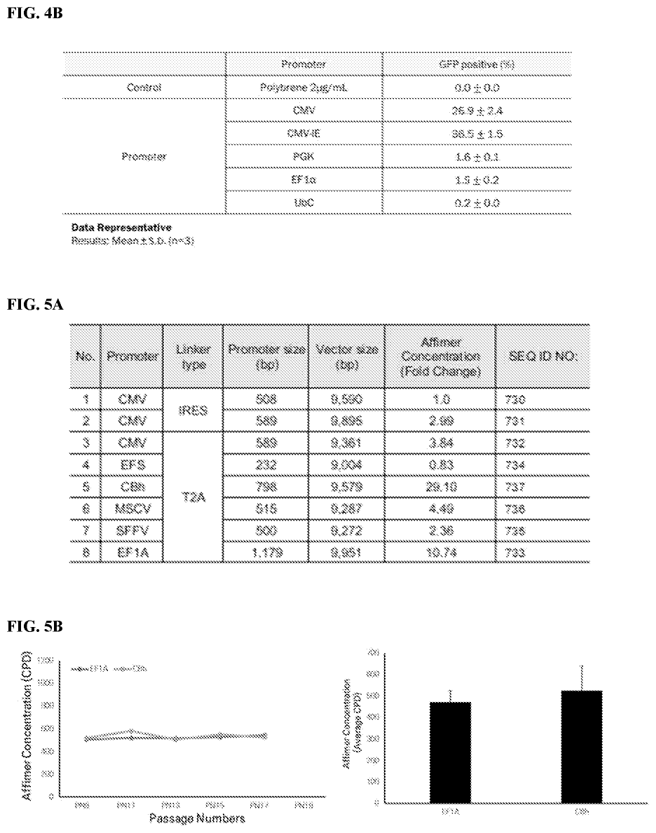

In order to introduce the stefin A protein variant gene into PSC-derived MSC, eight types of lentiviral vectors having characteristics shown in FIG. 5A were constructed, and a promoter capable of inducing stable and high expression was selected.

A cell line was constructed with lentiviral particles including a vector designed such that the stefin A protein variant (AFFIMER®) was expressed downstream of each promoter and in which an antibiotic gene was inserted downstream of the IRES or T2A sequence. The cells were treated with a mixture of 1-5 MOI of lentivirus and 2-8 μg/mL of polybrene, followed by culture at 37° C. and 5% CO₂ for 16 to 20 hours. Thereafter, the culture medium containing the lentivirus was thoroughly removed and then replaced with a fresh culture medium, followed by culture at 37° C. and 5% CO₂ for 48 hours. After 48 hours, the cells were harvested and expression efficiency of the stefin A protein variant was evaluated through ELISA. As shown in FIG. 5A, two promoters (EF1A and CBh) in which the expression level of the stefin A protein variant was increased 10-fold or more compared to the control (SEQ ID NO: 95) were confirmed.

Each cell line in which the expression of the stefin A protein variant (AFFIMERR) was regulated by the EF1A promoter (SEQ ID NO: 9898) or the CBh promoter (SEQ ID NO: 102102) was constructed, followed by long-term subculture, and culture stability, expression level, and activity of the stefin A protein variant (AFFIMERR) were evaluated (through binding ELISA, competition ELISA, or functional cell assay). As shown in FIG. 5B, by confirming that expression of the stefin A protein variant was maintained stable from PN9 to PN17 in both cell lines, an expression cassette combination suitable for expression of the stefin A protein variant in PSC-derived MSC was identified.

In order to confirm maintenance of stable stefin A protein variant expression depending on long-term passage, each gene-introduced cell line in which the expression of the stefin A protein variant was regulated by the EF1A promoter (SEQ ID NO: 98) or CBh promoter (SEQ ID NO: 102) was constructed using the method described in Example 1-5. Each of the two gene-introduced cell lines thus constructed was thawed and then inoculated into a T175 flask using a culture medium. After culture at 37° C. and 5% CO₂ for 18 to 24 hours, the culture medium was thoroughly replaced with a fresh culture medium every 2 days, followed by culture. When cell confluency reached 70-80%, the cells were harvested and inoculated again into a T175 flask, followed by continuous subculture. Continuous culture was carried out until the PDL of the cells dropped to 3.0 or less. The cell culture fluid was harvested and frozen during subculture at each passage, and the cells remaining after subculture were frozen. After termination of continuous culture, the frozen cell culture fluid was completely thawed and diluted, after which the stefin A protein variant in the cell culture fluid was quantified through sandwich ELISA. Since the secreted amount thereof may vary depending on the number of cells and the culture period, the daily amount of stefin A protein variant that was secreted per cell was calculated by division by the number of cells harvested during subculture and the time required to harvest the culture fluid after medium replacement. As shown in Table 8 below, it was confirmed that expression of the stefin A protein variant was stably maintained even when long-term subculture was continued.

TABLE 8

| | Passage | Affimer | Affimer secretion |
|---|---|---|---|
| ID | # | (ng/mL) | (fg/cells/day) |
| eMSC | PN9 | 219.2 | 407.18 |
| (w/EF1A) | PN11 | 196.9 | 462.19 |
| | PN13 | 211.5 | 445.78 |
| | PN15 | 214.0 | 492.88 |
| | PN17 | 188.2 | 548.66 |
| eMSC | PN9 | 216.0 | 462.12 |
| (w/CBh) | PN11 | 296.4 | 696.16 |
| | PN13 | 176.5 | 400.73 |
| | PN15 | 229.5 | 577.59 |
| | PN17 | 177.7 | 486.39 |

### Examples 1-7: Confirmation of Long-Term Passage Stability when Using Additional Promoter

The anti-CD40L stefin A protein variant gene-introduced cell lines (eMSC) including two promoters were constructed and then compared for long-term subculture stability with Naïve MSC in the same manner as in Example 1-6.

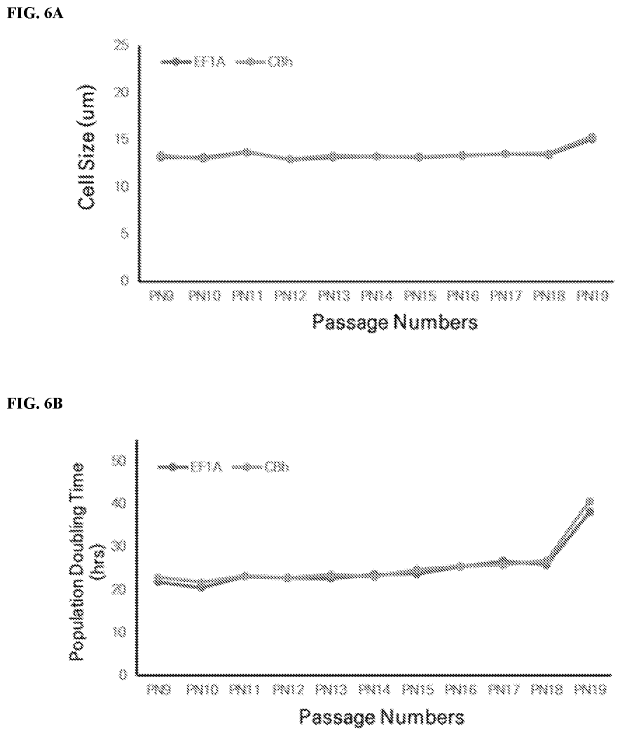

During subculture, cell size, viability, total cell number, PDT, and PDL were measured, and cell morphology was observed using a phase-contrast microscope. Based on the results of morphological observation, two gene-introduced cell lines and Naïve MSC were confirmed to maintain the spindle shape from PN9 to PN19. Cell viability was maintained at 95% or more from PN9 to PN19 in both gene-introduced cell lines, and the amount of the stefin A protein variant that was secreted, PDT, and PDL levels were similar (FIGS. 6A to 6C). PDT was gradually increased from 19.21 hours at PN9 to 37.23 hours at PN19 in Naïve MSC, and showed a tendency to increase from 21.78 hours and 22.86 hours at PN9 to 38.11 hours and 40.67 hours at PN19 in the two gene-introduced cell lines. Also, PDL was maintained at 3.0 or more until PN18 in all of Naïve MSC and the two gene-introduced cell lines, but was decreased to 3.0 or less at PN19. Therefore, the long-term subculture stability of PSC-derived MSC was maintained even when the stefin A protein variant gene was introduced, confirming that the present invention is platform technology suitable for the development of cell gene therapies (Table 9).

TABLE 9

| | Passage | Total Cell | Viability | Cell size | | |
|---|---|---|---|---|---|---|
| | number | (×10⁶ cells) | (%) | (μm) | PDT (h) | PDL |
| Naïve | PN9 | 6.87 ± 0.37 | 99.6 ± 0.1 | 12.9 ± 0.1 | 19.21 ± 0.44 | 3.52 ± 0.08 |
| MSC | PN10 | 6.43 ± 0.13 | 99.7 ± 0.1 | 13.7 ± 0.1 | 21.33 ± 0.18 | 3.42 ± 0.03 |
| | PN11 | 6.57 ± 0.40 | 99.6 ± 0.4 | 13.3 ± 0.1 | 21.75 ± 0.56 | 3.45 ± 0.09 |
| | PN12 | 7.38 ± 1.72 | 99.6 ± 0.1 | 13.6 ± 0.2 | 20.53 ± 0.31 | 3.41 ± 0.05 |
| | PN13 | 5.15 ± 0.31 | 99.4 ± 0.3 | 13.3 ± 0.1 | 23.24 ± 0.64 | 3.10 ± 0.09 |
| | PN14 | 5.58 ± 0.18 | 99.5 ± 0.1 | 13.5 ± 0.0 | 22.39 ± 0.32 | 3.22 ± 0.05 |
| | PN15 | 5.00 ± 0.25 | 99.1 ± 0.5 | 13.8 ± 0.2 | 23.55 ± 0.57 | 3.06 ± 0.07 |
| | PN16 | 5.51 ± 0.15 | 99.0 ± 0.3 | 13.9 ± 0.1 | 22.51 ± 0.28 | 3.20 ± 0.04 |
| | PN17 | 3.98 ± 0.23 | 99.1 ± 0.4 | 14.1 ± 0.2 | 26.39 ± 0.79 | 2.73 ± 0.08 |
| | PN18 | 6.36 ± 0.30 | 99.1 ± 0.2 | 14.9 ± 0.1 | 28.20 ± 0.54 | 3.40 ± 0.07 |
| | PN19 | 3.58 ± 0.06 | 97.7 ± 0.4 | 15.3 ± 0.1 | 37.23 ± 0.33 | 2.58 ± 0.02 |
| eMSC | PN9 | 13.51 ± 0.12 | 99.60 ± 0.14 | 13.10 ± 0.00 | 21.78 ± 0.07 | 4.27 ± 0.01 |
| (w/CBh) | PN10 | 9.14 ± 0.55 | 99.65 ± 0.21 | 13.10 ± 0.00 | 20.52 ± 0.48 | 3.71 ± 0.09 |
| | PN11 | 10.70 ± 0.65 | 99.53 ± 0.12 | 13.67 ± 0.06 | 23.15 ± 0.50 | 3.93 ± 0.09 |
| | PN12 | 6.88 ± 0.19 | 99.60 ± 0.10 | 12.90 ± 0.10 | 22.75 ± 0.28 | 3.30 ± 0.04 |
| | PN13 | 5.96 ± 0.04 | 99.53 ± 0.15 | 13.13 ± 0.15 | 22.66 ± 0.07 | 3.09 ± 0.01 |
| | PN14 | 5.82 ± 0.22 | 99.67 ± 0.06 | 13.23 ± 0.12 | 23.58 ± 0.42 | 3.05 ± 0.05 |
| | PN15 | 5.44 ± 0.41 | 99.73 ± 0.06 | 13.10 ± 0.17 | 23.70 ± 0.87 | 2.96 ± 0.11 |
| | PN16 | 5.90 ± 0.13 | 99.63 ± 0.21 | 13.30 ± 0.17 | 25.37 ± 0.26 | 3.08 ± 0.03 |
| | PN17 | 4.32 ± 0.14 | 99.33 ± 0.32 | 13.47 ± 0.12 | 26.68 ± 0.46 | 2.62 ± 0.05 |
| | PN18 | 8.27 ± 0.36 | 99.63 ± 0.06 | 13.37 ± 0.15 | 25.84 ± 0.46 | 3.56 ± 0.06 |
| | PN19 | 4.32 ± 0.10 | 99.10 ± 0.00 | 15.03 ± 0.15 | 38.11 ± 0.49 | 2.62 ± 0.03 |
| eMSC | PN9 | 11.75 ± 0.42 | 99.45 ± 0.07 | 13.35 ± 0.21 | 22.86 ± 0.29 | 4.07 ± 0.05 |
| (w/EF1A) | PN10 | 7.92 ± 0.57 | 99.70 ± 0.00 | 12.95 ± 0.07 | 21.73 ± 0.64 | 3.50 ± 0.10 |
| | PN11 | 10.68 ± 0.64 | 99.63 ± 0.12 | 13.60 ± 0.00 | 23.16 ± 0.51 | 3.93 ± 0.09 |
| | PN12 | 6.91 ± 0.63 | 99.83 ± 0.12 | 12.93 ± 0.06 | 22.71 ± 0.09 | 3.30 ± 0.01 |
| | PN13 | 5.53 ± 0.28 | 99.50 ± 0.10 | 13.30 ± 0.10 | 23.49 ± 0.57 | 2.98 ± 0.07 |
| | PN14 | 6.12 ± 0.52 | 99.67 ± 0.06 | 13.20 ± 0.00 | 23.02 ± 0.09 | 3.13 ± 0.01 |
| | PN15 | 4.98 ± 0.24 | 99.83 ± 0.06 | 13.20 ± 0.17 | 24.76 ± 0.60 | 2.83 ± 0.07 |
| | PN16 | 5.85 ± 0.28 | 99.80 ± 0.00 | 13.33 ± 0.06 | 25.48 ± 0.57 | 3.06 ± 0.07 |
| | PN17 | 4.61 ± 0.33 | 99.10 ± 0.17 | 13.47 ± 0.15 | 25.80 ± 1.01 | 2.72 ± 0.10 |
| | PN18 | 7.57 ± 0.36 | 99.47 ± 0.15 | 13.53 ± 0.15 | 26.81 ± 0.56 | 3.43 ± 0.07 |
| | PN19 | 3.85 ± 0.09 | 99.03 ± 0.06 | 15.30 ± 0.17 | 40.67 ± 0.55 | 2.46 ± 0.03 |

### Example 2. Analysis of Binding and Inhibitory Activity of Anti-CD40L Stefin a Protein Variant

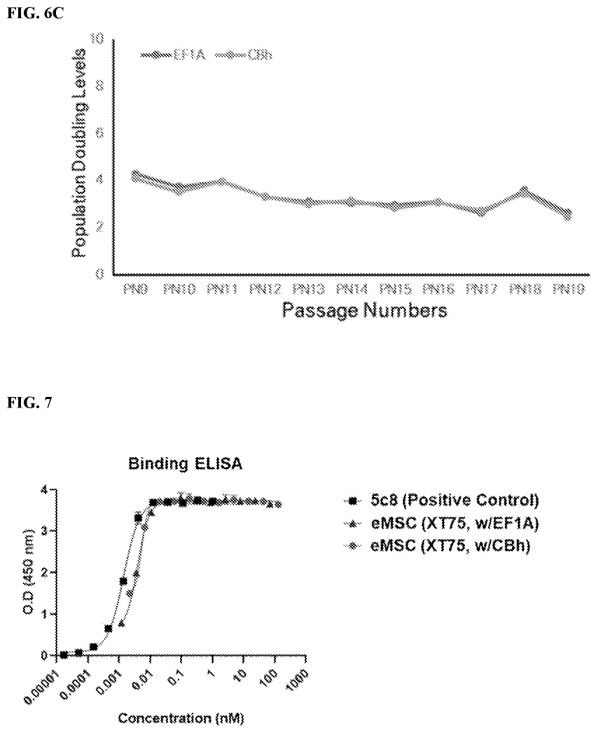

Whether the stefin A protein variant secreted from the constructed anti-CD40L stefin A protein variant gene-introduced cell line had the ability to bind to CD40L and to inhibit binding thereof was evaluated. The anti-CD40L stefin A protein variant gene-introduced cell lines including two promoters were constructed using a lentivirus in the same manner as in Example 1-6. Each cell line was inoculated into a T175 flask at a cell density of 4.0×10³ cells/cm² using a culture medium. After culture at 37° C. and 5% CO₂ for 18 to 24 hours, the culture medium was thoroughly replaced with a fresh culture medium every 2 days, followed by culture. After culture for 3 days, the cells were harvested, and 7.0×10⁶ cells were inoculated into a T175 flask, followed by culture at 37° C. and 5% CO₂ for 18 to 24 hours. The culture medium was thoroughly removed, and the remaining medium was completely removed through washing with 10 mL of MEM alpha. 30 mL of an MEM alpha medium was added to a T175 flask, followed by culture for 48 hours. After 48 hours, all of the cell culture fluid was harvested and then centrifuged to remove residual cells and impurities. The cell culture fluid was transferred to a Vivaspin 20 and centrifuged, the culture fluid was concentrated, and the stefin A protein variant was quantified through sandwich ELISA (LSBio, LS-F4620). For binding ELISA, a 96-well plate was coated with recombinant human CD40L (R&D Systems, 6420-CL-025/CF), after which each of the two concentrated culture fluids was subjected to serial dilution by 1/3 and allowed to react with CD40L. The unbound stefin A protein variant was removed through washing, after which the extent of binding between the stefin A protein variant and CD40L was detected based on a difference in absorbance resulting from reaction with a TMB substrate. Consequently, EC50 values of the stefin A protein variant secreted by the two gene-introduced cell lines were determined to be 0.0039 and 0.0053 nM through bELISA (FIG. 7).

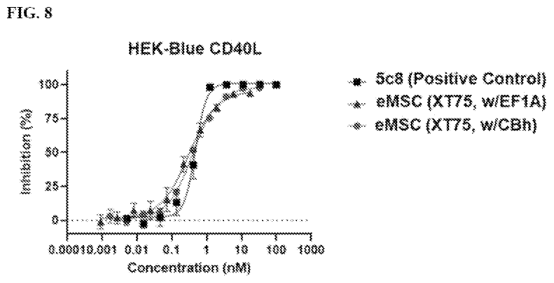

In order to confirm whether the stefin A protein variant secreted by the two gene-introduced cell lines had inhibitory activity on CD40L, a HEK-Blue CD40L cell line (InvivoGen, hkb-cd40) was used. The HEK-Blue CD40L cell line was inoculated at 2.0×10⁴ cells/well into a 96-well plate, followed by culture at 37° C. and 5% CO₂ for 18 to 24 hours. Thereafter, whether the HEK-Blue CD40L cell line was attached well was confirmed, and then 50 μL of the medium was removed. Each of the two concentrated culture fluids was subjected to serial dilution by 1/3, and mixed with a human Mega CD40L recombinant protein (Enzo Lifesciences, ALX-522-100-C010), and 50 μL thereof was placed in the 96-well plate inoculated with HEK-Blue CD40L cells, followed by reaction at 37° C. and 5% CO₂ for 20 to 22 hours. Thereafter, the HEK-Blue CD40L culture fluid was harvested and then allowed to react with a HEK-Blue solution at 37° C. for 3 hours. By measuring the absorbance of each well using a multiplate reader, whether the secreted stefin A protein variant had CD40L-binding inhibitory activity was analyzed. Based on the results of analysis, IC50 for hMega CD40L was 0.470 nM in a benchmark molecule, and IC50 values of the stefin A protein variant secreted by the two gene-introduced cell lines were 0.303 and 0.400 nM, showing similar activities. Therefore, it was concluded that the stefin A protein variant secreted by the gene-introduced cell line had CD40L-binding ability and activity inhibitory ability similar to the reference material (FIG. 8).

### Example 3. Confirmation of Immunosuppressive Activity of MSC into which Anti-CD40L Stefin A Protein Variant Gene was Introduced

### Example 3-1: Confirmation of Effect of Inhibiting T-Cell Activity

The stefin A protein variant secreted by the anti-CD40L stefin A protein variant gene-introduced cell line was confirmed to have an effect of inhibiting immune cell activity due to the antagonistic effect on CD40L.

Example Stefin A protein variant amino acid
sequence
(SEQ ID NO: 104)
IPGGLSEAKPATPEIQEIVDKVKPQLEEKTGETYGKLEAVQYKTQVVHYY
VHYNDQGTNYYIKVRAGDNKYMHLKVFKSLWGENLFAKWEDLVLTGYQVD
KNKDDELTGFGGGGSGGGGSGGGGSGGGGSGGGGSGGGGSMIPGGLSEAK
PATPEIQEIVDKVKPQLEEKTGETYGKLEAVQYKTQVVHYYVHYNDQGTN
YYIKVRAGDNKYMHLKVFKSLWGENLFAKWEDLVLTGYQVDKNKDDELTG
FGGGGSGGGGSGGGGSGGGGSGGGGSGGGGSMIPGGLSEAKPATPEIQEI
VDKVKPQLEEKTGETYGKLEAVQYKTQVVHYYVHYNDQGTNYYIKVRAGD
NKYMHLKVFKSLWGENLFAKWEDLVLTGYQVDKNKDDELTGFGGGGSGGG
GSGGGGSGGGGSGGGGSGGGGSMIPRGLSEAKPATPEIQEIVDKVKPQLE
EKTGETYGKLEAVQYKTQVLANFFQRRWPGSTNYYIKVRAGDNKYMHLKV
FNGPWKFRNTDRGADRVLTGYQVDKNKDDELTGF
Example Stefin A protein variant amino acid
sequence
(SEQ ID NO: 105)
IPGGLSEAKPATPEIQEIVDKVKPQLEEKTGETYGKLEAVQYKTQVVHYY
VHYNDQGTNYYIKVRAGDNKYMHLKVFKSLWGENLFAKWEDLVLTGYQVD
KNKDDELTGFAEAAAKEAAAKEAAAKEAAAKEAAAKEAAAKMIPGGLSEA
KPATPEIQEIVDKVKPQLEEKTGETYGKLEAVQYKTQVVHYYVHYNDQGT
NYYIKVRAGDNKYMHLKVFKSLWGENLFAKWEDLVLTGYQVDKNKDDELT
GFAEAAAKEAAAKEAAAKEAAAKEAAAKEAAAKMIPGGLSEAKPATPEIQ
EIVDKVKPQLEEKTGETYGKLEAVQYKTQVVHYYVHYNDQGTNYYIKVRA
GDNKYMHLKVFKSLWGENLFAKWEDLVLTGYQVDKNKDDELTGFAEAAAK
EAAAKEAAAKEAAAKEAAAKEAAAKMIPRGLSEAKPATPEIQEIVDKVKP
QLEEKTGETYGKLEAVQYKTQVLANFFQRRWPGSTNYYIKVRAGDNKYMH
LKVFNGPWKFRNTDRGADRVLTGYQVDKNKDDELTGF

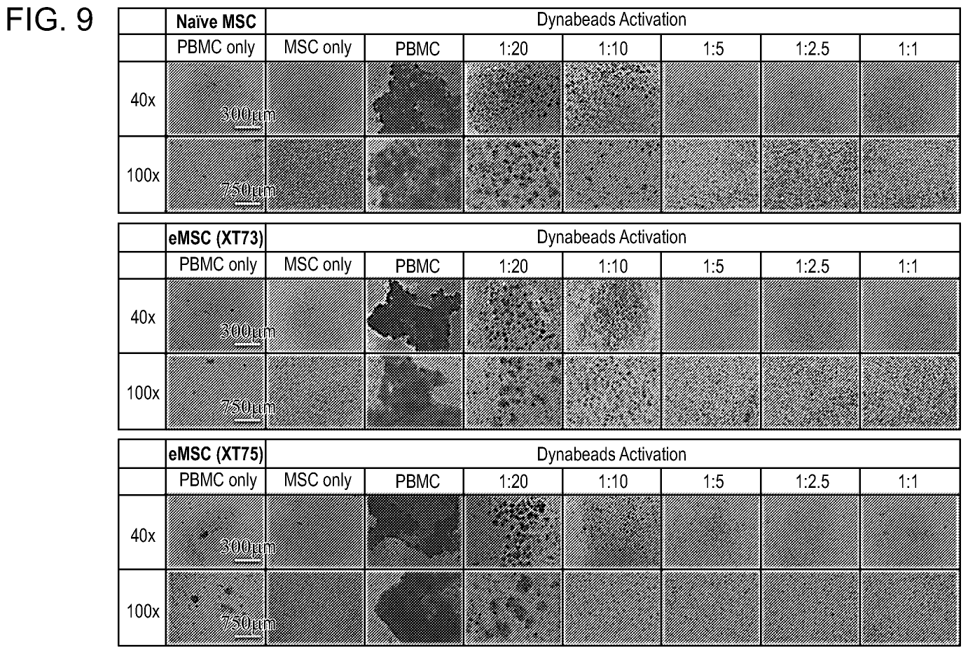

Each of the constructed example stefin A protein variant gene (SEQ ID NO: 104 or 105)-introduced cell line and Naïve MSC was co-cultured with PBMC (Stem Cell Technologies, 70025), and the activation rate of T cells in PBMC was compared. In a 24-well plate, PBMC at 5.0×10⁵ cells/well and each of two anti-CD40L stefin A protein variant gene-introduced cell lines (SEQ ID NO: 104 or 105) and Naïve MSC were mixed in ratios of 1:20, 1:10, 1:5, 1:2.5, and 1:1, followed by cell inoculation. As such, PBMC was inoculated after staining with CFSE capable of confirming cell proliferation, and anti-CD3, CD28 Dynabeads (Gibco, 11161D), and IL-2 (Gibco, PHC0023) were added thereto, followed by culture for 7 days. On the 4^{th} day after inoculation, the culture medium was replaced with a medium containing anti-CD3, CD28 Dynabeads, and IL-2. Based on the results of observation of PBMC clustering capable of evaluating T-cell activity using a phase-contrast microscope, the largest amount of distinct clustering was confirmed when inducing the activation of PBMC alone with Dynabeads. It was observed that clustering of PBMC was decreased with an increase in the proportion of each of the two gene-introduced cell lines and Naïve MSC and was further decreased in the gene-introduced cell lines than in Naïve MSC (FIG. 9).

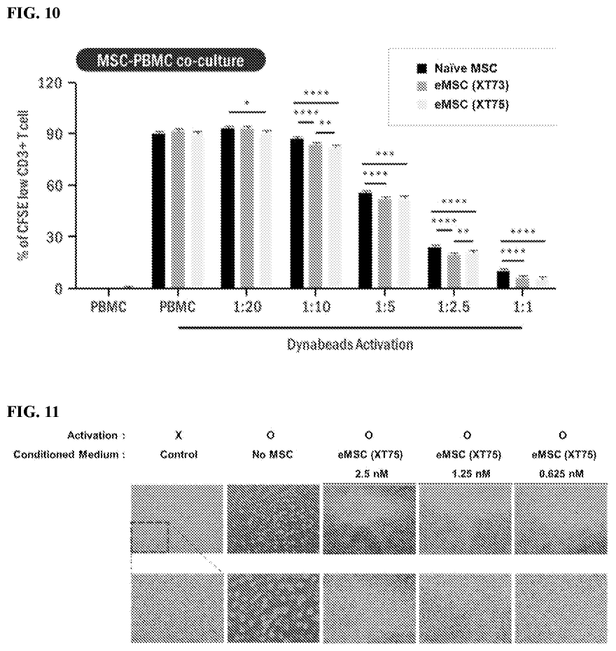

Only PBMC was harvested and the T-cell activity was compared through marker analysis using a flow cytometer. Consequently, CFSE low/CD3 positive cells, corresponding to the population of proliferating T cells, showed a tendency to decrease with an increase in the mixing ratio with each of the two gene-introduced cell lines and Naïve MSC. Moreover, it was confirmed that, when co-cultured with the two gene-introduced cell lines than when co-cultured with Naïve MSC, the T-cell activity was further inhibited (FIG. 10).

### Example 3-2: Confirmation of Effect of Inhibiting B-Cell Activity

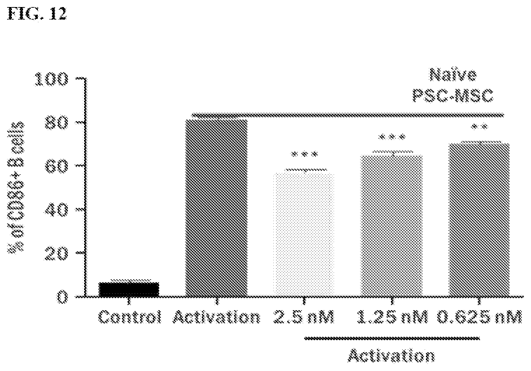

The effect of inhibiting T-cell-mediated B-cell activity by the stefin A protein variant secreted from the anti-CD40L stefin A protein variant gene-introduced cell line was confirmed. A gene-introduced cell line into which the anti-CD40L stefin A protein variant gene (SEQ ID NO: 105) was introduced was constructed using a lentivirus and then inoculated into a T175 flask at a cell density of 4.0×10³ cells/cm² using a culture medium, followed by culture. The culture medium was thoroughly replaced with a fresh culture medium every 2 days, followed by culture for 3 days. Thereafter, the cells were harvested and 7.0×10⁶ cells were inoculated into a T175 flask, followed by culture at 37° C. and 5% CO₂ for 18 to 24 hours. Thereafter, the culture medium was thoroughly removed, and the remaining medium was completely removed through washing with MEM-alpha. 30 mL of an MEM alpha medium was added to a T175 flask, followed by culture for 48 hours. After 48 hours, all of the cell culture fluid was harvested and then centrifuged to remove residual cells and impurities. The cell culture fluid was transferred to a Vivaspin 20 and concentrated by centrifugation, and the stefin A protein variant was quantified through sandwich ELISA. The frozen B cells (Lonza, 4W-601) were thawed, stabilized in a T75 flask for one day, and then inoculated at 5.0×10⁵ cells/well into a 24-well plate. Here, MEGACD40L (Enzo Lifesciences, ALX-522-110-C010), IgM (Jackson Immuno Research Laboratories, 109-006-129), and IL-21 (Peprotech, 200-21) were added to the medium to induce activation of B cells, and simultaneously, the concentrated culture fluid was used together after serial dilution. After culture for 30 hours, B-cell clustering capable of evaluating B-cell activity was observed using a phase-contrast microscope. Consequently, when the cells were treated with MEGA CD40L, IgM, and IL-21 alone, clustering was clearly observed. However, clustering was decreased when the cells were additionally treated with the concentrated culture fluid, confirming that B-cell activity was inhibited (FIG. 11).

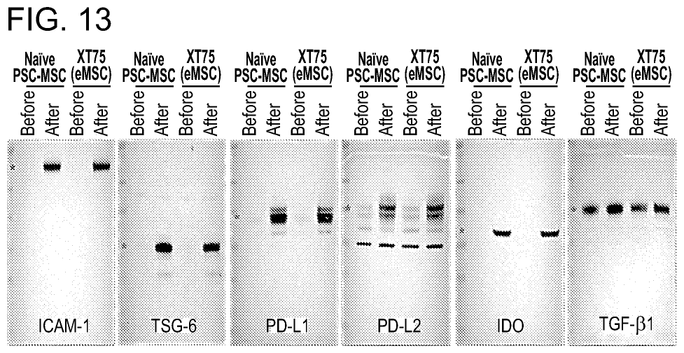

B cells were harvested and the B-cell activity was analyzed using a cell surface marker through flow cytometry. Based on the results of measurement of the population of CD19 positive/CD86 positive cells, which are markers of activated B cells, it was confirmed that the B-cell population, which increased by about 80% when the activity thereof was induced, was significantly decreased when the concentrated culture fluid was further added thereto. Moreover, as the concentrated culture fluid was diluted, the inhibitory effect was decreased, indicating concentration dependence. These results were not confirmed in the culture fluid of Naïve MSC into which the stefin A protein variant gene was not introduced. Therefore, it was concluded that the CD40L signal was suppressed by the stefin A protein variant secreted from the stefin A protein variant gene-introduced cell line, thereby inhibiting B-cell activation (FIG. 12).

### Example 3-3: Confirmation of Immunomodulatory Factor Expression of Anti-CD40L Stefin A Protein Variant Gene-Introduced MSC (eMSC)

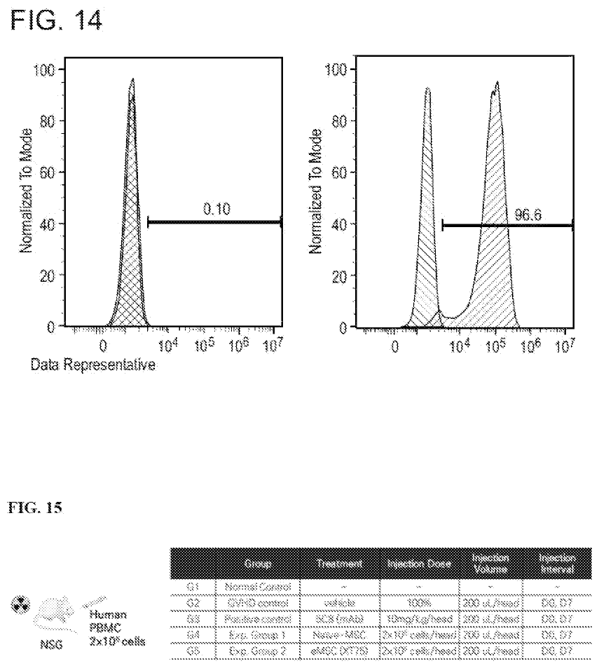

In order to evaluate whether the expression of immunomodulatory factors in an inflammatory environment varies depending on whether the stefin A protein variant gene was introduced, an inflammatory environment was induced in Naïve MSC and the stefin A protein variant gene (SEQ ID NO: 105)-introduced cell line. Naïve MSC of the same passage number as the frozen anti-CD40L stefin A protein variant gene-introduced cell line was thawed, and then inoculated into a T175 flask at a cell density of 4.0×10³ cells/cm² using a culture medium, followed by culture. The culture medium was thoroughly replaced with a fresh culture medium every 2 days, followed by culture for 3 days, after which the cells were harvested. Each of the two cell lines thus harvested was inoculated at 1.5×10⁶ cells in a 100 mm dish, followed by culture for 18 to 24 hours. Thereafter, the culture medium was thoroughly removed and then replaced with a culture medium containing 20 ng/mL of IFN-gamma (PEPROTECH, AF-300-02) and 10 ng/mL of TNF-alpha (PEPROTECH, AF-300-01A), followed by culture for 48 hours to induce an inflammatory environment. After 48 hours, the culture medium was thoroughly removed, washing was performed with PBS, and the cells were harvested with a scraper. The cell pellets were isolated and protein whole lysis was performed using RIPA buffer. The protein was quantified with BCA, loaded in the same amount on SDS-PAGE, and then transferred to a membrane, followed by reaction with TGF-beta1 (Abcam, ab179695), IDO (Abcam, ab76157), IL-10 (Abcam, ab133575), MCP-1 (Abcam, ab214819), and TSG-6, which were secretory factors induced in an inflammatory environment (Abcam, ab267469), and cell surface expression factors such as ICAM-1 (Abcam, ab282575), VCAM-1 (Abcam, ab174279), PD-L1 (Abcam, ab243877), and PD-L2 (Abcam, ab283344) antibodies, after which expression thereof was confirmed. Consequently, when Naïve MSC was treated with IFN-gamma and TNF-alpha to induce an inflammatory environment, expression of immunomodulatory secretion factors such as TGF-beta1, IDO, and TSG-6 was increased, and expression of cell surface expression factors, such as ICAM-1, PD-L1, and PD-L2, was also increased (FIG. 13). When an inflammatory environment was induced in the stefin A protein variant gene-introduced cell line, in addition to Naïve MSC, expression of the immunomodulatory factors was also increased, confirming that there was no difference in expression between Naïve MSC and the stefin A protein variant gene-introduced cell line (FIG. 13).

### Example 4. Verification of Purity of Anti-CD40L Stefin A Protein Variant Gene-Introduced MSC (eMSC)

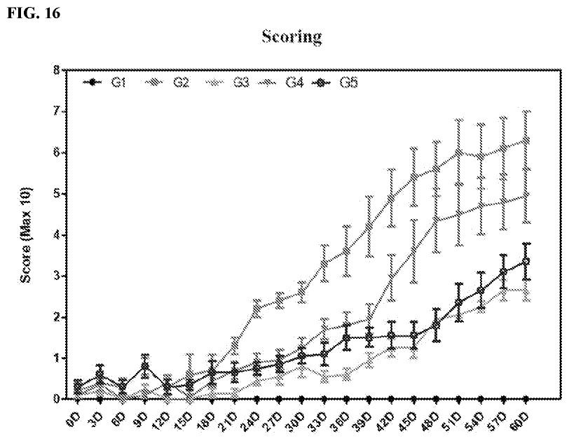

Purity was verified by analyzing the proportion of cells expressing the stefin A protein variant gene (SEQ ID NO: 105) transferred into the constructed stefin A protein variant gene-introduced mesenchymal stem cells (non-target cell proportion). When constructing the stefin A protein variant gene-introduced cell line, a signal peptide sequence MPLLLLLPLLWAGALA (SEQ ID NO: 26) was added so that the stefin A protein variant could be secreted out of the cells. Accordingly, since the stefin A protein variant gene-introduced cell line secretes all of the stefin A protein variant out of the cells, it is necessary to suppress the secretion of the secreted protein in order to identify the cells into which the stefin A protein variant gene is transferred. The secreted protein was accumulated in the Golgi apparatus, which is an intracellular organelle, using Golgiplug (Brefeldin A; BD Pharmingen, 555029), and the proportion of cells expressing the stefin A protein variant therein was determined. The expression of the introduced stefin A protein variant after intracellular protein accumulation by inhibiting the secretion of the secreted protein through Golgiplug was confirmed. Naïve MSC of the same passage number as the frozen anti-CD40L stefin A protein variant gene-introduced cell line was thawed and then inoculated into a 100 mm dish at a cell density of 3.5×10⁴ cells/cm² using a culture medium, followed by culture. The next day, the culture medium was removed and then replaced with a culture medium containing 0.1 μg/mL of Golgiplug, followed by reaction at 37° C. and 5% CO₂ for 4 hours. Thereafter, the cells were harvested, followed by permeabilization and then reaction with an antibody (Novus Biologicals, NBP2-59470) capable of identifying the stefin A protein variant to stain the cells. Using a flow cytometer, the proportion of the cells expressing the stefin A protein variant was determined. Consequently, the proportion of the stefin A protein variant gene-transferred cells in the stefin A protein variant gene-introduced cell line was determined to be 96.6%. It was found that the proportion of the cells into which the stefin A protein variant was introduced in the constructed stefin A protein variant gene-introduced cell line was maintained very high (FIG. 14).

### Example 5. Therapeutic Effect of Anti-CD40L Stefin A Protein-Secreting Cell Line on GVHD Animal Model

### Example 5-1: Experimental Method

NOD.Cg-Prkdcscid Il2rgtmlWjl/SzJ (NSG) 5- to 6-week-old mice were purchased from Jackson Laboratory and JABio, and after acclimatization for 1 to 2 weeks, 7-week-old mice were used for the experiment.

For xenotrasplantation GVHD induction, 4-5 NSG mice were irradiated with 1.5 Gy of radiation, and then injected intravenously (i.v.) with human PBMC (Lonza) at 2×10⁶ cells/head the next day and thus GVHD was induced.

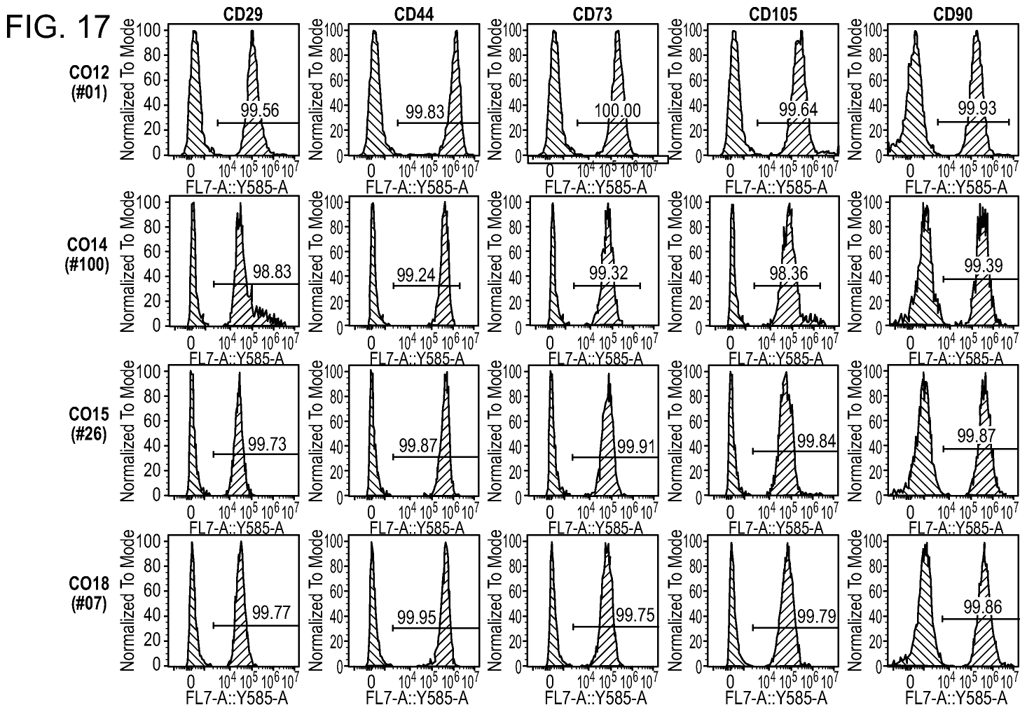

The drug was administered intravenously to the cell-injected group at D0 and D7 based on 24 hours after PBMC administration, and was administered intraperitoneally (i.p.) to a reference material 5c8 antibody-injected group at D0 and D7 based on 24 hours after PBMC administration (FIG. 15).

The animals in this experiment were randomly grouped before irradiation depending on the body weight for the experiment, and analysis of variance (ANOVA) was performed to determine homogeneity between groups.

The animals were observed for general symptoms every day, and particularly observed before and after injection of cells and materials, and animals in severe pain or a moribund state during the observation period were euthanized after symptoms thereof were recorded. The body weight of the mice was measured twice a week, and the GVHD clinical score was measured every 3 days based on the drug administration day 0 and followed-up for 60 days.

### Example 5-2: Experiment Result

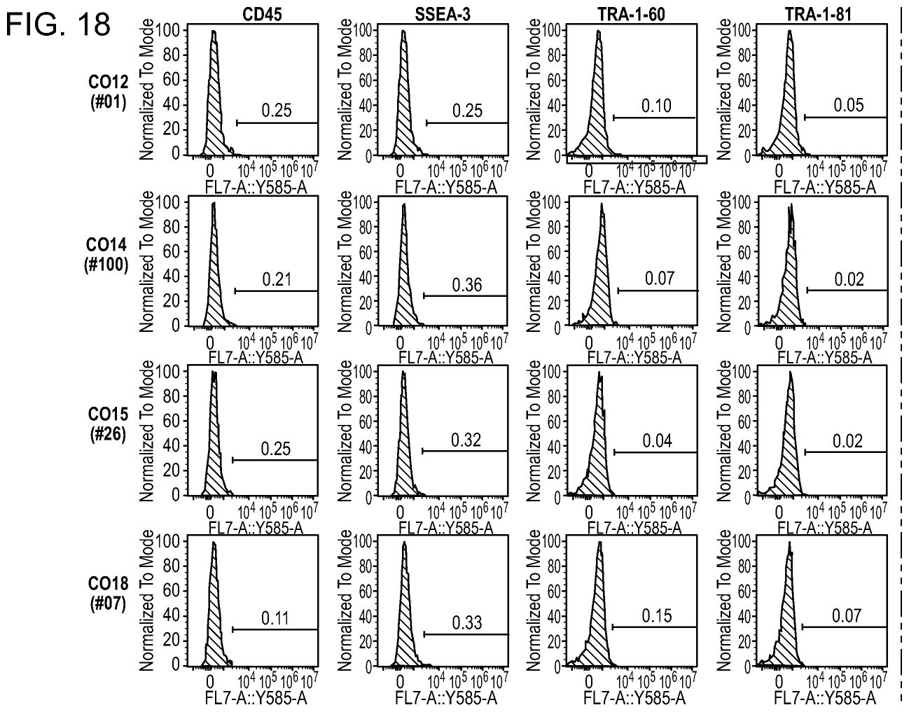

GVHD clinical scoring was given 0 to 2 scores each for five items of weight loss, posture, activity, fur texture, and skin integrity, and the summed values are shown in FIG. 28. The XT75 gene-expressing engineered MSC (eMSC) administration group showed low score values compared to the control, and statistical significance was confirmed through two-way ANOVA, and the interaction, column factor, and row factor all showed P<0.0001. Compared to the positive control administered with 5c8, the XT75-expressing eMSC administration group showed similar score values (FIG. 16).

### Example 6: Characteristics of an Example Binding Protein Expressed on the Cell Surface of MSCs

### Stable Genetic Modification of PSC-Derived MSCs (Pluripotent Stem Cell-Derived Mesenchymal Stromal Cells) Using Lentiviral Vectors

Numerous stefin A protein variants that specifically bind to human TNFR2 were generated. Among the those stefin A protein variant specifically binding to human TNFR2 clones, the top 7 clone genes with high activity were selected: clones 1, 7, 9, 12, 26, 44, and 100, which are shown in Table 10 below. These TNFR2-binding stefin A variants were each cloned into a plasmid vector so that they could be expressed by the CBh promoter, and lentiviral vectors were produced respectively. Lentiviral vectors were constructed using a vector including a nucleic acid sequence encoding an example binding protein, namely, a stefin A protein variant that specifically binds to TNFR2 fused to a transmembrane domain (derived from PDGFR, AVGQDTQEVIVVPHSLPFKVVVISAILALVVLTIISLIILIMLWQKKPR (SEQ ID NO: 106), Biotechnology and Bioengineering, 73(4), 313-323) and also encoded a neomycin resistance gene (Gene Volume 85, Issue 2, 28 Dec. 1989, Pages 421-426). The frozen Naïve PSC-derived MSC was thawed, mixed with 1 MOI of lentiviral vectors and 2-8 g/mL of polybrene, and then inoculated into a cell culture flask. After culture at 37° C. and 5% C02 for 16 to 20 hours, the culture medium containing the lentivirus was thoroughly removed and then replaced with a fresh culture medium, followed by culture at 37° C. and 5% C02 for 48 hours. Thereafter, the cells were harvested and inoculated again at a cell density of 0.4-1.0×10⁴ cells/cm2, followed by culture at 37° C. and 5% C02 for 18 to 24 hours. The culture medium was replaced with a culture medium containing 100-250 μg/mL of G418, followed by culture for 5 days, and the culture medium was replaced with a culture medium containing G418 every 2 days. When cell confluency reached 90% or more, the cells were harvested and then frozen.

TABLE 10
Example TNFR2-binding stefin A variant sequences

| | | Loop 2 | | Loop 4 | | Protein |
|---|---|---|---|---|---|---|
| Clone | | SEQ ID | | SEQ ID | | SEQ ID |
| Name | Loop 2 | NO: | Loop 4 | NO: | Protein sequence | NO: |
| CLONE | GQDQWGNYA | 107 | PDIPHNHWH | 114 | MIPGGLSEAKPATPEIQ | 121 |
| 001 | | | | | EIVDKVKPQLEEKTGET | |
| | | | | | YGKLEAVQYKTQVVGQD | |
| | | | | | QWGNYAGTNYYIKVRAG | |
| | | | | | DNKYMHLKVFKSLPDIP | |
| | | | | | HNHWHEDLVLTGYQVDK | |
| | | | | | NKDDELTGFAAAEQKLI | |
| | | | | | SEEDLAAHHHHHH | |
| CLONE | GLDIWGNNA | 108 | ADTARTEEA | 115 | MIPGGLSEAKPATPEIQ | 122 |
| 007 | | | | | EIVDKVKPQLEEKTGET | |
| | | | | | YGKLEAVQYKTQVVGLD | |
| | | | | | IWGNNAGTNYYIKVRAG | |
| | | | | | DNKYMHLKVFKSLADTA | |
| | | | | | RTEEAEDLVLTGYQVDK | |
| | | | | | NKDDELTGFAAAEQKLI | |
| | | | | | SEEDLAAHHHHHH | |
| CLONE | RLFHIWRWW | 109 | IHIYTDSIQ | 116 | MIPGGLSEAKPATPEIQ | 123 |
| 009 | | | | | EIVDKVKPQLEEKTGET | |
| | | | | | YGKLEAVQYKTQVVRLF | |
| | | | | | HIWRWWGTNYYIKVRAG | |
| | | | | | DNKYMHLKVFKSLIHIY | |
| | | | | | TDSIQEDLVLTGYQVDK | |
| | | | | | NKDDELTGFAAAEQKLI | |
| | | | | | SEEDLAAHHHHHH | |
| CLONE | YWGVQGALD | 110 | NWNHEFDLV | 117 | MIPGGLSEAKPATPEIQ | 124 |
| 012 | | | | | EIVDKVKPQLEEKTGET | |
| | | | | | YGKLEAVQYKTQVVYWG | |
| | | | | | VQGALDGTNYYIKVRAG | |
| | | | | | DNKYMHLKVFKSLNWNH | |
| | | | | | EFDLVEDLVLTGYQVDK | |
| | | | | | NKDDELTGFAAAEQKLI | |
| | | | | | SEEDLAAHHHHHH | |
| CLONE | LEIPSAIIY | 111 | YFWGDQEHW | 118 | MIPRGLSEAKPATPEIQ | 125 |
| 026 | | | | | EIVDKVKPQLEEKTGET | |
| | | | | | YGKLEAVQYKTQVLALE | |
| | | | | | IPSAIIYSTNYYIKVRA | |
| | | | | | GDNKYMHLKVFNGPYFW | |
| | | | | | GDQEHWADRVLTGYQVD | |
| | | | | | KNKDDELTGFAAAEQKL | |
| | | | | | ISEEDLAAHHHHHH | |
| CLONE | LFPEAVYWV | 112 | IWNDNAAQH | 119 | MIPGGLSEAKPATPEIQ | 126 |
| 044 | | | | | EIVDKVKPQLEEKTGET | |
| | | | | | YGKLEAVQYKTQVVLFP | |
| | | | | | EAVYWVGTNYYIKVRAG | |
| | | | | | DNKYMHLKVFKSLIWND | |
| | | | | | NAAQHEDLVLTGYQVDK | |
| | | | | | NKDDELTGFAAAEQKLI | |
| | | | | | SEEDLAAHHHHHH | |
| CLONE | QSWDKHYQLRE | 113 | EVPHHAHGV | 120 | MIPRGLSEAKPATPEIQ | 127 |
| 100 | | | | | EIVDKVKPQLEEKTGET | |
| | | | | | YGKLEAVQYKTQVLAQS | |
| | | | | | WDKHYQLRESTNYYIKV | |
| | | | | | RAGDNKYMHLKVENGPE | |
| | | | | | VPHHAHGVADRVLTGYQ | |
| | | | | | VDKNKDDELTGFAAAEQ | |
| | | | | | KLISEEDLAAHHHHHH | |

* The C' terminal sequence of SEQ ID NOs: 121 to 127, AAAEQKLISEEDLAHHHHHH, is a sequence for purification of Stefin A protein variant.

PSC-Derived MSCs Retain their Sternness after Lentiviral Transduction with the Nucleic Acids Encoding Stefin a Protein Variant Specifically Binding to TNFR2

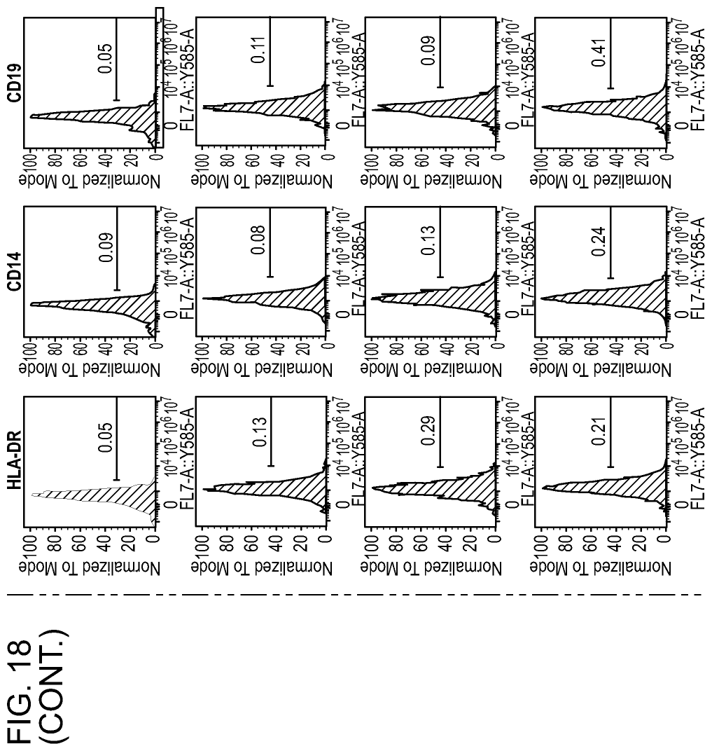

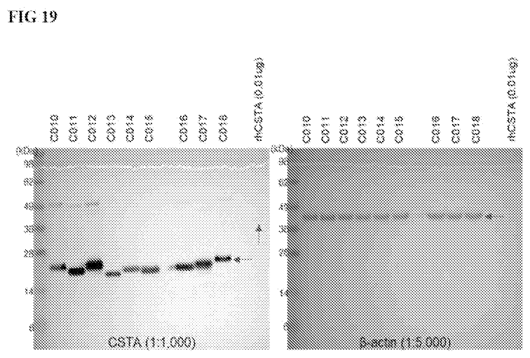

Flow cytometric analysis of eMSCs, 5 passages after their transduction with the lentivirus encoding stefin A protein variant specifically binding to TNFR2, indicate their sternness, which was comparable to Naïve PSC-derived MSCs. The frozen cells at each passage were thawed and analyzed for purity and immune markers through flow cytometry. Expression of mesenchymal stromal cell surface markers CD29, CD44, CD73, and CD105, and expression of cell surface markers for hematopoietic stem cell-specific marker CD45, embryonic stem cell-specific markers SSEA-3, TRA-1-60, and TRA-1-81, and immune marker HLA-DR were comparatively analyzed with Naïve MSCs. As shown in FIG. 17 and FIG. 18, it was confirmed that the expression of the mesenchymal stromal cell surface markers CD29, CD44, CD73, and CD105 was maintained at 95%, regardless of gene introduction. Also, expression of CD45, SSEA-3, TRA-1-60, TRA-1-81, and HLA-DR was maintained at less than 1%, indicating that the characteristics of mesenchymal stromal cells were maintained well.

Assessment of Expression of an Example Stefin a Protein Variant that Specifically Binds to TNFR2 in eMSCs

Extracellular expression of an example target binding fusion protein in eMSCs was confirmed. The human PDGFR (platelet-derived growth factor receptor) transmembrane domain was fused to the C-terminus of stefin A protein variant specifically binding to TNFR2 as an extracellularly facing plasma membrane protein. Western blot and flow cytometry analyses were carried out to confirm functional expression of the recombinant fusion proteins PDGFR and stefin A protein variant specifically binding to TNFR2 in PSC-derived MSCs.

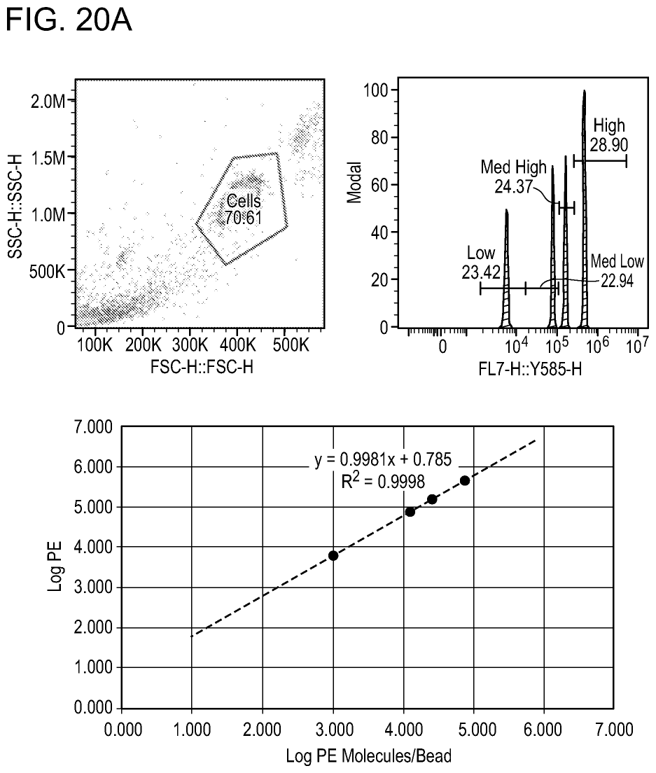

eMSCs transduced with lentiviral vectors were harvested and protein lysates prepared after five passages. In Western analysis, all cells transduced with PDGFR transmembrane domain fused stefin A protein variant specifically binding to TNFR2 demonstrated high levels expression of stefin A protein variant specifically binding to TNFR2 (FIG. 19).

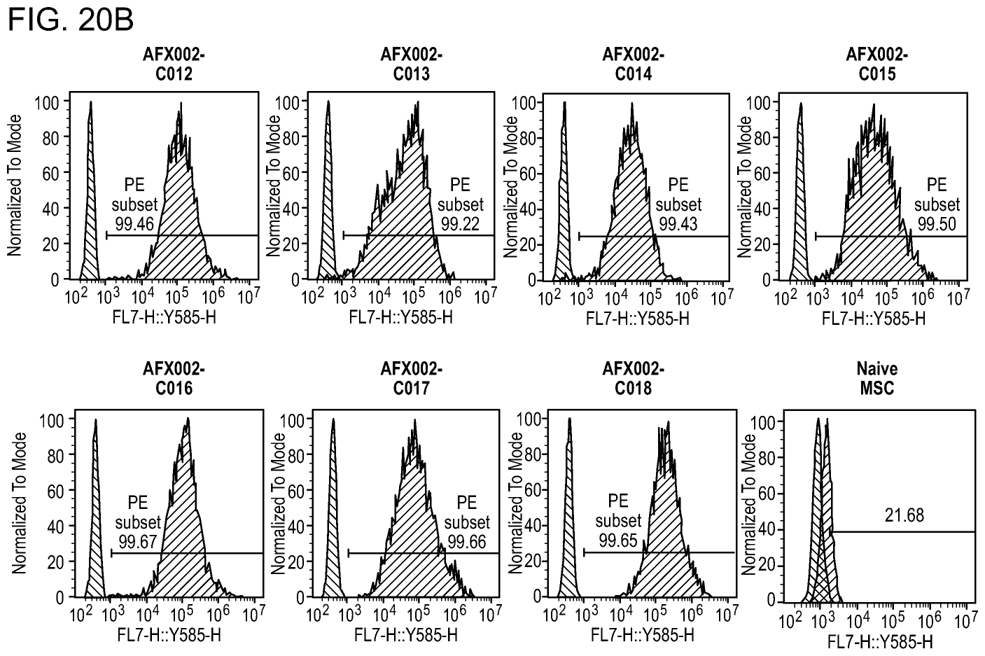

The percentage of cells that express stefin A protein variant are determined by flow cytometry using phycoerythrin (PE)-labelled antibodies to the AFFIMER®, and the mean absolute number of membrane-anchored AFFIMER® proteins per cell using QuantiBRITE PE calibration beads (BD Biosciences, San Jose, CA, USA). The FACS analysis shows that the AFFIMER® is introduced and expressed in more than 95% of MSCs (FIG. 20B). To estimate the absolute number of membrane-anchored proteins of AFFIMER®, we used QuantiBRITE PE beads. Tubes of QuantiBRITE PE beads contain a lyophilized pellet of beads that have been conjugated with four concentrations of PE. Each type of beads has a known number of PE molecules per bead. Using this kit, we plotted a calibration curve and established a formula that enabled MFI values to be converted into the number of AFFIMER® molecules on the cell surface. When Naïve PSC-derived MSCs (non-genetically modified MSCs) were compared, the highest levels of AFFIMER® proteins were found in eMSC transduced with TNFR2 AFFIMER® proteins fused hPDGFR, respectively (Table 11 and FIG. 20A and FIG. 20B).

TABLE 11
Result of Antibodies Bound per Cell

| | AFFIMER ® | | | | Antibodies |
|---|---|---|---|---|---|
| Cell line | Clone | Geometric | Log Geometric | x = Log₁₀ | Bound/Cell |
| ID | Number | Mean of PE | Mean of PE (y) | PE/Cell | (ABC) |
| AFX-MSC-MCB | — | 577 | 2.761 | 2.312 | 205 |
| 001 | | | | | |
| AFX002-C010 | 3t0 | 85,833 | 4.934 | 4.538 | 34,524 |
| AFX002-C011 | SQT | 59,756 | 4.776 | 4.377 | 23,823 |
| AFX002-C012 | 001 | 71,955 | 4.857 | 4.460 | 28,817 |
| AFX002-C013 | 009 | 16,053 | 4.206 | 3.792 | 6,197 |
| AFX002-C014 | 100 | 7,739 | 3.889 | 3.468 | 2,935 |
| AFX002-C015 | 026 | 29,299 | 4.467 | 4.060 | 11,478 |
| AFX002-C016 | 012 | 39,587 | 4.598 | 4.194 | 15,624 |
| AFX002-C017 | 044 | 49,784 | 4.697 | 4.296 | 19,759 |
| AFX002-C018 | 007 | 38,518 | 4.586 | 4.182 | 15,192 |

eMSC—HEK-Blue TNFα Cell Co-Culture for TNFR2 Agonistic Function Assessment

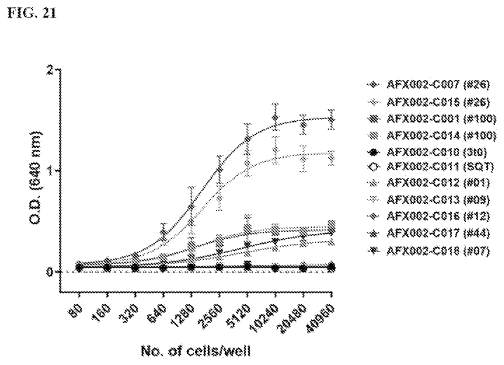

The 7 lead clones were stably transduced in PSC-derived MSCs, along with 2 controls, SQT-Gly and 3t0-Gly which has shown to be negative. The stefin A protein variant expressing eMSCs were co-cultured with HEK Blue reporter cells at 10 different cell densities from 40,960 to 80 cell/well with a fixed number of HEK Blue cells of 20,000 cells/well. After 24 h, the release of SEAP in the supernatant was quantified using Quanti-Blue and measurement of Absorbance 640 nm. Among the seven types of AFFIMER® transduced eMSCs, AFX002-C013, AFX002-C016, and AFX002-C017 cells did not show TNFR2 agonist activity, and AFX002-C010 and AFX002-C011 cells used as negative control also did not show agonist activity. HEK-Blue TNF-α cell activation was detected in four candidate cell lines: AFX002-C012, AFX002-C014, AFX002-C015, and AFX002-C018 (FIG. 21). The soluble form stefin A protein variants (AFFIMER®) specifically binding to human TNFR2 failed to induce activation of HEK-Blue TNF a cells. However, stefin A protein variant genes transduced PSC-derived MSCs induce activation of TNFR2. Reportedly, triggering of TNFR2-associated signaling pathways requires secondary clustering of initially formed trimeric TNF-TNFR2 complexes (Front. Immunol. 10:2040, 2019). Our results support the fact that the expression of AFFIMER® on the cell membrane induces clustering between AFFIMER® and TNFR2, which can transduce signals.

### Additional Embodiments

Although specific embodiments of the present disclosure have been described illustratively, those skilled in the art will appreciate that the present disclosure may be embodied in other specific forms without changing the technical spirit or essential features thereof. Thus, the embodiments described above should be understood to be non-limiting and illustrative in every way.

1. A population of genetically modified mesenchymal stromal cells (MSCs), wherein the MSCs comprise an exogenous nucleic acid comprising a coding sequence that encodes a binding agent that binds a target protein, wherein the binding agent comprises one or more binding domains from an antibody or antibody mimetic.

2. The population of genetically modified MSCs of claim 1, wherein the binding agent comprises a stefin A protein variant, Fab, Fab′, F(ab′)2, Fv, Fd, scFv, sdFv), VL, VH, Camel Ig, V-NAR, VHH, trispecific (Fab3), bispecific (Fab2), diabody ((VL-VH)2 or (VH-VL)2), triabody (trivalent), tetrabody (tetravalent), minibody ((scFv-CH3)2), bispecific single-chain Fv (Bis-scFv), a shark heavy-chain-only antibody (VNAR), a microprotein (cysteine knot protein, knottin), affibody, aptamer, avimer, nanobody, unibody, a single domain antibody, affilin, affitin, adnectin, atrimer, evasin, DARPin, anticalin, avimer, fynomer, versabody, repebody, or a duocalin.

3. The population of genetically modified MSCs of claim 1, wherein the binding agent comprises a stefin A protein variant.

4.-5. (canceled)

6. The population of genetically modified MSCs of claim 1, wherein the binding agent is secreted, wherein the (i) the population of genetically modified MSCs secrete the binding agent at an average level of 200 fg/cell/day or more; and/or (ii) the population of genetically modified MSCs secrete the binding agent at an average level of 100 to 1100 fg/cell/day.

7. (canceled)

8. The population of genetically modified MSCs of claim 1, wherein the binding agent is membrane-anchored or cell-surface-expressed, wherein the (i) the genetically modified MSCs express the binding agent at a level averaging at least 10,000 molecules or more of the binding agent molecules per cell; and/or (ii) the genetically modified MSCs express the binding agent at a level averaging 2500 to 35,000 binding agent molecules per cell.

9. (canceled)

10. The population of genetically modified MSCs of claim 1, wherein the binding agent is a target binding fusion protein comprising:
(a) at least one selected from the group consisting of a transmembrane domain, a hinge domain, a coiled coil domain, a virus-derived domain, an intracellular signaling domain, and a localization domain; and/or
(b) at least one selected from the group consisting of a signal peptide, a Fc domain, a binding domain, a cytokine, a half-life extension domain, a growth factor, an enzyme, a cell-penetrating domain, a therapeutic peptide, and a therapeutic protein.

11.-13. (canceled)

14. The population of genetically modified MSCs of claim 10, wherein the target binding fusion protein comprises a transmembrane domain, wherein the transmembrane domain is derived from group consisted of CD3, CD4, CD5, CD8, CD28, CD99, PDGFR, and PTGFRN, wherein the target binding fusion protein comprises a hinge domain derived from an immunoglobulin.

15. The population of genetically modified MSCs of claim 3, wherein the binding agent comprises a trimer or tetramer of stefin A protein variants.

16. The population of genetically modified MSCs of claim 1, wherein the exogenous nucleic acid comprises a transcriptional regulatory sequence that is operably linked to the coding sequence, wherein the transcriptional regulatory sequence is a promoter selected from a cytomegalovirus (CMV) promoter, a PGK promoter, an EF1α promoter, an EFS promoter, a CBh promoter, an MSCV promoter, an SFFV promoter, and a UbC promoter.

17. (canceled)

18. The population of genetically modified MSCs of claim 1, wherein the exogenous nucleic acid comprises (i) an IRES or 2A sequence and/or (ii) a selection gene.

19. (canceled)

20. The population of genetically modified MSCs of claim 1, wherein the MSCs are derived from induced pluripotent stem cells or embryonic stem cells.

21. The population of genetically modified MSCs of claim 1, wherein (i) the MSCs express at least one cell surface marker selected from CD29, CD44, CD73, CD90 and CD105; and/or (ii) the MSCs do not express a cell surface marker selected from CD11b, CD14, CD34, CD45, CD79, HLA-DR, TRA-1-60, and TRA-1-81.

22. The population of genetically modified MSCs of claim 21, wherein at least 90% of expression of the cell surface marker is maintained in the population of MSCs after at least 15 passages.

23. (canceled)

24. The population of genetically modified MSCs of claim 1, wherein at least 95% of the MSCs are CD73+ and CD105+; and less than 1% express CD45, SSEA-3, TRA-1-60, TRA-1-81, and HLA-DR.

25. The population of genetically modified MSCs of claim 1, wherein the target protein is express on the surface of an immune cell.

26.-27. (canceled)

28. The population of genetically modified MSCs of claim 1, wherein the target protein is a TNF Receptor (e.g., TNFR2) or an immunostimulatory TNF receptor ligand (e.g., CD27L, CD40L, 41BBL, or GITRL).

29. The population of genetically modified MSCs of claim 1, wherein the target protein is a proinflammatory cytokine (e.g., IL-1, IL-6, IL-12, and IL-18, TNF-α, IFNγ, GM-CSF).

30.-31. (canceled)

32. A method of producing a population of genetically modified mesenchymal stromal cells (MSCs) of claim 1, comprising:
contacting a population of MSCs with a lentiviral vector comprising the exogenous nucleic acid comprising the coding sequence that encodes a binding agent, and
culturing the population of MSCs.

33.-37. (canceled)

38. A population of genetically modified pluripotent stem cells (PSCs), wherein the cells comprise an exogenous nucleic acid comprising a coding sequence that encodes a target binding agent, wherein the target binding agent comprises one or more binding domains from an antibody or antibody mimetic.

39.-52. (canceled)

53. A method of treating or preventing an immune disease, comprising administering to a subject in need thereof an effective amount of the population of genetically modified MSCs of claim 1.

54. (canceled)

55. A method of treating cancer, comprising administering to a subject in need thereof an effective amount of the population of genetically modified MSCs of claim 1.

56.-59. (canceled)


SEQUENCE LISTING (448 sequences)

SEQ ID NO: 1 - protein, 48 aa, synthetic construct
  VARIANT 48: note = X can be V or D
MIPGGLSEAKPATPEIQEIVDKVKPQLEEKTGETYGKLEAVQYKTQVX

SEQ ID NO: 2 - protein, 24 aa, synthetic construct
GTNYYIKVRAGDNKYMHLKVFKSL

SEQ ID NO: 3 - protein, 21 aa, synthetic construct
EDLVLTGYQVDKNKDDELTGF

SEQ ID NO: 4 - protein, 99 aa, synthetic construct
  VARIANT 4: note = X can be G, A, V, R, K, D or E
  VARIANT 48: note = X can be V, D or L-A
  VARIANT 49..51: note = X can be any amino acid
  VARIANT 52: note = X can be G, A, V, S or T
  VARIANT 74: note = X can be G, A, V, S or T
  VARIANT 73: note = X can be R, K, N, Q, S or T
  VARIANT 75: note = X can be A, V, I, L, G or P
  VARIANT 76..78: note = X can be any amino acid
  VARIANT 79: note = X can be G, A, V, D or E
  VARIANT 81: note = X can be A, V, I, L, R or K
  VARIANT 51: note = X residue can be repeated up to seventeen times
  VARIANT 78: note = X residue can be repeated up to seventeen times
MIPXGLSEAKPATPEIQEIVDKVKPQLEEKTGETYGKLEAVQYKTQVXXXXXTNYYIKVRAGDNKYMHLKVFXXXXXXXDXVLTGYQVDKNKDDELTGF

SEQ ID NO: 5 - protein, 99 aa, synthetic construct
  VARIANT 49..51: note = X can be any amino acid
  VARIANT 76..78: note = X can be any amino acid
  VARIANT 51: note = X residue can be repeated up to seventeen times
  VARIANT 78: note = X residue can be repeated up to seventeen times
MIPGGLSEAKPATPEIQEIVDKVKPQLEEKTGETYGKLEAVQYKTQVDXXXGTNYYIKVRAGDNKYMHLKVFKSLXXXEDLVLTGYQVDKNKDDELTGF

SEQ ID NO: 6 - protein, 49 aa, synthetic construct
MIPGGLSEAKPATPEIQEIVDKVKPQLEEKTNETYGKLEAVQYKTQVLA

SEQ ID NO: 7 - protein, 99 aa, synthetic construct
  VARIANT 49..51: note = X can be any amino acid
  VARIANT 76..78: note = X can be any amino acid
  VARIANT 51: note = X residue can be repeated up to seventeen times
  VARIANT 78: note = X residue can be repeated up to seventeen times
MIPGGLSEAKPATPEIQEIVDKVKPQLEEKTGETYGKLEAVQYKTQVVXXXGTNYYIKVRAGDNKYMHLKVFKSLXXXEDLVLTGYQVDKNKDDELTGF

SEQ ID NO: 8 - protein, 100 aa, synthetic construct
  VARIANT 50..52: note = X can be any amino acid
  VARIANT 77..79: note = X can be any amino acid
  VARIANT 52: note = X residue can be repeated up to seventeen times
  VARIANT 79: note = X residue can be repeated up to seventeen times
MIPGGLSEAKPATPEIQEIVDKVKPQLEEKTGETYGKLEAVQYKTQVLAXXXGTNYYIKVRAGDNKYMHLKVFKSLXXXEDLVLTGYQVDKNKDDELTGF

SEQ ID NO: 9 - protein, 18 aa, Homo sapiens
MKWVTFISLLFLFSSAYS

SEQ ID NO: 10 - protein, 22 aa, synthetic construct
MDMRAPAGIFGFLLVLFPGYRS

SEQ ID NO: 11 - protein, 19 aa, Homo sapiens
MTRLTVLALLAGLLASSRA

SEQ ID NO: 12 - protein, 19 aa, synthetic construct
MELGLSWIFLLAILKGVQC

SEQ ID NO: 13 - protein, 19 aa, synthetic construct
MELGLRWVFLVAILEGVQC

SEQ ID NO: 14 - protein, 19 aa, synthetic construct
MKHLWFFLLLVAAPRWVLS

SEQ ID NO: 15 - protein, 19 aa, synthetic construct
MDWTWRILFLVAAATGAHS

SEQ ID NO: 16 - protein, 19 aa, synthetic construct
MDWTWRFLFVVAAATGVQS

SEQ ID NO: 17 - protein, 19 aa, synthetic construct
MEFGLSWLFLVAILKGVQC

SEQ ID NO: 18 - protein, 19 aa, synthetic construct
MEFGLSWVFLVALFRGVQC

SEQ ID NO: 19 - protein, 26 aa, synthetic construct
MDLLHKNMKHLWFFLLLVAAPRWVLS

SEQ ID NO: 20 - protein, 22 aa, synthetic construct
MDMRVPAQLLGLLLLWLSGARC

SEQ ID NO: 21 - protein, 22 aa, synthetic construct
MKYLLPTAAAGLLLLAAQPAMA

SEQ ID NO: 22 - protein, 17 aa, synthetic construct
MGVKVLFALICIAVAEA

SEQ ID NO: 23 - protein, 18 aa, Homo sapiens
MAFLWLLSCWALLGTTFG

SEQ ID NO: 24 - protein, 14 aa, Homo sapiens
MQLLSCIALILALV

SEQ ID NO: 25 - protein, 15 aa, Homo sapiens
MNLLLILTFVAAAVA

SEQ ID NO: 26 - protein, 16 aa, Homo sapiens
MPLLLLLPLLWAGALA

SEQ ID NO: 27 - protein, 30 aa, synthetic construct
MDSKGSSQKGSRLLLLLVVSNLLLCQGVVS

SEQ ID NO: 28 - protein, 23 aa, Homo sapiens
MDAMKRGLCCVLLLCGAVFVSPS

SEQ ID NO: 29 - protein, 16 aa, synthetic construct
MLLLLLLLLLLALALA

SEQ ID NO: 30 - protein, 21 aa, synthetic construct
MWWRLWWLLLLLLLLWPMVWA

SEQ ID NO: 31 - protein, 5 aa, synthetic construct
  VARIANT 1..5: note = The entire sequence of amino acids 1-5 can be repeated one to six times
GGGGS

SEQ ID NO: 32 - protein, 8 aa, synthetic construct
GGGGGGGG

SEQ ID NO: 33 - protein, 6 aa, synthetic construct
GGGGGG

SEQ ID NO: 34 - protein, 18 aa, synthetic construct
KESGSVSSEQLAQFRSLD

SEQ ID NO: 35 - protein, 14 aa, synthetic construct
EGKSSGSGSESKST

SEQ ID NO: 36 - protein, 12 aa, synthetic construct
GSAGSAAGSGEF

SEQ ID NO: 37 - protein, 5 aa, synthetic construct
  VARIANT 1..5: note = The entire sequence of amino acids 1-5 can be repeated one to six times
EAAAK

SEQ ID NO: 38 - protein, 46 aa, synthetic construct
AEAAAKEAAAKEAAAKEAAAKALEAEAAAKEAAAKEAAAKEAAAKA

SEQ ID NO: 39 - protein, 5 aa, synthetic construct
PAPAP

SEQ ID NO: 40 - protein, 12 aa, synthetic construct
AEAAAKEAAAKA

SEQ ID NO: 41 - protein, 20 aa, synthetic construct
  VARIANT 9..10: note = AP residues can be repeated up to twenty-four times
APAPAPAPAPAPAPAPAPAP

SEQ ID NO: 42 - protein, 4 aa, synthetic construct
GGSG

SEQ ID NO: 43 - protein, 4 aa, synthetic construct
GSGS

SEQ ID NO: 44 - protein, 4 aa, synthetic construct
GGGS

SEQ ID NO: 45 - protein, 4 aa, synthetic construct
  VARIANT 2..4: note = GGS residues can be repeated one to seven times
SGGS

SEQ ID NO: 46 - protein, 7 aa, synthetic construct
GGGSGGG

SEQ ID NO: 47 - protein, 8 aa, synthetic construct
ESGGGGVT

SEQ ID NO: 48 - protein, 9 aa, synthetic construct
LESGGGGVT

SEQ ID NO: 49 - protein, 6 aa, synthetic construct
GRAQVT

SEQ ID NO: 50 - protein, 6 aa, synthetic construct
WRAQVT

SEQ ID NO: 51 - protein, 8 aa, synthetic construct
ARGRAQVT

SEQ ID NO: 52 - protein, 33 aa, synthetic construct
SGTTSGTTRLLSGHTCFTLTGLLGTLVTMGLLT

SEQ ID NO: 53 - protein, 26 aa, synthetic construct
SGTSPGLSAGATVGIMIGVLVGVALI

SEQ ID NO: 54 - protein, 24 aa, synthetic construct
SAPVLSAVATVGITIGVLARVALI

SEQ ID NO: 55 - protein, 24 aa, synthetic construct
SSPDLSAGTAVSIMIGVLAGMALI

SEQ ID NO: 56 - protein, 25 aa, synthetic construct
TLGGNSASYTFVSLLFSAVTLLLLC

SEQ ID NO: 57 - protein, 26 aa, synthetic construct
SGTSPGLSAGATVGIMIGVLVGVALI

SEQ ID NO: 58 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 16 aa, synthetic construct
DKTHTCPPCPAPELLG

SEQ ID NO: 60 - protein, 16 aa, synthetic construct
GSTHTCPPCPAPELLG

SEQ ID NO: 61 - protein, 21 aa, synthetic construct
EPKSCDKTHTCPPCPAPELLG

SEQ ID NO: 62 - protein, 24 aa, synthetic construct
EPKSCDKTHTCPPCPAPELLGGPS

SEQ ID NO: 63 - protein, 24 aa, synthetic construct
EPKSSDKTHTCPPCPAPELLGGPS

SEQ ID NO: 64 - protein, 24 aa, synthetic construct
EPKSSDKTHTCPPCPAPELLGGSS

SEQ ID NO: 65 - protein, 24 aa, synthetic construct
EPKSSGSTHTCPPCPAPELLGGSS

SEQ ID NO: 66 - protein, 19 aa, synthetic construct
DKTHTCPPCPAPELLGGPS

SEQ ID NO: 67 - protein, 19 aa, synthetic construct
DKTHTCPPCPAPELLGGSS

SEQ ID NO: 68 - protein, 24 aa, synthetic construct
EPKSSDKTHTCPPCPAPELLGGSS

SEQ ID NO: 69 - protein, 24 aa, synthetic construct
EPKSSGSTHTCPPCPAPELLGGSS

SEQ ID NO: 70 - protein, 19 aa, synthetic construct
DKTHTCPPCPAPELLGGSS

SEQ ID NO: 71 - protein, 227 aa, synthetic construct; identical to SEQ ID NO: 58

SEQ ID NO: 72 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 73 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 227 aa, synthetic construct
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 232 aa, synthetic construct
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYHSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 233 aa, synthetic construct
DKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 83 - protein, 233 aa, synthetic construct
DKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - protein, 233 aa, synthetic construct
SEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 18 aa, Homo sapiens
MKWVTFISLLFLFSSAYS

SEQ ID NO: 86 - protein, 6 aa, Homo sapiens
RGVFRR

SEQ ID NO: 87 - protein, 112 aa, synthetic construct
MIPRGLSEAKPATPEIQEIVDKVKPQLEEKTNETYGKLEAVQYKTQVLADWWQAKWPHSTNYYIKVRAGDNKYMHLKVFNGPYKVHQSSGGADRVLTGYQVDKNKDDELTGF

SEQ ID NO: 88 - protein, 112 aa, synthetic construct
MIPRGLSEAKPATPEIQEIVDKVKPQLEEKTNETYGKLEAVQYKTQVLAGYWAAKWPGSTNYYIKVRAGDNKYMHLKVFNGPFPNTSYDLQADRVLTGYQVDKNKDDELTGF

SEQ ID NO: 89 - protein, 112 aa, synthetic construct
MIPRGLSEAKPATPEIQEIVDKVKPQLEEKTNETYGKLEAVQYKTQVLANWYQQRWPGSTNYYIKVRAGDNKYMHLKVFNGPWHNYGESSGADRVLTGYQVDKNKDDELTGF

SEQ ID NO: 90 - protein, 112 aa, synthetic construct
MIPRGLSEAKPATPEIQEIVDKVKPQLEEKTNETYGKLEAVQYKTQVLAVWGPEYQHQSTNYYIKVRAGDNKYMHLKVFNGPNAGWPLVPEADRVLTGYQVDKNKDDELTGF

SEQ ID NO: 91 - protein, 112 aa, synthetic construct
MIPRGLSEAKPATPEIQEIVDKVKPQLEEKTNETYGKLEAVQYKTQVLAGHYARYWPGSTNYYIKVRAGDNKYMHLKVFNGPWAQKSKVHQADRVLTGYQVDKNKDDELTGF

SEQ ID NO: 92 - protein, 112 aa, synthetic construct
MIPRGLSEAKPATPEIQEIVDKVKPQLEEKTNETYGKLEAVQYKTQVLAGFWASKWPGSTNYYIKVRAGDNKYMHLKVFNGPFTAVSKKDAADRVLTGYQVDKNKDDELTGF

SEQ ID NO: 93 - protein, 112 aa, synthetic construct
MIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 94 - DNA, 8566 bp, synthetic construct
  source 1..8566: mol_type = other DNA
aatattttgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggccctcctatagtgagtcgtattatactatgccgatatactatgccgatgattaattgtcaacacgtgctgcagactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagagctagcctagcggatccgaattctcgatcacaagtttgtacaaaaaagcaggctttaaaggaaccaattcagtcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaattagcgctactagtgatatcaagatgatctagacccagctttcttgtacaaagtggtatcgagatatctgcatttaattaatctagagtttaaacgcggccgcaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggttattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc

SEQ ID NO: 95 - DNA, 9590 bp, synthetic construct
  source 1..9590: mol_type = other DNA
aatattttgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcctagcggatccgaattctcgatcacaagtttgtacaaaaaagcaggcttcgctagccgccaccatgcccctgctcctgctgctgcctctgctgtgggccggtgcattggccatccctggagggctgtccgaggccaagcctgctacgccggaaattcaagaaatcgtcgataaggttaagccacagctcgaagagaaaactggggaaacatacggcaaacttgaggcggttcagtacaaaactcaggtcgtgcactactacgtgcactacaacgatcaagggaccaactactatatcaaggtccgcgcgggagacaacaaatacatgcatctcaaagtgttcaaatcgctgtggggcgagaaccttttcgcgaagtgggaggatttggtgctgactggatatcaggtcgataaaaacaaggatgacgagcttactggctttgccgaggctgcagccaaagaagccgcggcgaaggaagcggcggcaaaggaggcagccgcaaaggaagctgccgctaaagaggccgccgcaaagatgattcccggaggcctctcagaagccaaaccagccactcctgagattcaggaaattgtcgacaaggtcaagccgcagctggaagagaagactggcgaaacctacgggaagctagaggcggtccagtacaagacccaagtcgtccattactatgtacattacaacgaccagggaaccaattactacattaaagtgcgggccggagataataagtatatgcatcttaaggtgtttaagtcgttgtggggcgaaaacctgtttgccaagtgggaagatcttgtgctcaccggttaccaagtggacaagaacaaggacgacgagctgaccgggttcgctgaggctgccgccaaggaagccgcagcgaaggaggccgctgcgaaagaagcggcagccaaggaggcggctgccaaagaggcggccgcgaagatgatacctggcggactgtcggaggcgaaaccggccacccctgaaatccaggagattgtggacaaggtgaaaccccaactcgaggaaaaaaccggcgaaacgtacggtaaactcgaagccgtgcagtataagacccaggtcgtacactactatgtgcactataacgatcagggaacgaactactacatcaaggtcagagctggagacaataagtacatgcacctcaaggttttcaagtcgttatggggagaaaacctcttcgcgaaatgggaggatctcgtgctgaccggctaccaggtggacaaaaataaggatgatgaactcaccggtttcgctgaagcagcggcgaaagaggcagcggccaaggaagccgctgccaaggaagccgccgctaaggaggctgcggctaaggaagcagccgccaagatgatcccacggggtctgtccgaagccaagccagccaccccagagatccaagaaatagtggataaggtgaagccccagcttgaggaaaagactggagagacttacggaaagctcgaggctgtccaatacaaaacacaggtgctggcgaacttcttccaaaggagatggccaggcagtactaactattacatcaaagtgcgcgcaggagataacaaatatatgcacttgaaggtgttcaatggaccgtggaagttccggaacaccgacagaggcgccgacagggtcttgaccgggtaccaagtcgataagaacaaagacgacgaactgaccggattctgaatctagagacccagctttcttgtacaaagtggtatcgagatatctgcatttaattaatctagagtttaaacgcggccgcaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggttattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc

SEQ ID NO: 96 - DNA, 9895 bp, synthetic construct
  source 1..9895: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtatcaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatgccattgcttcttctgctgccactcctttgggctggtgcgctggctattccggggggtttgtcagaagcaaagcccgcgaccccagaaattcaggagatcgtggataaagtaaaacctcagcttgaagaaaaaaccggggagacatacggcaagctggaagcagtacaatataaaacgcaggtagtacattactacgtccattataacgatcaaggtacaaattattatataaaagtacgggcaggggacaacaaatacatgcatctgaaagtatttaagagtctgtggggtgagaacttgtttgcaaagtgggaagatttggtgcttaccggctaccaggtcgataagaataaggatgacgagcttacaggttttgcagaagctgcagcgaaagaggctgccgcaaaagaagcagcggctaaggaagcggccgctaaagaagcggccgcgaaggaagcggccgctaagatgatcccgggtggtctgtccgaagccaaaccggcgacgccggaaatccaggagatagtcgataaggtcaagccacaactcgaagagaagactggagaaacatatggaaaacttgaggccgttcaatacaaaacgcaggtggtccactactacgttcactataacgatcaaggaacaaactattacattaaagtcagggcgggagacaacaagtacatgcatcttaaagtgtttaaatctctttggggggagaatcttttcgccaagtgggaagacttggtcctcaccggataccaggtcgacaaaaacaaagacgatgaactcaccggctttgccgaagcggctgcgaaagaagcggctgccaaagaggctgcagccaaagaagctgcggcaaaggaggccgccgccaaagaagcggcggccaaaatgatccccggaggacttagcgaggctaaacccgcgacacctgaaatacaggagatcgtggataaggttaaaccacaattggaggagaaaactggtgagacctatggcaagctggaagcggttcagtataaaacgcaagttgtacactactatgtacattacaacgatcaggggacgaactactatattaaggtcagggcaggagataataaatatatgcacttgaaagttttcaaaagcctgtggggggaaaacttgttcgccaagtgggaagatctcgtcttgactgggtaccaggttgataagaacaaagacgatgaactgacaggcttcgctgaagcggctgcaaaggaggcggcagcgaaggaggctgccgcgaaagaagcggcggctaaagaagcggccgcaaaggaagcagcagcgaagatgataccgaggggtttgagcgaagcaaagcccgcaacgcctgagatacaagaaatcgttgacaaagtaaaaccacaattggaagagaagacgggagaaacgtatggaaagcttgaggccgtacaatacaagacacaggtgttggcaaattttttccaaaggcgatggcccggatcaacgaattactatatcaaggtacgcgcgggggataacaagtacatgcatctcaaggttttcaacggtccgtggaaattccgaaatacagaccgcggagccgacagggtcctcaccggctatcaggtcgacaagaataaggatgatgagttgacaggtttcgccgccgccggtgagcagaaattgatctccgaagaggatctcggtgccgcgcaccaccaccaccatcattaaacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacaactttattatacatagttgatcaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 97 - DNA, 9361 bp, synthetic construct
  source 1..9361: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatgccattgcttcttctgctgccactcctttgggctggtgcgctggctattccggggggtttgtcagaagcaaagcccgcgaccccagaaattcaggagatcgtggataaagtaaaacctcagcttgaagaaaaaaccggggagacatacggcaagctggaagcagtacaatataaaacgcaggtagtacattactacgtccattataacgatcaaggtacaaattattatataaaagtacgggcaggggacaacaaatacatgcatctgaaagtatttaagagtctgtggggtgagaacttgtttgcaaagtgggaagatttggtgcttaccggctaccaggtcgataagaataaggatgacgagcttacaggttttgcagaagctgcagcgaaagaggctgccgcaaaagaagcagcggctaaggaagcggccgctaaagaagcggccgcgaaggaagcggccgctaagatgatcccgggtggtctgtccgaagccaaaccggcgacgccggaaatccaggagatagtcgataaggtcaagccacaactcgaagagaagactggagaaacatatggaaaacttgaggccgttcaatacaaaacgcaggtggtccactactacgttcactataacgatcaaggaacaaactattacattaaagtcagggcgggagacaacaagtacatgcatcttaaagtgtttaaatctctttggggggagaatcttttcgccaagtgggaagacttggtcctcaccggataccaggtcgacaaaaacaaagacgatgaactcaccggctttgccgaagcggctgcgaaagaagcggctgccaaagaggctgcagccaaagaagctgcggcaaaggaggccgccgccaaagaagcggcggccaaaatgatccccggaggacttagcgaggctaaacccgcgacacctgaaatacaggagatcgtggataaggttaaaccacaattggaggagaaaactggtgagacctatggcaagctggaagcggttcagtataaaacgcaagttgtacactactatgtacattacaacgatcaggggacgaactactatattaaggtcagggcaggagataataaatatatgcacttgaaagttttcaaaagcctgtggggggaaaacttgttcgccaagtgggaagatctcgtcttgactgggtaccaggttgataagaacaaagacgatgaactgacaggcttcgctgaagcggctgcaaaggaggcggcagcgaaggaggctgccgcgaaagaagcggcggctaaagaagcggccgcaaaggaagcagcagcgaagatgataccgaggggtttgagcgaagcaaagcccgcaacgcctgagatacaagaaatcgttgacaaagtaaaaccacaattggaagagaagacgggagaaacgtatggaaagcttgaggccgtacaatacaagacacaggtgttggcaaattttttccaaaggcgatggcccggatcaacgaattactatatcaaggtacgcgcgggggataacaagtacatgcatctcaaggttttcaacggtccgtggaaattccgaaatacagaccgcggagccgacagggtcctcaccggctatcaggtcgacaagaataaggatgatgagttgacaggtttcgccgccgccggtgagcagaaattgatctccgaagaggatctcggtgccgcgcaccaccaccaccatcatggaagcggagagggcaggggaagtcttctaacatgcggggacgtggaggaaaatcccggccccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 98 - DNA, 9951 bp, synthetic construct
  source 1..9951: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgccattgcttcttctgctgccactcctttgggctggtgcgctggctattccggggggtttgtcagaagcaaagcccgcgaccccagaaattcaggagatcgtggataaagtaaaacctcagcttgaagaaaaaaccggggagacatacggcaagctggaagcagtacaatataaaacgcaggtagtacattactacgtccattataacgatcaaggtacaaattattatataaaagtacgggcaggggacaacaaatacatgcatctgaaagtatttaagagtctgtggggtgagaacttgtttgcaaagtgggaagatttggtgcttaccggctaccaggtcgataagaataaggatgacgagcttacaggttttgcagaagctgcagcgaaagaggctgccgcaaaagaagcagcggctaaggaagcggccgctaaagaagcggccgcgaaggaagcggccgctaagatgatcccgggtggtctgtccgaagccaaaccggcgacgccggaaatccaggagatagtcgataaggtcaagccacaactcgaagagaagactggagaaacatatggaaaacttgaggccgttcaatacaaaacgcaggtggtccactactacgttcactataacgatcaaggaacaaactattacattaaagtcagggcgggagacaacaagtacatgcatcttaaagtgtttaaatctctttggggggagaatcttttcgccaagtgggaagacttggtcctcaccggataccaggtcgacaaaaacaaagacgatgaactcaccggctttgccgaagcggctgcgaaagaagcggctgccaaagaggctgcagccaaagaagctgcggcaaaggaggccgccgccaaagaagcggcggccaaaatgatccccggaggacttagcgaggctaaacccgcgacacctgaaatacaggagatcgtggataaggttaaaccacaattggaggagaaaactggtgagacctatggcaagctggaagcggttcagtataaaacgcaagttgtacactactatgtacattacaacgatcaggggacgaactactatattaaggtcagggcaggagataataaatatatgcacttgaaagttttcaaaagcctgtggggggaaaacttgttcgccaagtgggaagatctcgtcttgactgggtaccaggttgataagaacaaagacgatgaactgacaggcttcgctgaagcggctgcaaaggaggcggcagcgaaggaggctgccgcgaaagaagcggcggctaaagaagcggccgcaaaggaagcagcagcgaagatgataccgaggggtttgagcgaagcaaagcccgcaacgcctgagatacaagaaatcgttgacaaagtaaaaccacaattggaagagaagacgggagaaacgtatggaaagcttgaggccgtacaatacaagacacaggtgttggcaaattttttccaaaggcgatggcccggatcaacgaattactatatcaaggtacgcgcgggggataacaagtacatgcatctcaaggttttcaacggtccgtggaaattccgaaatacagaccgcggagccgacagggtcctcaccggctatcaggtcgacaagaataaggatgatgagttgacaggtttcgccgccgccggtgagcagaaattgatctccgaagaggatctcggtgccgcgcaccaccaccaccatcatggaagcggagagggcaggggaagtcttctaacatgcggggacgtggaggaaaatcccggccccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 99 - DNA, 9004 bp, synthetic construct
  source 1..9004: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcaagtttgtacaaaaaagcaggctgccaccatgccattgcttcttctgctgccactcctttgggctggtgcgctggctattccggggggtttgtcagaagcaaagcccgcgaccccagaaattcaggagatcgtggataaagtaaaacctcagcttgaagaaaaaaccggggagacatacggcaagctggaagcagtacaatataaaacgcaggtagtacattactacgtccattataacgatcaaggtacaaattattatataaaagtacgggcaggggacaacaaatacatgcatctgaaagtatttaagagtctgtggggtgagaacttgtttgcaaagtgggaagatttggtgcttaccggctaccaggtcgataagaataaggatgacgagcttacaggttttgcagaagctgcagcgaaagaggctgccgcaaaagaagcagcggctaaggaagcggccgctaaagaagcggccgcgaaggaagcggccgctaagatgatcccgggtggtctgtccgaagccaaaccggcgacgccggaaatccaggagatagtcgataaggtcaagccacaactcgaagagaagactggagaaacatatggaaaacttgaggccgttcaatacaaaacgcaggtggtccactactacgttcactataacgatcaaggaacaaactattacattaaagtcagggcgggagacaacaagtacatgcatcttaaagtgtttaaatctctttggggggagaatcttttcgccaagtgggaagacttggtcctcaccggataccaggtcgacaaaaacaaagacgatgaactcaccggctttgccgaagcggctgcgaaagaagcggctgccaaagaggctgcagccaaagaagctgcggcaaaggaggccgccgccaaagaagcggcggccaaaatgatccccggaggacttagcgaggctaaacccgcgacacctgaaatacaggagatcgtggataaggttaaaccacaattggaggagaaaactggtgagacctatggcaagctggaagcggttcagtataaaacgcaagttgtacactactatgtacattacaacgatcaggggacgaactactatattaaggtcagggcaggagataataaatatatgcacttgaaagttttcaaaagcctgtggggggaaaacttgttcgccaagtgggaagatctcgtcttgactgggtaccaggttgataagaacaaagacgatgaactgacaggcttcgctgaagcggctgcaaaggaggcggcagcgaaggaggctgccgcgaaagaagcggcggctaaagaagcggccgcaaaggaagcagcagcgaagatgataccgaggggtttgagcgaagcaaagcccgcaacgcctgagatacaagaaatcgttgacaaagtaaaaccacaattggaagagaagacgggagaaacgtatggaaagcttgaggccgtacaatacaagacacaggtgttggcaaattttttccaaaggcgatggcccggatcaacgaattactatatcaaggtacgcgcgggggataacaagtacatgcatctcaaggttttcaacggtccgtggaaattccgaaatacagaccgcggagccgacagggtcctcaccggctatcaggtcgacaagaataaggatgatgagttgacaggtttcgccgccgccggtgagcagaaattgatctccgaagaggatctcggtgccgcgcaccaccaccaccatcatggaagcggagagggcaggggaagtcttctaacatgcggggacgtggaggaaaatcccggccccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 100 - DNA, 9272 bp, synthetic construct
  source 1..9272: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgctgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggcaagtttgtacaaaaaagcaggctgccaccatgccattgcttcttctgctgccactcctttgggctggtgcgctggctattccggggggtttgtcagaagcaaagcccgcgaccccagaaattcaggagatcgtggataaagtaaaacctcagcttgaagaaaaaaccggggagacatacggcaagctggaagcagtacaatataaaacgcaggtagtacattactacgtccattataacgatcaaggtacaaattattatataaaagtacgggcaggggacaacaaatacatgcatctgaaagtatttaagagtctgtggggtgagaacttgtttgcaaagtgggaagatttggtgcttaccggctaccaggtcgataagaataaggatgacgagcttacaggttttgcagaagctgcagcgaaagaggctgccgcaaaagaagcagcggctaaggaagcggccgctaaagaagcggccgcgaaggaagcggccgctaagatgatcccgggtggtctgtccgaagccaaaccggcgacgccggaaatccaggagatagtcgataaggtcaagccacaactcgaagagaagactggagaaacatatggaaaacttgaggccgttcaatacaaaacgcaggtggtccactactacgttcactataacgatcaaggaacaaactattacattaaagtcagggcgggagacaacaagtacatgcatcttaaagtgtttaaatctctttggggggagaatcttttcgccaagtgggaagacttggtcctcaccggataccaggtcgacaaaaacaaagacgatgaactcaccggctttgccgaagcggctgcgaaagaagcggctgccaaagaggctgcagccaaagaagctgcggcaaaggaggccgccgccaaagaagcggcggccaaaatgatccccggaggacttagcgaggctaaacccgcgacacctgaaatacaggagatcgtggataaggttaaaccacaattggaggagaaaactggtgagacctatggcaagctggaagcggttcagtataaaacgcaagttgtacactactatgtacattacaacgatcaggggacgaactactatattaaggtcagggcaggagataataaatatatgcacttgaaagttttcaaaagcctgtggggggaaaacttgttcgccaagtgggaagatctcgtcttgactgggtaccaggttgataagaacaaagacgatgaactgacaggcttcgctgaagcggctgcaaaggaggcggcagcgaaggaggctgccgcgaaagaagcggcggctaaagaagcggccgcaaaggaagcagcagcgaagatgataccgaggggtttgagcgaagcaaagcccgcaacgcctgagatacaagaaatcgttgacaaagtaaaaccacaattggaagagaagacgggagaaacgtatggaaagcttgaggccgtacaatacaagacacaggtgttggcaaattttttccaaaggcgatggcccggatcaacgaattactatatcaaggtacgcgcgggggataacaagtacatgcatctcaaggttttcaacggtccgtggaaattccgaaatacagaccgcggagccgacagggtcctcaccggctatcaggtcgacaagaataaggatgatgagttgacaggtttcgccgccgccggtgagcagaaattgatctccgaagaggatctcggtgccgcgcaccaccaccaccatcatggaagcggagagggcaggggaagtcttctaacatgcggggacgtggaggaaaatcccggccccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 101 - DNA, 9287 bp, synthetic construct
  source 1..9287: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgtgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcattcaagtttgtacaaaaaagcaggctgccaccatgccattgcttcttctgctgccactcctttgggctggtgcgctggctattccggggggtttgtcagaagcaaagcccgcgaccccagaaattcaggagatcgtggataaagtaaaacctcagcttgaagaaaaaaccggggagacatacggcaagctggaagcagtacaatataaaacgcaggtagtacattactacgtccattataacgatcaaggtacaaattattatataaaagtacgggcaggggacaacaaatacatgcatctgaaagtatttaagagtctgtggggtgagaacttgtttgcaaagtgggaagatttggtgcttaccggctaccaggtcgataagaataaggatgacgagcttacaggttttgcagaagctgcagcgaaagaggctgccgcaaaagaagcagcggctaaggaagcggccgctaaagaagcggccgcgaaggaagcggccgctaagatgatcccgggtggtctgtccgaagccaaaccggcgacgccggaaatccaggagatagtcgataaggtcaagccacaactcgaagagaagactggagaaacatatggaaaacttgaggccgttcaatacaaaacgcaggtggtccactactacgttcactataacgatcaaggaacaaactattacattaaagtcagggcgggagacaacaagtacatgcatcttaaagtgtttaaatctctttggggggagaatcttttcgccaagtgggaagacttggtcctcaccggataccaggtcgacaaaaacaaagacgatgaactcaccggctttgccgaagcggctgcgaaagaagcggctgccaaagaggctgcagccaaagaagctgcggcaaaggaggccgccgccaaagaagcggcggccaaaatgatccccggaggacttagcgaggctaaacccgcgacacctgaaatacaggagatcgtggataaggttaaaccacaattggaggagaaaactggtgagacctatggcaagctggaagcggttcagtataaaacgcaagttgtacactactatgtacattacaacgatcaggggacgaactactatattaaggtcagggcaggagataataaatatatgcacttgaaagttttcaaaagcctgtggggggaaaacttgttcgccaagtgggaagatctcgtcttgactgggtaccaggttgataagaacaaagacgatgaactgacaggcttcgctgaagcggctgcaaaggaggcggcagcgaaggaggctgccgcgaaagaagcggcggctaaagaagcggccgcaaaggaagcagcagcgaagatgataccgaggggtttgagcgaagcaaagcccgcaacgcctgagatacaagaaatcgttgacaaagtaaaaccacaattggaagagaagacgggagaaacgtatggaaagcttgaggccgtacaatacaagacacaggtgttggcaaattttttccaaaggcgatggcccggatcaacgaattactatatcaaggtacgcgcgggggataacaagtacatgcatctcaaggttttcaacggtccgtggaaattccgaaatacagaccgcggagccgacagggtcctcaccggctatcaggtcgacaagaataaggatgatgagttgacaggtttcgccgccgccggtgagcagaaattgatctccgaagaggatctcggtgccgcgcaccaccaccaccatcatggaagcggagagggcaggggaagtcttctaacatgcggggacgtggaggaaaatcccggccccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 102 - DNA, 9570 bp, synthetic construct
  source 1..9570: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcaagtttgtacaaaaaagcaggctgccaccatgccattgcttcttctgctgccactcctttgggctggtgcgctggctattccggggggtttgtcagaagcaaagcccgcgaccccagaaattcaggagatcgtggataaagtaaaacctcagcttgaagaaaaaaccggggagacatacggcaagctggaagcagtacaatataaaacgcaggtagtacattactacgtccattataacgatcaaggtacaaattattatataaaagtacgggcaggggacaacaaatacatgcatctgaaagtatttaagagtctgtggggtgagaacttgtttgcaaagtgggaagatttggtgcttaccggctaccaggtcgataagaataaggatgacgagcttacaggttttgcagaagctgcagcgaaagaggctgccgcaaaagaagcagcggctaaggaagcggccgctaaagaagcggccgcgaaggaagcggccgctaagatgatcccgggtggtctgtccgaagccaaaccggcgacgccggaaatccaggagatagtcgataaggtcaagccacaactcgaagagaagactggagaaacatatggaaaacttgaggccgttcaatacaaaacgcaggtggtccactactacgttcactataacgatcaaggaacaaactattacattaaagtcagggcgggagacaacaagtacatgcatcttaaagtgtttaaatctctttggggggagaatcttttcgccaagtgggaagacttggtcctcaccggataccaggtcgacaaaaacaaagacgatgaactcaccggctttgccgaagcggctgcgaaagaagcggctgccaaagaggctgcagccaaagaagctgcggcaaaggaggccgccgccaaagaagcggcggccaaaatgatccccggaggacttagcgaggctaaacccgcgacacctgaaatacaggagatcgtggataaggttaaaccacaattggaggagaaaactggtgagacctatggcaagctggaagcggttcagtataaaacgcaagttgtacactactatgtacattacaacgatcaggggacgaactactatattaaggtcagggcaggagataataaatatatgcacttgaaagttttcaaaagcctgtggggggaaaacttgttcgccaagtgggaagatctcgtcttgactgggtaccaggttgataagaacaaagacgatgaactgacaggcttcgctgaagcggctgcaaaggaggcggcagcgaaggaggctgccgcgaaagaagcggcggctaaagaagcggccgcaaaggaagcagcagcgaagatgataccgaggggtttgagcgaagcaaagcccgcaacgcctgagatacaagaaatcgttgacaaagtaaaaccacaattggaagagaagacgggagaaacgtatggaaagcttgaggccgtacaatacaagacacaggtgttggcaaattttttccaaaggcgatggcccggatcaacgaattactatatcaaggtacgcgcgggggataacaagtacatgcatctcaaggttttcaacggtccgtggaaattccgaaatacagaccgcggagccgacagggtcctcaccggctatcaggtcgacaagaataaggatgatgagttgacaggtttcgccgccgccggtgagcagaaattgatctccgaagaggatctcggtgccgcgcaccaccaccaccatcatggaagcggagagggcaggggaagtcttctaacatgcggggacgtggaggaaaatcccggccccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 103 - DNA, 9581 bp, synthetic construct
  source 1..9581: mol_type = other DNA
aatattttgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcctagcggatccgaattctcgatcacaagtttgtacaaaaaagcaggcttcgctagccgccaccatgccgctcctgctgctgctgcccctgctgtgggcaggagctctggccatccctggagggctgtccgaggccaagcctgctacgccggaaattcaagaaatcgtcgataaggttaagccacagctcgaagagaaaactggggaaacatacggcaaacttgaggcggttcagtacaaaactcaggtcgtgcactactacgtgcactacaacgatcaagggaccaactactatatcaaggtccgcgcgggagacaacaaatacatgcatctcaaagtgttcaaatcgctgtggggcgagaaccttttcgcgaagtgggaggatttggtgctgactggatatcaggtcgataaaaacaaggatgacgagcttactggctttggaggaggagggtccggtggaggaggctctggtggaggcggaagcggcggaggaggatcgggtggcggaggaagcggtggtggaggctccatgattcccggaggcctctcagaagccaaaccagccactcctgagattcaggaaattgtcgacaaggtcaagccgcagctggaagagaagactggcgaaacctacgggaagctagaggcggtccagtacaagacccaagtcgtccattactatgtacattacaacgaccagggaaccaattactacattaaagtgcgggccggagataataagtatatgcatcttaaggtgtttaagtcgttgtggggcgaaaacctgtttgccaagtgggaagatcttgtgctcaccggttaccaagtggacaagaacaaggacgacgagctgaccgggttcggaggaggcggatcgggaggtggaggatccggtggcggtggaagcggaggtggcggatctgggggagggggatcaggaggcggcggatccatgatacctggcggactgtcggaggcgaaaccggccacccctgaaatccaggagattgtggacaaggtgaaaccccaactcgaggaaaaaaccggcgaaacgtacggtaaactcgaagccgtgcagtataagacccaggtcgtacactactatgtgcactataacgatcagggaacgaactactacatcaaggtcagagctggagacaataagtacatgcacctcaaggttttcaagtcgttatggggagaaaacctcttcgcgaaatgggaggatctcgtgctgaccggctaccaggtggacaaaaataaggatgatgaactcaccggtttcggaggcggaggatcaggcggagggggttcaggagggggtggctcaggaggagggggtagtggaggcggtggatccggaggaggtggcagcatgatcccacggggtctgtccgaagccaagccagccaccccagagatccaagaaatagtggataaggtgaagccccagcttgaggaaaagactggagagacttacggaaagctcgaggctgtccaatacaaaacacaggtgctggcgaacttcttccaaaggagatggccaggcagtactaactattacatcaaagtgcgcgcaggagataacaaatatatgcacttgaaggtgttcaatggaccgtggaagttccggaacaccgacagaggcgccgacagggtcttgaccgggtaccaagtcgataagaacaaagacgacgaactgaccggtttctgaatctagagacccagctttcttgtacaaagtggtatcgagatatctgcatttaattaatctagagtttaaacgcggccgcaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggttattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc

SEQ ID NO: 104 - protein, 534 aa, synthetic construct
IPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSMIPRGLSEAKPATPEIQEIVDKVKPQLEEKTGETYGKLEAVQYKTQVLANFFQRRWPGSTNYYIKVRAGDNKYMHLKVFNGPWKFRNTDRGADRVLTGYQVDKNKDDELTGF

SEQ ID NO: 105 - protein, 537 aa, synthetic construct
IPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKMIPRGLSEAKPATPEIQEIVDKVKPQLEEKTGETYGKLEAVQYKTQVLANFFQRRWPGSTNYYIKVRAGDNKYMHLKVFNGPWKFRNTDRGADRVLTGYQVDKNKDDELTGF

SEQ ID NO: 106 - protein, 49 aa, synthetic construct
AVGQDTQEVIVVPHSLPFKVVVISAILALVVLTIISLIILIMLWQKKPR

SEQ ID NO: 107 - protein, 9 aa, synthetic construct
GQDQWGNYA

SEQ ID NO: 108 - protein, 9 aa, synthetic construct
GLDIWGNNA

SEQ ID NO: 109 - protein, 9 aa, synthetic construct
RLFHIWRWW

SEQ ID NO: 110 - protein, 9 aa, synthetic construct
YWGVQGALD

SEQ ID NO: 111 - protein, 9 aa, synthetic construct
LEIPSAIIY

SEQ ID NO: 112 - protein, 9 aa, synthetic construct
LFPEAVYWV

SEQ ID NO: 113 - protein, 11 aa, synthetic construct
QSWDKHYQLRE

SEQ ID NO: 114 - protein, 9 aa, synthetic construct
PDIPHNHWH

SEQ ID NO: 115 - protein, 9 aa, synthetic construct
ADTARTEEA

SEQ ID NO: 116 - protein, 9 aa, synthetic construct
IHIYTDSIQ

SEQ ID NO: 117 - protein, 9 aa, synthetic construct
NWNHEFDLV

SEQ ID NO: 118 - protein, 9 aa, synthetic construct
YFWGDQEHW

SEQ ID NO: 119 - protein, 9 aa, synthetic construct
IWNDNAAQH

SEQ ID NO: 120 - protein, 9 aa, synthetic construct
EVPHHAHGV

SEQ ID NO: 121 - protein, 132 aa, synthetic construct
MIPGGLSEAKPATPEIQEIVDKVKPQLEEKTGETYGKLEAVQYKTQVVGQDQWGNYAGTNYYIKVRAGDNKYMHLKVFKSLPDIPHNHWHEDLVLTGYQVDKNKDDELTGFAAAEQKLISEEDLAAHHHHHH

SEQ ID NO: 122 - protein, 132 aa, synthetic construct
MIPGGLSEAKPATPEIQEIVDKVKPQLEEKTGETYGKLEAVQYKTQVVGLDIWGNNAGTNYYIKVRAGDNKYMHLKVFKSLADTARTEEAEDLVLTGYQVDKNKDDELTGFAAAEQKLISEEDLAAHHHHHH

SEQ ID NO: 123 - protein, 132 aa, synthetic construct
MIPGGLSEAKPATPEIQEIVDKVKPQLEEKTGETYGKLEAVQYKTQVVRLFHIWRWWGTNYYIKVRAGDNKYMHLKVFKSLIHIYTDSIQEDLVLTGYQVDKNKDDELTGFAAAEQKLISEEDLAAHHHHHH

SEQ ID NO: 124 - protein, 132 aa, synthetic construct
MIPGGLSEAKPATPEIQEIVDKVKPQLEEKTGETYGKLEAVQYKTQVVYWGVQGALDGTNYYIKVRAGDNKYMHLKVFKSLNWNHEFDLVEDLVLTGYQVDKNKDDELTGFAAAEQKLISEEDLAAHHHHHH

SEQ ID NO: 125 - protein, 133 aa, synthetic construct
MIPRGLSEAKPATPEIQEIVDKVKPQLEEKTGETYGKLEAVQYKTQVLALEIPSAIIYSTNYYIKVRAGDNKYMHLKVFNGPYFWGDQEHWADRVLTGYQVDKNKDDELTGFAAAEQKLISEEDLAAHHHHHH

SEQ ID NO: 126 - protein, 132 aa, synthetic construct
MIPGGLSEAKPATPEIQEIVDKVKPQLEEKTGETYGKLEAVQYKTQVVLFPEAVYWVGTNYYIKVRAGDNKYMHLKVFKSLIWNDNAAQHEDLVLTGYQVDKNKDDELTGFAAAEQKLISEEDLAAHHHHHH

SEQ ID NO: 127 - protein, 135 aa, synthetic construct
MIPRGLSEAKPATPEIQEIVDKVKPQLEEKTGETYGKLEAVQYKTQVLAQSWDKHYQLRESTNYYIKVRAGDNKYMHLKVFNGPEVPHHAHGVADRVLTGYQVDKNKDDELTGFAAAEQKLISEEDLAAHHHHHH

SEQ ID NO: 128-445 - skipped (318 entries)

SEQ ID NO: 446 - protein, 24 aa, synthetic construct
EPKSSDKTHTCPPCPAPELLGGPS

SEQ ID NO: 447 - protein, 6 aa, synthetic construct
HHHHHH

SEQ ID NO: 448 - protein, 21 aa, synthetic construct
AAAEQKLISEEDLAAHHHHHH